(19) United States
(12) Patent Application Publication
(10) Pub. No.: US 2023/0041060 A1
(43) Pub. Date: Feb. 9, 2023

(54) Treatment of Varicose Veins With Piezo Type Mechanosensitive Ion Channel Component 1 (PIEZO1) Agonists

(71) Applicant: Regeneron Pharmaceuticals, Inc., Tarrytown, NY (US)

(72) Inventors: Manuel Allen Revez Ferreira, Tarrytown, NY (US); Joshua Backman, Tarrytown, NY (US); Alexander Li, Tarrytown, NY (US); Aris Baras, Tarrytown, NY (US); Goncalo Abecasis, Tarrytown, NY (US)

(21) Appl. No.: 17/810,820

(22) Filed: Jul. 5, 2022

Related U.S. Application Data
(60) Provisional application No. 63/219,462, filed on Jul. 8, 2021.

(51) Int. Cl. C12Q 1/6883

(52) U.S. Cl. CPC C12Q 1/6883; C12Q 2600/158; C12Q 2600/106

(57) ABSTRACT

The present disclosure provides methods of treating subjects having varicose veins or at risk of developing varicose veins, methods of identifying subjects having an increased risk of developing varicose veins, and methods of detecting Piezo Type Mechanosensitive Ion Channel Component 1 (PIEZO1) variant nucleic acid molecules and variant polypeptides.

### REFERENCE TO SEQUENCE LISTING

This application includes a Sequence Listing submitted electronically as an XML file named 381203528SEQ220906, created on Sep. 6, 2022, with a size of 381 kilobytes. The Sequence Listing is incorporated herein by reference.

### FIELD

The present disclosure relates generally to the treatment of subjects having varicose veins or at risk of developing varicose veins with Piezo Type Mechanosensitive Ion Channel Component 1 (PIEZO1) agonists, and methods of identifying subjects having an increased risk of developing varicose veins.

### BACKGROUND

Varicose veins is a common multifactorial disease with largely unknown genetic drivers that is often seen in patients with chronic venous insufficiency, together classified as chronic venous disease. Dysfunction of venous valves is associated with varicose veins, venous hypertension, and thrombosis. Several processes, such as changes in hemodynamic forces, endothelial activation, inflammation, hypoxia, and dysregulation of matrix metalloproteinases and their tissue agonists have been associated with varicose vein development. Varicose vein risk factors include increased age, female sex, number of pregnancies, obesity, history of deep venous thrombosis, and standing occupation. varicose veins has also been linked to insufficient lymph drainage and chronic venous insufficiency. In addition, several genome-wide association studies (GWAS) displayed about 18.5% varicose vein heritability.

PIEZO1 is encoded by a 70 kb gene located at 16q24.3 and is present in five potential isoforms. PIEZO1 protein is 2,521 amino acids long, and is a 286 kDa transmembrane protein that contains 38 transmembrane domains and functions as a homo-tetramer. PIEZO1 encodes an evolutionarily conserved endothelial mechanosensitive cation channel, which generates currents characterized by a linear current-voltage relationship that are sensitive to ruthenium red and gadolinium. PIEZO1 is ubiquitously expressed and plays a role in epithelial cell adhesion by maintaining integrin activation through R-Ras recruitment to the endoplasmic reticulum, most probably in its activated state, and subsequent stimulation of calpain signaling. In vasculature, PIEZO1 is involved in endothelial cell migration and sprouting angiogenesis. Specifically, PIEZO1 acts as a sensor for bloodflow-associated shear stress and promotes endothelial cell organization and alignment in the direction of blood flow ensuring proper vessel formation, remodeling, and maturation. PIEZO1 also appears to be required for lymphatic valve formation. Other reported functions include blood pressure regulation, urine osmolarity, erythrocyte integrity, pressure sensing, and collecting duct osmoregulation.

### SUMMARY

The present disclosure provides methods of treating a subject having varicose veins or at risk of developing varicose veins, the methods comprising administering a PIEZO1 agonist to the subject.

The present disclosure also provides methods of treating a subject having saphenous varicose veins or at risk of developing saphenous varicose veins, the methods comprising administering a PIEZO1 agonist to the subject.

The present disclosure also provides methods of treating a subject having veticular varicose veins or at risk of developing veticular varicose veins, the methods comprising administering a PIEZO1 agonist to the subject.

The present disclosure also provides methods of treating a subject having spider veins or at risk of developing spider veins, the methods comprising administering a PIEZO1 agonist to the subject.

The present disclosure also provides methods of treating a subject having pregnancy-related varicose veins or at risk of developing pregnancy-related varicose veins, the methods comprising administering a PIEZO1 agonist to the subject.

The present disclosure also provides methods of treating a subject with a therapeutic agent that treats or prevents varicose veins, wherein the subject has varicose veins or is at risk for developing varicose veins, the methods comprising: determining whether the subject has a PIEZO1 variant nucleic acid molecule encoding a PIEZO1 predicted gain-of-function polypeptide by: obtaining or having obtained a biological sample from the subject; and performing or having performed a sequence analysis on the biological sample to determine if the subject has a genotype comprising the PIEZO1 variant nucleic acid molecule encoding the PIEZO1 predicted gain-of-function polypeptide; and administering or continuing to administer the therapeutic agent that treats or prevents varicose veins in a standard dosage amount to a subject that is PIEZO1 reference, and/or administering a PIEZO1 agonist to the subject; and administering or continuing to administer the therapeutic agent that treats or prevents varicose veins in an amount that is the same as or less than a standard dosage amount to a subject that is heterozygous for the PIEZO1 variant nucleic acid molecule, and/or administering a PIEZO1 agonist to the subject; wherein the presence of a genotype having the PIEZO1 variant nucleic acid molecule encoding the PIEZO1 predicted gain-of-function polypeptide indicates the subject has a decreased risk of developing varicose veins.

The present disclosure also provides methods of identifying a subject having an increased risk of developing varicose veins, the methods comprising: determining or having determined the presence or absence of a PIEZO1 variant nucleic acid molecule encoding a PIEZO1 predicted gain-of-function polypeptide in a biological sample obtained from the subject; wherein: when the subject is PIEZO1 reference, then the subject has an increased risk of developing varicose veins; and when the subject is heterozygous or homozygous for a PIEZO1 variant nucleic acid molecule encoding the PIEZO1 predicted gain-of-function polypeptide, then the subject has a decreased risk of developing varicose veins.

The present disclosure also provides therapeutic agents that treat or prevent varicose veins for use in the treatment or prevention of varicose veins in a subject identified as having: i) a genomic nucleic acid molecule encoding a PIEZO1 predicted gain-of-function polypeptide, or the complement thereof, wherein the genomic nucleic acid molecule has a nucleotide sequence comprising: i) a thymine at a position corresponding to position 69,579 according to SEQ ID NO:2, or the complement thereof; ii) an mRNA molecule encoding a PIEZO1 predicted gain-of-function polypeptide, or the complement thereof, wherein the mRNA molecule has a nucleotide sequence comprising: a uracil at a position corresponding to position 7,785 according to SEQ ID NO:13, or the complement thereof; a uracil at a position corresponding to position 1,095 according to SEQ ID NO:14, or the complement thereof; a uracil at a position corresponding to position 6,073 according to SEQ ID NO:15, or the complement thereof; a uracil at a position corresponding to position 1,336 according to SEQ ID NO:16, or the complement thereof; a uracil at a position corresponding to position 6,073 according to SEQ ID NO:17, or the complement thereof; a uracil at a position corresponding to position 6,236 according to SEQ ID NO:18, or the complement thereof; a uracil at a position corresponding to position 7,526 according to SEQ ID NO:19, or the complement thereof; a uracil at a position corresponding to position 1,336 according to SEQ ID NO:20, or the complement thereof; a uracil at a position corresponding to position 361 according to SEQ ID NO:21, or the complement thereof; or a uracil at a position corresponding to position 7,776 according to SEQ ID NO:22, or the complement thereof; or iii) a cDNA molecule encoding a PIEZO1 predicted gain-of-function polypeptide, or the complement thereof, wherein the cDNA molecule has a nucleotide sequence comprising: a thymine at a position corresponding to position 7,785 according to SEQ ID NO:33, or the complement thereof; a thymine at a position corresponding to position 1,095 according to SEQ ID NO:34, or the complement thereof; a thymine at a position corresponding to position 6,073 according to SEQ ID NO:35, or the complement thereof; a thymine at a position corresponding to position 1,336 according to SEQ ID NO:36, or the complement thereof; a thymine at a position corresponding to position 6,073 according to SEQ ID NO:37, or the complement thereof; a thymine at a position corresponding to position 6,236 according to SEQ ID NO:38, or the complement thereof; a thymine at a position corresponding to position 7,526 according to SEQ ID NO:39, or the complement thereof; a thymine at a position corresponding to position 1,336 according to SEQ ID NO:40, or the complement thereof; a thymine at a position corresponding to position 361 according to SEQ ID NO:41, or the complement thereof; or a thymine at a position corresponding to position 7,776 according to SEQ ID NO:42, or the complement thereof.

The present disclosure also provides PIEZO1 agonists for use in the treatment or prevention of varicose veins in a subject that: a) is reference for a PIEZO1 genomic nucleic acid molecule, a PIEZO1 mRNA molecule, or a PIEZO1 cDNA molecule; or b) is heterozygous for: i) a genomic nucleic acid molecule encoding a PIEZO1 predicted gain-of-function polypeptide, or the complement thereof, wherein the genomic nucleic acid molecule has a nucleotide sequence comprising: a thymine at a position corresponding to position 69,579 according to SEQ ID NO:2, or the complement thereof; ii) an mRNA molecule encoding a PIEZO1 predicted gain-of-function polypeptide, or the complement thereof, wherein the mRNA molecule has a nucleotide sequence comprising: a uracil at a position corresponding to position 7,785 according to SEQ ID NO:13, or the complement thereof; a uracil at a position corresponding to position 1,095 according to SEQ ID NO:14, or the complement thereof; a uracil at a position corresponding to position 6,073 according to SEQ ID NO:15, or the complement thereof; a uracil at a position corresponding to position 1,336 according to SEQ ID NO:16, or the complement thereof; a uracil at a position corresponding to position 6,073 according to SEQ ID NO:17, or the complement thereof; a uracil at a position corresponding to position 6,236 according to SEQ ID NO:18, or the complement thereof; a uracil at a position corresponding to position 7,526 according to SEQ ID NO:19, or the complement thereof; a uracil at a position corresponding to position 1,336 according to SEQ ID NO:20, or the complement thereof; a uracil at a position corresponding to position 361 according to SEQ ID NO:21, or the complement thereof; or a uracil at a position corresponding to position 7,776 according to SEQ ID NO:22, or the complement thereof; or iii) a cDNA molecule encoding a PIEZO1 predicted gain-of-function polypeptide, or the complement thereof, wherein the cDNA molecule has a nucleotide sequence comprising: a thymine at a position corresponding to position 7,785 according to SEQ ID NO:33, or the complement thereof; a thymine at a position corresponding to position 1,095 according to SEQ ID NO:34, or the complement thereof; a thymine at a position corresponding to position 6,073 according to SEQ ID NO:35, or the complement thereof; a thymine at a position corresponding to position 1,336 according to SEQ ID NO:36, or the complement thereof; a thymine at a position corresponding to position 6,073 according to SEQ ID NO:37, or the complement thereof; a thymine at a position corresponding to position 6,236 according to SEQ ID NO:38, or the complement thereof; a thymine at a position corresponding to position 7,526 according to SEQ ID NO:39, or the complement thereof; a thymine at a position corresponding to position 1,336 according to SEQ ID NO:40, or the complement thereof; a thymine at a position corresponding to position 361 according to SEQ ID NO:41, or the complement thereof; or a thymine at a position corresponding to position 7,776 according to SEQ ID NO:42, or the complement thereof.

### DESCRIPTION

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is not intended that any method or aspect set forth herein be construed as requiring that its steps be performed in a specific order. Accordingly, where a method claim does not specifically state in the claims or descriptions that the steps are to be limited to a specific order, it is not intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by ±10% and remain within the scope of the disclosed embodiments.

As used herein, the term “comprising” may be replaced with “consisting” or “consisting essentially of” in particular embodiments as desired.

As used herein, the term “isolated”, in regard to a nucleic acid molecule or a polypeptide, means that the nucleic acid molecule or polypeptide is in a condition other than its native environment, such as apart from blood and/or other tissue. In some embodiments, an isolated nucleic acid molecule or polypeptide is substantially free of other nucleic acid molecules or other polypeptides, particularly other nucleic acid molecules or polypeptides of animal origin. In some embodiments, the nucleic acid molecule or polypeptide can be in a highly purified form, i.e., greater than 95% pure or greater than 99% pure. When used in this context, the term “isolated” does not exclude the presence of the same nucleic acid molecule or polypeptide in alternative physical forms, such as dimers or alternatively phosphorylated or derivatized forms.

As used herein, the terms “nucleic acid”, “nucleic acid molecule”, “nucleic acid sequence”, “polynucleotide”, or “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the term “subject” includes any animal, including mammals. Mammals include, but are not limited to, farm animals (such as, for example, horse, cow, pig), companion animals (such as, for example, dog, cat), laboratory animals (such as, for example, mouse, rat, rabbits), and non-human primates (such as, for example, apes and monkeys). In some embodiments, the subject is a human. In some embodiments, the subject is a patient under the care of a physician.

A putative gain-of-function (GOF) missense variant in the PIEZO1 gene associated with a decreased risk of developing varicose veins in humans has been identified in accordance with the present disclosure. Specifically, a genetic alteration that changes the cytosine at position 69,579 in the PIEZO1 reference genomic nucleic acid molecule (see, SEQ ID NO:1) to a thymine, has been observed to indicate that the subject having such an alteration may have a decreased risk of developing varicose veins. Altogether, the genetic analyses described herein surprisingly indicate that the PIEZO1 gene and, in particular, pGOFs in the PIEZO1 gene, associates with a decreased risk of developing varicose veins. Therefore, subjects that are PIEZO1 reference that have an increased risk of developing varicose veins, such as saphenous varicose veins, veticular varicose veins, spider veins, or pregnancy-related varicose veins, may be treated such that the varicose veins is prevented, the symptoms thereof are reduced, and/or development of symptoms is repressed. Accordingly, the present disclosure provides methods of leveraging the identification of such variants in subjects to identify or stratify risk in such subjects of developing varicose veins, such as saphenous varicose veins, veticular varicose veins, spider veins, or pregnancy-related varicose veins, or to diagnose subjects as having an increased risk of developing varicose veins, such as saphenous varicose veins, veticular varicose veins, spider veins, or pregnancy-related varicose veins, such that subjects at risk or subjects with active disease may be treated accordingly.

It has been further observed in accordance with the present disclosure that a PIEZO1 variant nucleic acid molecule encoding a PIEZO1 predicted gain-of-function polypeptide (whether these variations are homozygous or heterozygous in a particular subject) associate with a decreased risk of developing varicose veins. Moreover, the identification by the present disclosure of the association between additional variants and gene burden masks indicates that PIEZO1 itself (rather than linkage disequilibrium with variants in another gene) is responsible for a protective effect in varicose veins.

For purposes of the present disclosure, any particular subject can be categorized as having one of three PIEZO1 genotypes: i) PIEZO1 reference; ii) heterozygous for a PIEZO1 variant nucleic acid molecule encoding a PIEZO1 predicted gain-of-function polypeptide; or iii) homozygous for a PIEZO1 variant nucleic acid molecule encoding a PIEZO1 predicted gain-of-function polypeptide. A subject is PIEZO1 reference when the subject does not have a copy of a PIEZO1 variant nucleic acid molecule encoding a PIEZO1 predicted gain-of-function polypeptide. A subject is heterozygous for a PIEZO1 variant nucleic acid molecule encoding a PIEZO1 predicted gain-of-function polypeptide when the subject has a single copy of a PIEZO1 variant nucleic acid molecule. As used herein, a PIEZO1 variant nucleic acid molecule is any PIEZO1 nucleic acid molecule (such as, a genomic nucleic acid molecule, an mRNA molecule, or a cDNA molecule) encoding a PIEZO1 polypeptide having a partial gain-of-function, a complete gain-of-function, a predicted partial gain-of-function, or a predicted complete gain-of-function. A subject who has a PIEZO1 variant nucleic acid molecule encoding a PIEZO1 predicted gain-of-function polypeptide having a partial gain-of-function (or predicted partial gain-of-function) is hypomorphic for PIEZO1. The PIEZO1 variant nucleic acid molecule encoding a PIEZO1 predicted gain-of-function polypeptide can be any nucleic acid molecule encoding a PIEZO1 Pro2,510Leu, Pro2,024Leu, Pro2,079Leu, or Pro92Leu. In some embodiments, the PIEZO1 variant nucleic acid molecule encodes PIEZO1 Pro2,510Leu. A subject is homozygous for a PIEZO1 variant nucleic acid molecule encoding a PIEZO1 predicted gain-of-function polypeptide when the subject has two copies of a PIEZO1 variant nucleic acid molecule encoding a PIEZO1 predicted gain-of-function polypeptide.

For subjects that are genotyped or determined to be PIEZO1 reference, such subjects have an increased risk of developing varicose veins, such as saphenous varicose veins, veticular varicose veins, spider veins, or pregnancy-related varicose veins. For subjects that are genotyped or determined to be either PIEZO1 reference or heterozygous for a PIEZO1 variant nucleic acid molecule encoding a PIEZO1 predicted gain-of-function polypeptide, such subjects can be treated with a PIEZO1 agonist.

In any of the embodiments described throughout the present disclosure, the PIEZO1 variant nucleic acid molecule can be any PIEZO1 nucleic acid molecule (such as, for example, genomic nucleic acid molecule, mRNA molecule, or cDNA molecule) encoding a PIEZO1 polypeptide having a partial gain-of-function, a complete gain-of-function, a predicted partial gain-of-function, or a predicted complete gain-of-function. For example, the PIEZO1 variant nucleic acid molecule can be any nucleic acid molecule encoding PIEZO1 Pro2,510Leu, Pro2,024Leu, Pro2,079Leu, or Pro92Leu. In some embodiments, the PIEZO1 variant nucleic acid molecule encodes PIEZO1 Pro2,510Leu.

In any of the embodiments described throughout the present disclosure, the PIEZO1 predicted gain-of-function polypeptide can be any PIEZO1 polypeptide having a partial gain-of-function, a complete gain-of-function, a predicted partial gain-of-function, or a predicted complete gain-of-function. In any of the embodiments described throughout the present disclosure, the PIEZO1 predicted gain-of-function polypeptide can be any of the PIEZO1 polypeptides described herein including, for example, PIEZO1 Pro2,510Leu, Pro2,024Leu, Pro2,079Leu, or Pro92Leu. In any of the embodiments described throughout the present disclosure, the PIEZO1 predicted gain-of-function polypeptide can be PIEZO1 Pro2,510Leu.

In any of the embodiments described throughout the present disclosure, the varicose veins is saphenous varicose veins, veticular varicose veins, spider veins, or pregnancy-related varicose veins. In any of the embodiments described throughout the present disclosure, the varicose veins is saphenous varicose veins. In any of the embodiments described throughout the present disclosure, the varicose veins is veticular varicose veins. In any of the embodiments described throughout the present disclosure, the varicose veins is spider veins. In any of the embodiments described throughout the present disclosure, the varicose veins is pregnancy-related varicose veins.

Symptoms of varicose veins include, but are not limited to, heavy legs, appearance of spider veins (telangiectasia) in the affected leg, ankle swelling, especially in the evening, brownish-yellow shiny skin discoloration near the affected veins, redness, dryness, and itchiness of areas of skin (termed stasis dermatitis or venous eczema), cramps especially developing when making a sudden move such as standing up, minor injuries to the affected area, bleeding more than normal or taking a long time to heal, shrinking of the skin above the ankle (lipodermatosclerosis), restless legs syndrome, whitened, irregular scar-like patches appearing at the ankles (atrophie blanche), or any combination thereof.

The present disclosure provides methods of treating a subject having varicose veins or at risk of developing varicose veins, the methods comprising administering a PIEZO1 agonist to the subject.

The present disclosure also provides methods of treating a subject having saphenous varicose veins or at risk of developing saphenous varicose veins, the methods comprising administering a PIEZO1 agonist to the subject.

The present disclosure also provides methods of treating a subject having veticular varicose veins or at risk of developing veticular varicose veins, the methods comprising administering a PIEZO1 agonist to the subject.

The present disclosure also provides methods of treating a subject having spider veins or at risk of developing spider veins, the methods comprising administering a PIEZO1 agonist to the subject.

The present disclosure also provides methods of treating a subject having pregnancy-related varicose veins or at risk of developing pregnancy-related varicose veins, the methods comprising administering a PIEZO1 agonist to the subject.

In some embodiments, the methods of treatment further comprise detecting the presence or absence of a PIEZO1 variant nucleic acid molecule encoding a PIEZO1 predicted gain-of-function polypeptide in a biological sample obtained from the subject. As used throughout the present disclosure, “a PIEZO1 variant nucleic acid molecule” is any PIEZO1 nucleic acid molecule (such as, for example, genomic nucleic acid molecule, mRNA molecule, or cDNA molecule) encoding a PIEZO1 polypeptide having a partial gain-of-function, a complete gain-of-function, a predicted partial gain-of-function, or a predicted complete gain-of-function.

The present disclosure also provides methods of treating a subject with a therapeutic agent that treats or prevents varicose veins. In some embodiments, the subject has varicose veins. In some embodiments, the subject is at risk of developing varicose veins. In some embodiments, the methods comprise determining whether the subject has a PIEZO1 variant nucleic acid molecule encoding a PIEZO1 predicted gain-of-function polypeptide by obtaining or having obtained a biological sample obtained from the subject, and performing or having performed a sequence analysis on the biological sample to determine if the subject has a genotype comprising the PIEZO1 variant nucleic acid molecule. When the subject is PIEZO1 reference, the therapeutic agent that treats or prevents varicose veins is administered or continued to be administered to the subject in a standard dosage amount, and/or a PIEZO1 agonist is administered to the subject. When the subject is heterozygous for a PIEZO1 variant, the therapeutic agent that treats or prevents varicose veins is administered or continued to be administered to the subject in an amount that is the same as or less than a standard dosage amount, and/or a PIEZO1 agonist is administered to the subject. The presence of a genotype having the PIEZO1 variant nucleic acid molecule encoding the PIEZO1 predicted gain-of-function polypeptide indicates the subject has a decreased risk of developing varicose veins. In some embodiments, the subject is PIEZO1 reference. In some embodiments, the subject is heterozygous for the PIEZO1 variant nucleic acid molecule encoding a PIEZO1 predicted gain-of-function polypeptide.

For subjects that are genotyped or determined to be either PIEZO1 reference or heterozygous for the PIEZO1 variant nucleic acid molecule encoding a PIEZO1 predicted gain-of-function polypeptide, such subjects can be treated with a PIEZO1 agonist, as described herein.

Detecting the presence or absence of a PIEZO1 variant nucleic acid molecule encoding a PIEZO1 predicted gain-of-function polypeptide in a biological sample obtained from a subject and/or determining whether a subject has a PIEZO1 variant nucleic acid molecule encoding a PIEZO1 predicted gain-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the PIEZO1 variant nucleic acid molecule encoding a PIEZO1 predicted gain-of-function polypeptide can be present within a cell obtained from the subject.

In some embodiments, when the subject is PIEZO1 reference, the subject is also administered a therapeutic agent that treats or prevents varicose veins in a standard dosage amount. In some embodiments, when the subject is heterozygous for a PIEZO1 variant nucleic acid molecule encoding a PIEZO1 predicted gain-of-function polypeptide, the subject is administered a therapeutic agent that treats or prevents varicose veins in a dosage amount that is the same as or less than a standard dosage amount.

In some embodiments, the treatment methods further comprise detecting the presence or absence of a PIEZO1 predicted gain-of-function polypeptide in a biological sample obtained from the subject. In some embodiments, when the subject does not have a PIEZO1 predicted gain-of-function polypeptide, the subject is administered a therapeutic agent that treats or prevents varicose veins in a standard dosage amount. In some embodiments, when the subject has a PIEZO1 predicted gain-of-function polypeptide, the subject is administered a therapeutic agent that treats or prevents varicose veins in a dosage amount that is the same as or less than a standard dosage amount.

The present disclosure also provides methods of treating a subject with a therapeutic agent that treats or prevents varicose veins. In some embodiments, the subject has varicose veins. In some embodiments, the subject is at risk of developing varicose veins. In some embodiments, the methods comprise determining whether the subject has a PIEZO1 predicted gain-of-function polypeptide by obtaining or having obtained a biological sample from the subject, and performing or having performed an assay on the biological sample to determine if the subject has a PIEZO1 predicted gain-of-function polypeptide. When the subject does not have a PIEZO1 predicted gain-of-function polypeptide, the therapeutic agent that treats or prevents varicose veins is administered or continued to be administered to the subject in a standard dosage amount, and/or a PIEZO1 agonist is administered to the subject. When the subject has a PIEZO1 predicted gain-of-function polypeptide, the therapeutic agent that treats or prevents varicose veins is administered or continued to be administered to the subject in an amount that is the same as or less than a standard dosage amount, and/or a PIEZO1 agonist is administered to the subject. The presence of a PIEZO1 predicted gain-of-function polypeptide indicates the subject has a decreased risk of developing varicose veins. In some embodiments, the subject has a PIEZO1 predicted gain-of-function polypeptide. In some embodiments, the subject does not have a PIEZO1 predicted gain-of-function polypeptide.

Detecting the presence or absence of a PIEZO1 predicted gain-of-function polypeptide in a biological sample obtained from a subject and/or determining whether a subject has a PIEZO1 predicted gain-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the PIEZO1 predicted gain-of-function polypeptide can be present within a cell obtained from the subject.

Examples of therapeutic agents that treat or inhibit varicose veins include, but are not limited to flavonoids, such as diosmin or hesperidin, and anti-inflammatory agents, such as ibuprofen and aspirin.

Examples of PIEZO1 agonists include, but are not limited to, Yoda1, Jedi1, and Jedi2. In some embodiments, the PIEZO1 agonist comprises a small molecule.

In some embodiments, the dose of the therapeutic agents that treat or prevent varicose veins and/or the PIEZO1 agonists can be reduced by about 10%, by about 20%, by about 30%, by about 40%, by about 50%, by about 60%, by about 70%, by about 80%, or by about 90% for subjects that are heterozygous for a PIEZO1 variant nucleic acid molecule encoding a PIEZO1 predicted gain-of-function polypeptide (i.e., less than the standard dosage amount) compared to subjects that are PIEZO1 reference (who may receive a standard dosage amount). In some embodiments, the dose of the therapeutic agents that treat or prevent varicose veins and/or the PIEZO1 agonists can be reduced by about 10%, by about 20%, by about 30%, by about 40%, or by about 50%. In addition, the dose of therapeutic agents that treat or prevent varicose veins and/or the PIEZO1 agonists in subjects that are heterozygous for a PIEZO1 variant nucleic acid molecule encoding a PIEZO1 predicted gain-of-function polypeptide can be administered less frequently compared to subjects that are PIEZO1 reference.

Administration of the therapeutic agents that treat or prevent varicose veins and/or PIEZO1 agonists can be repeated, for example, after one day, two days, three days, five days, one week, two weeks, three weeks, one month, five weeks, six weeks, seven weeks, eight weeks, two months, or three months. The repeated administration can be at the same dose or at a different dose. The administration can be repeated once, twice, three times, four times, five times, six times, seven times, eight times, nine times, ten times, or more. For example, according to certain dosage regimens a subject can receive therapy for a prolonged period of time such as, for example, 6 months, 1 year, or more. In addition, the therapeutic agents that treat or prevent varicose veins and/or PIEZO1 agonists can be administered sequentially or at the same time. In addition, the therapeutic agents that treat or prevent varicose veins and/or PIEZO1 agonists can be administered in separate compositions or can be administered together in the same composition.

Administration of the therapeutic agents that treat or prevent varicose veins and/or PIEZO1 agonists can occur by any suitable route including, but not limited to, parenteral, intravenous, oral, subcutaneous, intra-arterial, intracranial, intrathecal, intraperitoneal, topical, intranasal, or intramuscular. Pharmaceutical compositions for administration are desirably sterile and substantially isotonic and manufactured under GMP conditions. Pharmaceutical compositions can be provided in unit dosage form (i.e., the dosage for a single administration). Pharmaceutical compositions can be formulated using one or more physiologically and pharmaceutically acceptable carriers, diluents, excipients or auxiliaries. The formulation depends on the route of administration chosen. The term “pharmaceutically acceptable” means that the carrier, diluent, excipient, or auxiliary is compatible with the other ingredients of the formulation and not substantially deleterious to the recipient thereof.

The terms “treat”, “treating”, and “treatment” and “prevent”, “preventing”, and “prevention” as used herein, refer to eliciting the desired biological response, such as a therapeutic and prophylactic effect, respectively. In some embodiments, a therapeutic effect comprises one or more of a decrease/reduction in varicose veins, a decrease/reduction in the severity of varicose veins (such as, for example, a reduction or inhibition of development of varicose veins), a decrease/reduction in symptoms and varicose veins-related effects, delaying the onset of symptoms and varicose veins-related effects, reducing the severity of symptoms of varicose veins-related effects, reducing the severity of an acute episode, reducing the number of symptoms and varicose veins-related effects, reducing the latency of symptoms and varicose veins-related effects, an amelioration of symptoms and varicose veins-related effects, reducing secondary symptoms, reducing secondary infections, preventing relapse to varicose veins, decreasing the number or frequency of relapse episodes, increasing latency between symptomatic episodes, increasing time to sustained progression, expediting remission, inducing remission, augmenting remission, speeding recovery, or increasing efficacy of or decreasing resistance to alternative therapeutics, and/or an increased survival time of the affected host animal, following administration of the agent or composition comprising the agent. A prophylactic effect may comprise a complete or partial avoidance/inhibition or a delay of varicose veins development/progression (such as, for example, a complete or partial avoidance/inhibition or a delay), and an increased survival time of the affected host animal, following administration of a therapeutic protocol. Treatment of varicose veins encompasses the treatment of subjects already diagnosed as having any form of varicose veins at any clinical stage or manifestation, the delay of the onset or evolution or aggravation or deterioration of the symptoms or signs of varicose veins, and/or preventing and/or reducing the severity of varicose veins.

The present disclosure also provides methods of identifying a subject having an increased risk of developing varicose veins. In some embodiments, the methods comprise determining or having determined the presence or absence of a PIEZO1 variant nucleic acid molecule (such as a genomic nucleic acid molecule, mRNA molecule, and/or cDNA molecule) encoding a PIEZO1 predicted gain-of-function polypeptide in a biological sample obtained from the subject. When the subject lacks a PIEZO1 variant nucleic acid molecule encoding a PIEZO1 predicted gain-of-function polypeptide (i.e., the subject is genotypically categorized as PIEZO1 reference), then the subject has an increased risk of developing varicose veins. When the subject has a PIEZO1 variant nucleic acid molecule encoding a PIEZO1 predicted gain-of-function polypeptide (i.e., the subject is heterozygous or homozygous for a PIEZO1 variant nucleic acid molecule), then the subject has a decreased risk of developing varicose veins compared to a subject that is PIEZO1 reference.

Having a single copy of a PIEZO1 variant nucleic acid molecule encoding a PIEZO1 predicted gain-of-function polypeptide is more protective of a subject from developing varicose veins than having no copies of a PIEZO1 variant nucleic acid molecule encoding a PIEZO1 predicted gain-of-function polypeptide. Without intending to be limited to any particular theory or mechanism of action, it is believed that a single copy of a PIEZO1 variant nucleic acid molecule (i.e., heterozygous for a PIEZO1 variant nucleic acid molecule) is protective of a subject from developing varicose veins, and it is also believed that having two copies of a PIEZO1 variant nucleic acid molecule encoding a PIEZO1 predicted gain-of-function polypeptide (i.e., homozygous for a PIEZO1 variant nucleic acid molecule) may be more protective of a subject from developing varicose veins, relative to a subject with a single copy. Thus, in some embodiments, a single copy of a PIEZO1 variant nucleic acid molecule encoding a PIEZO1 predicted gain-of-function polypeptide may not be completely protective, but instead, may be partially or incompletely protective of a subject from developing varicose veins. While not desiring to be bound by any particular theory, there may be additional factors or molecules involved in the development of varicose veins that are still present in a subject having a single copy of a PIEZO1 variant nucleic acid molecule encoding a PIEZO1 predicted gain-of-function polypeptide, thus resulting in less than complete protection from the development of varicose veins.

Detecting the presence or absence of a PIEZO1 variant nucleic acid molecule encoding a PIEZO1 predicted gain-of-function polypeptide in a biological sample obtained from a subject and/or determining whether a subject has a PIEZO1 variant nucleic acid molecule encoding a PIEZO1 predicted gain-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the PIEZO1 variant nucleic acid molecule encoding a PIEZO1 predicted gain-of-function polypeptide can be present within a cell obtained from the subject.

Any one or more (i.e., any combination) of the PIEZO1 variant nucleic acid molecules encoding a PIEZO1 predicted gain-of-function polypeptide can be used within any of the methods described herein to determine whether a subject has an increased risk of developing varicose veins. The combinations of particular variants can form a mask used for statistical analysis of the particular correlation of PIEZO1 and decreased risk of developing varicose veins.

In some embodiments, when a subject is identified as having an increased risk of developing varicose veins, the subject is further treated with a therapeutic agent that treats or prevents varicose veins and/or a PIEZO1 agonist, as described herein. For example, when the subject is PIEZO1 reference, and therefore has an increased risk for developing varicose veins, the subject is administered a PIEZO1 agonist. In some embodiments, such a subject is also administered a therapeutic agent that treats or prevents varicose veins. In some embodiments, when the subject is heterozygous for a PIEZO1 variant nucleic acid molecule encoding a PIEZO1 predicted gain-of-function polypeptide, the subject is administered the therapeutic agent that treats or prevents varicose veins in a dosage amount that is the same as or less than a standard dosage amount, and/or is administered a PIEZO1 agonist. In some embodiments, the subject is PIEZO1 reference. In some embodiments, the subject is heterozygous for a PIEZO1 variant nucleic acid molecule encoding a PIEZO1 predicted gain-of-function polypeptide.

In some embodiments, when a subject is identified as having an increased risk of developing varicose veins, the subject is further administered a therapeutic agent that treats or prevents varicose veins, and/or a PIEZO1 agonist, as described herein. For example, when the subject is PIEZO1 reference, and therefore has an increased risk of developing varicose veins, the subject is administered a PIEZO1 agonist. In some embodiments, such a subject is also administered a therapeutic agent that treats or prevents varicose veins. In some embodiments, when the subject is heterozygous for a PIEZO1 variant nucleic acid molecule encoding a PIEZO1 predicted gain-of-function polypeptide, the subject is administered the therapeutic agent that treats or prevents varicose veins in a dosage amount that is the same as or less than a standard dosage amount, and/or is administered a PIEZO1 agonist. In some embodiments, the subject is PIEZO1 reference. In some embodiments, the subject is heterozygous for a PIEZO1 variant nucleic acid molecule encoding a PIEZO1 predicted gain-of-function polypeptide.

In some embodiments, any of the methods described herein can further comprise determining the subject's aggregate burden of having a PIEZO1 variant nucleic acid molecule encoding a PIEZO1 predicted gain-of-function polypeptide, and/or a PIEZO1 predicted gain-of-function variant polypeptide associated with a decreased risk of developing varicose veins. The aggregate burden is the aggregate of all variants in the PIEZO1 gene, which can be carried out in an association analysis with varicose veins. In some embodiments, the subject is homozygous for one or more PIEZO1 variant nucleic acid molecules encoding a PIEZO1 predicted gain-of-function polypeptide associated with a decreased risk of developing varicose veins. In some embodiments, the subject is heterozygous for one or more PIEZO1 variant nucleic acid molecules encoding a PIEZO1 predicted gain-of-function polypeptide associated with a decreased risk of developing varicose veins. The result of the association analysis suggests that PIEZO1 variant nucleic acid molecules encoding a PIEZO1 predicted gain-of-function polypeptide are associated with decreased risk of developing varicose veins. When the subject has a lower aggregate burden, the subject is at a higher risk of developing varicose veins and the subject is administered or continued to be administered the therapeutic agent that treats or prevents varicose veins in a standard dosage amount, and/or a PIEZO1 agonist. When the subject has a greater aggregate burden, the subject is at a lower risk of developing varicose veins and the subject is administered or continued to be administered the therapeutic agent that treats or prevents varicose veins in an amount that is the same as or less than the standard dosage amount. The greater the aggregate burden, the lower the risk of developing varicose veins.

In some embodiments, the subject's aggregate burden of having any one or more PIEZO1 variant nucleic acid molecules encoding a PIEZO1 predicted gain-of-function polypeptide represents a weighted aggregate of a plurality of any of the PIEZO1 variant nucleic acid molecules encoding a PIEZO1 predicted gain-of-function polypeptide. In some embodiments, the aggregate burden is calculated using at least about 2, at least about 3, at least about 4, at least about 5, at least about 10, at least about 20, at least about 30, at least about 40, at least about 50, at least about 60, at least about 70, at least about 80, at least about 100, at least about 120, at least about 150, at least about 200, at least about 250, at least about 300, at least about 400, at least about 500, at least about 1,000, at least about 10,000, at least about 100,000, or at least about or more than 1,000,000 genetic variants present in or around (up to 10 Mb) the PIEZO1 gene where the genetic burden is the number of alleles multiplied by the association estimate with varicose veins or related outcome for each allele (e.g., a weighted polygenic burden score). This can include any genetic variants, regardless of their genomic annotation, in proximity to the PIEZO1 gene (up to 10 Mb around the gene) that show a non-zero association with varicose veins-related traits in a genetic association analysis. In some embodiments, when the subject has an aggregate burden above a desired threshold score, the subject has a decreased risk of developing varicose veins. In some embodiments, when the subject has an aggregate burden below a desired threshold score, the subject has an increased risk of developing varicose veins.

In some embodiments, the aggregate burden may be divided into quintiles, e.g., top quintile, intermediate quintile, and bottom quintile, wherein the top quintile of aggregate burden corresponds to the lowest risk group and the bottom quintile of aggregate burden corresponds to the highest risk group. In some embodiments, a subject having a greater aggregate burden comprises the highest weighted aggregate burdens, including, but not limited to the top 10%, top 20%, top 30%, top 40%, or top 50% of aggregate burdens from a subject population. In some embodiments, the genetic variants comprise the genetic variants having association with varicose veins in the top 10%, top 20%, top 30%, top 40%, or top 50% of p-value range for the association. In some embodiments, each of the identified genetic variants comprise the genetic variants having association with varicose veins with p-value of no more than about 10⁻², about 10⁻³, about 10⁻⁴, about 10⁻⁵, about 10⁻⁶, about 10⁻⁷, about 10⁻⁸, about 10⁻⁹, about 10⁻¹⁰, about 10⁻¹¹, about 10⁻¹², about 10⁻¹³, about 10⁻¹⁴, about or 10⁻¹⁵. In some embodiments, the identified genetic variants comprise the genetic variants having association with varicose veins with p-value of less than 5×10⁻⁸. In some embodiments, the identified genetic variants comprise genetic variants having association with varicose veins in high-risk subjects as compared to the rest of the reference population with odds ratio (OR) about 1.5 or greater, about 1.75 or greater, about 2.0 or greater, or about 2.25 or greater for the top 20% of the distribution; or about 1.5 or greater, about 1.75 or greater, about 2.0 or greater, about 2.25 or greater, about 2.5 or greater, or about 2.75 or greater. In some embodiments, the odds ratio (OR) may range from about 1.0 to about 1.5, from about 1.5 to about 2.0, from about 2.0 to about 2.5, from about 2.5 to about 3.0, from about 3.0 to about 3.5, from about 3.5 to about 4.0, from about 4.0 to about 4.5, from about 4.5 to about 5.0, from about 5.0 to about 5.5, from about 5.5 to about 6.0, from about 6.0 to about 6.5, from about 6.5 to about 7.0, or greater than 7.0. In some embodiments, high-risk subjects comprise subjects having aggregate burdens in the bottom decile, quintile, or tertile in a reference population. The threshold of the aggregate burden is determined on the basis of the nature of the intended practical application and the risk difference that would be considered meaningful for that practical application.

In some embodiments, when a subject is identified as having an increased risk of developing varicose veins, the subject is further administered a therapeutic agent that treats or prevents varicose veins, and/or a PIEZO1 agonist, as described herein. For example, when the subject is PIEZO1 reference, and therefore has an increased risk of developing varicose veins, the subject is administered a PIEZO1 agonist. In some embodiments, such a subject is also administered a therapeutic agent that treats or prevents varicose veins. In some embodiments, when the subject is heterozygous for a PIEZO1 variant nucleic acid molecule encoding a PIEZO1 predicted gain-of-function polypeptide, the subject is administered the therapeutic agent that treats or prevents varicose veins in a dosage amount that is the same as or less than a standard dosage amount, and/or is administered a PIEZO1 agonist. In some embodiments, the subject is PIEZO1 reference. In some embodiments, the subject is heterozygous for a PIEZO1 variant nucleic acid molecule encoding a PIEZO1 predicted gain-of-function polypeptide. Furthermore, when the subject has a lower aggregate burden for having a PIEZO1 variant nucleic acid molecule encoding a PIEZO1 predicted gain-of-function polypeptide, and therefore has an increased risk of developing varicose veins, the subject is administered a therapeutic agent that treats or prevents varicose veins. In some embodiments, when the subject has a lower aggregate burden for having a PIEZO1 variant nucleic acid molecule encoding a PIEZO1 predicted gain-of-function polypeptide, the subject is administered the therapeutic agent that treats or prevents varicose veins in a dosage amount that is the same as or greater than the standard dosage amount administered to a subject who has a greater aggregate burden for having a PIEZO1 variant nucleic acid molecule encoding a PIEZO1 predicted gain-of-function polypeptide.

The present disclosure also provides methods of detecting the presence or absence of a PIEZO1 variant genomic nucleic acid molecule encoding a PIEZO1 predicted gain-of-function polypeptide in a biological sample obtained from a subject, and/or a PIEZO1 variant mRNA molecule encoding a PIEZO1 predicted gain-of-function polypeptide in a biological sample obtained from a subject, and/or a PIEZO1 variant cDNA molecule encoding a PIEZO1 predicted gain-of-function polypeptide produced from an mRNA molecule in a biological sample obtained from a subject. It is understood that gene sequences within a population and mRNA molecules encoded by such genes can vary due to polymorphisms such as single nucleotide polymorphisms (SNPs). The sequences provided herein for the PIEZO1 variant genomic nucleic acid molecules, PIEZO1 variant mRNA molecules, and PIEZO1 variant cDNA molecules are only exemplary sequences. Other sequences for the PIEZO1 variant genomic nucleic acid molecules, variant mRNA molecules, and variant cDNA molecules are also possible.

The biological sample can be derived from any cell, tissue, or biological fluid from the subject. The biological sample may comprise any clinically relevant tissue such as, for example, a bone marrow sample, a tumor biopsy, a fine needle aspirate, or a sample of bodily fluid, such as blood, gingival crevicular fluid, plasma, serum, lymph, ascitic fluid, cystic fluid, or urine. In some embodiments, the biological sample comprises a buccal swab. The biological sample used in the methods disclosed herein can vary based on the assay format, nature of the detection method, and the tissues, cells, or extracts that are used as the sample. A biological sample can be processed differently depending on the assay being employed. For example, when detecting any PIEZO1 variant nucleic acid molecule, preliminary processing designed to isolate or enrich the biological sample for the PIEZO1 variant nucleic acid molecule can be employed. A variety of techniques may be used for this purpose. When detecting the level of any PIEZO1 variant mRNA molecule, different techniques can be used enrich the biological sample with mRNA molecules. Various methods to detect the presence or level of an mRNA molecule or the presence of a particular variant genomic DNA locus can be used.

The present disclosure also provides methods of detecting a PIEZO1 variant nucleic acid molecule, or the complement thereof, encoding a PIEZO1 predicted gain-of-function polypeptide in a subject. The methods comprise assaying a biological sample obtained from the subject to determine whether a nucleic acid molecule in the biological sample is a PIEZO1 variant nucleic acid molecule encoding a PIEZO1 predicted gain-of-function polypeptide.

In some embodiments, the PIEZO1 variant nucleic acid molecule encoding the PIEZO1 predicted gain-of-function polypeptide, or the complement thereof, is a genomic nucleic acid molecule having a nucleotide sequence comprising a thymine at a position corresponding to position 69,579 according to SEQ ID NO:2, or the complement thereof.

In some embodiments, the PIEZO1 variant nucleic acid molecule encoding the PIEZO1 predicted gain-of-function polypeptide, or the complement thereof, is an mRNA molecule having a nucleotide sequence comprising: a uracil at a position corresponding to position 7,785 according to SEQ ID NO:13, or the complement thereof; a uracil at a position corresponding to position 1,095 according to SEQ ID NO:14, or the complement thereof; a uracil at a position corresponding to position 6,073 according to SEQ ID NO:15, or the complement thereof; a uracil at a position corresponding to position 1,336 according to SEQ ID NO:16, or the complement thereof; a uracil at a position corresponding to position 6,073 according to SEQ ID NO:17, or the complement thereof; a uracil at a position corresponding to position 6,236 according to SEQ ID NO:18, or the complement thereof; a uracil at a position corresponding to position 7,526 according to SEQ ID NO:19, or the complement thereof; a uracil at a position corresponding to position 1,336 according to SEQ ID NO:20, or the complement thereof; a uracil at a position corresponding to position 361 according to SEQ ID NO:21, or the complement thereof; or a uracil at a position corresponding to position 7,776 according to SEQ ID NO:22, or the complement thereof.

In some embodiments, the PIEZO1 variant nucleic acid molecule encoding the PIEZO1 predicted gain-of-function polypeptide, or the complement thereof, is a cDNA molecule produced from an mRNA molecule in the biological sample having a nucleotide sequence comprising: a thymine at a position corresponding to position 7,785 according to SEQ ID NO:33, or the complement thereof; a thymine at a position corresponding to position 1,095 according to SEQ ID NO:34, or the complement thereof; a thymine at a position corresponding to position 6,073 according to SEQ ID NO:35, or the complement thereof; a thymine at a position corresponding to position 1,336 according to SEQ ID NO:36, or the complement thereof; a thymine at a position corresponding to position 6,073 according to SEQ ID NO:37, or the complement thereof; a thymine at a position corresponding to position 6,236 according to SEQ ID NO:38, or the complement thereof; a thymine at a position corresponding to position 7,526 according to SEQ ID NO:39, or the complement thereof; a thymine at a position corresponding to position 1,336 according to SEQ ID NO:40, or the complement thereof; a thymine at a position corresponding to position 361 according to SEQ ID NO:41, or the complement thereof; or a thymine at a position corresponding to position 7,776 according to SEQ ID NO:42, or the complement thereof.

In some embodiments, the PIEZO1 variant nucleic acid molecule has a nucleotide sequence comprising: a thymine at a position corresponding to position 69,579 according to SEQ ID NO:2, or the complement thereof, (for genomic nucleic acid molecules); a uracil at a position corresponding to position 7,785 according to SEQ ID NO:13, or the complement thereof, (for mRNA molecules); or a thymine at a position corresponding to position 7,785 according to SEQ ID NO:33, or the complement thereof, (for cDNA molecules obtained from mRNA molecules).

In some embodiments, the biological sample comprises a cell or cell lysate. Such methods can further comprise, for example, obtaining a biological sample from the subject comprising a PIEZO1 genomic nucleic acid molecule or mRNA molecule, and if mRNA, optionally reverse transcribing the mRNA into cDNA. Such assays can comprise, for example determining the identity of these positions of the particular PIEZO1 nucleic acid molecule. In some embodiments, the method is an in vitro method.

In some embodiments, the assay comprises sequencing at least a portion of the nucleotide sequence of the PIEZO1 nucleic acid molecule, or the complement thereof, in the biological sample. In some embodiments, the assay comprises sequencing at least a portion of: the nucleotide sequence of the PIEZO1 genomic nucleic acid molecule in the biological sample, wherein the sequenced portion comprises a position corresponding to position 69,579 according to SEQ ID NO:2, or the complement thereof; the nucleotide sequence of the PIEZO1 mRNA molecule in the biological sample, wherein the sequenced portion comprises a position corresponding to position 7,785 according to SEQ ID NO:22, or the complement thereof; and/or the nucleotide sequence of the PIEZO1 cDNA molecule produced from the mRNA in the biological sample, wherein the sequenced portion comprises a position corresponding to position 7,785 according to SEQ ID NO:33, or the complement thereof. When the sequenced portion of the PIEZO1 nucleic acid molecule in the biological sample comprises: a thymine at a position corresponding to position 69,579 according to SEQ ID NO:2, or the complement thereof; a uracil at a position corresponding to position 7,785 according to SEQ ID NO:13, or the complement thereof; or a thymine at a position corresponding to position 7,785 according to SEQ ID NO:33, or the complement thereof; then the PIEZO1 nucleic acid molecule in the biological sample is a PIEZO1 variant nucleic acid molecule encoding a PIEZO1 predicted gain-of-function polypeptide.

In some embodiments, the assay comprises sequencing at least a portion of the nucleotide sequence of the PIEZO1 genomic nucleic acid molecule, or the complement thereof, in the biological sample, wherein the sequenced portion comprises a position corresponding to position 69,579 according to SEQ ID NO:2, or the complement thereof. When the sequenced portion of the PIEZO1 genomic nucleic acid molecule in the biological sample comprises: a thymine at a position corresponding to position 69,579 according to SEQ ID NO:2, or the complement thereof, then the PIEZO1 genomic nucleic acid molecule in the biological sample is a PIEZO1 variant genomic nucleic acid molecule encoding a PIEZO1 predicted gain-of-function polypeptide.

In some embodiments, the assay comprises sequencing at least a portion of the nucleotide sequence of the PIEZO1 mRNA molecule in the biological sample, wherein the sequenced portion comprises a position corresponding to: position 7,785 according to SEQ ID NO:13, or the complement thereof; position 1,095 according to SEQ ID NO:14, or the complement thereof; position 6,073 according to SEQ ID NO:15, or the complement thereof; position 1,336 according to SEQ ID NO:16, or the complement thereof; position 6,073 according to SEQ ID NO:17, or the complement thereof; position 6,236 according to SEQ ID NO:18, or the complement thereof; position 7,526 according to SEQ ID NO:19, or the complement thereof; position 1,336 according to SEQ ID NO:20, or the complement thereof; position corresponding to position 361 according to SEQ ID NO:21, or the complement thereof; or position 7,776 according to SEQ ID NO:22, or the complement thereof. When the sequenced portion of the PIEZO1 mRNA molecule in the biological sample comprises: a uracil at a position corresponding to position 7,785 according to SEQ ID NO:13, or the complement thereof; a uracil at a position corresponding to position 1,095 according to SEQ ID NO:14, or the complement thereof; a uracil at a position corresponding to position 6,073 according to SEQ ID NO:15, or the complement thereof; a uracil at a position corresponding to position 1,336 according to SEQ ID NO:16, or the complement thereof; a uracil at a position corresponding to position 6,073 according to SEQ ID NO:17, or the complement thereof; a uracil at a position corresponding to position 6,236 according to SEQ ID NO:18, or the complement thereof; a uracil at a position corresponding to position 7,526 according to SEQ ID NO:19, or the complement thereof; a uracil at a position corresponding to position 1,336 according to SEQ ID NO:20, or the complement thereof; a uracil at a position corresponding to position 361 according to SEQ ID NO:21, or the complement thereof; or a uracil at a position corresponding to position 7,776 according to SEQ ID NO:22, or the complement thereof; then the PIEZO1 mRNA molecule in the biological sample is a PIEZO1 variant mRNA molecule encoding a PIEZO1 predicted gain-of-function polypeptide.

In some embodiments, the assay comprises sequencing at least a portion of the nucleotide sequence of the PIEZO1 cDNA molecule produced from an mRNA molecule in the biological sample, wherein the sequenced portion comprises a position corresponding to: position 7,785 according to SEQ ID NO:33, or the complement thereof; position 1,095 according to SEQ ID NO:34, or the complement thereof; position 6,073 according to SEQ ID NO:35, or the complement thereof; position 1,336 according to SEQ ID NO:36, or the complement thereof; position 6,073 according to SEQ ID NO:37, or the complement thereof; position 6,236 according to SEQ ID NO:38, or the complement thereof; position 7,526 according to SEQ ID NO:39, or the complement thereof; position 1,336 according to SEQ ID NO:40, or the complement thereof; position 361 according to SEQ ID NO:41, or the complement thereof; or position 7,776 according to SEQ ID NO:42, or the complement thereof. When the sequenced portion of the PIEZO1 cDNA molecule in the biological sample comprises: a thymine at a position corresponding to position 7,785 according to SEQ ID NO:33, or the complement thereof; a thymine at a position corresponding to position 1,095 according to SEQ ID NO:34, or the complement thereof; a thymine at a position corresponding to position 6,073 according to SEQ ID NO:35, or the complement thereof; a thymine at a position corresponding to position 1,336 according to SEQ ID NO:36, or the complement thereof; a thymine at a position corresponding to position 6,073 according to SEQ ID NO:37, or the complement thereof; a thymine at a position corresponding to position 6,236 according to SEQ ID NO:38, or the complement thereof; a thymine at a position corresponding to position 7,526 according to SEQ ID NO:39, or the complement thereof; a thymine at a position corresponding to position 1,336 according to SEQ ID NO:40, or the complement thereof; a thymine at a position corresponding to position 361 according to SEQ ID NO:41, or the complement thereof; or a thymine at a position corresponding to position 7,776 according to SEQ ID NO:42, or the complement thereof; then the PIEZO1 cDNA molecule produced from an mRNA molecule in the biological sample is a PIEZO1 variant cDNA molecule encoding a PIEZO1 predicted gain-of-function polypeptide.

In some embodiments, the assay comprises: a) contacting the biological sample with a primer hybridizing to a portion of the nucleotide sequence of the PIEZO1: genomic nucleic acid molecule, or the complement thereof, that is proximate to a position corresponding to position 69,579 according to SEQ ID NO:2, or the complement thereof; mRNA molecule, or the complement thereof, that is proximate to a position corresponding to position 7,785 according to SEQ ID NO:13, or the complement thereof; and/or cDNA molecule, or the complement thereof, that is proximate to a position corresponding to position 7,785 according to SEQ ID NO:33, or the complement thereof; b) extending the primer at least through the position of the nucleotide sequence of the PIEZO1: genomic nucleic acid molecule, or the complement thereof, corresponding to position 69,579 according to SEQ ID NO:2, or the complement thereof; mRNA molecule, or the complement thereof, corresponding to position 7,785 according to SEQ ID NO:13, or the complement thereof; and/or cDNA molecule, or the complement thereof, corresponding to position 7,785 according to SEQ ID NO:33, or the complement thereof; and c) determining whether the extension product of the primer comprises: a thymine at a position corresponding to position 69,579 according to SEQ ID NO:2, or the complement thereof; a uracil at a position corresponding to position 7,785 according to SEQ ID NO:13, or the complement thereof; and/or a thymine at a position corresponding to position 7,785 according to SEQ ID NO:33, or the complement thereof.

In some embodiments, the assay comprises: a) contacting the biological sample with a primer hybridizing to a portion of the nucleotide sequence of the PIEZO1: mRNA molecule, or the complement thereof, that is proximate to a position corresponding to position 1,095 according to SEQ ID NO:14, or the complement thereof; and/or cDNA molecule, or the complement thereof, that is proximate to a position corresponding to position 1,095 according to SEQ ID NO:34, or the complement thereof; b) extending the primer at least through the position of the nucleotide sequence of the PIEZO1: mRNA molecule, or the complement thereof, corresponding to position 1,095 according to SEQ ID NO:14, or the complement thereof; and/or cDNA molecule, or the complement thereof, corresponding to position 1,095 according to SEQ ID NO:34, or the complement thereof; and c) determining whether the extension product of the primer comprises: a uracil at a position corresponding to position 1,095 according to SEQ ID NO:14, or the complement thereof; and/or a thymine at a position corresponding to position 1,095 according to SEQ ID NO:34, or the complement thereof.

In some embodiments, the assay comprises: a) contacting the biological sample with a primer hybridizing to a portion of the nucleotide sequence of the PIEZO1: mRNA molecule, or the complement thereof, that is proximate to a position corresponding to position 6,073 according to SEQ ID NO:15, or the complement thereof; and/or cDNA molecule, or the complement thereof, that is proximate to a position corresponding to position 6,073 according to SEQ ID NO:35, or the complement thereof; b) extending the primer at least through the position of the nucleotide sequence of the PIEZO1: mRNA molecule, or the complement thereof, corresponding to position 6,073 according to SEQ ID NO:15, or the complement thereof; and/or cDNA molecule, or the complement thereof, corresponding to position 6,073 according to SEQ ID NO:35, or the complement thereof; and c) determining whether the extension product of the primer comprises: a uracil at a position corresponding to position 6,073 according to SEQ ID NO:15, or the complement thereof; and/or a thymine at a position corresponding to position 6,073 according to SEQ ID NO:35, or the complement thereof.

In some embodiments, the assay comprises: a) contacting the biological sample with a primer hybridizing to a portion of the nucleotide sequence of the PIEZO1: mRNA molecule, or the complement thereof, that is proximate to a position corresponding to position 1,336 according to SEQ ID NO:16, or the complement thereof; and/or cDNA molecule, or the complement thereof, that is proximate to a position corresponding to position 1,336 according to SEQ ID NO:36, or the complement thereof; b) extending the primer at least through the position of the nucleotide sequence of the PIEZO1: mRNA molecule, or the complement thereof, corresponding to position 1,336 according to SEQ ID NO:16, or the complement thereof; and/or cDNA molecule, or the complement thereof, corresponding to position 1,336 according to SEQ ID NO:36, or the complement thereof; and c) determining whether the extension product of the primer comprises: a uracil at a position corresponding to position 1,336 according to SEQ ID NO:16, or the complement thereof; and/or a thymine at a position corresponding to position 1,336 according to SEQ ID NO:36, or the complement thereof.

In some embodiments, the assay comprises: a) contacting the biological sample with a primer hybridizing to a portion of the nucleotide sequence of the PIEZO1: mRNA molecule, or the complement thereof, that is proximate to a position corresponding to position 6,073 according to SEQ ID NO:17, or the complement thereof; and/or cDNA molecule, or the complement thereof, that is proximate to a position corresponding to position 6,073 according to SEQ ID NO:37, or the complement thereof; b) extending the primer at least through the position of the nucleotide sequence of the PIEZO1: mRNA molecule, or the complement thereof, corresponding to position 6,073 according to SEQ ID NO:17, or the complement thereof; and/or cDNA molecule, or the complement thereof, corresponding to position 6,073 according to SEQ ID NO:37, or the complement thereof; and c) determining whether the extension product of the primer comprises: a uracil at a position corresponding to position 6,073 according to SEQ ID NO:17, or the complement thereof; and/or a thymine at a position corresponding to position 6,073 according to SEQ ID NO:37, or the complement thereof.

In some embodiments, the assay comprises: a) contacting the biological sample with a primer hybridizing to a portion of the nucleotide sequence of the PIEZO1: mRNA molecule, or the complement thereof, that is proximate to a position corresponding to position 6,236 according to SEQ ID NO:18, or the complement thereof; and/or cDNA molecule, or the complement thereof, that is proximate to a position corresponding to position 6,236 according to SEQ ID NO:38, or the complement thereof; b) extending the primer at least through the position of the nucleotide sequence of the PIEZO1: mRNA molecule, or the complement thereof, corresponding to position 6,236 according to SEQ ID NO:18, or the complement thereof; and/or cDNA molecule, or the complement thereof, corresponding to position 6,236 according to SEQ ID NO:38, or the complement thereof; and c) determining whether the extension product of the primer comprises: a uracil at a position corresponding to position 6,236 according to SEQ ID NO:18, or the complement thereof; and/or a thymine at a position corresponding to position 6,236 according to SEQ ID NO:38, or the complement thereof.

In some embodiments, the assay comprises: a) contacting the biological sample with a primer hybridizing to a portion of the nucleotide sequence of the PIEZO1: mRNA molecule, or the complement thereof, that is proximate to a position corresponding to position 7,526 according to SEQ ID NO:19, or the complement thereof; and/or cDNA molecule, or the complement thereof, that is proximate to a position corresponding to position 7,526 according to SEQ ID NO:39, or the complement thereof; b) extending the primer at least through the position of the nucleotide sequence of the PIEZO1: mRNA molecule, or the complement thereof, corresponding to position 7,526 according to SEQ ID NO:19, or the complement thereof; and/or cDNA molecule, or the complement thereof, corresponding to position 7,526 according to SEQ ID NO:39, or the complement thereof; and c) determining whether the extension product of the primer comprises: a uracil at a position corresponding to position 7,526 according to SEQ ID NO:19, or the complement thereof; and/or a thymine at a position corresponding to position 7,526 according to SEQ ID NO:39, or the complement thereof.

In some embodiments, the assay comprises: a) contacting the biological sample with a primer hybridizing to a portion of the nucleotide sequence of the PIEZO1: mRNA molecule, or the complement thereof, that is proximate to a position corresponding to position 1,336 according to SEQ ID NO:20, or the complement thereof; and/or cDNA molecule, or the complement thereof, that is proximate to a position corresponding to position 1,336 according to SEQ ID NO:40, or the complement thereof; b) extending the primer at least through the position of the nucleotide sequence of the PIEZO1: mRNA molecule, or the complement thereof, corresponding to position 1,336 according to SEQ ID NO:20, or the complement thereof; and/or cDNA molecule, or the complement thereof, corresponding to position 1,336 according to SEQ ID NO:40, or the complement thereof; and c) determining whether the extension product of the primer comprises: a uracil at a position corresponding to position 1,336 according to SEQ ID NO:20, or the complement thereof; and/or a thymine at a position corresponding to position 1,336 according to SEQ ID NO:40, or the complement thereof.

In some embodiments, the assay comprises: a) contacting the biological sample with a primer hybridizing to a portion of the nucleotide sequence of the PIEZO1: mRNA molecule, or the complement thereof, that is proximate to a position corresponding to position 361 according to SEQ ID NO:21, or the complement thereof; and/or cDNA molecule, or the complement thereof, that is proximate to a position corresponding to position 361 according to SEQ ID NO:41, or the complement thereof; b) extending the primer at least through the position of the nucleotide sequence of the PIEZO1: mRNA molecule, or the complement thereof, corresponding to position 361 according to SEQ ID NO:21, or the complement thereof; and/or cDNA molecule, or the complement thereof, corresponding to position 361 according to SEQ ID NO:41, or the complement thereof; and c) determining whether the extension product of the primer comprises: a uracil at a position corresponding to position 361 according to SEQ ID NO:21, or the complement thereof; and/or a thymine at a position corresponding to position 361 according to SEQ ID NO:41, or the complement thereof.

In some embodiments, the assay comprises: a) contacting the biological sample with a primer hybridizing to a portion of the nucleotide sequence of the PIEZO1: mRNA molecule, or the complement thereof, that is proximate to a position corresponding to position 7,776 according to SEQ ID NO:22, or the complement thereof; and/or cDNA molecule, or the complement thereof, that is proximate to a position corresponding to position 7,776 according to SEQ ID NO:42, or the complement thereof; b) extending the primer at least through the position of the nucleotide sequence of the PIEZO1: mRNA molecule, or the complement thereof, corresponding to position 7,776 according to SEQ ID NO:22, or the complement thereof; and/or cDNA molecule, or the complement thereof, corresponding to position 7,776 according to SEQ ID NO:42, or the complement thereof; and c) determining whether the extension product of the primer comprises: a uracil at a position corresponding to position 7,776 according to SEQ ID NO:22, or the complement thereof; and/or a thymine at a position corresponding to position 7,776 according to SEQ ID NO:42, or the complement thereof.

In some embodiments, the assay comprises: a) contacting the biological sample with a primer hybridizing to a portion of the nucleotide sequence of the PIEZO1 genomic nucleic acid molecule, or the complement thereof, that is proximate to a position corresponding to position 69,579 according to SEQ ID NO:2, or the complement thereof; b) extending the primer at least through the position of the nucleotide sequence of the PIEZO1 genomic nucleic acid molecule, or the complement thereof, corresponding to: position 69,579 according to SEQ ID NO:2, or the complement thereof; and c) determining whether the extension product of the primer comprises: a thymine at a position corresponding to position 69,579 according to SEQ ID NO:2.

In some embodiments, the assay comprises: a) contacting the biological sample with a primer hybridizing to a portion of the nucleotide sequence of the PIEZO1 mRNA molecule, or the complement thereof, that is proximate to a position corresponding to: position 7,785 according to SEQ ID NO:13, or the complement thereof; position 1,095 according to SEQ ID NO:14, or the complement thereof; position 6,073 according to SEQ ID NO:15, or the complement thereof; position 1,336 according to SEQ ID NO:16, or the complement thereof; position 6,073 according to SEQ ID NO:17, or the complement thereof; position 6,236 according to SEQ ID NO:18, or the complement thereof; position 7,526 according to SEQ ID NO:19, or the complement thereof; position 1,336 according to SEQ ID NO:20, or the complement thereof; position corresponding to position 361 according to SEQ ID NO:21, or the complement thereof; or position 7,776 according to SEQ ID NO:22, or the complement thereof; b) extending the primer at least through the position of the nucleotide sequence of the PIEZO1 mRNA molecule, or the complement thereof, corresponding to: position 7,785 according to SEQ ID NO:13, or the complement thereof; position 1,095 according to SEQ ID NO:14, or the complement thereof; position 6,073 according to SEQ ID NO:15, or the complement thereof; position 1,336 according to SEQ ID NO:16, or the complement thereof; position 6,073 according to SEQ ID NO:17, or the complement thereof; position 6,236 according to SEQ ID NO:18, or the complement thereof; position 7,526 according to SEQ ID NO:19, or the complement thereof; position 1,336 according to SEQ ID NO:20, or the complement thereof; position corresponding to position 361 according to SEQ ID NO:21, or the complement thereof; or position 7,776 according to SEQ ID NO:22, or the complement thereof; and c) determining whether the extension product of the primer comprises: a uracil at a position corresponding to position 7,785 according to SEQ ID NO:13, or the complement thereof; a uracil at a position corresponding to position 1,095 according to SEQ ID NO:14, or the complement thereof; a uracil at a position corresponding to position 6,073 according to SEQ ID NO:15, or the complement thereof; a uracil at a position corresponding to position 1,336 according to SEQ ID NO:16, or the complement thereof; a uracil at a position corresponding to position 6,073 according to SEQ ID NO:17, or the complement thereof; a uracil at a position corresponding to position 6,236 according to SEQ ID NO:18, or the complement thereof; a uracil at a position corresponding to position 7,526 according to SEQ ID NO:19, or the complement thereof; a uracil at a position corresponding to position 1,336 according to SEQ ID NO:20, or the complement thereof; a uracil at a position corresponding to position 361 according to SEQ ID NO:21, or the complement thereof; or a uracil at a position corresponding to position 7,776 according to SEQ ID NO:22, or the complement thereof.

In some embodiments, the assay comprises: a) contacting the biological sample with a primer hybridizing to a portion of the nucleotide sequence of the PIEZO1 cDNA molecule, or the complement thereof, that is proximate to a position corresponding to: position 7,785 according to SEQ ID NO:33, or the complement thereof; position 1,095 according to SEQ ID NO:34, or the complement thereof; position 6,073 according to SEQ ID NO:35, or the complement thereof; position 1,336 according to SEQ ID NO:36, or the complement thereof; position 6,073 according to SEQ ID NO:37, or the complement thereof; position 6,236 according to SEQ ID NO:38, or the complement thereof; position 7,526 according to SEQ ID NO:39, or the complement thereof; position 1,336 according to SEQ ID NO:40, or the complement thereof; position 361 according to SEQ ID NO:41, or the complement thereof; or position 7,776 according to SEQ ID NO:42, or the complement thereof; b) extending the primer at least through the position of the nucleotide sequence of the PIEZO1 cDNA molecule, or the complement thereof, corresponding to: position 7,785 according to SEQ ID NO:33, or the complement thereof; position 1,095 according to SEQ ID NO:34, or the complement thereof; position 6,073 according to SEQ ID NO:35, or the complement thereof; position 1,336 according to SEQ ID NO:36, or the complement thereof; position 6,073 according to SEQ ID NO:37, or the complement thereof; position 6,236 according to SEQ ID NO:38, or the complement thereof; position 7,526 according to SEQ ID NO:39, or the complement thereof; position 1,336 according to SEQ ID NO:40, or the complement thereof; position 361 according to SEQ ID NO:41, or the complement thereof; or position 7,776 according to SEQ ID NO:42, or the complement thereof; and c) determining whether the extension product of the primer comprises: a thymine at a position corresponding to position 7,785 according to SEQ ID NO:33, or the complement thereof; a thymine at a position corresponding to position 1,095 according to SEQ ID NO:34, or the complement thereof; a thymine at a position corresponding to position 6,073 according to SEQ ID NO:35, or the complement thereof; a thymine at a position corresponding to position 1,336 according to SEQ ID NO:36, or the complement thereof; a thymine at a position corresponding to position 6,073 according to SEQ ID NO:37, or the complement thereof; a thymine at a position corresponding to position 6,236 according to SEQ ID NO:38, or the complement thereof; a thymine at a position corresponding to position 7,526 according to SEQ ID NO:39, or the complement thereof; a thymine at a position corresponding to position 1,336 according to SEQ ID NO:40, or the complement thereof; a thymine at a position corresponding to position 361 according to SEQ ID NO:41, or the complement thereof; or a thymine at a position corresponding to position 7,776 according to SEQ ID NO:42, or the complement thereof.

In some embodiments, the assay comprises sequencing the entire nucleic acid molecule. In some embodiments, only a PIEZO1 genomic nucleic acid molecule is analyzed. In some embodiments, only a PIEZO1 mRNA is analyzed. In some embodiments, only a PIEZO1 cDNA obtained from a PIEZO1 mRNA is analyzed.

In some embodiments, the assay comprises: a) amplifying at least a portion of the PIEZO1 nucleic acid molecule, or the complement thereof, in the biological sample, wherein the amplified portion comprises: a thymine at a position corresponding to position 69,579 according to SEQ ID NO:2, or the complement thereof; a uracil at a position corresponding to position 7,785 according to SEQ ID NO:13, or the complement thereof; and/or a thymine at a position corresponding to position 7,785 according to SEQ ID NO:33, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleotide sequence of the amplified nucleic acid molecule comprising: a thymine at a position corresponding to position 69,579 according to SEQ ID NO:2, or the complement thereof; a uracil at a position corresponding to position 7,785 according to SEQ ID NO:13, or the complement thereof; and/or a thymine at a position corresponding to position 7,785 according to SEQ ID NO:33, or the complement thereof; and d) detecting the detectable label.

In some embodiments, the assay comprises: a) amplifying at least a portion of the PIEZO1 nucleic acid molecule, or the complement thereof, in the biological sample, wherein the amplified portion comprises: a uracil at a position corresponding to position 1,095 according to SEQ ID NO:14, or the complement thereof; and/or a thymine at a position corresponding to position 1,095 according to SEQ ID NO:34, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleotide sequence of the amplified nucleic acid molecule comprising: a uracil at a position corresponding to position 1,095 according to SEQ ID NO:14, or the complement thereof; and/or a thymine at a position corresponding to position 1,095 according to SEQ ID NO:34, or the complement thereof; and d) detecting the detectable label.

In some embodiments, the assay comprises: a) amplifying at least a portion of the PIEZO1 nucleic acid molecule, or the complement thereof, in the biological sample, wherein the amplified portion comprises: a uracil at a position corresponding to position 6,073 according to SEQ ID NO:15, or the complement thereof; and/or a thymine at a position corresponding to position 6,073 according to SEQ ID NO:35, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleotide sequence of the amplified nucleic acid molecule comprising: a uracil at a position corresponding to position 6,073 according to SEQ ID NO:15, or the complement thereof; and/or a thymine at a position corresponding to position 6,073 according to SEQ ID NO:35, or the complement thereof; and d) detecting the detectable label.

In some embodiments, the assay comprises: a) amplifying at least a portion of the PIEZO1 nucleic acid molecule, or the complement thereof, in the biological sample, wherein the amplified portion comprises: a uracil at a position corresponding to position 1,336 according to SEQ ID NO:16, or the complement thereof; and/or a thymine at a position corresponding to position 1,336 according to SEQ ID NO:36, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleotide sequence of the amplified nucleic acid molecule comprising: a uracil at a position corresponding to position 1,336 according to SEQ ID NO:16, or the complement thereof; and/or a thymine at a position corresponding to position 1,336 according to SEQ ID NO:36, or the complement thereof; and d) detecting the detectable label.

In some embodiments, the assay comprises: a) amplifying at least a portion of the PIEZO1 nucleic acid molecule, or the complement thereof, in the biological sample, wherein the amplified portion comprises: a uracil at a position corresponding to position 6,073 according to SEQ ID NO:17, or the complement thereof; and/or a thymine at a position corresponding to position 6,073 according to SEQ ID NO:37, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleotide sequence of the amplified nucleic acid molecule comprising: a uracil at a position corresponding to position 6,073 according to SEQ ID NO:17, or the complement thereof; and/or a thymine at a position corresponding to position 6,073 according to SEQ ID NO:37, or the complement thereof; and d) detecting the detectable label.

In some embodiments, the assay comprises: a) amplifying at least a portion of the PIEZO1 nucleic acid molecule, or the complement thereof, in the biological sample, wherein the amplified portion comprises: a uracil at a position corresponding to position 6,236 according to SEQ ID NO:18, or the complement thereof; and/or a thymine at a position corresponding to position 6,236 according to SEQ ID NO:38, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleotide sequence of the amplified nucleic acid molecule comprising: a uracil at a position corresponding to position 6,236 according to SEQ ID NO:18, or the complement thereof; and/or a thymine at a position corresponding to position 6,236 according to SEQ ID NO:38, or the complement thereof; and d) detecting the detectable label.

In some embodiments, the assay comprises: a) amplifying at least a portion of the PIEZO1 nucleic acid molecule, or the complement thereof, in the biological sample, wherein the amplified portion comprises: a uracil at a position corresponding to position 7,526 according to SEQ ID NO:19, or the complement thereof; and/or a thymine at a position corresponding to position 7,526 according to SEQ ID NO:39, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleotide sequence of the amplified nucleic acid molecule comprising: a uracil at a position corresponding to position 7,526 according to SEQ ID NO:19, or the complement thereof; and/or a thymine at a position corresponding to position 7,526 according to SEQ ID NO:39, or the complement thereof; and d) detecting the detectable label.

In some embodiments, the assay comprises: a) amplifying at least a portion of the PIEZO1 nucleic acid molecule, or the complement thereof, in the biological sample, wherein the amplified portion comprises: a uracil at a position corresponding to position 1,336 according to SEQ ID NO:20, or the complement thereof; and/or a thymine at a position corresponding to position 1,336 according to SEQ ID NO:40, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleotide sequence of the amplified nucleic acid molecule comprising: a uracil at a position corresponding to position 1,336 according to SEQ ID NO:20, or the complement thereof; and/or a thymine at a position corresponding to position 1,336 according to SEQ ID NO:40, or the complement thereof; and d) detecting the detectable label.

In some embodiments, the assay comprises: a) amplifying at least a portion of the PIEZO1 nucleic acid molecule, or the complement thereof, in the biological sample, wherein the amplified portion comprises: a uracil at a position corresponding to position 361 according to SEQ ID NO:21, or the complement thereof; and/or a thymine at a position corresponding to position 361 according to SEQ ID NO:41, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleotide sequence of the amplified nucleic acid molecule comprising: a uracil at a position corresponding to position 361 according to SEQ ID NO:21, or the complement thereof; and/or a thymine at a position corresponding to position 361 according to SEQ ID NO:41, or the complement thereof; and d) detecting the detectable label.

In some embodiments, the assay comprises: a) amplifying at least a portion of the PIEZO1 nucleic acid molecule, or the complement thereof, in the biological sample, wherein the amplified portion comprises: a uracil at a position corresponding to position 7,776 according to SEQ ID NO:22, or the complement thereof; and/or a thymine at a position corresponding to position 7,776 according to SEQ ID NO:42, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleotide sequence of the amplified nucleic acid molecule comprising: a uracil at a position corresponding to position 7,776 according to SEQ ID NO:22, or the complement thereof; and/or a thymine at a position corresponding to position 7,776 according to SEQ ID NO:42, or the complement thereof; and d) detecting the detectable label.

In some embodiments, the assay comprises: a) amplifying at least a portion of the PIEZO1 genomic nucleic acid molecule, or the complement thereof, in the biological sample, wherein the portion comprises: a thymine at a position corresponding to position 69,579 according to SEQ ID NO:2, or the complement thereof; and d) detecting the detectable label.

In some embodiments, the assay comprises: a) amplifying at least a portion of the PIEZO1 mRNA molecule, or the complement thereof, in the biological sample, wherein the portion comprises: a uracil at a position corresponding to position 7,785 according to SEQ ID NO:13, or the complement thereof; a uracil at a position corresponding to position 1,095 according to SEQ ID NO:14, or the complement thereof; a uracil at a position corresponding to position 6,073 according to SEQ ID NO:15, or the complement thereof; a uracil at a position corresponding to position 1,336 according to SEQ ID NO:16, or the complement thereof; a uracil at a position corresponding to position 6,073 according to SEQ ID NO:17, or the complement thereof; a uracil at a position corresponding to position 6,236 according to SEQ ID NO:18, or the complement thereof; a uracil at a position corresponding to position 7,526 according to SEQ ID NO:19, or the complement thereof; a uracil at a position corresponding to position 1,336 according to SEQ ID NO:20, or the complement thereof; a uracil at a position corresponding to position 361 according to SEQ ID NO:21, or the complement thereof; or a uracil at a position corresponding to position 7,776 according to SEQ ID NO:22, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleotide sequence of the amplified nucleic acid molecule comprising: a uracil at a position corresponding to position 7,785 according to SEQ ID NO:13, or the complement thereof; a uracil at a position corresponding to position 1,095 according to SEQ ID NO:14, or the complement thereof; a uracil at a position corresponding to position 6,073 according to SEQ ID NO:15, or the complement thereof; a uracil at a position corresponding to position 1,336 according to SEQ ID NO:16, or the complement thereof; a uracil at a position corresponding to position 6,073 according to SEQ ID NO:17, or the complement thereof; a uracil at a position corresponding to position 6,236 according to SEQ ID NO:18, or the complement thereof; a uracil at a position corresponding to position 7,526 according to SEQ ID NO:19, or the complement thereof; a uracil at a position corresponding to position 1,336 according to SEQ ID NO:20, or the complement thereof; a uracil at a position corresponding to position 361 according to SEQ ID NO:21, or the complement thereof; or a uracil at a position corresponding to position 7,776 according to SEQ ID NO:22, or the complement thereof; and d) detecting the detectable label.

In some embodiments, the assay comprises: a) amplifying at least a portion of the PIEZO1 cDNA molecule, or the complement thereof, produced from an mRNA molecule in the biological sample, wherein the portion comprises: a thymine at a position corresponding to position 7,785 according to SEQ ID NO:33, or the complement thereof; a thymine at a position corresponding to position 1,095 according to SEQ ID NO:34, or the complement thereof; a thymine at a position corresponding to position 6,073 according to SEQ ID NO:35, or the complement thereof; a thymine at a position corresponding to position 1,336 according to SEQ ID NO:36, or the complement thereof; a thymine at a position corresponding to position 6,073 according to SEQ ID NO:37, or the complement thereof; a thymine at a position corresponding to position 6,236 according to SEQ ID NO:38, or the complement thereof; a thymine at a position corresponding to position 7,526 according to SEQ ID NO:39, or the complement thereof; a thymine at a position corresponding to position 1,336 according to SEQ ID NO:40, or the complement thereof; a thymine at a position corresponding to position 361 according to SEQ ID NO:41, or the complement thereof; or a thymine at a position corresponding to position 7,776 according to SEQ ID NO:42, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleotide sequence of the amplified nucleic acid molecule comprising: a thymine at a position corresponding to position 7,785 according to SEQ ID NO:33, or the complement thereof; a thymine at a position corresponding to position 1,095 according to SEQ ID NO:34, or the complement thereof; a thymine at a position corresponding to position 6,073 according to SEQ ID NO:35, or the complement thereof; a thymine at a position corresponding to position 1,336 according to SEQ ID NO:36, or the complement thereof; a thymine at a position corresponding to position 6,073 according to SEQ ID NO:37, or the complement thereof; a thymine at a position corresponding to position 6,236 according to SEQ ID NO:38, or the complement thereof; a thymine at a position corresponding to position 7,526 according to SEQ ID NO:39, or the complement thereof; a thymine at a position corresponding to position 1,336 according to SEQ ID NO:40, or the complement thereof; a thymine at a position corresponding to position 361 according to SEQ ID NO:41, or the complement thereof; or a thymine at a position corresponding to position 7,776 according to SEQ ID NO:42, or the complement thereof; and d) detecting the detectable label.

In some embodiments, the nucleic acid molecule in the sample is mRNA and the mRNA is reverse-transcribed into cDNA prior to the amplifying step.

In some embodiments, the assay comprises: contacting the PIEZO1 nucleic acid molecule, or the complement thereof, in the biological sample with an alteration-specific probe comprising a detectable label, wherein the alteration-specific probe comprises a nucleotide sequence which hybridizes under stringent conditions to the nucleotide sequence of the PIEZO1 nucleic acid molecule, or the complement thereof, comprising: a thymine at a position corresponding to position 69,579 according to SEQ ID NO:2, or the complement thereof; a uracil at a position corresponding to position 7,785 according to SEQ ID NO:13, or the complement thereof; and/or a thymine at a position corresponding to position 7,785 according to SEQ ID NO:33, or the complement thereof; and detecting the detectable label.

In some embodiments, the assay comprises: contacting the PIEZO1 nucleic acid molecule, or the complement thereof, in the biological sample with an alteration-specific probe comprising a detectable label, wherein the alteration-specific probe comprises a nucleotide sequence which hybridizes under stringent conditions to the nucleotide sequence of the PIEZO1 nucleic acid molecule, or the complement thereof, comprising: a uracil at a position corresponding to position 1,095 according to SEQ ID NO:14, or the complement thereof; and/or a thymine at a position corresponding to position 1,095 according to SEQ ID NO:34, or the complement thereof; and detecting the detectable label.

In some embodiments, the assay comprises: contacting the PIEZO1 nucleic acid molecule, or the complement thereof, in the biological sample with an alteration-specific probe comprising a detectable label, wherein the alteration-specific probe comprises a nucleotide sequence which hybridizes under stringent conditions to the nucleotide sequence of the PIEZO1 nucleic acid molecule, or the complement thereof, comprising: a uracil at a position corresponding to position 6,073 according to SEQ ID NO:15, or the complement thereof; and/or a thymine at a position corresponding to position 6,073 according to SEQ ID NO:35, or the complement thereof; and detecting the detectable label.

In some embodiments, the assay comprises: contacting the PIEZO1 nucleic acid molecule, or the complement thereof, in the biological sample with an alteration-specific probe comprising a detectable label, wherein the alteration-specific probe comprises a nucleotide sequence which hybridizes under stringent conditions to the nucleotide sequence of the PIEZO1 nucleic acid molecule, or the complement thereof, comprising: a uracil at a position corresponding to position 1,336 according to SEQ ID NO:16, or the complement thereof; and/or a thymine at a position corresponding to position 1,336 according to SEQ ID NO:36, or the complement thereof; and detecting the detectable label.

In some embodiments, the assay comprises: contacting the PIEZO1 nucleic acid molecule, or the complement thereof, in the biological sample with an alteration-specific probe comprising a detectable label, wherein the alteration-specific probe comprises a nucleotide sequence which hybridizes under stringent conditions to the nucleotide sequence of the PIEZO1 nucleic acid molecule, or the complement thereof, comprising: a uracil at a position corresponding to position 6,073 according to SEQ ID NO:17, or the complement thereof; and/or a thymine at a position corresponding to position 6,073 according to SEQ ID NO:37, or the complement thereof; and detecting the detectable label.

In some embodiments, the assay comprises: contacting the PIEZO1 nucleic acid molecule, or the complement thereof, in the biological sample with an alteration-specific probe comprising a detectable label, wherein the alteration-specific probe comprises a nucleotide sequence which hybridizes under stringent conditions to the nucleotide sequence of the PIEZO1 nucleic acid molecule, or the complement thereof, comprising: a uracil at a position corresponding to position 6,236 according to SEQ ID NO:18, or the complement thereof; and/or a thymine at a position corresponding to position 6,236 according to SEQ ID NO:38, or the complement thereof; and detecting the detectable label.

In some embodiments, the assay comprises: contacting the PIEZO1 nucleic acid molecule, or the complement thereof, in the biological sample with an alteration-specific probe comprising a detectable label, wherein the alteration-specific probe comprises a nucleotide sequence which hybridizes under stringent conditions to the nucleotide sequence of the PIEZO1 nucleic acid molecule, or the complement thereof, comprising: a uracil at a position corresponding to position 7,526 according to SEQ ID NO:19, or the complement thereof; and/or a thymine at a position corresponding to position 7,526 according to SEQ ID NO:39, or the complement thereof; and detecting the detectable label.

In some embodiments, the assay comprises: contacting the PIEZO1 nucleic acid molecule, or the complement thereof, in the biological sample with an alteration-specific probe comprising a detectable label, wherein the alteration-specific probe comprises a nucleotide sequence which hybridizes under stringent conditions to the nucleotide sequence of the PIEZO1 nucleic acid molecule, or the complement thereof, comprising: a uracil at a position corresponding to position 1,336 according to SEQ ID NO:20, or the complement thereof; and/or a thymine at a position corresponding to position 1,336 according to SEQ ID NO:40, or the complement thereof; and detecting the detectable label.

In some embodiments, the assay comprises: contacting the PIEZO1 nucleic acid molecule, or the complement thereof, in the biological sample with an alteration-specific probe comprising a detectable label, wherein the alteration-specific probe comprises a nucleotide sequence which hybridizes under stringent conditions to the nucleotide sequence of the PIEZO1 nucleic acid molecule, or the complement thereof, comprising: a uracil at a position corresponding to position 361 according to SEQ ID NO:21, or the complement thereof; and/or a thymine at a position corresponding to position 361 according to SEQ ID NO:41, or the complement thereof; and detecting the detectable label.

In some embodiments, the assay comprises: contacting the PIEZO1 nucleic acid molecule, or the complement thereof, in the biological sample with an alteration-specific probe comprising a detectable label, wherein the alteration-specific probe comprises a nucleotide sequence which hybridizes under stringent conditions to the nucleotide sequence of the PIEZO1 nucleic acid molecule, or the complement thereof, comprising: a uracil at a position corresponding to position 7,776 according to SEQ ID NO:22, or the complement thereof; and/or a thymine at a position corresponding to position 7,776 according to SEQ ID NO:42, or the complement thereof; and detecting the detectable label.

In some embodiments, the assay comprises: contacting the PIEZO1 genomic nucleic acid molecule, or the complement thereof, in the biological sample with an alteration-specific probe comprising a detectable label, wherein the alteration-specific probe comprises a nucleotide sequence which hybridizes under stringent conditions to the nucleotide sequence of the PIEZO1 genomic nucleic acid molecule, or the complement thereof, comprising: a thymine at a position corresponding to position 69,579 according to SEQ ID NO:2, or the complement thereof; and detecting the detectable label.

In some embodiments, the assay comprises: contacting the PIEZO1 mRNA molecule, or the complement thereof, in the biological sample with an alteration-specific probe comprising a detectable label, wherein the alteration-specific probe comprises a nucleotide sequence which hybridizes under stringent conditions to the nucleotide sequence of the PIEZO1 mRNA molecule, or the complement thereof, comprising: a uracil at a position corresponding to position 7,785 according to SEQ ID NO:13, or the complement thereof; a uracil at a position corresponding to position 1,095 according to SEQ ID NO:14, or the complement thereof; a uracil at a position corresponding to position 6,073 according to SEQ ID NO:15, or the complement thereof; a uracil at a position corresponding to position 1,336 according to SEQ ID NO:16, or the complement thereof; a uracil at a position corresponding to position 6,073 according to SEQ ID NO:17, or the complement thereof; a uracil at a position corresponding to position 6,236 according to SEQ ID NO:18, or the complement thereof; a uracil at a position corresponding to position 7,526 according to SEQ ID NO:19, or the complement thereof; a uracil at a position corresponding to position 1,336 according to SEQ ID NO:20, or the complement thereof; a uracil at a position corresponding to position 361 according to SEQ ID NO:21, or the complement thereof; or a uracil at a position corresponding to position 7,776 according to SEQ ID NO:22, or the complement thereof; and detecting the detectable label.

In some embodiments, the assay comprises: contacting the PIEZO1 cDNA molecule, or the complement thereof, produced from an mRNA molecule in the biological sample with an alteration-specific probe comprising a detectable label, wherein the alteration-specific probe comprises a nucleotide sequence which hybridizes under stringent conditions to the nucleotide sequence of the PIEZO1 cDNA molecule, or the complement thereof, comprising: a thymine at a position corresponding to position 7,785 according to SEQ ID NO:33, or the complement thereof; a thymine at a position corresponding to position 1,095 according to SEQ ID NO:34, or the complement thereof; a thymine at a position corresponding to position 6,073 according to SEQ ID NO:35, or the complement thereof; a thymine at a position corresponding to position 1,336 according to SEQ ID NO:36, or the complement thereof; a thymine at a position corresponding to position 6,073 according to SEQ ID NO:37, or the complement thereof; a thymine at a position corresponding to position 6,236 according to SEQ ID NO:38, or the complement thereof; a thymine at a position corresponding to position 7,526 according to SEQ ID NO:39, or the complement thereof; a thymine at a position corresponding to position 1,336 according to SEQ ID NO:40, or the complement thereof; a thymine at a position corresponding to position 361 according to SEQ ID NO:41, or the complement thereof; or a thymine at a position corresponding to position 7,776 according to SEQ ID NO:42, or the complement thereof; and detecting the detectable label.

In some embodiments, the PIEZO1 nucleic acid molecule is present within a cell obtained from the subject.

Alteration-specific polymerase chain reaction techniques can be used to detect mutations such as SNPs in a nucleotide sequence. Alteration-specific primers can be used because the DNA polymerase will not extend when a mismatch with the template is present.

In some embodiments, the assay comprises RNA sequencing (RNA-Seq). In some embodiments, the assays also comprise reverse transcribing mRNA into cDNA, such as by the reverse transcriptase polymerase chain reaction (RT-PCR).

In some embodiments, the methods utilize probes and primers of sufficient nucleotide length to bind to the target nucleotide sequence and specifically detect and/or identify a polynucleotide comprising a PIEZO1 variant genomic nucleic acid molecule, variant mRNA molecule, or variant cDNA molecule. The hybridization conditions or reaction conditions can be determined by the operator to achieve this result. The nucleotide length may be any length that is sufficient for use in a detection method of choice, including any assay described or exemplified herein. Such probes and primers can hybridize specifically to a target nucleotide sequence under high stringency hybridization conditions. Probes and primers may have complete nucleotide sequence identity of contiguous nucleotides within the target nucleotide sequence, although probes differing from the target nucleotide sequence and that retain the ability to specifically detect and/or identify a target nucleotide sequence may be designed by conventional methods. Probes and primers can have about 80%, about 85%, about 90%, about 91%, about 92%, about 93%, about 94%, about 95%, about 96%, about 97%, about 98%, about 99%, or 100% sequence identity or complementarity with the nucleotide sequence of the target nucleic acid molecule.

In some embodiments, to determine whether a PIEZO1 nucleic acid molecule (genomic nucleic acid molecule, mRNA molecule, or cDNA molecule), or complement thereof, within a biological sample comprises a nucleotide sequence comprising a thymine at a position corresponding to position 69,579 according to SEQ ID NO:2 (genomic nucleic acid molecule), a uracil at a position corresponding to position 7,785 according to SEQ ID NO:13 (mRNA molecule), or a thymine at a position corresponding to position 7,785 according to SEQ ID NO:33 (cDNA molecule), the biological sample can be subjected to an amplification method using a primer pair that includes a first primer derived from the 5′ flanking sequence adjacent to a thymine at a position corresponding to position 69,579 according to SEQ ID NO:2, a uracil at a position corresponding to position 7,785 according to SEQ ID NO:13, or a thymine at a position corresponding to position 7,785 according to SEQ ID NO:33, and a second primer derived from the 3′ flanking sequence adjacent to a thymine at a position corresponding to position 69,579 according to SEQ ID NO:2, a uracil at a position corresponding to position 7,785 according to SEQ ID NO:13, or a thymine at a position corresponding to position 7,785 according to SEQ ID NO:33 to produce an amplicon that is indicative of the presence of the SNP at positions encoding a thymine at a position corresponding to position 69,579 according to SEQ ID NO:2, a uracil at a position corresponding to position 7,785 according to SEQ ID NO:13, or a thymine at a position corresponding to position 7,785 according to SEQ ID NO:33. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a thymine at a position corresponding to position 69,579 according to SEQ ID NO:2, a uracil at a position corresponding to position 7,785 according to SEQ ID NO:13, or a thymine at a position corresponding to position 7,785 according to SEQ ID NO:33, and at least 1, 2, 3, 4, 5, 6, 7, 8, 9, 10, or more nucleotides on each side of positions comprising a thymine at a position corresponding to position 69,579 according to SEQ ID NO:2, a uracil at a position corresponding to position 7,785 according to SEQ ID NO:13, or a thymine at a position corresponding to position 7,785 according to SEQ ID NO:33.

In some embodiments, to determine whether a PIEZO1 nucleic acid molecule (genomic nucleic acid molecule, mRNA molecule, or cDNA molecule), or complement thereof, within a biological sample comprises a nucleotide sequence comprising, a uracil at a position corresponding to position 1,095 according to SEQ ID NO:14 (mRNA molecule), or a thymine at a position corresponding to position 1,095 according to SEQ ID NO:34 (cDNA molecule), the biological sample can be subjected to an amplification method using a primer pair that includes a first primer derived from the 5′ flanking sequence adjacent to a uracil at a position corresponding to position 1,095 according to SEQ ID NO:14, or a thymine at a position corresponding to position 1,095 according to SEQ ID NO:34, and a second primer derived from the 3′ flanking sequence adjacent to a uracil at a position corresponding to position 1,095 according to SEQ ID NO:14, or a thymine at a position corresponding to position 1,095 according to SEQ ID NO:34 to produce an amplicon that is indicative of the presence of the SNP at positions encoding a uracil at a position corresponding to position 1,095 according to SEQ ID NO:14, or a thymine at a position corresponding to position 1,095 according to SEQ ID NO:34. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a uracil at a position corresponding to position 1,095 according to SEQ ID NO:14, or a thymine at a position corresponding to position 1,095 according to SEQ ID NO:34, and at least 1, 2, 3, 4, 5, 6, 7, 8, 9, 10, or more nucleotides on each side of positions comprising a uracil at a position corresponding to position 1,095 according to SEQ ID NO:14, or a thymine at a position corresponding to position 1,095 according to SEQ ID NO:34.

In some embodiments, to determine whether a PIEZO1 nucleic acid molecule (genomic nucleic acid molecule, mRNA molecule, or cDNA molecule), or complement thereof, within a biological sample comprises a nucleotide sequence comprising a uracil at a position corresponding to position 6,073 according to SEQ ID NO:15 (mRNA molecule), or a thymine at a position corresponding to position 6,073 according to SEQ ID NO:35 (cDNA molecule), the biological sample can be subjected to an amplification method using a primer pair that includes a first primer derived from the 5′ flanking sequence adjacent to a uracil at a position corresponding to position 6,073 according to SEQ ID NO:15, or a thymine at a position corresponding to position 6,073 according to SEQ ID NO:35, and a second primer derived from the 3′ flanking sequence adjacent to a uracil at a position corresponding to position 6,073 according to SEQ ID NO:15, or a thymine at a position corresponding to position 6,073 according to SEQ ID NO:35 to produce an amplicon that is indicative of the presence of the SNP at positions encoding a uracil at a position corresponding to position 6,073 according to SEQ ID NO:15, or a thymine at a position corresponding to position 6,073 according to SEQ ID NO:35. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a uracil at a position corresponding to position 6,073 according to SEQ ID NO:15, or a thymine at a position corresponding to position 6,073 according to SEQ ID NO:35, and at least 1, 2, 3, 4, 5, 6, 7, 8, 9, 10, or more nucleotides on each side of positions comprising a uracil at a position corresponding to position 6,073 according to SEQ ID NO:15, or a thymine at a position corresponding to position 6,073 according to SEQ ID NO:35.

In some embodiments, to determine whether a PIEZO1 nucleic acid molecule (genomic nucleic acid molecule, mRNA molecule, or cDNA molecule), or complement thereof, within a biological sample comprises a nucleotide sequence comprising a uracil at a position corresponding to position 1,336 according to SEQ ID NO:16 (mRNA molecule), or a thymine at a position corresponding to position 1,336 according to SEQ ID NO:36 (cDNA molecule), the biological sample can be subjected to an amplification method using a primer pair that includes a first primer derived from the 5′ flanking sequence adjacent to a uracil at a position corresponding to position 1,336 according to SEQ ID NO:16, or a thymine at a position corresponding to position 1,336 according to SEQ ID NO:36, and a second primer derived from the 3′ flanking sequence adjacent to a uracil at a position corresponding to position 1,336 according to SEQ ID NO:16, or a thymine at a position corresponding to position 1,336 according to SEQ ID NO:36 to produce an amplicon that is indicative of the presence of the SNP at positions encoding a uracil at a position corresponding to position 1,336 according to SEQ ID NO:16, or a thymine at a position corresponding to position 1,336 according to SEQ ID NO:36. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a uracil at a position corresponding to position 1,336 according to SEQ ID NO:16, or a thymine at a position corresponding to position 1,336 according to SEQ ID NO:36, and at least 1, 2, 3, 4, 5, 6, 7, 8, 9, 10, or more nucleotides on each side of positions comprising a uracil at a position corresponding to position 1,336 according to SEQ ID NO:16, or a thymine at a position corresponding to position 1,336 according to SEQ ID NO:36.

In some embodiments, to determine whether a PIEZO1 nucleic acid molecule (genomic nucleic acid molecule, mRNA molecule, or cDNA molecule), or complement thereof, within a biological sample comprises a nucleotide sequence, a uracil at a position corresponding to position 6,073 according to SEQ ID NO:17 (mRNA molecule), or a thymine at a position corresponding to position 6,073 according to SEQ ID NO:37 (cDNA molecule), the biological sample can be subjected to an amplification method using a primer pair that includes a first primer derived from the 5′ flanking sequence adjacent to a uracil at a position corresponding to position 6,073 according to SEQ ID NO:17, or a thymine at a position corresponding to position 6,073 according to SEQ ID NO:37, and a second primer derived from the 3′ flanking sequence adjacent to a uracil at a position corresponding to position 6,073 according to SEQ ID NO:17, or a thymine at a position corresponding to position 6,073 according to SEQ ID NO:37 to produce an amplicon that is indicative of the presence of the SNP at positions encoding a uracil at a position corresponding to position 6,073 according to SEQ ID NO:17, or a thymine at a position corresponding to position 6,073 according to SEQ ID NO:37. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a uracil at a position corresponding to position 6,073 according to SEQ ID NO:17, or a thymine at a position corresponding to position 6,073 according to SEQ ID NO:37, and at least 1, 2, 3, 4, 5, 6, 7, 8, 9, 10, or more nucleotides on each side of positions comprising a uracil at a position corresponding to position 6,073 according to SEQ ID NO:17, or a thymine at a position corresponding to position 6,073 according to SEQ ID NO:37.

In some embodiments, to determine whether a PIEZO1 nucleic acid molecule (genomic nucleic acid molecule, mRNA molecule, or cDNA molecule), or complement thereof, within a biological sample comprises a nucleotide sequence comprising, a uracil at a position corresponding to position 6,236 according to SEQ ID NO:18 (mRNA molecule), or a thymine at a position corresponding to position 6,236 according to SEQ ID NO:38 (cDNA molecule), the biological sample can be subjected to an amplification method using a primer pair that includes a first primer derived from the 5′ flanking sequence adjacent to a uracil at a position corresponding to position 6,236 according to SEQ ID NO:18, or a thymine at a position corresponding to position 6,236 according to SEQ ID NO:38, and a second primer derived from the 3′ flanking sequence adjacent to a uracil at a position corresponding to position 6,236 according to SEQ ID NO:18, or a thymine at a position corresponding to position 6,236 according to SEQ ID NO:38 to produce an amplicon that is indicative of the presence of the SNP at positions encoding a uracil at a position corresponding to position 6,236 according to SEQ ID NO:18, or a thymine at a position corresponding to position 6,236 according to SEQ ID NO:38. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a uracil at a position corresponding to position 6,236 according to SEQ ID NO:18, or a thymine at a position corresponding to position 6,236 according to SEQ ID NO:38, and at least 1, 2, 3, 4, 5, 6, 7, 8, 9, 10, or more nucleotides on each side of positions comprising a uracil at a position corresponding to position 6,236 according to SEQ ID NO:18, or a thymine at a position corresponding to position 6,236 according to SEQ ID NO:38.

In some embodiments, to determine whether a PIEZO1 nucleic acid molecule (genomic nucleic acid molecule, mRNA molecule, or cDNA molecule), or complement thereof, within a biological sample comprises a nucleotide sequence a uracil at a position corresponding to position 7,526 according to SEQ ID NO:19 (mRNA molecule), or a thymine at a position corresponding to position 7,526 according to SEQ ID NO:39 (cDNA molecule), the biological sample can be subjected to an amplification method using a primer pair that includes a first primer derived from the 5′ flanking sequence adjacent to a uracil at a position corresponding to position 7,526 according to SEQ ID NO:19, or a thymine at a position corresponding to position 7,526 according to SEQ ID NO:39, and a second primer derived from the 3′ flanking sequence adjacent to a uracil at a position corresponding to position 7,526 according to SEQ ID NO:19, or a thymine at a position corresponding to position 7,526 according to SEQ ID NO:39 to produce an amplicon that is indicative of the presence of the SNP at positions encoding a uracil at a position corresponding to position 7,526 according to SEQ ID NO:19, or a thymine at a position corresponding to position 7,526 according to SEQ ID NO:39. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a uracil at a position corresponding to position 7,526 according to SEQ ID NO:19, or a thymine at a position corresponding to position 7,526 according to SEQ ID NO:39, and at least 1, 2, 3, 4, 5, 6, 7, 8, 9, 10, or more nucleotides on each side of positions comprising a uracil at a position corresponding to position 7,526 according to SEQ ID NO:19, or a thymine at a position corresponding to position 7,526 according to SEQ ID NO:39.

In some embodiments, to determine whether a PIEZO1 nucleic acid molecule (genomic nucleic acid molecule, mRNA molecule, or cDNA molecule), or complement thereof, within a biological sample comprises a nucleotide sequence a uracil at a position corresponding to position 1,336 according to SEQ ID NO:20 (mRNA molecule), or a thymine at a position corresponding to position 1,336 according to SEQ ID NO:40 (cDNA molecule), the biological sample can be subjected to an amplification method using a primer pair that includes a first primer derived from the 5′ flanking sequence adjacent to a uracil at a position corresponding to position 1,336 according to SEQ ID NO:20, or a thymine at a position corresponding to position 1,336 according to SEQ ID NO:40, and a second primer derived from the 3′ flanking sequence adjacent to a uracil at a position corresponding to position 1,336 according to SEQ ID NO:20, or a thymine at a position corresponding to position 1,336 according to SEQ ID NO:40 to produce an amplicon that is indicative of the presence of the SNP at positions encoding a uracil at a position corresponding to position 1,336 according to SEQ ID NO:20, or a thymine at a position corresponding to position 1,336 according to SEQ ID NO:40. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a uracil at a position corresponding to position 1,336 according to SEQ ID NO:20, or a thymine at a position corresponding to position 1,336 according to SEQ ID NO:40, and at least 1, 2, 3, 4, 5, 6, 7, 8, 9, 10, or more nucleotides on each side of positions comprising a uracil at a position corresponding to position 1,336 according to SEQ ID NO:20, or a thymine at a position corresponding to position 1,336 according to SEQ ID NO:40.

In some embodiments, to determine whether a PIEZO1 nucleic acid molecule (genomic nucleic acid molecule, mRNA molecule, or cDNA molecule), or complement thereof, within a biological sample comprises a nucleotide sequence a uracil at a position corresponding to position 361 according to SEQ ID NO:21 (mRNA molecule), or a thymine at a position corresponding to position 361 according to SEQ ID NO:41 (cDNA molecule), the biological sample can be subjected to an amplification method using a primer pair that includes a first primer derived from the 5′ flanking sequence adjacent to a uracil at a position corresponding to position 361 according to SEQ ID NO:21, or a thymine at a position corresponding to position 361 according to SEQ ID NO:41, and a second primer derived from the 3′ flanking sequence adjacent to a uracil at a position corresponding to position 361 according to SEQ ID NO:21, or a thymine at a position corresponding to position 361 according to SEQ ID NO:41 to produce an amplicon that is indicative of the presence of the SNP at positions encoding a uracil at a position corresponding to position 361 according to SEQ ID NO:21, or a thymine at a position corresponding to position 361 according to SEQ ID NO:41. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a uracil at a position corresponding to position 361 according to SEQ ID NO:21, or a thymine at a position corresponding to position 361 according to SEQ ID NO:41, and at least 1, 2, 3, 4, 5, 6, 7, 8, 9, 10, or more nucleotides on each side of positions comprising a uracil at a position corresponding to position 361 according to SEQ ID NO:21, or a thymine at a position corresponding to position 361 according to SEQ ID NO:41.

In some embodiments, to determine whether a PIEZO1 nucleic acid molecule (genomic nucleic acid molecule, mRNA molecule, or cDNA molecule), or complement thereof, within a biological sample comprises a nucleotide sequence comprising, a uracil at a position corresponding to position 7,776 according to SEQ ID NO:22 (mRNA molecule), or a thymine at a position corresponding to position 7,776 according to SEQ ID NO:42 (cDNA molecule), the biological sample can be subjected to an amplification method using a primer pair that includes a first primer derived from the 5′ flanking sequence adjacent to a uracil at a position corresponding to position 7,776 according to SEQ ID NO:22, or a thymine at a position corresponding to position 7,776 according to SEQ ID NO:42, and a second primer derived from the 3′ flanking sequence adjacent to a uracil at a position corresponding to position 7,776 according to SEQ ID NO:22, or a thymine at a position corresponding to position 7,776 according to SEQ ID NO:42 to produce an amplicon that is indicative of the presence of the SNP at positions encoding a uracil at a position corresponding to position 7,776 according to SEQ ID NO:22, or a thymine at a position corresponding to position 7,776 according to SEQ ID NO:42. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a uracil at a position corresponding to position 7,776 according to SEQ ID NO:22, or a thymine at a position corresponding to position 7,776 according to SEQ ID NO:42, and at least 1, 2, 3, 4, 5, 6, 7, 8, 9, 10, or more nucleotides on each side of positions comprising a uracil at a position corresponding to position 7,776 according to SEQ ID NO:22, or a thymine at a position corresponding to position 7,776 according to SEQ ID NO:42.

Similar amplicons can be generated from the mRNA and/or cDNA sequences. PCR primer pairs can be derived from a known sequence, for example, by using computer programs intended for that purpose, such as the PCR primer analysis tool in Vector NTI version 10 (Informax Inc., Bethesda Md.); PrimerSelect (DNASTAR Inc., Madison, Wis.); and Primer3 (Version 0.4.0.COPYRGT., 1991, Whitehead Institute for Biomedical Research, Cambridge, Mass.). Additionally, the sequence can be visually scanned and primers manually identified using known guidelines.

Illustrative examples of nucleic acid sequencing techniques include, but are not limited to, chain terminator (Sanger) sequencing and dye terminator sequencing. Other methods involve nucleic acid hybridization methods other than sequencing, including using labeled primers or probes directed against purified DNA, amplified DNA, and fixed cell preparations (fluorescence in situ hybridization (FISH)). In some methods, a target nucleic acid molecule may be amplified prior to or simultaneous with detection. Illustrative examples of nucleic acid amplification techniques include, but are not limited to, polymerase chain reaction (PCR), ligase chain reaction (LCR), strand displacement amplification (SDA), and nucleic acid sequence based amplification (NASBA). Other methods include, but are not limited to, ligase chain reaction, strand displacement amplification, and thermophilic SDA (tSDA).

In hybridization techniques, stringent conditions can be employed such that a probe or primer will specifically hybridize to its target. In some embodiments, a polynucleotide primer or probe under stringent conditions will hybridize to its target sequence to a detectably greater degree than to other non-target sequences, such as, at least 2-fold, at least 3-fold, at least 4-fold, or more over background, including over 10-fold over background. In some embodiments, a polynucleotide primer or probe under stringent conditions will hybridize to its target nucleotide sequence to a detectably greater degree than to other nucleotide sequences by at least 2-fold. In some embodiments, a polynucleotide primer or probe under stringent conditions will hybridize to its target nucleotide sequence to a detectably greater degree than to other nucleotide sequences by at least 3-fold. In some embodiments, a polynucleotide primer or probe under stringent conditions will hybridize to its target nucleotide sequence to a detectably greater degree than to other nucleotide sequences by at least 4-fold. In some embodiments, a polynucleotide primer or probe under stringent conditions will hybridize to its target nucleotide sequence to a detectably greater degree than to other nucleotide sequences by over 10-fold over background. Stringent conditions are sequence-dependent and will be different in different circumstances.

Appropriate stringency conditions which promote DNA hybridization, for example, 6× sodium chloride/sodium citrate (SSC) at about 45° C., followed by a wash of 2×SSC at 50° C., are known or can be found in Current Protocols in Molecular Biology, John Wiley & Sons, N.Y. (1989), 6.3.1-6.3.6. Typically, stringent conditions for hybridization and detection will be those in which the salt concentration is less than about 1.5 M Na⁺ ion, typically about 0.01 to 1.0 M Na⁺ ion concentration (or other salts) at pH 7.0 to 8.3 and the temperature is at least about 30° C. for short probes (such as, for example, 10 to 50 nucleotides) and at least about 60° C. for longer probes (such as, for example, greater than 50 nucleotides). Stringent conditions may also be achieved with the addition of destabilizing agents such as formamide. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

The present disclosure also provides methods of detecting the presence of a PIEZO1 predicted gain-of-function polypeptide comprising performing an assay on a biological sample obtained from the subject to determine whether a PIEZO1 polypeptide in the biological sample contains one or more variations that causes the polypeptide to have a gain-of-function (partial or complete) or predicted gain-of-function (partial or complete). The PIEZO1 predicted gain-of-function polypeptide can be any of the PIEZO1 predicted gain-of-function polypeptide described herein. In some embodiments, the methods detect the presence of PIEZO1 Pro2,510Leu, Pro2,024Leu, Pro2,079Leu, or Pro92Leu. In some embodiments, the methods detect the presence of PIEZO1 Pro2,510Leu.

In some embodiments, the methods comprise performing an assay on a biological sample obtained from a subject to determine whether a PIEZO1 polypeptide in the biological sample comprises: leucine at a position corresponding to position 2,510 according to SEQ ID NO:47; leucine at a position corresponding to position 2,024 according to SEQ ID NO:48; leucine at a position corresponding to position 2,079 according to SEQ ID NO:49; or leucine at a position corresponding to position 94 according to SEQ ID NO:50.

In some embodiments, the assay comprises sequencing at least a portion of the PIEZO1 polypeptide that comprises a position corresponding to: position 2,510 according to SEQ ID NO:47; position 2,024 according to SEQ ID NO:48; position 2,079 according to SEQ ID NO:49; or position 94 according to SEQ ID NO:50.

In some embodiments, the assay is an immunoassay for detecting the presence of a PIEZO1 polypeptide that comprises a position corresponding to: position 2,510 according to SEQ ID NO:47; position 2,024 according to SEQ ID NO:48; position 2,079 according to SEQ ID NO:49; or position 94 according to SEQ ID NO:50.

In some embodiments, when the subject does not have a PIEZO1 predicted gain-of-function polypeptide, the subject has an increased risk of developing varicose veins or any of saphenous varicose veins, veticular varicose veins, spider veins, or pregnancy-related varicose veins. In some embodiments, when the subject has a PIEZO1 predicted gain-of-function polypeptide, the subject has a decreased risk of developing varicose veins or any of saphenous varicose veins, veticular varicose veins, spider veins, or pregnancy-related varicose veins.

The present disclosure also provides isolated nucleic acid molecules that hybridize to PIEZO1 variant genomic nucleic acid molecules, PIEZO1 variant mRNA molecules, and/or PIEZO1 variant cDNA molecules (such as any of the genomic variant nucleic acid molecules, mRNA variant molecules, and cDNA variant molecules disclosed herein). In some embodiments, such isolated nucleic acid molecules hybridize to PIEZO1 variant nucleic acid molecules under stringent conditions. Such nucleic acid molecules can be used, for example, as probes, primers, alteration-specific probes, or alteration-specific primers as described or exemplified herein.

In some embodiments, the isolated nucleic acid molecules hybridize to a portion of the PIEZO1 nucleic acid molecule that includes a position corresponding to: position 69,579 according to SEQ ID NO:2, position 7,785 according to SEQ ID NO:13, or position 7,785 according to SEQ ID NO:33. In some embodiments, the isolated nucleic acid molecules hybridize to a portion of the PIEZO1 nucleic acid molecule that includes a position corresponding to: position 1,095 according to SEQ ID NO:14, or position 1,095 according to SEQ ID NO:34. In some embodiments, the isolated nucleic acid molecules hybridize to a portion of the PIEZO1 nucleic acid molecule that includes a position corresponding to: position 6,073 according to SEQ ID NO:15, or position 6,073 according to SEQ ID NO:35. In some embodiments, the isolated nucleic acid molecules hybridize to a portion of the PIEZO1 nucleic acid molecule that includes a position corresponding to: position 1,336 according to SEQ ID NO:16, or position 1,336 according to SEQ ID NO:36. In some embodiments, the isolated nucleic acid molecules hybridize to a portion of the PIEZO1 nucleic acid molecule that includes a position corresponding to: position 6,073 according to SEQ ID NO:17, or position 6,073 according to SEQ ID NO:37. In some embodiments, the isolated nucleic acid molecules hybridize to a portion of the PIEZO1 nucleic acid molecule that includes a position corresponding to: position 6,236 according to SEQ ID NO:18, or position 6,236 according to SEQ ID NO:38. In some embodiments, the isolated nucleic acid molecules hybridize to a portion of the PIEZO1 nucleic acid molecule that includes a position corresponding to: position 7,526 according to SEQ ID NO:19, or position 7,526 according to SEQ ID NO:39. In some embodiments, the isolated nucleic acid molecules hybridize to a portion of the PIEZO1 nucleic acid molecule that includes a position corresponding to: position 1,336 according to SEQ ID NO:20, or position 1,336 according to SEQ ID NO:20. In some embodiments, the isolated nucleic acid molecules hybridize to a portion of the PIEZO1 nucleic acid molecule that includes a position corresponding to: position 361 according to SEQ ID NO:21, or position 361 according to SEQ ID NO:21. In some embodiments, the isolated nucleic acid molecules hybridize to a portion of the PIEZO1 nucleic acid molecule that includes a position corresponding to: position 7,776 according to SEQ ID NO:22, or position 7,776 according to SEQ ID NO:42.

In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 200, at least about 300, at least about 400, at least about 500, at least about 600, at least about 700, at least about 800, at least about 900, at least about 1000, at least about 2000, at least about 3000, at least about 4000, or at least about 5000 nucleotides. In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, or at least about 25 nucleotides. In some embodiments, the isolated nucleic acid molecules comprise or consist of at least about 18 nucleotides. In some embodiments, the isolated nucleic acid molecules comprise or consists of at least about 15 nucleotides. In some embodiments, the isolated nucleic acid molecules consist of or comprise from about 10 to about 35, from about 10 to about 30, from about 10 to about 25, from about 12 to about 30, from about 12 to about 28, from about 12 to about 24, from about 15 to about 30, from about 15 to about 25, from about 18 to about 30, from about 18 to about 25, from about 18 to about 24, or from about 18 to about 22 nucleotides. In some embodiments, the isolated nucleic acid molecules consist of or comprise from about 18 to about 30 nucleotides. In some embodiments, the isolated nucleic acid molecules comprise or consist of at least about 15 nucleotides to at least about 35 nucleotides.

In some embodiments, the isolated alteration-specific probe or alteration-specific primer comprises at least about 15 nucleotides, wherein the alteration-specific probe or alteration-specific primer comprises a nucleotide sequence which is complementary to the nucleotide sequence of a portion of a PIEZO1 nucleic acid molecule encoding a PIEZO1 predicted gain-of-function polypeptide, or the complement thereof. In some embodiments, the portion comprises a position corresponding to: position 69,579 according to SEQ ID NO:2, or the complement thereof; position 7,785 according to SEQ ID NO:13, or the complement thereof; or position 7,785 according to SEQ ID NO:33, or the complement thereof. In some embodiments, the portion comprises a position corresponding to: position 1,095 according to SEQ ID NO:14, or the complement thereof; or position 1,095 according to SEQ ID NO:34, or the complement thereof. In some embodiments, the portion comprises a position corresponding to: position 6,073 according to SEQ ID NO:15, or the complement thereof; or position 6,073 according to SEQ ID NO:35, or the complement thereof. In some embodiments, the portion comprises a position corresponding to: position 1,336 according to SEQ ID NO:16, or the complement thereof; or position 1,336 according to SEQ ID NO:36, or the complement thereof. In some embodiments, the portion comprises a position corresponding to: position 6,073 according to SEQ ID NO:17, or the complement thereof; or position 6,073 according to SEQ ID NO:37, or the complement thereof. In some embodiments, the portion comprises a position corresponding to: position 6,236 according to SEQ ID NO:18, or the complement thereof; or position 6,236 according to SEQ ID NO:38, or the complement thereof. In some embodiments, the portion comprises a position corresponding to: position 7,526 according to SEQ ID NO:19, or the complement thereof; or position 7,526 according to SEQ ID NO:39, or the complement thereof. In some embodiments, the portion comprises a position corresponding to: position 1,336 according to SEQ ID NO:20, or the complement thereof; or position 1,336 according to SEQ ID NO:20, or the complement thereof. In some embodiments, the portion comprises a position corresponding to: position 361 according to SEQ ID NO:21, or the complement thereof; or position 361 according to SEQ ID NO:21, or the complement thereof. In some embodiments, the portion comprises a position corresponding to: position 7,776 according to SEQ ID NO:22, or the complement thereof; or position 7,776 according to SEQ ID NO:42, or the complement thereof.

In some embodiments, the isolated nucleic acid molecules hybridize to at least about 15 contiguous nucleotides of a nucleic acid molecule that is at least about 70%, at least about 75%, at least about 80%, at least about 85%, at least about 90%, at least about 95%, at least about 96%, at least about 97%, at least about 98%, at least about 99%, or 100% identical to PIEZO1 variant genomic nucleic acid molecules, PIEZO1 variant mRNA molecules, and/or PIEZO1 variant cDNA molecules. In some embodiments, the isolated nucleic acid molecules consist of or comprise from about 15 to about 100 nucleotides, or from about 15 to about 35 nucleotides. In some embodiments, the isolated nucleic acid molecules consist of or comprise from about 15 to about 100 nucleotides. In some embodiments, the isolated nucleic acid molecules consist of or comprise from about 15 to about 35 nucleotides.

In some embodiments, the isolated alteration-specific probes or alteration-specific primers comprise at least about 15 nucleotides, wherein the alteration-specific probe or alteration-specific primer comprises a nucleotide sequence which is complementary to a portion of a nucleotide sequence of a PIEZO1 variant nucleic acid molecule encoding a PIEZO1 predicted gain-of-function polypeptide, wherein the portion comprises a position corresponding to: position 69,579 according to SEQ ID NO:2, or the complement thereof; position 7,785 according to SEQ ID NO:13, or the complement thereof; or position 7,785 according to SEQ ID NO:33, or the complement thereof. In some embodiments, the portion comprises positions corresponding to: positions 69,578-69, 580 according to SEQ ID NO:2, or the complement thereof; positions 7,784-7,786 according to SEQ ID NO:13, or the complement thereof; and/or positions 7,784-7,786 according to SEQ ID NO:33, or the complement thereof.

In some embodiments, the isolated alteration-specific probes or alteration-specific primers comprise at least about 15 nucleotides, wherein the alteration-specific probe or alteration-specific primer comprises a nucleotide sequence which is complementary to a portion of a nucleotide sequence of a PIEZO1 variant nucleic acid molecule encoding a PIEZO1 predicted gain-of-function polypeptide, wherein the portion comprises a position corresponding to: position 1,095 according to SEQ ID NO:14, or the complement thereof; or position 1,095 according to SEQ ID NO:34, or the complement thereof. In some embodiments, the portion comprises positions corresponding to: positions 1,094-1,096 according to SEQ ID NO:14, or the complement thereof; and/or positions 1,094-1,096 according to SEQ ID NO:34, or the complement thereof.

In some embodiments, the isolated alteration-specific probes or alteration-specific primers comprise at least about 15 nucleotides, wherein the alteration-specific probe or alteration-specific primer comprises a nucleotide sequence which is complementary to a portion of a nucleotide sequence of a PIEZO1 variant nucleic acid molecule encoding a PIEZO1 predicted gain-of-function polypeptide, wherein the portion comprises a position corresponding to: position 6,073 according to SEQ ID NO:15, or the complement thereof; or position 6,073 according to SEQ ID NO:35, or the complement thereof. In some embodiments, the portion comprises positions corresponding to: positions 6,072-6,074 according to SEQ ID NO:15, or the complement thereof; and/or positions 6,072-6,074 according to SEQ ID NO:35, or the complement thereof.

In some embodiments, the isolated alteration-specific probes or alteration-specific primers comprise at least about 15 nucleotides, wherein the alteration-specific probe or alteration-specific primer comprises a nucleotide sequence which is complementary to a portion of a nucleotide sequence of a PIEZO1 variant nucleic acid molecule encoding a PIEZO1 predicted gain-of-function polypeptide, wherein the portion comprises a position corresponding to: position 1,336 according to SEQ ID NO:16, or the complement thereof; or position 1,336 according to SEQ ID NO:36, or the complement thereof. In some embodiments, the portion comprises positions corresponding to: positions 1,335-1,337 according to SEQ ID NO:16, or the complement thereof; and/or positions 1,335-1,337 according to SEQ ID NO:36, or the complement thereof.

In some embodiments, the isolated alteration-specific probes or alteration-specific primers comprise at least about 15 nucleotides, wherein the alteration-specific probe or alteration-specific primer comprises a nucleotide sequence which is complementary to a portion of a nucleotide sequence of a PIEZO1 variant nucleic acid molecule encoding a PIEZO1 predicted gain-of-function polypeptide, wherein the portion comprises a position corresponding to: position 6,073 according to SEQ ID NO:17, or the complement thereof; or position 6,073 according to SEQ ID NO:37, or the complement thereof. In some embodiments, the portion comprises positions corresponding to: positions 6,072-6,074 according to SEQ ID NO:17, or the complement thereof; and/or positions 6,072-6,074 according to SEQ ID NO:37, or the complement thereof.

In some embodiments, the isolated alteration-specific probes or alteration-specific primers comprise at least about 15 nucleotides, wherein the alteration-specific probe or alteration-specific primer comprises a nucleotide sequence which is complementary to a portion of a nucleotide sequence of a PIEZO1 variant nucleic acid molecule encoding a PIEZO1 predicted gain-of-function polypeptide, wherein the portion comprises a position corresponding to: position 6,236 according to SEQ ID NO:18, or the complement thereof; or position 6,236 according to SEQ ID NO:38, or the complement thereof. In some embodiments, the portion comprises positions corresponding to: positions 6,235-6,237 according to SEQ ID NO:18, or the complement thereof; and/or positions 6,235-6,237 according to SEQ ID NO:38, or the complement thereof.

In some embodiments, the isolated alteration-specific probes or alteration-specific primers comprise at least about 15 nucleotides, wherein the alteration-specific probe or alteration-specific primer comprises a nucleotide sequence which is complementary to a portion of a nucleotide sequence of a PIEZO1 variant nucleic acid molecule encoding a PIEZO1 predicted gain-of-function polypeptide, wherein the portion comprises a position corresponding to: position 7,526 according to SEQ ID NO:19, or the complement thereof; or position 7,526 according to SEQ ID NO:39, or the complement thereof. In some embodiments, the portion comprises positions corresponding to: positions 7,525-7,527 according to SEQ ID NO:19, or the complement thereof; and/or positions 7,525-7,527 according to SEQ ID NO:39, or the complement thereof.

In some embodiments, the isolated alteration-specific probes or alteration-specific primers comprise at least about 15 nucleotides, wherein the alteration-specific probe or alteration-specific primer comprises a nucleotide sequence which is complementary to a portion of a nucleotide sequence of a PIEZO1 variant nucleic acid molecule encoding a PIEZO1 predicted gain-of-function polypeptide, wherein the portion comprises a position corresponding to: position 1,336 according to SEQ ID NO:20 or the complement thereof; or position 1,336 according to SEQ ID NO:40, or the complement thereof. In some embodiments, the portion comprises positions corresponding to: positions 1,335-1,337 according to SEQ ID NO:20, or the complement thereof; and/or positions 1,335-1,337 according to SEQ ID NO:40, or the complement thereof.

In some embodiments, the isolated alteration-specific probes or alteration-specific primers comprise at least about 15 nucleotides, wherein the alteration-specific probe or alteration-specific primer comprises a nucleotide sequence which is complementary to a portion of a nucleotide sequence of a PIEZO1 variant nucleic acid molecule encoding a PIEZO1 predicted gain-of-function polypeptide, wherein the portion comprises a position corresponding to: position 1,336 according to SEQ ID NO:21 or the complement thereof; or position 1,336 according to SEQ ID NO:41, or the complement thereof. In some embodiments, the portion comprises positions corresponding to: positions 360-362 according to SEQ ID NO:21, or the complement thereof; and/or positions 360-362 according to SEQ ID NO:41, or the complement thereof.

In some embodiments, the isolated alteration-specific probes or alteration-specific primers comprise at least about 15 nucleotides, wherein the alteration-specific probe or alteration-specific primer comprises a nucleotide sequence which is complementary to a portion of a nucleotide sequence of a PIEZO1 variant nucleic acid molecule encoding a PIEZO1 predicted gain-of-function polypeptide, wherein the portion comprises a position corresponding to: position 7,776 according to SEQ ID NO:22, or the complement thereof; or position 7,776 according to SEQ ID NO:42, or the complement thereof. In some embodiments, the portion comprises positions corresponding to: positions 7,775-7,777 according to SEQ ID NO:22, or the complement thereof; and/or positions 7,775-7,777 according to SEQ ID NO:42, or the complement thereof.

In some embodiments, the isolated alteration-specific probe or alteration-specific primer comprises at least about 15 nucleotides, wherein the alteration-specific probe or alteration-specific primer comprises a nucleotide sequence which is complementary to the nucleotide sequence of a portion of a PIEZO1 nucleic acid molecule encoding a PIEZO1 predicted gain-of-function polypeptide, or the complement thereof. In some embodiments, the portion comprises a position corresponding to: position 69,579 according to SEQ ID NO:2, or the complement thereof.

In some embodiments, the portion comprises positions corresponding to: positions 69,578-69, 580 according to SEQ ID NO:2, or the complement thereof.

In some embodiments, the portion comprises a position corresponding to: position 7,785 according to SEQ ID NO:13, or the complement thereof; position 1,095 according to SEQ ID NO:14, or the complement thereof; position 6,073 according to SEQ ID NO:15, or the complement thereof; position 1,336 according to SEQ ID NO:16, or the complement thereof; position 6,073 according to SEQ ID NO:17, or the complement thereof; position 6,236 according to SEQ ID NO:18, or the complement thereof; position 7,526 according to SEQ ID NO:19, or the complement thereof; position 1,336 according to SEQ ID NO:20, or the complement thereof; position corresponding to position 361 according to SEQ ID NO:21, or the complement thereof; or position 7,776 according to SEQ ID NO:22, or the complement thereof.

In some embodiments, the portion comprises positions corresponding to: positions 7,784-7,786 according to SEQ ID NO:13, or the complement thereof; positions 1,094-1,096 according to SEQ ID NO:14, or the complement thereof; positions 6,072-6,074 according to SEQ ID NO:15, or the complement thereof; positions 1,335-1,337 according to SEQ ID NO:16, or the complement thereof; positions 6,072-6,074 according to SEQ ID NO:17, or the complement thereof; positions 6,235-6,237 according to SEQ ID NO:18, or the complement thereof; positions 7,525-7,527 according to SEQ ID NO:19, or the complement thereof; or positions 1,335-1,337 according to SEQ ID NO:20, or the complement thereof; positions 360-362 according to SEQ ID NO:21, or the complement thereof; or positions 7,775-7,777 according to SEQ ID NO:22, or the complement thereof.

In some embodiments, the portion comprises a position corresponding to: position 7,785 according to SEQ ID NO:33, or the complement thereof; position 1,095 according to SEQ ID NO:34, or the complement thereof; position 6,073 according to SEQ ID NO:35, or the complement thereof; position 1,336 according to SEQ ID NO:36, or the complement thereof; position 6,073 according to SEQ ID NO:37, or the complement thereof; position 6,236 according to SEQ ID NO:38, or the complement thereof; position 7,526 according to SEQ ID NO:39, or the complement thereof; position 1,336 according to SEQ ID NO:40, or the complement thereof; position 361 according to SEQ ID NO:41, or the complement thereof; or position 7,776 according to SEQ ID NO:42, or the complement thereof.

In some embodiments, the portion comprises positions corresponding to: positions 7,784-7,786 according to SEQ ID NO:33, or the complement thereof; positions 1,094-1,096 according to SEQ ID NO:34, or the complement thereof; positions 6,072-6,074 according to SEQ ID NO:35, or the complement thereof; positions 1,335-1,337 according to SEQ ID NO:36, or the complement thereof; positions 6,072-6,074 according to SEQ ID NO:37, or the complement thereof; positions 6,235-6,237 according to SEQ ID NO:38, or the complement thereof; positions 7,525-7,527 according to SEQ ID NO:39, or the complement thereof; positions 1,335-1,337 according to SEQ ID NO:40, or the complement thereof; positions 360-362 according to SEQ ID NO:41, or the complement thereof; or positions 7,775-7,777 according to SEQ ID NO:42, or the complement thereof.

In some embodiments, the alteration-specific probes and alteration-specific primers comprise DNA. In some embodiments, the alteration-specific probes and alteration-specific primers comprise RNA.

In some embodiments, the probes and primers described herein (including alteration-specific probes and alteration-specific primers) have a nucleotide sequence that specifically hybridizes to any of the nucleic acid molecules disclosed herein, or the complement thereof. In some embodiments, the probes and primers specifically hybridize to any of the nucleic acid molecules disclosed herein under stringent conditions.

In some embodiments, the primers, including alteration-specific primers, can be used in second generation sequencing or high throughput sequencing. In some instances, the primers, including alteration-specific primers, can be modified. In particular, the primers can comprise various modifications that are used at different steps of, for example, Massive Parallel Signature Sequencing (MPSS), Polony sequencing, and 454 Pyrosequencing. Modified primers can be used at several steps of the process, including biotinylated primers in the cloning step and fluorescently labeled primers used at the bead loading step and detection step. Polony sequencing is generally performed using a paired-end tags library wherein each molecule of DNA template is about 135 bp in length. Biotinylated primers are used at the bead loading step and emulsion PCR. Fluorescently labeled degenerate nonamer oligonucleotides are used at the detection step. An adaptor can contain a 5′-biotin tag for immobilization of the DNA library onto streptavidin-coated beads.

The probes and primers described herein can be used to detect a nucleotide variation within any of the PIEZO1 variant genomic nucleic acid molecules, PIEZO1 variant mRNA molecules, and/or PIEZO1 variant cDNA molecules disclosed herein. The primers described herein can be used to amplify the PIEZO1 variant genomic nucleic acid molecules, PIEZO1 variant mRNA molecules, or PIEZO1 variant cDNA molecules, or a fragment thereof.

The present disclosure also provides pairs of primers comprising any of the primers described above. For example, if one of the primers' 3′-ends hybridizes to cytosine at a position corresponding to position 69,579 according to SEQ ID NO:1 (rather than a thymine) in a particular PIEZO1 nucleic acid molecule, then the presence of the amplified fragment would indicate the presence of a PIEZO1 reference genomic nucleic acid molecule. Conversely, if one of the primers' 3′-ends hybridizes to a thymine at a position corresponding to position 69,579 according to SEQ ID NO:2 (rather than cytosine) in a particular PIEZO1 nucleic acid molecule, then the presence of the amplified fragment would indicate the presence of the PIEZO1 variant genomic nucleic acid molecule. In some embodiments, the nucleotide of the primer complementary to the thymine at a position corresponding to position 69,579 according to SEQ ID NO:2 can be at the 3′ end of the primer. In addition, if one of the primers' 3′-ends hybridizes to a cytosine at a position corresponding to position 7,785 according to SEQ ID NO:3 (rather than a uracil) in a particular PIEZO1 nucleic acid molecule, then the presence of the amplified fragment would indicate the presence of a PIEZO1 reference mRNA molecule. Conversely, if one of the primers' 3′-ends hybridizes to a uracil at a position corresponding to position 7,785 according to SEQ ID NO:13 (rather than a cytosine) in a particular PIEZO1 mRNA molecule, then the presence of the amplified fragment would indicate the presence of the PIEZO1 variant mRNA molecule. In some embodiments, the nucleotide of the primer complementary to the uracil at a position corresponding to position 7,785 according to SEQ ID NO:13 can be at the 3′ end of the primer. In addition, if one of the primers' 3′-ends hybridizes to a cytosine at a position corresponding to position 7,785 according to SEQ ID NO:23 (rather than a thymine) in a particular PIEZO1 nucleic acid molecule, then the presence of the amplified fragment would indicate the presence of a PIEZO1 reference cDNA molecule. Conversely, if one of the primers' 3′-ends hybridizes to a thymine at a position corresponding to position 7,785 according to SEQ ID NO:33 (rather than a cytosine) in a particular PIEZO1 cDNA molecule, then the presence of the amplified fragment would indicate the presence of the PIEZO1 variant cDNA molecule. In some embodiments, the nucleotide of the primer complementary to the thymine at a position corresponding to position 7,785 according to SEQ ID NO:33 can be at the 3′ end of the primer.

The present disclosure also provides pairs of primers comprising any of the primers described above. For example, if one of the primers' 3′-ends hybridizes to a cytosine at a position corresponding to position 1,095 according to SEQ ID NO:4 (rather than a uracil) in a particular PIEZO1 nucleic acid molecule, then the presence of the amplified fragment would indicate the presence of a PIEZO1 reference mRNA molecule. Conversely, if one of the primers' 3′-ends hybridizes to a uracil at a position corresponding to position 1,095 according to SEQ ID NO:14 (rather than a cytosine) in a particular PIEZO1 mRNA molecule, then the presence of the amplified fragment would indicate the presence of the PIEZO1 variant mRNA molecule. In some embodiments, the nucleotide of the primer complementary to the uracil at a position corresponding to position 1,095 according to SEQ ID NO:14 can be at the 3′ end of the primer. In addition, if one of the primers' 3′-ends hybridizes to a cytosine at a position corresponding to position 1,095 according to SEQ ID NO:24 (rather than a thymine) in a particular PIEZO1 nucleic acid molecule, then the presence of the amplified fragment would indicate the presence of a PIEZO1 reference cDNA molecule. Conversely, if one of the primers' 3′-ends hybridizes to a thymine at a position corresponding to position 1,095 according to SEQ ID NO:34 (rather than a cytosine) in a particular PIEZO1 cDNA molecule, then the presence of the amplified fragment would indicate the presence of the PIEZO1 variant cDNA molecule. In some embodiments, the nucleotide of the primer complementary to the thymine at a position corresponding to position 1,095 according to SEQ ID NO:34 can be at the 3′ end of the primer.

The present disclosure also provides pairs of primers comprising any of the primers described above. For example, if one of the primers' 3′-ends hybridizes to a cytosine at a position corresponding to position 6,073 according to SEQ ID NO:5 (rather than a uracil) in a particular PIEZO1 nucleic acid molecule, then the presence of the amplified fragment would indicate the presence of a PIEZO1 reference mRNA molecule. Conversely, if one of the primers' 3′-ends hybridizes to a uracil at a position corresponding to position 6,073 according to SEQ ID NO:15 (rather than a cytosine) in a particular PIEZO1 mRNA molecule, then the presence of the amplified fragment would indicate the presence of the PIEZO1 variant mRNA molecule. In some embodiments, the nucleotide of the primer complementary to the uracil at a position corresponding to position 6,073 according to SEQ ID NO:15 can be at the 3′ end of the primer. In addition, if one of the primers' 3′-ends hybridizes to a cytosine at a position corresponding to position 6,073 according to SEQ ID NO:25 (rather than a thymine) in a particular PIEZO1 nucleic acid molecule, then the presence of the amplified fragment would indicate the presence of a PIEZO1 reference cDNA molecule. Conversely, if one of the primers' 3′-ends hybridizes to a thymine at a position corresponding to position 6,073 according to SEQ ID NO:35 (rather than a cytosine) in a particular PIEZO1 cDNA molecule, then the presence of the amplified fragment would indicate the presence of the PIEZO1 variant cDNA molecule. In some embodiments, the nucleotide of the primer complementary to the thymine at a position corresponding to position 6,073 according to SEQ ID NO:35 can be at the 3′ end of the primer.

The present disclosure also provides pairs of primers comprising any of the primers described above. For example, if one of the primers' 3′-ends hybridizes to a cytosine at a position corresponding to position 1,336 according to SEQ ID NO:6 (rather than a uracil) in a particular PIEZO1 nucleic acid molecule, then the presence of the amplified fragment would indicate the presence of a PIEZO1 reference mRNA molecule. Conversely, if one of the primers' 3′-ends hybridizes to a uracil at a position corresponding to position 1,336 according to SEQ ID NO:16 (rather than a cytosine) in a particular PIEZO1 mRNA molecule, then the presence of the amplified fragment would indicate the presence of the PIEZO1 variant mRNA molecule. In some embodiments, the nucleotide of the primer complementary to the uracil at a position corresponding to position 1,336 according to SEQ ID NO:16 can be at the 3′ end of the primer. In addition, if one of the primers' 3′-ends hybridizes to a cytosine at a position corresponding to position 1,336 according to SEQ ID NO:26 (rather than a thymine) in a particular PIEZO1 nucleic acid molecule, then the presence of the amplified fragment would indicate the presence of a PIEZO1 reference cDNA molecule. Conversely, if one of the primers' 3′-ends hybridizes to a thymine at a position corresponding to position 1,336 according to SEQ ID NO:36 (rather than a cytosine) in a particular PIEZO1 cDNA molecule, then the presence of the amplified fragment would indicate the presence of the PIEZO1 variant cDNA molecule. In some embodiments, the nucleotide of the primer complementary to the thymine at a position corresponding to position 1,336 according to SEQ ID NO:36 can be at the 3′ end of the primer.

The present disclosure also provides pairs of primers comprising any of the primers described above. For example, if one of the primers' 3′-ends hybridizes to a cytosine at a position corresponding to position 6,073 according to SEQ ID NO:7 (rather than a uracil) in a particular PIEZO1 nucleic acid molecule, then the presence of the amplified fragment would indicate the presence of a PIEZO1 reference mRNA molecule. Conversely, if one of the primers' 3′-ends hybridizes to a uracil at a position corresponding to position 6,073 according to SEQ ID NO:17 (rather than a cytosine) in a particular PIEZO1 mRNA molecule, then the presence of the amplified fragment would indicate the presence of the PIEZO1 variant mRNA molecule. In some embodiments, the nucleotide of the primer complementary to the uracil at a position corresponding to position 6,073 according to SEQ ID NO:17 can be at the 3′ end of the primer. In addition, if one of the primers' 3′-ends hybridizes to a cytosine at a position corresponding to position 6,073 according to SEQ ID NO:27 (rather than a thymine) in a particular PIEZO1 nucleic acid molecule, then the presence of the amplified fragment would indicate the presence of a PIEZO1 reference cDNA molecule. Conversely, if one of the primers' 3′-ends hybridizes to a thymine at a position corresponding to position 6,073 according to SEQ ID NO:37 (rather than a cytosine) in a particular PIEZO1 cDNA molecule, then the presence of the amplified fragment would indicate the presence of the PIEZO1 variant cDNA molecule. In some embodiments, the nucleotide of the primer complementary to the thymine at a position corresponding to position 6,073 according to SEQ ID NO:37 can be at the 3′ end of the primer.

The present disclosure also provides pairs of primers comprising any of the primers described above. For example, if one of the primers' 3′-ends hybridizes to a cytosine at a position corresponding to position 6,236 according to SEQ ID NO:8 (rather than a uracil) in a particular PIEZO1 nucleic acid molecule, then the presence of the amplified fragment would indicate the presence of a PIEZO1 reference mRNA molecule. Conversely, if one of the primers' 3′-ends hybridizes to a uracil at a position corresponding to position 6,236 according to SEQ ID NO:18 (rather than a cytosine) in a particular PIEZO1 mRNA molecule, then the presence of the amplified fragment would indicate the presence of the PIEZO1 variant mRNA molecule. In some embodiments, the nucleotide of the primer complementary to the uracil at a position corresponding to position 6,236 according to SEQ ID NO:18 can be at the 3′ end of the primer. In addition, if one of the primers' 3′-ends hybridizes to a cytosine at a position corresponding to position 6,236 according to SEQ ID NO:28 (rather than a thymine) in a particular PIEZO1 nucleic acid molecule, then the presence of the amplified fragment would indicate the presence of a PIEZO1 reference cDNA molecule. Conversely, if one of the primers' 3′-ends hybridizes to a thymine at a position corresponding to position 6,236 according to SEQ ID NO:38 (rather than a cytosine) in a particular PIEZO1 cDNA molecule, then the presence of the amplified fragment would indicate the presence of the PIEZO1 variant cDNA molecule. In some embodiments, the nucleotide of the primer complementary to the thymine at a position corresponding to position 6,236 according to SEQ ID NO:38 can be at the 3′ end of the primer.

The present disclosure also provides pairs of primers comprising any of the primers described above. For example, if one of the primers' 3′-ends hybridizes to a cytosine at a position corresponding to position 7,526 according to SEQ ID NO:9 (rather than a uracil) in a particular PIEZO1 nucleic acid molecule, then the presence of the amplified fragment would indicate the presence of a PIEZO1 reference mRNA molecule. Conversely, if one of the primers' 3′-ends hybridizes to a uracil at a position corresponding to position 7,526 according to SEQ ID NO:19 (rather than a cytosine) in a particular PIEZO1 mRNA molecule, then the presence of the amplified fragment would indicate the presence of the PIEZO1 variant mRNA molecule. In some embodiments, the nucleotide of the primer complementary to the uracil at a position corresponding to position 7,526 according to SEQ ID NO:19 can be at the 3′ end of the primer. In addition, if one of the primers' 3′-ends hybridizes to a cytosine at a position corresponding to position 7,526 according to SEQ ID NO:29 (rather than a thymine) in a particular PIEZO1 nucleic acid molecule, then the presence of the amplified fragment would indicate the presence of a PIEZO1 reference cDNA molecule. Conversely, if one of the primers' 3′-ends hybridizes to a thymine at a position corresponding to position 7,526 according to SEQ ID NO:39 (rather than a cytosine) in a particular PIEZO1 cDNA molecule, then the presence of the amplified fragment would indicate the presence of the PIEZO1 variant cDNA molecule. In some embodiments, the nucleotide of the primer complementary to the thymine at a position corresponding to position 7,526 according to SEQ ID NO:39 can be at the 3′ end of the primer.

The present disclosure also provides pairs of primers comprising any of the primers described above. For example, if one of the primers' 3′-ends hybridizes to a cytosine at a position corresponding to position 1,336 according to SEQ ID NO:10 (rather than a uracil) in a particular PIEZO1 nucleic acid molecule, then the presence of the amplified fragment would indicate the presence of a PIEZO1 reference mRNA molecule. Conversely, if one of the primers' 3′-ends hybridizes to a uracil at a position corresponding to position 1,336 according to SEQ ID NO:20 (rather than a cytosine) in a particular PIEZO1 mRNA molecule, then the presence of the amplified fragment would indicate the presence of the PIEZO1 variant mRNA molecule. In some embodiments, the nucleotide of the primer complementary to the uracil at a position corresponding to position 1,336 according to SEQ ID NO:20 can be at the 3′ end of the primer. In addition, if one of the primers' 3′-ends hybridizes to a cytosine at a position corresponding to position 1,336 according to SEQ ID NO:30 (rather than a thymine) in a particular PIEZO1 nucleic acid molecule, then the presence of the amplified fragment would indicate the presence of a PIEZO1 reference cDNA molecule. Conversely, if one of the primers' 3′-ends hybridizes to a thymine at a position corresponding to position 1,336 according to SEQ ID NO:40 (rather than a cytosine) in a particular PIEZO1 cDNA molecule, then the presence of the amplified fragment would indicate the presence of the PIEZO1 variant cDNA molecule. In some embodiments, the nucleotide of the primer complementary to the thymine at a position corresponding to position 1,336 according to SEQ ID NO:40 can be at the 3′ end of the primer.

The present disclosure also provides pairs of primers comprising any of the primers described above. For example, if one of the primers' 3′-ends hybridizes to a cytosine at a position corresponding to position 361 according to SEQ ID NO:11 (rather than a uracil) in a particular PIEZO1 nucleic acid molecule, then the presence of the amplified fragment would indicate the presence of a PIEZO1 reference mRNA molecule. Conversely, if one of the primers' 3′-ends hybridizes to a uracil at a position corresponding to position 361 according to SEQ ID NO:21 (rather than a cytosine) in a particular PIEZO1 mRNA molecule, then the presence of the amplified fragment would indicate the presence of the PIEZO1 variant mRNA molecule. In some embodiments, the nucleotide of the primer complementary to the uracil at a position corresponding to position 361 according to SEQ ID NO:21 can be at the 3′ end of the primer. In addition, if one of the primers' 3′-ends hybridizes to a cytosine at a position corresponding to position 361 according to SEQ ID NO:31 (rather than a thymine) in a particular PIEZO1 nucleic acid molecule, then the presence of the amplified fragment would indicate the presence of a PIEZO1 reference cDNA molecule. Conversely, if one of the primers' 3′-ends hybridizes to a thymine at a position corresponding to position 361 according to SEQ ID NO:41 (rather than a cytosine) in a particular PIEZO1 cDNA molecule, then the presence of the amplified fragment would indicate the presence of the PIEZO1 variant cDNA molecule. In some embodiments, the nucleotide of the primer complementary to the thymine at a position corresponding to position 361 according to SEQ ID NO:41 can be at the 3′ end of the primer.

The present disclosure also provides pairs of primers comprising any of the primers described above. For example, if one of the primers' 3′-ends hybridizes to a cytosine at a position corresponding to position 7,776 according to SEQ ID NO:12 (rather than a uracil) in a particular PIEZO1 nucleic acid molecule, then the presence of the amplified fragment would indicate the presence of a PIEZO1 reference mRNA molecule. Conversely, if one of the primers' 3′-ends hybridizes to a uracil at a position corresponding to position 7,776 according to SEQ ID NO:22 (rather than a cytosine) in a particular PIEZO1 mRNA molecule, then the presence of the amplified fragment would indicate the presence of the PIEZO1 variant mRNA molecule. In some embodiments, the nucleotide of the primer complementary to the uracil at a position corresponding to position 7,776 according to SEQ ID NO:22 can be at the 3′ end of the primer. In addition, if one of the primers' 3′-ends hybridizes to a cytosine at a position corresponding to position 7,776 according to SEQ ID NO:32 (rather than a thymine) in a particular PIEZO1 nucleic acid molecule, then the presence of the amplified fragment would indicate the presence of a PIEZO1 reference cDNA molecule. Conversely, if one of the primers' 3′-ends hybridizes to a thymine at a position corresponding to position 7,776 according to SEQ ID NO:42 (rather than a cytosine) in a particular PIEZO1 cDNA molecule, then the presence of the amplified fragment would indicate the presence of the PIEZO1 variant cDNA molecule. In some embodiments, the nucleotide of the primer complementary to the thymine at a position corresponding to position 7,776 according to SEQ ID NO:42 can be at the 3′ end of the primer.

In the context of the present disclosure “specifically hybridizes” means that the probe or primer (such as, for example, the alteration-specific probe or alteration-specific primer) does not hybridize to a nucleotide sequence encoding a PIEZO1 reference genomic nucleic acid molecule, a PIEZO1 reference mRNA molecule, and/or a PIEZO1 reference cDNA molecule.

In any of the embodiments described throughout the present disclosure, the probes (such as, for example, an alteration-specific probe) can comprise a label. In some embodiments, the label is a fluorescent label, a radiolabel, or biotin.

The present disclosure also provides supports comprising a substrate to which any one or more of the probes disclosed herein is attached. Solid supports are solid-state substrates or supports with which molecules, such as any of the probes disclosed herein, can be associated. A form of solid support is an array. Another form of solid support is an array detector. An array detector is a solid support to which multiple different probes have been coupled in an array, grid, or other organized pattern. A form for a solid-state substrate is a microtiter dish, such as a standard 96-well type. In some embodiments, a multiwell glass slide can be employed that normally contains one array per well. In some embodiments, the support is a microarray.

The present disclosure also provides molecular complexes comprising or consisting of any of the PIEZO1 nucleic acid molecules (genomic nucleic acid molecules, mRNA molecules, or cDNA molecules), or complement thereof, described herein and any of the alteration-specific primers or alteration-specific probes described herein. In some embodiments, the PIEZO1 nucleic acid molecules (genomic nucleic acid molecules, mRNA molecules, or cDNA molecules), or complement thereof, in the molecular complexes are single-stranded. In some embodiments, the PIEZO1 nucleic acid molecule is any of the genomic nucleic acid molecules described herein. In some embodiments, the PIEZO1 nucleic acid molecule is any of the mRNA molecules described herein. In some embodiments, the PIEZO1 nucleic acid molecule is any of the cDNA molecules described herein. In some embodiments, the molecular complex comprises or consists of any of the PIEZO1 nucleic acid molecules (genomic nucleic acid molecules, mRNA molecules, or cDNA molecules), or complement thereof, described herein and any of the alteration-specific primers described herein. In some embodiments, the molecular complex comprises or consists of any of the PIEZO1 nucleic acid molecules (genomic nucleic acid molecules, mRNA molecules, or cDNA molecules), or complement thereof, described herein and any of the alteration-specific probes described herein.

In some embodiments, the molecular complex comprises an alteration-specific primer or an alteration-specific probe hybridized to a PIEZO1 genomic nucleic acid molecule encoding a PIEZO1 predicted gain-of-function polypeptide, wherein the alteration-specific primer or the alteration-specific probe is hybridized to the PIEZO1 genomic nucleic acid molecule at a position corresponding to: position 69,579 according to SEQ ID NO:2, or the complement thereof.

In some embodiments, the alteration-specific primer or the alteration-specific probe in the molecular complex is hybridized to: CTG codon at positions corresponding to positions 69,578-69, 580 according to SEQ ID NO:2.

In some embodiments, the genomic nucleic acid molecule in the molecular complex comprises SEQ ID NO:2.

In some embodiments, the molecular complex comprises an alteration-specific primer or an alteration-specific probe hybridized to a PIEZO1 mRNA molecule encoding a PIEZO1 predicted gain-of-function polypeptide, wherein the alteration-specific primer or the alteration-specific probe is hybridized to the PIEZO1 mRNA molecule at a position corresponding to: position 7,785 according to SEQ ID NO:13, or the complement thereof; position 1,095 according to SEQ ID NO:14, or the complement thereof; position 6,073 according to SEQ ID NO:15, or the complement thereof; position 1,336 according to SEQ ID NO:16, or the complement thereof; position 6,073 according to SEQ ID NO:17, or the complement thereof; position 6,236 according to SEQ ID NO:18, or the complement thereof; position 7,526 according to SEQ ID NO:19, or the complement thereof; position 1,336 according to SEQ ID NO:20, or the complement thereof; position corresponding to position 361 according to SEQ ID NO:21, or the complement thereof; or position 7,776 according to SEQ ID NO:22, or the complement thereof.

In some embodiments, the alteration-specific primer or the alteration-specific probe in the molecular complex is hybridized to: a CUG codon at positions corresponding to positions 7,784-7,786 according to SEQ ID NO:13, a CUG codon at positions corresponding to positions 1,094-1,096 according to SEQ ID NO:14, a CUG codon at positions corresponding to positions 6,072-6,074 according to SEQ ID NO:15, a CUG codon at positions corresponding to positions 1,335-1,337 according to SEQ ID NO:16, a CUG codon at positions corresponding to positions 6,072-6,074 according to SEQ ID NO:17, a CUG codon at positions corresponding to positions 6,235-6,237 according to SEQ ID NO:18, a CUG codon at positions corresponding to positions 7,525-7,527 according to SEQ ID NO:19, a CUG codon at positions corresponding to positions 1,335-1,337 according to SEQ ID NO:20; a CUG codon at positions corresponding to positions 360-362 according to SEQ ID NO:21; or a CUG codon at positions corresponding to positions 7,775-7,777 according to SEQ ID NO:22.

In some embodiments, the mRNA molecule in the molecular complex comprises SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, or SEQ ID NO:22.

In some embodiments, the molecular complex comprises an alteration-specific primer or an alteration-specific probe hybridized to a PIEZO1 cDNA molecule encoding a PIEZO1 predicted gain-of-function polypeptide, wherein the alteration-specific primer or the alteration-specific probe is hybridized to the PIEZO1 cDNA molecule at a position corresponding to: position 7,785 according to SEQ ID NO:33, or the complement thereof; position 1,095 according to SEQ ID NO:34, or the complement thereof; position 6,073 according to SEQ ID NO:35, or the complement thereof; position 1,336 according to SEQ ID NO:36, or the complement thereof; position 6,073 according to SEQ ID NO:37, or the complement thereof; position 6,236 according to SEQ ID NO:38, or the complement thereof; position 7,526 according to SEQ ID NO:39, or the complement thereof; position 1,336 according to SEQ ID NO:40, or the complement thereof; position 361 according to SEQ ID NO:41, or the complement thereof; or position 7,776 according to SEQ ID NO:42, or the complement thereof.

In some embodiments, the alteration-specific primer or the alteration-specific probe in the molecular complex is hybridized to: a CTG codon at positions corresponding to positions 7,784-7,786 according to SEQ ID NO:33, a CTG codon at positions corresponding to positions 1,094-1,096 according to SEQ ID NO:34, a CTG codon at positions corresponding to positions 6,072-6,074 according to SEQ ID NO:35, a CTG codon at positions corresponding to positions 1,335-1,337 according to SEQ ID NO:36, a CTG codon at positions corresponding to positions 6,072-6,074 according to SEQ ID NO:37, a CTG codon at positions corresponding to positions 6,235-6,237 according to SEQ ID NO:38, a CTG codon at positions corresponding to positions 7,525-7,527 according to SEQ ID NO:39, a CTG codon at positions corresponding to positions 1,335-1,337 according to SEQ ID NO:40; a CTG codon at positions corresponding to positions 360-362 according to SEQ ID NO:41, or a CTG codon at positions corresponding to positions 7,775-7,777 according to SEQ ID NO:42.

In some embodiments, the cDNA molecule in the molecular complex comprises SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, or SEQ ID NO:42.

In some embodiments, the molecular complex comprises an alteration-specific probe or an alteration-specific primer comprising a label. In some embodiments, the label is a fluorescent label, a radiolabel, or biotin. In some embodiments, the molecular complex further comprises a non-human polymerase.

The nucleotide sequence of a PIEZO1 reference genomic nucleic acid molecule is set forth in SEQ ID NO:1 (ENSG00000103335.22 encompassing chr16: 88,715,338-88,785,220 in the GRCh38/hg38 human genome assembly). Referring to SEQ ID NO:1, position 69,579 is cytosine.

A PIEZO1 variant genomic nucleic acid molecule exists, wherein the cytosine at position 69,579 is replaced with a thymine. The nucleotide sequence of this PIEZO1 variant genomic nucleic acid molecule is set forth in SEQ ID NO:2.

The nucleotide sequence of a PIEZO1 reference mRNA molecule is set forth in SEQ ID NO:3. Referring to SEQ ID NO:3, position 7,785 is a cytosine. The nucleotide sequence of another PIEZO1 reference mRNA molecule is set forth in SEQ ID NO:4. Referring to SEQ ID NO:4, position 1,095 is a cytosine. The nucleotide sequence of another PIEZO1 reference mRNA molecule is set forth in SEQ ID NO:5. Referring to SEQ ID NO:5, position 6,073 is a cytosine. The nucleotide sequence of another PIEZO1 reference mRNA molecule is set forth in SEQ ID NO:6. Referring to SEQ ID NO:6, position 1,336 is a cytosine. The nucleotide sequence of another PIEZO1 reference mRNA molecule is set forth in SEQ ID NO:7. Referring to SEQ ID NO:7, position 6,073 is a cytosine. The nucleotide sequence of another PIEZO1 reference mRNA molecule is set forth in SEQ ID NO:8. Referring to SEQ ID NO:8, position 6,236 is a cytosine. The nucleotide sequence of another PIEZO1 reference mRNA molecule is set forth in SEQ ID NO:9. Referring to SEQ ID NO:9, position 7,526 is a cytosine. The nucleotide sequence of another PIEZO1 reference mRNA molecule is set forth in SEQ ID NO:10. Referring to SEQ ID NO:10, position 1,336 is a cytosine. The nucleotide sequence of another PIEZO1 reference mRNA molecule is set forth in SEQ ID NO:11. Referring to SEQ ID NO:10, position 361 is a cytosine. The nucleotide sequence of another PIEZO1 reference mRNA molecule is set forth in SEQ ID NO:12. Referring to SEQ ID NO:12, position 7,776 is a cytosine.

A PIEZO1 variant mRNA molecule exists, wherein the cytosine at position 7,785 is replaced with a uracil. The nucleotide sequence of this PIEZO1 variant mRNA molecule is set forth in SEQ ID NO:13.

Another PIEZO1 variant mRNA molecule exists, wherein the cytosine at position 1,095 is replaced with a uracil. The nucleotide sequence of this PIEZO1 variant mRNA molecule is set forth in SEQ ID NO:14.

Another PIEZO1 variant mRNA molecule exists, wherein the cytosine at position 6,073 is replaced with a uracil. The nucleotide sequence of this PIEZO1 variant mRNA molecule is set forth in SEQ ID NO:15.

Another PIEZO1 variant mRNA molecule exists, wherein the cytosine at position 1,336 is replaced with a uracil. The nucleotide sequence of this PIEZO1 variant mRNA molecule is set forth in SEQ ID NO:16.

Another PIEZO1 variant mRNA molecule exists, wherein the cytosine at position 6,073 is replaced with a uracil. The nucleotide sequence of this PIEZO1 variant mRNA molecule is set forth in SEQ ID NO:17.

Another PIEZO1 variant mRNA molecule exists, wherein the cytosine at position 6,236 is replaced with a uracil. The nucleotide sequence of this PIEZO1 variant mRNA molecule is set forth in SEQ ID NO:18.

Another PIEZO1 variant mRNA molecule exists, wherein the cytosine at position 7,526 is replaced with a uracil. The nucleotide sequence of this PIEZO1 variant mRNA molecule is set forth in SEQ ID NO:19.

Another PIEZO1 variant mRNA molecule exists, wherein the cytosine at position 1,336 is replaced with a uracil. The nucleotide sequence of this PIEZO1 variant mRNA molecule is set forth in SEQ ID NO:20.

Another PIEZO1 variant mRNA molecule exists, wherein the cytosine at position 361 is replaced with a uracil. The nucleotide sequence of this PIEZO1 variant mRNA molecule is set forth in SEQ ID NO:21.

Another PIEZO1 variant mRNA molecule exists, wherein the cytosine at position 7,776 is replaced with a uracil. The nucleotide sequence of this PIEZO1 variant mRNA molecule is set forth in SEQ ID NO:22.

The nucleotide sequence of a PIEZO1 reference cDNA molecule is set forth in SEQ ID NO:23. Referring to SEQ ID NO:23, position 7,785 is a cytosine. The nucleotide sequence of another PIEZO1 reference cDNA molecule is set forth in SEQ ID NO:24. Referring to SEQ ID NO:24 position 1,095 is a cytosine. The nucleotide sequence of another PIEZO1 reference cDNA molecule is set forth in SEQ ID NO:25. Referring to SEQ ID NO:25, position 6,073 is a cytosine. The nucleotide sequence of another PIEZO1 reference cDNA molecule is set forth in SEQ ID NO:26. Referring to SEQ ID NO:26, position 1,336 is a cytosine. The nucleotide sequence of another PIEZO1 reference cDNA molecule is set forth in SEQ ID NO27. Referring to SEQ ID NO:27, position 6,073 is a cytosine. The nucleotide sequence of another PIEZO1 reference cDNA molecule is set forth in SEQ ID NO:28. Referring to SEQ ID NO:28, position 6,236 is a cytosine. The nucleotide sequence of another PIEZO1 reference cDNA molecule is set forth in SEQ ID NO:29. Referring to SEQ ID NO:29, position 7,526 is a cytosine. The nucleotide sequence of another PIEZO1 reference cDNA molecule is set forth in SEQ ID NO:30. Referring to SEQ ID NO:30, position 1,336 is a cytosine. The nucleotide sequence of another PIEZO1 reference cDNA molecule is set forth in SEQ ID NO:31. Referring to SEQ ID NO:31, position 371 is a cytosine. The nucleotide sequence of another PIEZO1 reference cDNA molecule is set forth in SEQ ID NO:32. Referring to SEQ ID NO:32, position 7,776 is a cytosine.

A PIEZO1 variant cDNA molecule exists, wherein the cytosine at position 7,785 is replaced with a thymine. The nucleotide sequence of this PIEZO1 variant cDNA molecule is set forth in SEQ ID NO:33.

Another PIEZO1 variant cDNA molecule exists, wherein the cytosine at position 1,095 is replaced with a thymine. The nucleotide sequence of this PIEZO1 variant cDNA molecule is set forth in SEQ ID NO:34.

Another PIEZO1 variant cDNA molecule exists, wherein the cytosine at position 6,073 is replaced with a thymine. The nucleotide sequence of this PIEZO1 variant cDNA molecule is set forth in SEQ ID NO:35.

Another PIEZO1 variant cDNA molecule exists, wherein the cytosine at position 1,336 is replaced with a thymine. The nucleotide sequence of this PIEZO1 variant cDNA molecule is set forth in SEQ ID NO:36.

Another PIEZO1 variant cDNA molecule exists, wherein the cytosine at position 6,073 is replaced with a thymine. The nucleotide sequence of this PIEZO1 variant cDNA molecule is set forth in SEQ ID NO:37.

Another PIEZO1 variant cDNA molecule exists, wherein the cytosine at position 6,236 is replaced with a thymine. The nucleotide sequence of this PIEZO1 variant cDNA molecule is set forth in SEQ ID NO:38.

Another PIEZO1 variant cDNA molecule exists, wherein the cytosine at position 7,526 is replaced with a thymine. The nucleotide sequence of this PIEZO1 variant cDNA molecule is set forth in SEQ ID NO:39.

Another PIEZO1 variant cDNA molecule exists, wherein the cytosine at position 1,336 is replaced with a thymine. The nucleotide sequence of this PIEZO1 variant cDNA molecule is set forth in SEQ ID NO:40.

Another PIEZO1 variant cDNA molecule exists, wherein the cytosine at position 361 is replaced with a thymine. The nucleotide sequence of this PIEZO1 variant cDNA molecule is set forth in SEQ ID NO:41.

Another PIEZO1 variant cDNA molecule exists, wherein the cytosine at position 7,776 is replaced with a thymine. The nucleotide sequence of this PIEZO1 variant cDNA molecule is set forth in SEQ ID NO:42.

The genomic nucleic acid molecules, mRNA molecules, and cDNA molecules can be from any organism. For example, the genomic nucleic acid molecules, mRNA molecules, and cDNA molecules can be human or an ortholog from another organism, such as a non-human mammal, a rodent, a mouse, or a rat. It is understood that gene sequences within a population can vary due to polymorphisms such as single-nucleotide polymorphisms. The examples provided herein are only exemplary sequences. Other sequences are also possible.

Also provided herein are functional polynucleotides that can interact with the disclosed nucleic acid molecules. Examples of functional polynucleotides include, but are not limited to, antisense molecules, aptamers, ribozymes, triplex forming molecules, and external guide sequences. The functional polynucleotides can act as effectors, agonists, modulators, and stimulators of a specific activity possessed by a target molecule, or the functional polynucleotides can possess a de novo activity independent of any other molecules.

The isolated nucleic acid molecules disclosed herein can comprise RNA, DNA, or both RNA and DNA. The isolated nucleic acid molecules can also be linked or fused to a heterologous nucleic acid sequence, such as in a vector, or a heterologous label. For example, the isolated nucleic acid molecules disclosed herein can be within a vector or as an exogenous donor sequence comprising the isolated nucleic acid molecule and a heterologous nucleic acid sequence. The isolated nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3×FLAG, 6×His or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

The isolated nucleic acid molecules, or the complement thereof, can also be present within a host cell. In some embodiments, the host cell can comprise the vector that comprises any of the nucleic acid molecules described herein, or the complement thereof. In some embodiments, the nucleic acid molecule is operably linked to a promoter active in the host cell. In some embodiments, the promoter is an exogenous promoter. In some embodiments, the promoter is an inducible promoter. In some embodiments, the host cell is a bacterial cell, a yeast cell, an insect cell, or a mammalian cell. In some embodiments, the host cell is a bacterial cell. In some embodiments, the host cell is a yeast cell. In some embodiments, the host cell is an insect cell. In some embodiments, the host cell is a mammalian cell.

The disclosed nucleic acid molecules can comprise, for example, nucleotides or non-natural or modified nucleotides, such as nucleotide analogs or nucleotide substitutes. Such nucleotides include a nucleotide that contains a modified base, sugar, or phosphate group, or that incorporates a non-natural moiety in its structure. Examples of non-natural nucleotides include, but are not limited to, dideoxynucleotides, biotinylated, aminated, deaminated, alkylated, benzylated, and fluorophor-labeled nucleotides.

The nucleic acid molecules disclosed herein can also comprise one or more nucleotide analogs or substitutions. A nucleotide analog is a nucleotide which contains a modification to either the base, sugar, or phosphate moieties. Modifications to the base moiety include, but are not limited to, natural and synthetic modifications of A, C, G, and T/U, as well as different purine or pyrimidine bases such as, for example, pseudouridine, uracil-5-yl, hypoxanthin-9-yl (I), and 2-aminoadenin-9-yl. Modified bases include, but are not limited to, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (such as, for example, 5-bromo), 5-trifluoromethyl and other 5-substituted uracils and cytosines, 7-methylguanine, 7-methyladenine, 8-azaguanine, 8-azaadenine, 7-deazaguanine, 7-deazaadenine, 3-deazaguanine, and 3-deazaadenine.

Nucleotide analogs can also include modifications of the sugar moiety. Modifications to the sugar moiety include, but are not limited to, natural modifications of the ribose and deoxy ribose as well as synthetic modifications. Sugar modifications include, but are not limited to, the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl, and alkynyl may be substituted or unsubstituted C₁₋₁₀alkyl or C₂₋₁₀ alkenyl, and C₂₋₁₀ alkynyl. Exemplary 2′ sugar modifications also include, but are not limited to, —O[(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m, independently, are from 1 to about 10. Other modifications at the 2′ position include, but are not limited to, C₁₋₁₀alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars can also include those that contain modifications at the bridging ring oxygen, such as CH₂ and S. Nucleotide sugar analogs can also have sugar mimetics, such as cyclobutyl moieties in place of the pentofuranosyl sugar.

Nucleotide analogs can also be modified at the phosphate moiety. Modified phosphate moieties include, but are not limited to, those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. These phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts, and free acid forms are also included. Nucleotide substitutes also include peptide nucleic acids (PNAs).

The present disclosure also provides vectors comprising any one or more of the nucleic acid molecules disclosed herein. In some embodiments, the vectors comprise any one or more of the nucleic acid molecules disclosed herein and a heterologous nucleic acid. The vectors can be viral or nonviral vectors capable of transporting a nucleic acid molecule. In some embodiments, the vector is a plasmid or cosmid (such as, for example, a circular double-stranded DNA into which additional DNA segments can be ligated). In some embodiments, the vector is a viral vector, wherein additional DNA segments can be ligated into the viral genome. Expression vectors include, but are not limited to, plasmids, cosmids, retroviruses, adenoviruses, adeno-associated viruses (AAV), plant viruses such as cauliflower mosaic virus and tobacco mosaic virus, yeast artificial chromosomes (YACs), Epstein-Barr (EBV)-derived episomes, and other expression vectors known in the art.

Desired regulatory sequences for mammalian host cell expression can include, for example, viral elements that direct high levels of polypeptide expression in mammalian cells, such as promoters and/or enhancers derived from retroviral LTRs, cytomegalovirus (CMV) (such as, for example, CMV promoter/enhancer), Simian Virus 40 (SV40) (such as, for example, SV40 promoter/enhancer), adenovirus, (such as, for example, the adenovirus major late promoter (AdMLP)), polyoma and strong mammalian promoters such as native immunoglobulin and actin promoters. Methods of expressing polypeptides in bacterial cells or fungal cells (such as, for example, yeast cells) are also well known. A promoter can be, for example, a constitutively active promoter, a conditional promoter, an inducible promoter, a temporally restricted promoter (such as, for example, a developmentally regulated promoter), or a spatially restricted promoter (such as, for example, a cell-specific or tissue-specific promoter).

Percent identity (or percent complementarity) between particular stretches of nucleotide sequences within nucleic acid molecules or amino acid sequences within polypeptides can be determined routinely using BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). Herein, if reference is made to percent sequence identity, the higher percentages of sequence identity are preferred over the lower ones.

The present disclosure also provides compositions comprising any one or more of the isolated nucleic acid molecules, genomic nucleic acid molecules, mRNA molecules, and/or cDNA molecules disclosed herein, or vectors comprising the same. In some embodiments, the composition is a pharmaceutical composition. In some embodiments, the compositions comprise a carrier and/or excipient. Examples of carriers include, but are not limited to, poly(lactic acid) (PLA) microspheres, poly(D,L-lactic-coglycolic-acid) (PLGA) microspheres, liposomes, micelles, inverse micelles, lipid cochleates, and lipid microtubules. A carrier may comprise a buffered salt solution such as PBS, HBSS, etc.

As used herein, the phrase “corresponding to” or grammatical variations thereof when used in the context of the numbering of a particular nucleotide or nucleotide sequence or position refers to the numbering of a specified reference sequence when the particular nucleotide or nucleotide sequence is compared to a reference sequence (such as, for example, SEQ ID NO:1, SEQ ID NO:3, or SEQ ID NO:40). In other words, the residue (such as, for example, nucleotide or amino acid) number or residue (such as, for example, nucleotide or amino acid) position of a particular polymer is designated with respect to the reference sequence rather than by the actual numerical position of the residue within the particular nucleotide or nucleotide sequence. For example, a particular nucleotide sequence can be aligned to a reference sequence by introducing gaps to optimize residue matches between the two sequences. In these cases, although the gaps are present, the numbering of the residue in the particular nucleotide or nucleotide sequence is made with respect to the reference sequence to which it has been aligned.

For example, a PIEZO1 nucleic acid molecule comprising a nucleotide sequence encoding a PIEZO1 predicted gain-of-function polypeptide, wherein the nucleotide sequence comprises a thymine at a position corresponding to position 69,579 according to SEQ ID NO:2 means that if the nucleotide sequence of the PIEZO1 genomic nucleic acid molecule is aligned to the sequence of SEQ ID NO:2, the PIEZO1 sequence has a thymine residue at the position that corresponds to position 69,579 of SEQ ID NO:2. The same applies for PIEZO1 mRNA molecules comprising a nucleotide sequence encoding a PIEZO1 predicted gain-of-function polypeptide, wherein the nucleotide sequence comprises a uracil at a position corresponding to position 7,785 according to SEQ ID NO:13, and PIEZO1 cDNA molecules comprising a nucleotide sequence encoding a PIEZO1 predicted gain-of-function polypeptide, wherein the nucleotide sequence comprises a thymine at a position corresponding to position 7,785 according to SEQ ID NO:33. These phrases refer to a PIEZO1 nucleic acid molecule encoding a PIEZO1 predicted gain-of-function polypeptide, wherein the genomic nucleic acid molecule has a nucleotide sequence that comprises a thymine residue that is homologous to the thymine residue at position 69,579 of SEQ ID NO:2 (or wherein the mRNA molecule has a nucleotide sequence that comprises a uracil residue that is homologous to the uracil residue at position 7,785 of SEQ ID NO:13, or wherein the cDNA molecule has a nucleotide sequence that comprises a thymine residue that is homologous to the thymine residue at position 7,785 of SEQ ID NO:33). Herein, such a sequence is also referred to as “PIEZO1 sequence with the Pro2,510Leu alteration” or “PIEZO1 sequence with the Pro2,510Leu variation.”

As described herein, a position within a PIEZO1 genomic nucleic acid molecule that corresponds to position 69,579 according to SEQ ID NO:2, for example, can be identified by performing a sequence alignment between the nucleotide sequence of a particular PIEZO1 nucleic acid molecule and the nucleotide sequence of SEQ ID NO:2. A variety of computational algorithms exist that can be used for performing a sequence alignment to identify a nucleotide position that corresponds to, for example, position 69,579 in SEQ ID NO:2. For example, by using the NCBI BLAST algorithm (Altschul et al., Nucleic Acids Res., 1997, 25, 3389-3402) or CLUSTALW software (Sievers and Higgins, Methods Mol. Biol., 2014, 1079, 105-116) sequence alignments may be performed. However, sequences can also be aligned manually.

The amino acid sequence of a PIEZO1 reference polypeptide is set forth in SEQ ID NO:43. Referring to SEQ ID NO:43, the PIEZO1 reference polypeptide is 2,521 amino acids in length. Referring SEQ ID NO:43, position 2,510 is a proline.

The amino acid sequence of anther PIEZO1 reference polypeptide is set forth in SEQ ID NO:44. Referring to SEQ ID NO:44, the PIEZO1 reference polypeptide is 2,035 amino acids in length. Referring to SEQ ID NO:44, position 2,024 is a proline.

The amino acid sequence of anther PIEZO1 reference polypeptide is set forth in SEQ ID NO:45. Referring to SEQ ID NO:45, the PIEZO1 reference polypeptide is 2,090 amino acids in length. Referring to SEQ ID NO:45, position 2,079 is a proline.

The amino acid sequence of anther PIEZO1 reference polypeptide is set forth in SEQ ID NO:46. Referring to SEQ ID NO:46, the PIEZO1 reference polypeptide is 105 amino acids in length. Referring to SEQ ID NO:46, position 94 is a proline.

A PIEZO1 predicted gain-of-function polypeptide exists (Pro2,510Leu), the amino acid sequence of which is set forth in SEQ ID NO:47. Referring to SEQ ID NO:47, the PIEZO1 predicted gain-of-function polypeptide is 2,521 amino acids in length. Referring to SEQ ID NO:86, position 2,510 is leucine.

Another PIEZO1 predicted gain-of-function polypeptide exists (Pro2,024Leu), the amino acid sequence of which is set forth in SEQ ID NO:48. Referring to SEQ ID NO:48, the PIEZO1 predicted gain-of-function polypeptide is 2,035 amino acids in length. Referring to SEQ ID NO:48, position 2,024 is leucine.

Another PIEZO1 predicted gain-of-function polypeptide exists (Pro2,079Leu), the amino acid sequence of which is set forth in SEQ ID NO:49. Referring to SEQ ID NO:49, the PIEZO1 predicted gain-of-function polypeptide is 2,090 amino acids in length. Referring to SEQ ID NO:49, position 2,079 is leucine.

Another PIEZO1 predicted gain-of-function polypeptide exists (Pro94Leu), the amino acid sequence of which is set forth in SEQ ID NO:50. Referring to SEQ ID NO:50, the PIEZO1 predicted gain-of-function polypeptide is 105 amino acids in length. Referring to SEQ ID NO:50, position 94 is leucine.

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. The amino acid sequence follows the standard convention of beginning at the amino terminus of the sequence and proceeding forward (i.e., from left to right in each line) to the carboxy terminus.

The present disclosure also provides therapeutic agents that treat or prevent varicose veins for use in the treatment or prevention of varicose veins (or for use in the preparation of a medicament for treating or preventing varicose veins) in a subject, wherein the subject has any of the PIEZO1 variant genomic nucleic acid molecules, variant mRNA molecules, and/or variant cDNA molecules encoding a PIEZO1 predicted gain-of-function polypeptide described herein. The therapeutic agents that treat or prevent varicose veins can be any of the therapeutic agents that treat or prevent varicose veins described herein. The varicose veins can be any of saphenous varicose veins, veticular varicose veins, spider veins, and pregnancy-related varicose veins.

In some embodiments, the subject is identified as having a genomic nucleic acid molecule encoding a PIEZO1 predicted gain-of-function polypeptide, or the complement thereof, wherein the genomic nucleic acid molecule has a nucleotide sequence comprising a thymine at a position corresponding to position 69,579 according to SEQ ID NO:2, or the complement thereof.

In some embodiments, the subject is identified as having an mRNA molecule encoding a PIEZO1 predicted gain-of-function polypeptide, or the complement thereof, wherein the mRNA molecule has a nucleotide sequence comprising: a uracil at a position corresponding to position 7,785 according to SEQ ID NO:13, or the complement thereof; a uracil at a position corresponding to position 1,095 according to SEQ ID NO:14, or the complement thereof; a uracil at a position corresponding to position 6,073 according to SEQ ID NO:15, or the complement thereof; a uracil at a position corresponding to position 1,336 according to SEQ ID NO:16, or the complement thereof; a uracil at a position corresponding to position 6,073 according to SEQ ID NO:17, or the complement thereof; a uracil at a position corresponding to position 6,236 according to SEQ ID NO:18, or the complement thereof; a uracil at a position corresponding to position 7,526 according to SEQ ID NO:19, or the complement thereof; a uracil at a position corresponding to position 1,336 according to SEQ ID NO:20, or the complement thereof; a uracil at a position corresponding to position 361 according to SEQ ID NO:21, or the complement thereof; or a uracil at a position corresponding to position 7,776 according to SEQ ID NO:22, or the complement thereof.

In some embodiments, the subject is identified as having a cDNA molecule encoding a PIEZO1 predicted gain-of-function polypeptide, or the complement thereof, wherein the cDNA molecule has a nucleotide sequence comprising: a thymine at a position corresponding to position 7,785 according to SEQ ID NO:33, or the complement thereof; a thymine at a position corresponding to position 1,095 according to SEQ ID NO:34, or the complement thereof; a thymine at a position corresponding to position 6,073 according to SEQ ID NO:35, or the complement thereof; a thymine at a position corresponding to position 1,336 according to SEQ ID NO:36, or the complement thereof; a thymine at a position corresponding to position 6,073 according to SEQ ID NO:37, or the complement thereof; a thymine at a position corresponding to position 6,236 according to SEQ ID NO:38, or the complement thereof; a thymine at a position corresponding to position 7,526 according to SEQ ID NO:39, or the complement thereof; a thymine at a position corresponding to position 1,336 according to SEQ ID NO:40, or the complement thereof; a thymine at a position corresponding to position 361 according to SEQ ID NO:41, or the complement thereof; or a thymine at a position corresponding to position 7,776 according to SEQ ID NO:42, or the complement thereof.

In some embodiments, the subject is identified as having: a genomic nucleic acid molecule having a nucleotide sequence encoding a PIEZO1 predicted gain-of-function polypeptide, wherein the nucleotide sequence comprises a thymine at a position corresponding to position 69,579 according to SEQ ID NO:2, or the complement thereof; an mRNA molecule having a nucleotide sequence encoding a PIEZO1 predicted gain-of-function polypeptide, wherein the nucleotide sequence comprises a uracil at a position corresponding to position 7,785 according to SEQ ID NO:13, or the complement thereof; a cDNA molecule having a nucleotide sequence encoding a PIEZO1 predicted gain-of-function polypeptide, wherein the nucleotide sequence comprises a thymine at a position corresponding to position 7,785 according to SEQ ID NO:33, or the complement thereof; or a PIEZO1 predicted gain-of-function polypeptide that comprises leucine at a position corresponding to position 2,510 according to SEQ ID NO:47.

In some embodiments, the subject is identified as having: a genomic nucleic acid molecule having a nucleotide sequence encoding a PIEZO1 predicted gain-of-function polypeptide, wherein the nucleotide sequence comprises an mRNA molecule having a nucleotide sequence encoding a PIEZO1 predicted gain-of-function polypeptide, wherein the nucleotide sequence comprises a uracil at a position corresponding to position 1,095 according to SEQ ID NO:14, or the complement thereof; a cDNA molecule having a nucleotide sequence encoding a PIEZO1 predicted gain-of-function polypeptide, wherein the nucleotide sequence comprises a thymine at a position corresponding to position 1,095 according to SEQ ID NO:34, or the complement thereof; or a PIEZO1 predicted gain-of-function polypeptide that comprises leucine at a position corresponding to position 2,024 according to SEQ ID NO:48.

In some embodiments, the subject is identified as having: a genomic nucleic acid molecule having a nucleotide sequence encoding a PIEZO1 predicted gain-of-function polypeptide, wherein the nucleotide sequence comprises an mRNA molecule having a nucleotide sequence encoding a PIEZO1 predicted gain-of-function polypeptide, wherein the nucleotide sequence comprises a uracil at a position corresponding to position 6,073 according to SEQ ID NO:15, or the complement thereof; a cDNA molecule having a nucleotide sequence encoding a PIEZO1 predicted gain-of-function polypeptide, wherein the nucleotide sequence comprises a thymine at a position corresponding to position 6,073 according to SEQ ID NO:35, or the complement thereof; or a PIEZO1 predicted gain-of-function polypeptide that comprises leucine at a position corresponding to position 2,079 according to SEQ ID NO:49.

In some embodiments, the subject is identified as having: a genomic nucleic acid molecule having a nucleotide sequence encoding a PIEZO1 predicted gain-of-function polypeptide, wherein the nucleotide sequence comprises an mRNA molecule having a nucleotide sequence encoding a PIEZO1 predicted gain-of-function polypeptide, wherein the nucleotide sequence comprises a uracil at a position corresponding to position 1,336 according to SEQ ID NO:16, or the complement thereof; a cDNA molecule having a nucleotide sequence encoding a PIEZO1 predicted gain-of-function polypeptide, wherein the nucleotide sequence comprises a thymine at a position corresponding to position 1,336 according to SEQ ID NO:36, or the complement thereof; or a PIEZO1 predicted gain-of-function polypeptide that comprises leucine at a position corresponding to position 94 according to SEQ ID NO:50.

In some embodiments, the subject is identified as having: a genomic nucleic acid molecule having a nucleotide sequence encoding a PIEZO1 predicted gain-of-function polypeptide, wherein the nucleotide sequence comprises an mRNA molecule having a nucleotide sequence encoding a PIEZO1 predicted gain-of-function polypeptide, wherein the nucleotide sequence comprises a uracil at a position corresponding to position 6,073 according to SEQ ID NO:17, or the complement thereof; or a cDNA molecule having a nucleotide sequence encoding a PIEZO1 predicted gain-of-function polypeptide, wherein the nucleotide sequence comprises a thymine at a position corresponding to position 6,073 according to SEQ ID NO:37, or the complement thereof.

In some embodiments, the subject is identified as having: a genomic nucleic acid molecule having a nucleotide sequence encoding a PIEZO1 predicted gain-of-function polypeptide, wherein the nucleotide sequence comprises an mRNA molecule having a nucleotide sequence encoding a PIEZO1 predicted gain-of-function polypeptide, wherein the nucleotide sequence comprises a uracil at a position corresponding to position 6,236 according to SEQ ID NO:18, or the complement thereof; or a cDNA molecule having a nucleotide sequence encoding a PIEZO1 predicted gain-of-function polypeptide, wherein the nucleotide sequence comprises a thymine at a position corresponding to position 6,236 according to SEQ ID NO:38, or the complement thereof.

In some embodiments, the subject is identified as having: a genomic nucleic acid molecule having a nucleotide sequence encoding a PIEZO1 predicted gain-of-function polypeptide, wherein the nucleotide sequence comprises an mRNA molecule having a nucleotide sequence encoding a PIEZO1 predicted gain-of-function polypeptide, wherein the nucleotide sequence comprises a uracil at a position corresponding to position 7,526 according to SEQ ID NO:19, or the complement thereof; or a cDNA molecule having a nucleotide sequence encoding a PIEZO1 predicted gain-of-function polypeptide, wherein the nucleotide sequence comprises a thymine at a position corresponding to position 7,526 according to SEQ ID NO:39, or the complement thereof.

In some embodiments, the subject is identified as having: a genomic nucleic acid molecule having a nucleotide sequence encoding a PIEZO1 predicted gain-of-function polypeptide, wherein the nucleotide sequence comprises an mRNA molecule having a nucleotide sequence encoding a PIEZO1 predicted gain-of-function polypeptide, wherein the nucleotide sequence comprises a uracil at a position corresponding to position 1,336 according to SEQ ID NO:20, or the complement thereof; or a cDNA molecule having a nucleotide sequence encoding a PIEZO1 predicted gain-of-function polypeptide, wherein the nucleotide sequence comprises a thymine at a position corresponding to position 1,336 according to SEQ ID NO:40, or the complement thereof.

In some embodiments, the subject is identified as having: a genomic nucleic acid molecule having a nucleotide sequence encoding a PIEZO1 predicted gain-of-function polypeptide, wherein the nucleotide sequence comprises an mRNA molecule having a nucleotide sequence encoding a PIEZO1 predicted gain-of-function polypeptide, wherein the nucleotide sequence comprises a uracil at a position corresponding to position 361 according to SEQ ID NO:21, or the complement thereof; or a cDNA molecule having a nucleotide sequence encoding a PIEZO1 predicted gain-of-function polypeptide, wherein the nucleotide sequence comprises a thymine at a position corresponding to position 361 according to SEQ ID NO:41, or the complement thereof.

In some embodiments, the subject is identified as having: a genomic nucleic acid molecule having a nucleotide sequence encoding a PIEZO1 predicted gain-of-function polypeptide, wherein the nucleotide sequence comprises an mRNA molecule having a nucleotide sequence encoding a PIEZO1 predicted gain-of-function polypeptide, wherein the nucleotide sequence comprises a uracil at a position corresponding to position 7,776 according to SEQ ID NO:22, or the complement thereof; or a cDNA molecule having a nucleotide sequence encoding a PIEZO1 predicted gain-of-function polypeptide, wherein the nucleotide sequence comprises a thymine at a position corresponding to position 7,776 according to SEQ ID NO:42, or the complement thereof.

The present disclosure also provides PIEZO1 agonists for use in the treatment or prevention of varicose veins (or for use in the preparation of a medicament for treating or preventing varicose veins) in a subject, wherein the subject is heterozygous for any of the PIEZO1 variant genomic nucleic acid molecules, variant mRNA molecules, and/or variant cDNA molecules encoding a PIEZO1 predicted gain-of-function polypeptides described herein, or wherein the subject is reference for a PIEZO1 genomic nucleic acid molecule, mRNA molecule, or cDNA molecule. The PIEZO1 agonists can be any of the PIEZO1 agonists described herein. The varicose veins can be any of saphenous varicose veins, veticular varicose veins, spider veins, and pregnancy-related varicose veins.

In some embodiments, the subject is reference for a PIEZO1 genomic nucleic acid molecule, a PIEZO1 mRNA molecule, or a PIEZO1 cDNA molecule.

In some embodiments, the subject is heterozygous for a genomic nucleic acid molecule encoding a PIEZO1 predicted gain-of-function polypeptide, or the complement thereof, wherein the genomic nucleic acid molecule has a nucleotide sequence comprising a thymine at a position corresponding to position 69,579 according to SEQ ID NO:2, or the complement thereof.

In some embodiments, the subject is heterozygous for an mRNA molecule encoding a PIEZO1 predicted gain-of-function polypeptide, or the complement thereof, wherein the mRNA molecule has a nucleotide sequence comprising: a uracil at a position corresponding to position 7,785 according to SEQ ID NO:13, or the complement thereof; a uracil at a position corresponding to position 1,095 according to SEQ ID NO:14, or the complement thereof; a uracil at a position corresponding to position 6,073 according to SEQ ID NO:15, or the complement thereof; a uracil at a position corresponding to position 1,336 according to SEQ ID NO:16, or the complement thereof; a uracil at a position corresponding to position 6,073 according to SEQ ID NO:17, or the complement thereof; a uracil at a position corresponding to position 6,236 according to SEQ ID NO:18, or the complement thereof; a uracil at a position corresponding to position 7,526 according to SEQ ID NO:19, or the complement thereof; a uracil at a position corresponding to position 1,336 according to SEQ ID NO:20, or the complement thereof; a uracil at a position corresponding to position 361 according to SEQ ID NO:21, or the complement thereof; or a uracil at a position corresponding to position 7,776 according to SEQ ID NO:22, or the complement thereof.

In some embodiments, the subject is heterozygous for a cDNA molecule encoding a PIEZO1 predicted gain-of-function polypeptide, or the complement thereof, wherein the cDNA molecule has a nucleotide sequence comprising: a thymine at a position corresponding to position 7,785 according to SEQ ID NO:33, or the complement thereof; a thymine at a position corresponding to position 1,095 according to SEQ ID NO:34, or the complement thereof; a thymine at a position corresponding to position 6,073 according to SEQ ID NO:35, or the complement thereof; a thymine at a position corresponding to position 1,336 according to SEQ ID NO:36, or the complement thereof; a thymine at a position corresponding to position 6,073 according to SEQ ID NO:37, or the complement thereof; a thymine at a position corresponding to position 6,236 according to SEQ ID NO:38, or the complement thereof; a thymine at a position corresponding to position 7,526 according to SEQ ID NO:39, or the complement thereof; a thymine at a position corresponding to position 1,336 according to SEQ ID NO:40, or the complement thereof; a thymine at a position corresponding to position 361 according to SEQ ID NO:41, or the complement thereof; or a thymine at a position corresponding to position 7,776 according to SEQ ID NO:42, or the complement thereof.

In some embodiments, the subject is identified as being heterozygous for: a genomic nucleic acid molecule having a nucleotide sequence encoding a PIEZO1 predicted gain-of-function polypeptide, wherein the nucleotide sequence comprises a thymine at a position corresponding to position 69,579 according to SEQ ID NO:2, or the complement thereof; an mRNA molecule having a nucleotide sequence encoding a PIEZO1 predicted gain-of-function polypeptide, wherein the nucleotide sequence comprises a uracil at a position corresponding to position 7,785 according to SEQ ID NO:13, or the complement thereof; a cDNA molecule having a nucleotide sequence encoding a PIEZO1 predicted gain-of-function polypeptide, wherein the nucleotide sequence comprises a thymine at a position corresponding to position 7,785 according to SEQ ID NO:33, or the complement thereof; or a PIEZO1 predicted gain-of-function polypeptide that comprises leucine at a position corresponding to position 2,510 according to SEQ ID NO:47. The PIEZO1 agonists can be any of the PIEZO1 agonists described herein.

In some embodiments, the subject is identified as being heterozygous for: a genomic nucleic acid molecule having a nucleotide sequence encoding a PIEZO1 predicted gain-of-function polypeptide, wherein the nucleotide sequence comprises an mRNA molecule having a nucleotide sequence encoding a PIEZO1 predicted gain-of-function polypeptide, wherein the nucleotide sequence comprises a uracil at a position corresponding to position 1,095 according to SEQ ID NO:14, or the complement thereof; a cDNA molecule having a nucleotide sequence encoding a PIEZO1 predicted gain-of-function polypeptide, wherein the nucleotide sequence comprises a thymine at a position corresponding to position 1,095 according to SEQ ID NO:34, or the complement thereof; or a PIEZO1 predicted gain-of-function polypeptide that comprises leucine at a position corresponding to position 2,024 according to SEQ ID NO:48. The PIEZO1 agonists can be any of the PIEZO1 agonists described herein.

In some embodiments, the subject is identified as being heterozygous for: a genomic nucleic acid molecule having a nucleotide sequence encoding a PIEZO1 predicted gain-of-function polypeptide, wherein the nucleotide sequence comprises an mRNA molecule having a nucleotide sequence encoding a PIEZO1 predicted gain-of-function polypeptide, wherein the nucleotide sequence comprises a uracil at a position corresponding to position 6,073 according to SEQ ID NO:15, or the complement thereof; a cDNA molecule having a nucleotide sequence encoding a PIEZO1 predicted gain-of-function polypeptide, wherein the nucleotide sequence comprises a thymine at a position corresponding to position 6,073 according to SEQ ID NO:35, or the complement thereof; or a PIEZO1 predicted gain-of-function polypeptide that comprises leucine at a position corresponding to position 2,079 according to SEQ ID NO:49. The PIEZO1 agonists can be any of the PIEZO1 agonists described herein.

In some embodiments, the subject is identified as being heterozygous for: a genomic nucleic acid molecule having a nucleotide sequence encoding a PIEZO1 predicted gain-of-function polypeptide, wherein the nucleotide sequence comprises an mRNA molecule having a nucleotide sequence encoding a PIEZO1 predicted gain-of-function polypeptide, wherein the nucleotide sequence comprises a uracil at a position corresponding to position 1,336 according to SEQ ID NO:16, or the complement thereof; a cDNA molecule having a nucleotide sequence encoding a PIEZO1 predicted gain-of-function polypeptide, wherein the nucleotide sequence comprises a thymine at a position corresponding to position 1,336 according to SEQ ID NO:36, or the complement thereof; or a PIEZO1 predicted gain-of-function polypeptide that comprises leucine at a position corresponding to position 94 according to SEQ ID NO:50. The PIEZO1 agonists can be any of the PIEZO1 agonists described herein.

In some embodiments, the subject is identified as being heterozygous for: a genomic nucleic acid molecule having a nucleotide sequence encoding a PIEZO1 predicted gain-of-function polypeptide, wherein the nucleotide sequence comprises an mRNA molecule having a nucleotide sequence encoding a PIEZO1 predicted gain-of-function polypeptide, wherein the nucleotide sequence comprises a uracil at a position corresponding to position 6,073 according to SEQ ID NO:17, or the complement thereof; or a cDNA molecule having a nucleotide sequence encoding a PIEZO1 predicted gain-of-function polypeptide, wherein the nucleotide sequence comprises a thymine at a position corresponding to position 6,073 according to SEQ ID NO:37, or the complement thereof. The PIEZO1 agonists can be any of the PIEZO1 agonists described herein.

In some embodiments, the subject is identified as being heterozygous for: a genomic nucleic acid molecule having a nucleotide sequence encoding a PIEZO1 predicted gain-of-function polypeptide, wherein the nucleotide sequence comprises an mRNA molecule having a nucleotide sequence encoding a PIEZO1 predicted gain-of-function polypeptide, wherein the nucleotide sequence comprises a uracil at a position corresponding to position 6,236 according to SEQ ID NO:18, or the complement thereof; or a cDNA molecule having a nucleotide sequence encoding a PIEZO1 predicted gain-of-function polypeptide, wherein the nucleotide sequence comprises a thymine at a position corresponding to position 6,236 according to SEQ ID NO:38, or the complement thereof. The PIEZO1 agonists can be any of the PIEZO1 agonists described herein.

In some embodiments, the subject is identified as being heterozygous for: a genomic nucleic acid molecule having a nucleotide sequence encoding a PIEZO1 predicted gain-of-function polypeptide, wherein the nucleotide sequence comprises an mRNA molecule having a nucleotide sequence encoding a PIEZO1 predicted gain-of-function polypeptide, wherein the nucleotide sequence comprises a uracil at a position corresponding to position 7,526 according to SEQ ID NO:19, or the complement thereof; or a cDNA molecule having a nucleotide sequence encoding a PIEZO1 predicted gain-of-function polypeptide, wherein the nucleotide sequence comprises a thymine at a position corresponding to position 7,526 according to SEQ ID NO:39, or the complement thereof. The PIEZO1 agonists can be any of the PIEZO1 agonists described herein.

In some embodiments, the subject is identified as being heterozygous for: a genomic nucleic acid molecule having a nucleotide sequence encoding a PIEZO1 predicted gain-of-function polypeptide, wherein the nucleotide sequence comprises an mRNA molecule having a nucleotide sequence encoding a PIEZO1 predicted gain-of-function polypeptide, wherein the nucleotide sequence comprises a uracil at a position corresponding to position 1,336 according to SEQ ID NO:20, or the complement thereof; or a cDNA molecule having a nucleotide sequence encoding a PIEZO1 predicted gain-of-function polypeptide, wherein the nucleotide sequence comprises a thymine at a position corresponding to position 1,336 according to SEQ ID NO:40, or the complement thereof.

In some embodiments, the subject is identified as being heterozygous for: a genomic nucleic acid molecule having a nucleotide sequence encoding a PIEZO1 predicted gain-of-function polypeptide, wherein the nucleotide sequence comprises an mRNA molecule having a nucleotide sequence encoding a PIEZO1 predicted gain-of-function polypeptide, wherein the nucleotide sequence comprises a uracil at a position corresponding to position 361 according to SEQ ID NO:21, or the complement thereof; or a cDNA molecule having a nucleotide sequence encoding a PIEZO1 predicted gain-of-function polypeptide, wherein the nucleotide sequence comprises a thymine at a position corresponding to position 361 according to SEQ ID NO:41, or the complement thereof.

In some embodiments, the subject is identified as being heterozygous for: a genomic nucleic acid molecule having a nucleotide sequence encoding a PIEZO1 predicted gain-of-function polypeptide, wherein the nucleotide sequence comprises an mRNA molecule having a nucleotide sequence encoding a PIEZO1 predicted gain-of-function polypeptide, wherein the nucleotide sequence comprises a uracil at a position corresponding to position 7,776 according to SEQ ID NO:22, or the complement thereof; or a cDNA molecule having a nucleotide sequence encoding a PIEZO1 predicted gain-of-function polypeptide, wherein the nucleotide sequence comprises a thymine at a position corresponding to position 7,776 according to SEQ ID NO:42, or the complement thereof.

In some embodiments, the subject is identified as having: a PIEZO1 reference genomic nucleic acid molecule comprising SEQ ID NO:1, a PIEZO1 reference mRNA molecules comprising one or more SEQ ID NOs:3-12, a PIEZO1 reference cDNA molecules comprising one or more SEQ ID NOs:23-32, or a PIEZO1 reference polypeptide comprising one or more SEQ ID NOs:43-46. The PIEZO1 agonists can be any of the PIEZO1 agonists described herein.

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

The following examples are provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated, and are intended to be purely exemplary and are not intended to limit the scope of any claims. Efforts have been made to ensure accuracy with respect to numbers (such as, for example, amounts, temperature, etc.), but some errors and deviations may be accounted for. Unless indicated otherwise, parts are parts by weight, temperature is in ° C. or is at ambient temperature, and pressure is at or near atmospheric.

### EXAMPLES

### Example 1: Association of Varicose Veins with PIEZO1 GOF Variants

The exomes of 454,787 UKB study participants were sequenced, with 95.8% of targeted bases covered at a depth of 20× or greater, as previously described (Szustakowski, Advancing Human Genetics Research and Drug Discovery through Exome Sequencing of the UK Biobank. bioRxiv, 2021; and Van Hout et al., Nature, 2020). Twelve million variants were identified in 39 million base pairs across the coding regions of 18,659 genes (data not shown). Among the variants identified were 3,375,252 (median of 10,260 per individual) synonymous 7,689,495 (9,284 per individual) missense and 889,957 (212 per individual) putative gain-of-function (pLOF) variants (data not shown), of which about half were observed only once in this dataset (singleton variants; data not shown).

It was discovered that a missense variant in PIEZO1 (rs61745086:A, Pro2,510Leu, MAF=0.98%) was associated with reduced risk of varicose veins (7,455 carriers; OR=0.69, 95% Cl 0.61 to 0.70, P=2.61×10⁻⁸). PIEZO1 encodes a mechanosensitive cation channel with a key role in venous and lymphatic valve formation. It was previously shown that rare pLOFs in this gene increase the risk of asymptomatic varicose veins of lower extremities by 4.9-fold (162 carriers; 95% Cl 2.8 to 8.6, P=3.2×10⁻⁸) in the first 50K exomes from the UKB, an association that is now estimated at 2.04-fold with ^{˜}8 times more data (1,302 carriers; OR=2.04, 95% Cl 1.63 to 2.56, P=5.3×10⁻¹⁰). The new protective association with rs61745086:A, which replicated in the GHS cohort (2,243 carriers; OR=0.66, 95% Cl 0.47 to 0.93, P=0.017), suggests that this missense variant likely has a gain-of-function effect. This is important because it suggests that activation of PIEZO1 may provide a therapeutic pathway for a common condition with no available pharmacological interventions.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety and for all purposes.

1. A method of treating a subject having varicose veins, the method comprising administering a Piezo Type Mechanosensitive Ion Channel Component 1 (PIEZO1) agonist to the subject.

2. The method according to claim 1, wherein the varicose veins comprises saphenous varicose veins, veticular varicose veins, spider veins, or pregnancy-related varicose veins.

3-5. (canceled)

6. The method according to claim 1, wherein the PIEZO1 agonist comprises a small molecule.

7. The method according to claim 6, wherein the small molecule agonist is Yoda1, Jedi1, or Jedi2.

8-9. (canceled)

10. The method according to claim 1, further comprising detecting the presence or absence of a PIEZO1 variant nucleic acid molecule encoding a PIEZO1 predicted gain-of-function polypeptide in a biological sample obtained from the subject.

11. The method according to claim 10, further comprising administering a therapeutic agent that treats or prevents varicose veins in a standard dosage amount to a subject wherein the PIEZO1 variant nucleic acid molecule is absent from the biological sample.

12. The method according to claim 10, further comprising administering a therapeutic agent that treats or prevents varicose veins in a dosage amount that is the same as or less than a standard dosage amount to a subject that is heterozygous for the PIEZO1 variant nucleic acid molecule.

13. The method according to claim 10, wherein the PIEZO1 variant nucleic acid molecule encodes Pro2,510Leu, Pro2,024Leu, Pro2,079Leu, or Pro92Leu.

14. The method according to claim 13, wherein the PIEZO1 variant nucleic acid molecule is:
a genomic nucleic acid molecule having a nucleotide sequence comprising a thymine at a position corresponding to position 69,579 according to SEQ ID NO:2, or the complement thereof;
an mRNA molecule having a nucleotide sequence comprising: a uracil at a position corresponding to position 7,785 according to SEQ ID NO:13, or the complement thereof; a uracil at a position corresponding to position 1,095 according to SEQ ID NO:14, or the complement thereof; a uracil at a position corresponding to position 6,073 according to SEQ ID NO:15, or the complement thereof; a uracil at a position corresponding to position 1,336 according to SEQ ID NO:16, or the complement thereof; a uracil at a position corresponding to position 6,073 according to SEQ ID NO:17, or the complement thereof; a uracil at a position corresponding to position 6,236 according to SEQ ID NO:18, or the complement thereof; a uracil at a position corresponding to position 7,526 according to SEQ ID NO:19, or the complement thereof; a uracil at a position corresponding to position 1,336 according to SEQ ID NO:20, or the complement thereof; a uracil at a position corresponding to position 361 according to SEQ ID NO:21, or the complement thereof; or a uracil at a position corresponding to position 7,776 according to SEQ ID NO:22, or the complement thereof; or
a cDNA molecule produced from an mRNA molecule, wherein the cDNA molecule has a nucleotide sequence comprising: a thymine at a position corresponding to position 7,785 according to SEQ ID NO:33, or the complement thereof; a thymine at a position corresponding to position 1,095 according to SEQ ID NO:34, or the complement thereof; a thymine at a position corresponding to position 6,073 according to SEQ ID NO:35, or the complement thereof; a thymine at a position corresponding to position 1,336 according to SEQ ID NO:36, or the complement thereof; a thymine at a position corresponding to position 6,073 according to SEQ ID NO:37, or the complement thereof; a thymine at a position corresponding to position 6,236 according to SEQ ID NO:38, or the complement thereof; a thymine at a position corresponding to position 7,526 according to SEQ ID NO:39, or the complement thereof; a thymine at a position corresponding to position 1,336 according to SEQ ID NO:40, or the complement thereof; a thymine at a position corresponding to position 361 according to SEQ ID NO:41, or the complement thereof; or a thymine at a position corresponding to position 7,776 according to SEQ ID NO:42, or the complement thereof.

15-29. (canceled)

30. A method of treating a subject with a therapeutic agent that treats or prevents varicose veins, wherein the subject has varicose veins or is at risk of developing varicose veins, the method comprising:
determining whether the subject has a Piezo Type Mechanosensitive Ion Channel Component 1 (PIEZO1) variant nucleic acid molecule encoding a PIEZO1 predicted gain-of-function polypeptide by:
obtaining or having obtained a biological sample from the subject; and
performing or having performed a sequence analysis on the biological sample to determine if the subject has a genotype comprising the PIEZO1 variant nucleic acid molecule encoding the PIEZO1 predicted gain-of-function polypeptide; and
administering or continuing to administer the therapeutic agent that treats or prevents varicose veins in a standard dosage amount to a subject that is PIEZO1 reference, and/or administering a PIEZO1 agonist to the subject; and
administering or continuing to administer the therapeutic agent that treats or prevents varicose veins in an amount that is the same as or less than a standard dosage amount to a subject that is heterozygous for the PIEZO1 variant nucleic acid molecule, and/or administering a PIEZO1 agonist to the subject;
wherein the presence of a genotype having the PIEZO1 variant nucleic acid molecule encoding the PIEZO1 predicted gain-of-function polypeptide indicates the subject has a decreased risk of developing varicose veins.

31. The method according to claim 30, wherein the subject is PIEZO1 reference, and the subject is administered or continued to be administered the therapeutic agent that treats or prevents varicose veins in a standard dosage amount, and is administered a PIEZO1 agonist.

32. The method according to claim 30, wherein the subject is heterozygous for a PIEZO1 variant nucleic acid molecule, and the subject is administered or continued to be administered the therapeutic agent that treats or prevents varicose veins in an amount that is the same as or less than a standard dosage amount, and is administered a PIEZO1 agonist.

33. The method according to claim 30, wherein the PIEZO1 variant nucleic acid molecule encodes Pro2,510Leu, Pro2,024Leu, Pro2,079Leu, or Pro92Leu.

34. The method according to claim 33, wherein the PIEZO1 variant nucleic acid molecule is:
a genomic nucleic acid molecule having a nucleotide sequence comprising a thymine at a position corresponding to position 69,579 according to SEQ ID NO:2;
an mRNA molecule having a nucleotide sequence comprising: a uracil at a position corresponding to position 7,785 according to SEQ ID NO:13, a uracil at a position corresponding to position 1,095 according to SEQ ID NO:14, a uracil at a position corresponding to position 6,073 according to SEQ ID NO:15, a uracil at a position corresponding to position 1,336 according to SEQ ID NO:16, a uracil at a position corresponding to position 6,073 according to SEQ ID NO:17, a uracil at a position corresponding to position 6,236 according to SEQ ID NO:18, a uracil at a position corresponding to position 7,526 according to SEQ ID NO:19, a uracil at a position corresponding to position 1,336 according to SEQ ID NO:20, a uracil at a position corresponding to position 361 according to SEQ ID NO:21, or a uracil at a position corresponding to position 7,776 according to SEQ ID NO:22; or
a cDNA molecule produced from an mRNA molecule, wherein the cDNA molecule has a nucleotide sequence comprising: a thymine at a position corresponding to position 7,785 according to SEQ ID NO:33, a thymine at a position corresponding to position 1,095 according to SEQ ID NO:34, a thymine at a position corresponding to position 6,073 according to SEQ ID NO:35, a thymine at a position corresponding to position 1,336 according to SEQ ID NO:36, a thymine at a position corresponding to position 6,073 according to SEQ ID NO:37, a thymine at a position corresponding to position 6,236 according to SEQ ID NO:38, a thymine at a position corresponding to position 7,526 according to SEQ ID NO:39, a thymine at a position corresponding to position 1,336 according to SEQ ID NO:40, a thymine at a position corresponding to position 361 according to SEQ ID NO:41, or a thymine at a position corresponding to position 7,776 according to SEQ ID NO:42.

35-49. (canceled)

50. The method according to claim 30, wherein the PIEZO1 agonist comprises a small molecule.

51. The method according to claim 50, wherein the small molecule agonist is Yoda1, Jedi1, or Jedi2.

52-79. (canceled)


SEQUENCE LISTING (50 sequences)

SEQ ID NO: 1 - DNA, 69883 bp, Homo sapiens
  source 1..69883: mol_type = genomic DNA
gggagccgccgtccggcccagctcggccccagtgagccgagcgctgcgctccgccgaggggcagggcggtcgcctgagcgagcgcgggcccgggacgtcggcaccggcggggcggccgaaggagaaggaggaagaggagaaggcggcgcgcgggtccccgcgggtcagccatggcgcgccggccccggggcccccgcaccgccccatagcgccgcggcgtccgctcggtctgggccgggccctgggccctccagccatggagccgcacgtgctcggcgcggtcctgtactggctgctgctgccctgcgcgctgctggctggtgagtggggggcgggcgcctgggggcgacgggagggggctgcgtctcggctccccacggcctggacaccggacgacgccggccggggcgagggctgcgggcgagcgggcgcggaaattcccagggacgcgcgacccgggcgcccgcattcctgaagcatgagcgcgccaggcggcggcggggctcctgtcccagggccgggctggaagggcggcggcggctgggggagacggcaccgcgtgcccacgggggcggtcgagcgagcgccgggcatagcgcggctggcgtctccgccggggcgctgcggagaggaggccgccgggcgaggcggtgtttgccccggtgggaagggccgcggcggtggtggggggagcacgaatctctttttctctttcgggtttaaaaaaaaaagcgcaaagttgcatcaggacttcctgacaatctgggagaaggcgggcttcctgcctggagctgtttaatttggagcttcccgagcccaacgaacgtccgtgcccagggcccagccccgctcaccgctgcacccccctctgccggactgaggcggtcccacactttggaaaaaaatagtgtgggttcctccctggtcctcccttgccctactgggctcagtttcgcaggggcgggggccggcctctgccctggtctgggggaggggacacccccggaggctgtggcctggtgtcagggcggggcaggggtccccagtcctggcatctgtgttccctgcttgccgggcagtggtgcccctttcgcgaagcacacccgggtggcttggtgctgcacggcctggcacccctacccttccccgaccctggcctagccgggacccagggtccgcgccctccgcccgggggctccccacgtgtgattgatctgggaagcagtcggatggaattaacccacggacaagtgggacggtttgcattgggagtccgccatggacacggcaggtggggccttttgattgtaaaagccctttcggagcccttgcctcgctccaggtgggagctcgcccagcgctagctttggggatctagagccgcctgcctgaggctcccagacagactgcgttttgatcggtcgcacagaaaggtggtgaaacttggggaagattttctagacaggaatcaatgaaaaccattgaggctggagaggagaggttttgagcaactctcttcagtgcggtcagccctgtgtggactgggcagcctgggacctgctcccagtgcagggtcagatgggccgtaaacagggcccggctgtgttccttcctgtgccttgaaaacaagcaggacagcctggcacagaggcagagtctagagctgacaggccttagagagggaaacaggaaagcttctgaaacgtcccgttcacactgatcgttccatttcctcttgtgtctgagtgggagcgggtgtcctccctgcagggaatgccccccctctcagatggcagctgctccttgggcagagttggcaatgtttttctttaaatgaccagatggtaaatattttcatgtcacaaaatcttcttcttctggtatttttccagccattaaatgtaaaagccctcctgagttcatgggctgtacctaaacaggtgttgagcccgattctgtggcacatgtggtttgctgccccctgggcattggtcagggggcctgggttctgccttctcgattgctatccgcgtgggggatctgggggagggatcactgttcttcttgcttttggcctccttggggaggatggggaggtagcccaggggtgctcacccaggccccgtgtcagtcttctatgaaacttttaaagaatagtgatgactgactgtctgtctgtatggtactttccttaaacctaaaactggtcccaaataaagtctcttaatttgaaagatgctgaagcccgggccataccccacactgattctgtgtctggggatggggcgtggggcctgggcctcactcagtgttttctctcagtcacctgggggagatggaagtggagccggccaagaaccctgcctgcctgcctgctggccgggactcctgagtcaggctctctggccctggggtgtgggcagctccagatggacccgcgatgtgcaggttcagctggcctggccggaggtgggacactggctttgctgtctttggagtgccccctccctctctggcgagctttggctggaagcagttctaccgtgttttggaaatgaatgaggccttcagaaggcattagtcagtgtgtgcctgcgctggctcagacagtgcctggtgagggtttgagtcatcctggggtgcccctggcccccacgccctccctctccagtgcaggatcattacccaaaaatctggcagggagctgccccacccacagggagcaggggcctccttcagcagtctcacctaatgttgctggagccttgggggatcagggcccatctcttctagagagatgtcagggcagggctgggcgcgatggctcacacctgtaatcccagagcttcgggaggccaaggtgggaggattgcttgagcgtagccattcgagagcagcctgggcaacgtagagatccccatctctatgaaaaatatttaaaaattagctgggcatggtggtagtgcacctgtagtcccagctactcaggaggccaaggtgggatgattgtttgagtccaggagttggaggctacagtgagccatgattgtatgactgcactccagcctgggtgacagaccctgtctcaaaaaagaaaaaggaaaaaaaagggcaggacatttgtgattctgataatatgggaccgcacctccagttctatcagtgggaaatctagacagggctgcggaaagccagctggtgcgagaggagccaccgtgtcactgactgtgggacacccacgtgggctgacaatatggcttctgcttttcagggtgcctcgtggcacagcctaggggcacacccacggccggcaagctgggcgtcacctccttcaggctgattgtcactgagaagtgtcaccatttagcatgagggatgctgcctcctttttcagaacattgtcaccatcagggtctcaccactcctgggaggcggccgagaagctggggaacagcaggcactcggctcacagttgctcagcagtgcagaccctctgagctgagcatggcagagtcacccttcggaggcctgtgcccgggtctcaggacctgcacagaaccctggcctgtcccatccgagggtgctgggaagagcatggccgtggcagagtagggtgggagctgctttcctctgtggcttgggggccccttctgagcatcagctccctggtgtgacagaggggcgcactctgtccccatgctgggcctggaggctggatgagtcagcaggagagcctggggcctgcctcacagcaccaagggctgcaggtgtgagtgtgcacatgtgtgcgtgtttggggaaggggccagggactgcccaggagctgaggatgggtcacagcgggtgctcgtcccgcagcgggtcactggtgcccaggacacagggagctccagccccagctgccagggtcccacagagaggaagtttcctctgggggtgggtgggggcgcacagtctctgatcctggccccaaggcagcttcctgggcggtgtctctcctgtgctgactcggcagtgcatttgctttcggtgctcaaagatgaaggggaaccaccgtgggccttgacggcctcatctgcccgctgcagcccactcctgagatgggaccaccgcagtcgtcagggtccagtgagagccgcatcttgcaggaagccattcctggcctctctggcctcagaaatccccttttcaattcaacaaaatgttagtcttctgtttagctattttagaaatagacagttaggcttttttctcttttttcttaaagacagaggctcactctgtcgcccaggctggagtgtagcggcgtgattttggctccctacaacttcagcctccagggctcaagccatcctcctgcctcagcctcctgagtagctgagattacaggtgtgtagcaccatacctaatttttgtattttttgtagagacggggtttcaccactttggccaggctggccttgaactcctgaccttaagcgatctgcccgcctcagcctccaaaagtgctggggttacaggcatgagccaccgcgcccggctgacagttagacttttgcttctttgtttatataagctttttcttctggttccaaaagcaagtttgcccttccttgtggtagagaatcctagctcacagagcagtttagaagccagcacagtatcccacacacacatctggcatggacagacccttccttgctgggtgtgggtcctgtgttcttctgaaaggcaagctgttcccagccaacccctgcccctctctgccttagcctgcctggaggcctgagtctcctgggtgactgtgaggtgggacccccccttcccctacccccacccacatcctctgtatctgccttctgtcctgctcttgacctttgagctcctctgctggcttcagggtgcggctgttgagcctattttttggattaggactctggggtgagggaagttaattcacacacccaagatcacactggtgggaagggacaggcccggggtgaaggcttctcctctccttggcggttgagtcccacacctgctggccgaggcacctgaaggggacttggggtccagggtcactgggaggacgggggcaggcagaggggtggccgacctggtggcggctcgtgggcagcagccgaccctatcttgctctgaacgtgtggggccctcacccccttctctgggtctgggtttcctcccctgtaagtgacaccgtaaaagcttcacagccgcttccaagtctcaggtctctcgggctttggatctcacacccaggctgggtgggggtaggggcgggacagccgtcccccccggaaggctcagaattcctcgcacaatcgtggggccaggagacccgcaacacaggctttcccagctgcgctgagtgccgcggtggccgggggtccgtcgggcctccatggagctgaggggaaggggccactcaccgcctggtcccggagcacacagggcagctcccaggagcacagaggctccttgggacctgtggggctgtcggcctccctcatgctgcacacacagcgcgtccccaggggtgtctgcagcccagcccatcccactgcagattcccgaccactcagattcattcatgcatcctctcacgccggtcctgtgggacagagctctgggcagcagccagaagtccaagttctggttcagggccagtggaggtgggtgttggggtggggctggagctccctgctctccctcccaagctagccaggaaaggaggttgggggccccgcacggtcattgctgtttattcacaaagcgcgatgctgagcacaggcggggaaagaaaagtgcgatcagtgccaggaaaatggggctcccccgacgccgtccaaaatgggatcccttgccgggcgcggtggctcacacctgtaatcccagcactttgggaagccgaggcaggtggatcacctgaggtcaggagttcgagaccagcctgaccaacatggcaaaactccatctcagctaaaaatacaaaaattagctgggcatggtggcgggcacctgtattcccagctgggaaggctgagacaggagaatcgccggaacccaggaggtagaggttgcagtgagccacagtcgtgccactgcactccagcttgggggcttgggggacagcaagactccctctcaaaaaaaaaaaaaaaaagtgaagtcgtaaatcagattaaattccctttttaaccctttgaacctctgtcctcccctgttcccagcgggaagcctctggtgaacgcgccatgcaccccacctgcccccgctctctgggtctctctcccagctggaacggccgcttccccaggtgccttccctgggccacagccttgtgcctcggcggctgctggatgcctgggtgtgtgggtggctcccagtgtgtgggatggcacacgagcctctcgcccttctgtgtggggtcgcacacccaccgcaggccgtatttttgctcacgttcatgtttctccacggtggacgctgggttgcagggacccttcctgtgtgcgggtgaggatctgggcagctgctggtgccgggcccatggggacgctgaccgtcccgggtgccggctctgaggtgtgcagtggacggctgtcctgccgggcgctgccagggccccttaggccgacgtgcgtggccacccgattccccgccgttgtctcaggaacctttgccgagtggggtggatcaatttttcgggtgtgttttaatagcataattacagggagtatttcaggctccctctgacgggccggcagggtttggctgccggctgtttaccaggctccaatctgcacactatttttctgtgggtatatatagctggggctgcttctccttcctcaggttcaggctaaagagggacagcagccgcctcagccaccccctgtggtttcctttgcctgtggatgggcggctaaaatgggcccaggaagagtcaagaacaaggccggctctcggtgccacagctctacccccaaaagcaggaagggggctcgggccatgcccatctgtgagctacaccggtccgggagcggcatcaggcaggggagtcctggacccccgcagtgctggggtgtgtttgtccgccctccctcccgtgtgtctagaagcctccagcctcggggaaaacaatgaaactcaactgtgacttaaacagattcccaggcccgcaggagctcccggaggcttgtggctgtggcgagacctggagggccatgcgggagggacagacgcaggtttgcggaggccgcctgcccaggaggggcgtcaaaggaggggacagatgtgggtttagggaggccacctgcccgggaggagcctcgaaggaaggcacaggcgtgggtttggggctgcctgccctggaggggcctcaaggaccccaggtctggtcttggtctcactcacctcctggaccccccaaggcctgcagtttgcaatctgtcgcctgggacccccactgtctgcctttacgcagctcagccaccacgcggccctcgctccctcatttacttgatttctgtttatggttaaagtaccgtttaaaacgacacatcattaaagcaacatgaaagggagttttgaaaagggaagccatcgtccatcccactgcccctgcctcagcggggatttacttttcctttcctgtctgcgggccagtgacaatgaggaccccgcaatgtgtctgcgggccagtgacaatgaggaccccgcaatgtgtctgcgggccagtgacagtgaggaccccgcaatgtgtctgcgggccagtgacagtgaggaccctgcaatgtgtctgcgggccagtgacaatgaggaccctgcaatgggcggcctgtaaggctctgccctggcctccgctgccttcgctttctccctccttggtgggtgcacgcccttgtgcttttcctaaaagagcaggtcctccgggcatggtggctcacgccggtaatcccagcactttgggaggccgagacgggtggatcccaaggccaggagttcaagaccagcctggccaacatggcaaaaccctgtccctactgaaaatacaaaaattagctgggtatggtgacaggcatctgtaatcctagctactcgggaggctgaggcaggagaatcacttgaatccgggagacggagtttgcagtgagccgagatcacaccattgcactccagcctgggcaacaagagcgaaactccatctcaaaaaagaagaagaagaaaatcctgacacttcagcctggtgcaggcccttccctccttctagtccctgccaagaagtgagccgggcccagatctcctgccgggcggggaatgagcacacacattcccctcttgggacagacagcagcagcagccctgttgcacacatgaggacgtacaggctcaggggccgtgggtggcagagaggctatcagcgccggactggcccgccccgagccagggtccagccccacagtcctgtccccaagccctggcccttctgcggtcactcccgtctgctgaatcccctactctgcccctggtgtgtggccccccagttccctcctgtgttcattccctgctaacctcccgtggcttcgcctccccagatgccctgagcacacagcctctccccttcctcccctcctagatgtgcacataggagccgcccaaaggctggggcagctagtggggcccctccaagggaagctgggccccgggcaatgccctgagccaccaggtcctggccctgcgtctcatcccttcttttttttttttgagacagagtctcgctctgtcgccctggctggagtgcagtggcccagtctgggctcactgcaagctctgcctcccaggttcacgccattctccttcctcagcctcccgagtagctgggactacaggcgcccaccaccacactccgctaattttttgtatttttagtagagacggggtttcaccgtgttaaccaggatggtctcaatctcctgacctcatgatccacccgcctcggcctcccagaatgctgggatgacaggcgtgagccaccgcgcccggcctcatttggttccttctgtgcaccacagtgtgggatctcgggtcctgggtggcacgtgtttaacctgaaaggaaactgcccctgcgctccccagcttgtagcccgtgggcctggctctgaggccccgactgccccacggcctgtgctgcgcatgctggagccaggctccggctctggttggcgcctcccgtggctttaatctgcagtgacctgggtgcttaacgagggcctttgcctctgcgtgtgcactgccttcttctgaggagtctcctcaagtctcccagctgtttaaacaatggggtcttttgtcttttgacctgttggtgccatcagccagcctcctgcacatactctccctacccttggctgccactcctgtcctgcccttcgccatgtcttttttcttttcttttttttttttttttttgacagcgtcttattctgccacccaggctgtagtggagtggcacgatcttggctcactgcaccctccgcctccctggttcaaccgattctcctgccttagcctcccaagtagctaggactacaggtgcccaccaccacacccggctaatttttgtattgttagtggagatggagtttcaccattttggccaggctggtctcaaactcctgaccttaggtgatccgcctgccttggcctcccaaagtgctgggattacaggcgtgagccactgcgccgggcccccgccatgtgtttttaagagcaggcatttcattcagatgaagcccggacactcttgggggttctgctcaggggctctacctgccactgaacgtcctctccctgtgtgggtggccatggcattcagcctgtgttgggccttgttctgcctggctactggccgctaggtctgccccggcatcctctgtgtcctgtgtgggcgcctctgcctcctgctccagcctgtgccaggcaatcctgctcaccttccaggagccaggcctctccccagggcctgcgtcctgtcagggtcagggacggcccctctgccatgctccggagtccctggtcccctcactccgttacgtcctgggtgtcacgggtacggccgggaactctgtcgtcttcactgtctgggccggggcctccctgggtgtctgctggatggagtgggtgcctttgggtccctgcaaagtgagcctgcctcccagcaccgccctgtcgttacatagccactatctttgcgcctgtttttccttcctttgactggttcctctggggttaattcccaggcctggtattaccatctctgaatgcctgggtggtttcagcgcccaggaggctgtgctgaggtatcttagaccatgtgggcaccgttcgctcctgcgcagccggctccggggtgccctgtttttgcggaatcctgcagggaaccccatgtaccctaagagtgctctccccagccactgtggcataagacaagcggtctctttgcccttgggccccatccttgtctggtcggcccttcttcatgggtccagcgcgggattgccggcttccttttcaggcttcctgggacccccactcagacctgcagctgggccagcgatgcccacccgtttctcctccacgtggtatacagaggtgcccaggctgctgctggggactctggagcccaggagtgagtctccttgaccctgagctgtcctggctatcacagctgggtcctgttttccccttcagcacccacgggtgtctttctccagtttatattgttatttttatttatttacttatcgagacagagtctcgctctgtcgcccaagctggtgtgcagtggcacaatctcgactcatcgcaaactccgcctcccgggttcaagcgattctcatgcctcagcctcctgagtagctgggattacaggcatgtatcaccaggctggacctctagtttatattattacagcctggtcagggagtcactgggcgtcgccatcctgtgggtgggaagggggcccaggcagaggccaggaggaggtgacagtcatgcgcttagaagctcaggccacgcccacccagccctgcctggtgaccggttcctgctgtgttgggagctgcatcccagaccttatcgccgggcacagaacttccaagccaggggagagggaggcctggaggggccccaatctctgaaggtcagcagtggcggggaggaggcttgcggtgctgaagggactcgggggacctgcagggaggtgttggtggtcagggatggtggcacctgaggggacttttggcagggccagaagtgcccagagcaagctccggtggggccctgcactggagggctggggtcgagtgaccctcttcctaagaccacccaggaggacactgggtgcagggtggccggagccctcacccccagtaggcagctgctgtccactccgccgacctgcctgtcacccagtaggcagctgctgtccactcccccgacctgcctgtcacccagtaggcagctgctgtccactcccccgacctgcctgtcacccaataggcagctgctgtccagtcccccgacctgcctgtcagccttttccctgtcaggccctgttcctaggagcctggagacctcaggggtggccttgagcccccagggtttttttgaggggaagcgccagctgctgtcttcacccttcccctagtgaggccaggctgtgcagggccacgtggaggcagtctgtgctgcgcccatcggtcgcctggcttcctgccgaccctcggcccccagccacctctggtctcgggcaaggcccctccccctgcccacctcctccctggcccccacgccaggtgggcagacctcctctgcggtttttattcagggggtccctcgttggctgcccactcttggagggctgtcctgactcaggcctcccctctactcagatcccctgagcgagggcctgggcgtgacgccgggagttactggggggccagagggggaggctcagcctgaatcaatgagacccaggaggaaggaggcacgtggacctgagggctggctggagccgctggtgacagatggaggagtaattgctgcttccagagcacagcgagctcgagctccctggagtgccagaagcttctgggtggacagacaggccgcctcacattccagaggctgacacagtcttccaggcacccctggggccagctggaagccatgtgcctccactcacgtgtcccgtgggtgtttggaggggaggctggccctgccatggcccaccccagcctgcggcctcaggagcttgcacatttgcaagggggtgacatgaagactgagccagggctgcggcgggcatccccctcagagaacagggaggagggggcacaggcctttttttttttttttttgacagagtctcactctttggccaggctggaatgcagtggcacgatctcagctcactgccaccttcgcctcccaggttcaagcgattctcctgcctcagcctccccagtagctgagattacaggcacccaccatcacgcccggctcatttttgtatttttagtagagacggggttttaccatgttgtccaggatggtcttgatctgttgacctcgtgatctgcttgcctcggcctcccaaagtgctgggattacaggcgtgagccactgtgccctgccaggcacgggcctttttatgagggaggaagccgtgccctctgccacctgcctgtgggctggggcctgcaggctcccagggccctcagctccagcgtggctgttggttattggcacctgggcctcatgtccacatggaaagtatattccgagctggtggctaagccacaagagcctgcttacctgtcccaacccccggctcacttgctgccatgatacgtctgggattctgggccctccttctgagcttggacacagacttgggacttcagcgctggacacaggtgctggatctggggtctcaatggcaccagggatggtcattctgacagccatgcccagagtctcaggagggcctggggccgcaggtggacaggcatcctgaccagagtctcaggagggcctggggccgcgggtggacaggcatagagtctcaggtgggcctggggccgcgggtggacgggcatagagtctcaggagggcctggggccgcgggtggatgggcatagagtctcaggagggcctggggccgcgggtggacgggcatagagtctcaggtgggcctggggccgcgggtggacgggcatagagtctcaggagggcctggggccgcgggtggacgggcatagagtctcaggagggcctggggccgcgggtggacgggcatagagtctcaggagggcctggggccgcgggtggacgggcatagagtctcaggagggcctggggccgcgggtggacgggcatagagtctcaggagggcctggggccgcgggtggacgggcatctggacgtcggctccactagtgtcacctctgcaaccttgtcccacccagcacctccctatggccctgctctgactgtgggctgagggggacccccatggctagcttgggacctccgctggacactgtcatagggcacttgtcgagtggccctgctgaggttcaggcttgggcgtgtgtggcccctgagcccagcagctgcgtgagccgtggggagccgagcccccagtcaagaaagctgtgtgctccttaccctgcctgctgggcttggggcctgcagggcaagggaggcggtgggatgggagccccgcctggcctggtttcttgttgtcgccgcactcctagctgtatgtagtcagatgaggctcccagtcctggctctgctgctgagcaccattgggccggtggttgggtgaatctctcagcctctctgagcctccagcacttcgtccttaagagtggaactggtctggacctcccaagggggggtcactgggtgcagagttcctgcaaggctgggcccggtgccagtagcggtcgctctgtggggcctttcgcacggttgatttttatctccctgtgagattagcgcctgccctgctctgcctcacggctctggctctccgggactgagaccacctgctaagtgacaggggcggaaggaccttgcaacacagagcaagacgtgatagggtggagctgctccgcccaggccccgcctgcagatgagagggcccagagcctcttaatcccctgcgctcagggcatgtgacggcagggagtctctccagtgccgcccaccggattcctatagttagagctcctcgcccaagcccccagggcacctcactggcctcccaggtgaggacatgaggcccagcgactctggtggtggctggtgtcacctatggcagggccgcgcggggcccagtggaggacgcctggcccggacctgactgctccgtgggaaccgtggggcgtgctcagggtgacttggtgcggctgggctgccagccggcctggctgagtcactggcagtgaaggcaccaggtggtggacgtgctcggcagctggcgggagagtacccccacccaggaccgtgttcaggctgagggggaagggagcggcccaggcaggcaccgcccatggcggggcccccgcactaacggccacctctcccgatggactgcagttctggagccgcccctgaggagaacgccctgctcagtgtcctggcagactctggactcttcaccgtgccatcctcgctacagcctggccaggcagccgctcccgtccccccaccccacagacaggaactgcagactcctaaaggtcgagccagctcccaagtccatagccagaaagtgatgcaggcggggcccggctggggggcaggtgtgaccgggtcatgggggagcgggctgtgcacaggcatcaggctgagaggtctcggggatacgtcctgggagccgcctgccaggcgtcctgtggggctgcctgcaggcctcctctggctttggcaggttctgtcccgacagaccgaggcttgggaagaccggattgtgcactctctgcctggagagagggtggggaggggctggggcgtgtgggcagggagaacaccctgggaggcatcagtcctcttccctggggacgggccatgcgtcaacagtccctcacttcctctctcaggggcagaggcgtgggggtcccgagcctccaggttggagtaggttaggagggcctcagagccccctgctgccccgccatgtggcattggaaggggtgtgaccccctaggggatctgttaaccacactccgccccatgccggcctctctgttccctctaacctgcgcaggagcagggccagctctgagctagcagaggtgacatgaggctcgtcctggccccgcccaccctggcccctccctgtttcccaccacgctccgcccaccagagctacatgttgtgtttgagcctcagccccagacctggctcaggtcctcaggaggaagcagtgacagccaggcccggggtcctctccctgctctgctccctgcgtgctgactggaggctgcaggtccccaggcttggccctgaccctcaggaatggagccggccgatgtgaggtgggggctccggtcatgtgcagtggtaggagaggaggcgggaccggtcccacagctccattgctgccgaggcgttccgcaggtctgtcttcatattggtaaggaaaatgcgaggatggtgtagcctgggcatcccagtccccaagtccgaaatctaaatccaagcggaaaattccaggcctgacttcatacgccaggtcacagtcagaagccagaattattgaaagtatgctctgggcgtggtggcctcacacctgtaatcccagcactttgggaggccatggtgggaggatcacttgaggccaggagtttgagagcagcctgggtaacatagtgagacccccgtcattatttacacaaaattatgtgactgcctttagggtatgtgtacgaggtatatattgaaatgtgagtagaattttgtgttgagtcccatccccaagatacctcattatgcgtgtgcaaatattccaaaatccagaaaatcctaaactggaaatacttctggcccctttggatgaggggcccccactctgcatagcaagtgtgggcgcgggtgctgctgtgtcctagcgtacttcagtgtgtggcctttgcagtgaatagggctggtgtcctcactgtacagatgaggaaactgaggcccagcttgctttgccaaggtgcatccggccccggccatggctattctggctccagatcccatggtctgcagccacaatactgctgtgccccggactgggccctgcagctcgcggtgtcgctggcctgcccattgtgggcaccgcccccaccccagactggccgaggcctagaagggagcagggcctggctgaggctgcaggggtggggacggtcagccagcccctcactgccaggaagggcgcatccatcctggcctctccccagggagaagggaggagcggctgagagggaagcgctcttgccctgtggacgagctcctgccccacggactagggagcccccgcccacaacctgcttgtcagggccacccgggacccccgggagttcggctgctcgctctgctgttaggaattggattagttttccataaaaacaggatgtggtgggtgagagggcagtgtgtccgtctttctcactcccctttttccaggaactgagcacgcgcataggttttagccagggccgtccagtcccctccccaccccccacagggaacaatccactctctgctcttaagtggccacttaatcagcttctcctcctggcccggggagcttcttggagccggcctgccgtggtgggaacagctatggggacaccctgccataaggtccagcagctaagctgggatgtggggggaggggctgcgaggcccaggcagtgtgccaggccgcacaagaggagcccagctcttgccccaccagctggcagccctggaccgaggttgggcccgtgaggttggctgggccctgggccctgggcccccctccccaggacacgactgtggtggcacatggctttgggggctcgtgggtcccactttgcagacctctgctttaaggggtctggtccacggggtcccctctggagggcctgggggaaatctcagggacctggggtctggacccgggggagggagcgggagaagcatgtgcgtgagtctcgtgctgtcagggagcccgggaagtctgcgagggcttgggggttgtgtcagggagtgtgggttttgcccttcagtggtggaagctggcttgagtcccctcgatccctcaaggctgtagtcctgactcggggctgtagggcagggcagggtggggtcaccctgaggcagaaggctcagcggagatgttctggcttggctctgcccctccctggctgggtggcctcaccctgtgacctttggggaccctggttcctctgagccaggggacagcagtgactccgccttcctaggtggctgaggatgacatgggctccccctgcaaatgtgggttctggctcagtggccaagtgtatgatggtatgtggctcttggggtcctgagagagatgggagaggagcaggggtttgtagggaagctggcggcttccaccccagccagtcacctgcagtgggggagttccaaagctgactgaagcttcgaccttgtggctggtccccttccttcctgcctcagtcattctggtctctgggggatcagggctggggggctctgggctgtgggggcctgtttttgtgacttaaagctctcccagcacagccccctgacctccttcctcatgggcaggacctggcccaggggtctcagcacagccacaggccagggatgcccttgcagatggccctggatggaattccagaactcagaaatgtctccttcccgtaaggatgtcccgagactcatgagaccgtttccttctgggaagggggaggaatggggagatgagtgaagagcgcactgcagctcaatccgggaagaagctaatcaatcaatcagggaagccaatcgatcggagaagctgatgggaaagctgtgcttggtagaataggctcgtgcgggcagagcagtcgcggcactcacaggctgactgtgaggaccttgggtgttactttgtgcttctctgcattttaaaaacttttgaaagtggagaagaggaaagtgaatgttctctgagatttcatggaaaggggaaactgaggcccactctagccagtttgggcccagggttccaacctggggtggccccggccctcgtggcctgaggtgatcgtccctgtggctctgagagcagctggggccgggtccccgttctggggctggtgatcctggggaagagccaggcagtgccctgcccacctagtggttatgagcccagaatgttgatttttttcccttggttgcttcatgactttgttgaatttccagagtatgtgtgggggcccccggcgtcccactcgccccagccctgtggcagcagagctggctgtcaagctcagtcagctgggcccagggccccggaggtaggtgggtgtgtgcctgagcttcccctttgggccctgccaggtgctgggagggaccacacaggcggcaggaactcggggtccccaggcctcggccacaccagcctggtgcttgttatatattgatatgtctctctacctgtgaaatgggtatttattttaaggagctgacgcacgcgattgtggggctgtcacgtctgaagttcgcagggcgcgctggctgtcaagagccgctgttgcagtctcgagtccaggctgtgcgctcaggccgggcttctctgtggcggtcctgggaattcctcctccgggcccttaggcgacactcccctgccttcagctgattggatgaggccctcatgccgtctgctgatttaaatcaatctcattttaaaaacacctgcacaccaaattgcgattggcgtttgactaaactgggcagcgtggcccagcagcgctgacacatgaatcaaccctcacaccccacccgcaggcgtgaccacggcacccacgctgccgactgagaacacagccgtgcgctgactcgcatgtgatgtctctggtcgcacctcctccatcctcagcaccctgttcagaatggagatagcggccgggtgcagtgctcacacctgtatccccagcaccttgggaggcctgggtgggcgggtcacctgaggtcaggagttcgagaccagcctggccaacatggtgaaaccccatctctactgaaaatagaaaaattagccgggtgtggtggcaggtgtctgtagtcccagctactcgggaggctgaggcaggagaatcgcttgaacccaggaggtagaggttgtagtgcgcgagattgtgccactgcactccagcctgggcgggcgacagagtgagactctgtctcaaaaaaaaaaaaaaaaaaaattagagataacgacttgggctctctagagaccagggagccaggctccaggcgctgctgtccagccccttgccagctatacgaggtgctcacctggccacgtgtccgggcagtgcttctggggtggcatccactgggagggcaagatggttctggtacaggtgcccaattctgtccccatttcacttactgggaactcaaggcacagagaggggagggctgagctaggaccagaccccagtctcctgcagaagtacacagacatgatgtacgaccatcctggacacctgccctgagattcccctccctctcctgccctgtcccagtggcctggggggaaggggaagcaaggttctgaagggagtgtgaccagacacctgcccgtgacaccccctctccaggctgcctccgagtggctggtgactcccctcctgcctgcgagggaggtggccaggttgcattcctctctgagtgccggggaagtccctagagagcaggccagcctgtgactgggccctggggcagtctagacaggccagactggacaggccagggggctgggtgccgctgggtaaatcacagggtgagggctctgagtcagcaccccatcttctgtcctgggtccagcaccgctgaggacacagtgggcagccgggtctgccagggccaggtagctgtgttgagaaggcagtgctcctgagaggcggctaccgggaggttttcaatggccaggcttcttaggaagcccttgttgcctctctggggtgagttgctggggccatggttggagtggtcgccagtgtctgcccctggtgccgagggcggagtcctcgttttgggaggtcacggcatgatgctgggagtcaaaggcaggccgtggcaggggcattccttttttctttcttttttttttttttttgagacggagtttcactcttgtcgcccaggctggagtgcagtggtgcgatcccagctcactgggatcccaggctcccaggttcaagcgattctcctgcctcagcctcccgagtagctgggattacaggtacgtgccaccatgcccggctaatttttgtatttttagtagcgacggggtttcgtcatgttggccaggctggtctcgaactcctgacctcatgatccacccgccttagcttcccaaagtgttggcattacaggtgtgagccaccgcgcccggcctggcaggggaatgtcgacgcgtgatctctgcctggagagcacgttcatgtttcccagaggacactttagaacatggcgcctgggtttggatgaacctcagcctaagaatctacctgctcaggatccagcgacgctggtggtgtggacttcagctctggagaatgggttatatggaacctgggcgccgggagggcattgccacgtgcttgctgctggggcttcaagaggaccccatctcctgtggccgagaccccgtgtctcaaggcacatcccctttcgtaccccgccccaccctccgcagcttcatgacctctgggtttcccccaggaccttcgcatctgatgttcccagatccttcctgccactgggtcctgctctggtgccccccggggaagccttccctgaggacccagctcagcatctggtgctaactgtggctcatcgtgcactttggcccccaggaggctgtgggctcctgaaacccctctgaaccacgggcttccaaccacacctgaccccctgcgtgccgaccctgtgtgcaggtgtacaggtgtgcctggggcaggatggctgtgacggcctcacagagcccggagagctgcctcctagcttccaaagccttcatttcaggaatcttaccctctcaattagtctggaatgctgggggcggggccagctccaggtcacagagcgaccttgtttacccagaccttaacatcggcccttccatgctaatcaatgtaatcattgtggtccatgcggcctctggaatgtgctgtccactccctgggtcagccagagagtgtcagggagcacctaccggctgttacccagtgctgccctgacctggttcttcactctgcacatttgtatcacgccagaccctggctggcagctccaggtgacgaggcatgtcagtgccttcctgttctgttctgttttgttttgttttttaaatcgggatgaggccttcctgtgttgcccaggttggtcttgaactctcagggtcaagcgaaccttctgccttggcctcataaactgctggattacaggcaggagtcaccatacctggcccactgctactttctagatgaagagacagaatcccagagaagaagcaggggtttggctgctggtctggagccggttctgctcacctccagcttctgccttgggccgccctgttcacacaggagctgctcacaggctgagacctcgagcagggccctcctagaggaactgggcccccgtaagtgccctgagccgccaggagccggccctgcgtctcatccctatctccggaggacattggctgctagctcaccagctggcccctgggcaggcttgaatcatgacctggaacgccaggtgtctctggctctacccctggacctgcaccctgtccaagtgccccagggccagactgttttggttgcaccctctggacgggctacccccatgatggctgctcatggaaagctgtggttcttagggagctgccaattcctagtcctgcagcctggagctcctgggtataaggtgggggctcgggcgtattggggactgggggtctcaggaaagagcctgtgggacctgtgaactcatagctgctggccgagggaccaccttgtggctgtccttctcagctaggcctggtcagggcttgtgtgcagggcggctgaagctgtgggaggccacactgtccacacagtgccctgtaagcccaccgtgcctcagtttccccgtctgacaagtggcacaacagaagccacttcctggcacacagctcagggtcagggccgacacagcacttgtgggctgccgggagacccgagaggctgccccttccttgccttggctgccacgggtgacctggcaaacccctctggccgtggcaccactgggggtctacccttggcagtcagggttggccgcttggctgggagccccttctcctccccagacacatcctctccttggggctggagggggtcctgccgtccccgggattgtcgagcagcaggaatccaggagggcagtgcctgcagctcagatgggggccagtgggcagggccgatccaagggtggcaggaaagtgcccatcactgacctcaggtgggggaggccatggtgtgtgaaggaaggagagctgagtgggaggtcttactttgtcaccgcccctctgagtgcctgctgtgtgcaaaggccagcgggggccctttcttcagctgggctctgcccagacccctgagctctgggtgggccgggagggagaccttgctgctcacagaggtggctctgcctgacccagctcccttcccagggcacagtgggggcatagggtcggctccgtcagacattccgggacctgcgtccttcctggggccacaccctctacccactgtcccccacctatttacctgtatctgcgccagagatggctgcccagataagccctggtttcctccctttctggagaggctgcgggggctggcgaggaacccacctgcgcagagagtcaggggattgctctgtgtggaacgcaggcctcacccatgccctggaatctgtccccttctctgttgctgagggatgagtcccagatccctgaccctgcaggggagcccagcacagaaaaactctgaggcctccacaccctggcagcgctgctggtcgtctgtggggaaggacaggccctgggagggagggggaggtgcggagggcagtggggagggtcaggaggaagtgggggaagggccacccaggccccgatgtgggggatgtctcacgcgtggggtggggcattctcatctctgcttggtctcctgccatgctgggggtcgttcactgcggaccccaagtaccatgaagatggggatgggatgctgagcagcatcggggagaacgcaaaggcacctcccagctcacccgcccccaccccgcaagcacaaccattgccatggtgtggggaccggaaggcggcggctttgggaccagactgcttctgcctcgggccgtgccgctgggcctttggtcagcaccatcgtgccctgcaagtcactcttagcctggtgccctcctgggcagggcagtgccacaagagctcaggcccagatgtgcaggtgccactgttccacccaccagcaggtcacctggggaaccctcccctcctgcagcctctgtgtgctcatctgtgaaatgggcgtggtggagtcacccctggcttgtgggagaatcccaggggctgatgcctgcaggaccctgtgggctttgccccgctccctgggcagagacagttcccccagtcccacccacgtggctgtgtgcagcaggtgcctgctgaccctctgttcctgcacagtcaccttcttcacagacggacccccaccctgccctgcaaacccctccaggggctgcgggctgagtgtgtccgggagggtgtcctgactctccacgccagcaggtctgagagcagatggctgtggcaggtgcggtgggtgcccagcccacagcagccaccaggcctgcaggaccctgccccgtgtaggtcagatgagccataaaactgagtttcctggacactgagctaattaaacctggacaccgagctaattaaatggtccagaagctcctcagtgccccaggctgctggccgggctccaagtaggtgaggaacattccgtttacctcctgctgcgtcgaaggcgggtggctcccctcgggcccctgcctgtcccgggccccctgggtgctgctgcgtcaaaggcgggtgtctccgcttgggaccctgcctgtcctgggcttcctgggtgctgctgcctcgaagacgggtggctcccctcgggcccctgcctgtcctgggctccctgggtcctgctgccttaaaggcgggtcgttcccctcggatccctgcctgtcttgagctccctgggtgctgcctgccctgtggccacgtcccactgttgcctttggacaaagctctcctagggcctggggttctcccatgagatgacaggggttggttcaaaggtctctgaggtttttagagctttgtgaagtgttctagaatccattttctctctcattgctggacagagagtatagactgggctttttcttgagtttctctcaactcttctttgtccatgaggcaggaagaggggcttcctcagtcctcagcttggggtggattgggatggacagagaaccgtgcaggatcccagcctgagacggtccagcgccccgggggagcggagcccgtggctcagccgtctgtagccacggccggggtcactgtcactgagggcacggaggccccgccggcgaggtccgaggtgggcgtgtggggtggtgggcgctggaggcaggacctctgcttgtggagggtgggccaggcgtggaccaggtgtcacgtccgctggggcctttgaggggcaggtggggattggcgcggagaaggagtgaagcagctggggttttgggagcctgtgcggagcaggcccggatgccagggtggctggtgaaggtgggcctggccgggctcggcctccattgggggagccccccaggtcccctccccacacagcttccccctctgtctgctgtccaaggctcctgtgctggcactctggggtggtaggccatgcagcccgtgtgaacctccatagaccttgctgtgaacgctgcacggggcgtctgggggcgggctggcttccccccgtcccctgggccaaccttgccagccttcttcttccataaaagtgggatccgtctgagccctcatggcccctctgggtctgggtttgctttttgtgctctgcctggggtcatggcagggaaggcccagcaggctcccttgcagagcagggcagagcagcggccgccaggagctgcctgtaccacgtctgctcttcttcctctcttcctctgcccggccccgcccaccgtcgaaagcactagcacgggagtgtttatcggtttctcttccaagccaaataaggcagagagcgctcttgcaggagttggagcaggccagggggagggcagctggggcttggaccaggaccccggctccctgtagctgccctggcctggcagctgtcatccgcgaactctgatcctcgcctgccctcacccggcccctttcagagcttcccatgctcagctggtagcaaaagaagtcaagacctagcaaggtgcctgccccggccaggaggcccaggttctagccctgcagctgccgctgtgagctctggggactcagacgactgatgtccttcccagcttctgtttcctttgatgagaggaaggttggaccaagcgacctgccctgcttctgtctgtttcctccagtccttttcctttttgaccagttcacttgttgataactccagcaacgcatgaaaacattccccttgtaaaaaagggaaatgccgtgggtaggggggaccaaggacctcttctgcaggagcagacgcaacagcttggtgtggatcttcctcagcctttttgtccatcctagacatgtggaatttcagttttacacaaaaggaatggtaggcgtctgactctctgtggcttgtatcacttggcagtgccacagggattggtgcatgtttgctctctcatgctatgtgtcaggcactgttgtctctgcgacaatcctgtgaggagggagttatggttcttgatttatagacgaacaaactaagacagaggagattgggctcatctgtggtcatctcagcagttgtgagaggtcagggccagagcccagtggtgtcattctgcagggcacatacagacacgacccacagggttctgccgcaccccctgccgcttggcctgctctgcaggtgggtgccgaggctgttcctgcatctgctgtgtcttgtgagtccgtgggtgcctgtggagtgggggcagtgggtttgcatttccaagcactgcctctgtcataggctcagggagaagcagttacgcatgagctgctatgagccacgggtatgactggcatcacagcaggaggagccctggggatggctgagtgacgatgggtggggacagagctcacagaagccagggttgcccccaggccaggccactgctggggccaaggctgggatgcagcacgatgttggtctgtcagggagtggcaggaagcccacagggtcagctgacacttaacccagagtggtgggagccttgaatgccagactgagggccacacagttggtctcggtgcacaaggacttgggctgagagggctgttgctcctcaatggccatctccttgtgggaggagagtccgggcagccgcttccaggaacccacccatgcttgaaatagtgggaccacagggctgaggaggctcctggggattcctcccaggattcagctgctctgtccccaaccccggagggcctattcgtctagccctatatcctctctgccagttaggtcagggcagagaagtagaggcacagagggtggcggaggggtgtccagccaggcctttctgtggcaccttctgaggctggctggagtctgtgaaatctgtgataactggcccagacccttcctgcctcctggggcatctgctggagctgaggctgctgggggagtctgcctgtgacttgagtctcttgctgggccatgctgggctcagttcgcctgtctgtggggtgggatggtgccacccttaggttgttgggaggacccaaggagagtgatgcctccagccatggcagctggcccagctccggcccgcagcctggctccttcagggccaggaacccccaggtcatggactccaaccctcgggctctcctgcttcccaggtgacgaagcttcaacccatgggctctccttcctgggtgtggaagccctggagaaggtggagatgggatctcagtccagcccattggctccacttcctttgagaagctctttcccccgtcttggcccccagcccttgttcaagcacctgtccctctcctgtctcctaggccccgtcagtccttttggagacctgttcccacccgcccctgctctcacaggctgccagtgtccacacactctcaggcactgtcattggagccttttaaccacacgggaaagcaggcaggttaggtaactaccccccacccccccgccagcaacgccccctgcatacccagggcctgcaccaggtgctgctgcgctgccagctctctcgaggtccctcctcctaacctcaatgcatcgcgtcttccagcccccggctccgagggctcagcctccaggtggtctaacctggagtctaaacctggattgccatctgcctgcctcagcccttcccttccccagggaccgagggccagtgcggctccccagccacccattttttgtttgttttgagacggagtctcgctctgtcgcccaggctggagtgcagtgtcctgatctcggctcactgcaagctccatctcctgggttcacgccattctcctgcctcagccccccaagtagctgggactacaggtgcccgccaccacacccggctacttttttgtatttttagtagagacagggtttcaccatgttggccaggatggtctcgatctcctgaccttctgatctgcccgcctcggcctcccaaagtgctgggattacaggcatgagccactgcacctggcttgtttgtttttatttttattgttttttgagacggaatcttgctctattgcccaggctggagtgcagtgacataatctcagctcactgcaacctccacctcccaggttcaagcgattctcttgcctcagcctcccaagtagctgggactacaggcacccaccaccacacccagatacattttgtatttttagtagagatggggtttcaccatgttggccaggctggtttcgaactcctgaccatgagatggcgggcttgagccatcttcccgcctcggcctcccaaagtgctgggattacaggcgtgagccacggtgcccggccccagccaccagttttgtacagcactcaggtgggtgtggttcctgaggacatgtcacacgagatccttccggtccattagcccaccccgccccatcagtggccagtgcggccattgtggggtccagagtcccgaggcagaacgactgactccaggttgtggaggggcctcttaggatccttctaggcacccaggttgggcaggtgcctccatgcctaagactgagctcctggtggacatcgggctgggtgtgggcgtcctgcacaccctccctgtggcctggtgcccgtgaggagtggacagtcaccccgagagcatagaccaccctcgaggctcccagatggcggccgcagagccccacttccctgcctggtgtgtgataccttccatggctgaggacacctctgactgctcactcctgtgaccctggacccaagtgctgggccacacctggactccaggccccagtccttccttcttctgctgtcctggctcctgggtggcccccgccctccccgctccgaggctctgccctgtgctgggaacccaggaattgccaccccctggccccggtttctcatgtcaggatctgctggccaggttgactcctgagcgccactgtctcctcagacttgctggtcccacccggtccccacctgtccaccgcccgaggctctcctgtgaggccgctctgcagctcttggcctgggggcccccggcctctcatggtgccttctgcactgtcccctttggagatgggagctttgagggtaggcgccaccgttcggtgaaaaggcggtataagcaaagcaacccccagatactgaaggagccgggaaataaaaggaggcagacagagcaagtgtgttggtacccgcctatttactggcaggaacttacagacggaaacatggcctcaggcagcctccaggcaggtagagctctgaacccaaacgcccgatctggggctcgtatcgtgccctgggaggaatgtgcaggtggctgggaacgtggcgggtggggtggccagattcctgctgcagcaccgggtttgtttaggaagaaacgtacgaggactagacactcttttttttctttcctgcacgtgaatgccagggagtaggtgctcctgcaggaagatgatgcatcaaccagccagtctggaggcattccgagacccggggttaatcagcagattagcatttaaataaagttactctgtccccacagccagctaggcccgagccacatggcgggggcggcgggggcggctgttcgccgcatcccaggcttgctccatctcctcgtctgcaatgtggaggtgacagtcgtggtcctcggctagtgccacaggactgtacgggacagatcacccagccccgtgcctgcactgggggcggcggtgacgtgatggccgcggcggtgacctgatggccgcaggtgtgtccaccctctggttaggcctctcctctccctggggcgcaacaggtgtcctcgggtgggaggttcagctgctgggcctggggccccagctttcagctcctgtgcaggcgtggagcgggacggcacgccggggcgttcgttcacacgatgcccccagctcgtctctgcctgctctgtgatgcagcctcagctcctgtgggtcccgggtcacccacccgtgtgggccgcagtctttctgcaccacgtcctgctctcggccagcgctggcctgcgggccccggcagtgtggggtcacacacaggccgcaccggctgtttcacggagcccccctgctcaaaatcctggccttgggtctctgtgtggctcctggagggagcatgtatgaggtgtccaggttgtgaaagccccatccggcagccctccctgtgacagtccttcaggcctcgtcaggcttggttcagcccgtgttagaggaggccagacagctcctcctcgatcaccacagggaacgagggttgggcagtttctcctctcccacccgggcgtgggtcagctgcaagggtctcatcccaggaactggatcctggcaggggggctcaggcatctgacatcccctggcaggtcttcctatgtgccagtgggagtgggtggtgtccagccctctttgtggaatgttccggttgagtggtgtgtggtcctggttaccaccatgtggcatttgtggctttcctggccagcacccactgaaagcagaggccagcccagcccttggacacccaggggcccagcagctaccgtgggggcagcctctcccctcctgactgtgctcctctcagctgtaggatgaaggtctgtggatgggtgatggctgggctctctgcttcccaaggagctggatgtgggccatagtcaggcgtgtgctggtgaggcgagggggttgggggttgggaaccacagctctctctgaccccatcccattgcagctgactcaggaaaaggcattacccccacggggtcacaggcccagcctgggcagctcctgatgggaacatgggagccacagggcagggcaggctcctgtccccgctccttggattgtctcaggccccgcctcccctttggctgaatttcttccttctctgcctgagcctgatctgcgaagcttccgacctgccctgagttggagccagtcttcagggccaaaagcaatgccgtctcctccaggcagcctgctctgaagtctccccctctggctttagcccacaaagtcacagctgaggccacatcaacacacttagaccaagagcactgccggctgcatagacagcacaggtagatccaaggagtgtttcgaaaagccgtggccctgcggccgggatgcccggacctgggtcctgaccactcttgggagctcctcccctctcagagcccatcccatctgaaaatgggggcattggagcattggaggagtgctcctcactcctgccctgctagccggctactccgaggaccccaagaccagatgatgggagaagtggtgggctgggctgtgaccctgagcccccttctcatcccacccgtgaggctgcccctgcttggtgtctgtggcaaggctgacagaagatggcgccagggaccagccagggccctcctgtctcagcccgtccttgggtgagtgggtgtgctctgggggtggcaggtgtgctccgggggcgggaggtgtgctgcggggggccgggtacgctctgggggggcgggtgagctctggggggctgggtgcgctctgggggggctggtgtgctctggggatggacaggtgtgctctggggggcggggtgtgctctggggatggataggtgtgctctgggggggcggttgtgctctgggggggcaggggtgctccgggggtgagtgtgctctggggggcggtgggtgtgctctgggggcggtgggtgggctttgggctaagtagccctcttcctctctgagccttttccttgtctgtgttgttggtgagtggccgccacctgggacaagccttagctgtctgtccaactgtccaccatgtccccggaccctagaccaagcccgctgcctggtggggcctgcttggtattgaatgaatgaatgaatggatgaatggatgaatggatgaatgaatgagtgaacaaactctggggggcgtgttgctgatcaccccagggcaggcccgtggtgggtcatggtctcaggcaggggacttgccctgccgggtgacgttggcctgccggggaacccctgccaaggtgcctcccatgcccccatcgtcctctgcctttgagtccactctgcccctcaaggttcccctctggctcggccaggggcagggcggtcagggaggcctccagctccagcccctccctcatctccatcctcagcccctgcacccgcctcatctccagccccagccccttgcagcctcaggctcgggcttctgagaatcctttgcagatttcagggctggttatttttttattctggcaaaatatacatccataaagctttgtttcctgagatgatctcaccctgtcgcccaggctggagtgcggcagcgtgatcatggctcactgcggcctcagcctcctgggctcaagtgatcctcctgctccagcctcctgagtagctgggaccacaggcatgcaccatcatccccggctaatttttgaatttttttgtagagatggggtctctgtgttgcctggtctggtctcgaactcctgggctcaagtgatccgcctgccttggcctcccaaagtgctgggattacaggcctcagccaccacacccggcctaaagtttgtcacgttagccgcattgaagggcacaggtcagcagcggtaagcatgttcgcattgttgtgcagccatcactgacacccatctctagaacttcttcgttttcccaaaggaaactgcacgcacggcaccacctccctggcctccccacagcctcagggtctggtgaccttcatccccaggacctacgcttctgactccaggagccctgcctggtgtccacaccacactaaggtgccctgccacgggcttcgaacccaaccctggtgcacttgttgagcattggacgttcctggcaggtcagaccgtgagctcctctgctctgacggatgcatggacggctgccacctggctggacaggtgcagaggggaggtgccaccttctctggggacaggactggggcagaactcacccggctgaaaagtgcagctgtggagaacttcagaacttcagaagcctttgctgtgaatgcattttcctcctgcctcttgggccagatcttagggattttttttttttttaatgaaaatgtataatcgtcaaaaaactttagatttaaaagcatggagccgatggctatgcttggtgcgttaatgggaggaagaggggatcttaacttcagggagggctcctgggggagctgggggaggctgctgtgtccagcagggcgggcggtgccccccggagcccggcactccgcgatgtgtgcgctaagccgaggccgccttgagcccgtagcagcgccgagcgcgattctttcgtgtctgcttccgggagggtggaagggtgaagctgttgagagtgggaagggaggggctgtgcttcttgagtttgccgtgtgcccctgaccctttcaggtgcaagcgtcagctccacggtgccaatggggagatgggtctggaggccccaccaggctgtgaaaggcccggcctcctgtgctgtccgtggaggtcggggtctccctctgccctgcgcctcctctaacttgccctgcgttttccgtgcccctggtctagggtgaggctcgggtcttggggactgaggggcaggtgggtgtcaaggtgagagctgggctgggctgggcctccctccagaagacttcccggggctcctttggcaccaaggatggggcagatggtggcacgcatggcctcccctgggcacagagccagactatggagggatctcgttggctacatcctggctggggctttggggtcatggtgggagcagcagccaggggtctcgcttggacacgggtctgagctcactgtggcctccttcctcagcctcctgaccggggtcctgaggctggagggtgggtggagggtggagggtgggtggatagtgggctggaggatgggctgctggcgccccttaggcagcatgaggcgcctttccagtcccagtgtcctccctggtagccccggaaaggctgagtggtccctcaggtcccctgcgaggagacgacatcaggccccacccctcatctcctgacaaacctcacattcctaggaggaagcaggcctggctagcacgtggcaggcacagcctccctgactccagaccttatcagatcagtgcaagtgccagggagaccccgggtgggagctgtgtggttgggactgatgcaggcactggccggactctcaggatgagggtgggaggccaggccccgggaccttctgggacaggcaggagggcagacacgggacatggtgtgggggctgtggcgagtgcggccacgcagagcatttgggctgaaggcgcgctgtgtcccactgccccatacgtgtgcgggggctcccagacacgtgctgggggcaggaatcatagcatttttttgtttgattgttttttgagatggagtctcgctctgtcgcccaggctggagtgcagtggctcaatctcggctcactgcaacctccacctcccgggttcaagcgattctcctgcctcagcctcctgagtagctggaattacaggtgcccgccaccacacccagctaatttttgtatttttttgtcaagacagggattcaccatattggccaggctggtcttgaactcctgacctcaggtgatcctcctgctgcagcctcccaaactgctgggattacaggcgtgagccaccacacccagccaagaatagcattttctttctttctctttttgaggcggagtcttgctctgttgcccaggctggagtgcagtggtgccatctcagtgtaacctccgcctcccggtttcaagcaattcttctgcctcagcctcccgagtagctgggattacaggtgcccgccaccatgcccggctaatttttgtattttcggtggagacagggtttcaccacgttggccaggctggtcttgaactcctgacctcaagtgatccgcccgccttggcctcccaaagtgctgggatgacaggtgtgaaccactgcacccagcccaagaatagcatttttaaaggaaaaaaagcaaggtagaaagatgggtcaaaaccccagcgccccgctgggaggctggtgcccgtgtcgtggcgtgtgtgagatatgtacggaagcgcgaggcggcttccacggctctggctcagggcagcactcacagggcacacggcctcggcggtgtgacgagctgggccgtgggtgcgctgtcctctggggtggggctggccatcctctgttggcgacctagttaacgcccatctccccgcagcctgcctgctccgcttcagcggactctcgctggtctacctgctcttcctgctgctgctgccctggttccccggccccacccgatgcggcctccaaggtaaggccaggggaccctgcccacgctctcagggtgggagggggtgggcattcgtttggggccaaaattcgtacagctttcctcacctttaacagcccggggtggaagatgaggggtcccgaaataaaatttttttttttttttgagacggagtctcgctcagtcgcccaggctggagtgcagtggcgcgatctctgctcactgcaggcttcgcctcccaggttcacgccattctcctgcctcagcctcccgggtagctgggactacaggcgcccgccaccacgcctggcttatttttttttgtatttttagtagagacggggtttcaccgtgatagccaggatggtctcgatctcctgaccttgtgatccgcccgcctcggcctcccaaagtgctgggattacagacgtgagccaccgcgcccggcttttttttttttttttttttttttgagaccgagtctcgctctatctcccaggctggagtgcagtggcacaatcttggctcactgcaacctccgcatcccaggttcaagtaatcctcgcatcccagcctcccaagtagctgcaattacaggtgtgagccaccacgcccagccatcaaagtttcttattttcattgtagaccccaagctaagctcagaatcttttctgatccgtgttgcaggggctgtgaggggcgaggagggtcatgggggtggagtctctgtgtcccttgggaggccacagggagtggatgggccccaactgcccactctctgcaggctccaggcacgtttcctgcctgctgaggctctggcagcctgggagttccagccgcctggaccgccctcccgctgcccgctgtggggttggacctcagttgactttgatccacttgtgcgggggggggggggagctgagaccccccttgggaaccaccttggggtctgagctgggccaggactgtgttgctctcccgtatcctccaaacagagggcggtgaggccacgtgccaagcctgcagcctgggtgacgagggtagcagcaaaccctccatcctgtgacatgtcacctacctgccctgtgggggccatgtccattttgtgcccacgttttaggagagcgcgttttggagcaaacactcagagacctgtccccaaaggccatgggccaaacgtgggtggggccgggccctccgctggtctttgggtttggagggtgaagagccactgaaaggaaggaaggtccccagggagggcaagtgtaggcagcaagagaccttccccagcagagggcagagaggaggccaagcaggaccagggggcgtgactgtggagacaggctcttgggtgccgcccttctgggaggtgctggacgcatcaggctggcagcgtcggggcctgtccatggctacgaggtgtccaccgggctgcgtttttccaggggactctggggaggatctgcttcctcttccagctgctccaggtgcctaccttcctgggcttggggcccctccctccatctccagagccggtggggtggggtcaggatcacaccctgtctctcctgctccctctcccctgcggtaaccctgtggcttcgtcccatctgcctccccggctaatccaggccgacttccctggtttaaggtcagctgttgggcagcccccatttcctctgctgcctggactcgcctctgcatgtgagcttctgcgttcacagattctggggactggggtgcaggcatcattggggtccatcgttctgccagctgcaggtggaaggtcgctctccggcctcgcgacatctggctggaagcatcccagaggctcctcctagctcgtggtgctgtggggtgggcaatggtcttttttatttattttttttgagatggagtctcactctgtcacccaggctggagtgcaatggcacgatctgggctcactgcaacctccgcctcgcgggttcaagcgattctcccgcctcggcctcccgagtagctgggattacaggcgcccaccatcatgcctggctaatttttgtgtttttgtagagacaggggttttaccatgttggccaggctggtcttgaactcctgacctcaggtgatctgcccacctcggccgagactacaggcatgagctaccgtgcttggctttttttttttttttcgagtgggggtcttgcttggttgcccaggctggagagcagtggcatgatcatggctcactgcagcctccacctcctgggctcaagcgatcctcctgccttagcctaccaaagtgctgagattacaggtgtgagtcactgtgcccagcgcaggcactgatcttggatcatgggtagaattcaggggtgcagagaggcattttgggaggggcccagtgcggccgagtgctgggagtcgctggggcccgaggcccaaacccctccaggatgcatggctggggtgggtgggcacccccactcccggtccctatcctgggcctcccctttgctctgtggagccggttctgtctgttcccaggcccctggcgtttcgccgtttgttccgtaaatatttcatgcccagggcgcaattggaaacactttcctttaatcagcagtgggggaaggcaggcgcccagccaggccaggggaggagctggggtgggaagatttggagaggacccgggaggacttccctgcctgagcctcgtcagaggcccttcagagacggagatgctgcccagttttccgggagggagagaggaaagtgtgaggcttgcccgagccgagtgcccggggctttatgatttgtgcagctgctgggcttggcgtggccctggtggaagctctgacgccatctgagccttggtccccttgtaggcgtcagcctgatccttggggggctggaggttgagctcagactgaacagggaggagaggggtaggggctggggctggggcagagggaacagaccctggtgctccaggcagggctgcaggcagagccacagggggtggctcccagagacctgcttgtcatttagggactcagaagccccatcctgccttaggataaaacccacctccaagactgaccctgctgtctggagagagatcaccccccacctcttaccatgccccgtgcagggaggccctgccaccctctccagcttcactggtcccccggctctgccagccttgtgtagtctccatcctctgtctcaaaaggcccctctcgtggccctaccccgcagctcagccctagctgtcgctgggtgggcctcatggctcagcctcaaggcaccctgggtcccttggctggtcccatgcacaccccatggatcaggttccagggggctcccatgcacaccccacggatcagactcagggcaactccacactccccacagaacaatcaggcccagcccttgtccggcaggtgtggggatggtggctatggaccacgcatcaggatgctgccgtggtggggcccctcagcctttgcaggccgtgccggggtccaagggctccggctctgggggaggtccatgctctgtcaaccatccggtctgcaataccctggggcctccccgccccttcctgtttcctcctgagagccctgcaagctggagggggagccccggcccctttttttttttttttttttgagacggagtcttgttctgttgcccaggctggagtgcagtggcgcgatctcaactcactgcaacctccgcctcctgggtttaagtgaaagtgattctcctgcctcagcctcctgagtagctgagattacaggtgcccatcaccacgcctggctaatttttgtattttcagtagaaacagggtttcaccatgttggccaggctggtctcaaactcctgacctgaggtgatccacccacctcggcctcccaaaggtgtgaaccaccacgccaggccccctgcaccttcttctgggtgggcaagagccatgctgagcccctcccctgccatggataggggtggcaagaatagtccattccccatgggctccctccggcaggcagcggatgggttggggcccacacagtggttcaggaaaggctgaagggcggttgccggccagagctgggcgggtccctggcctgagcctcctctcactccctttcccaggatggaaaggccactgtgagctgggtgtgtcctgccaggagggagttggagtcgggggcaggagacccactgggtctcctgggctcctggaagaggcagcagtgggtccctgcggaggtggcctgatccccggctcagcctggttttcccagctttgcatttgggggtgggacctgcagggggagagtgacggggtgggcggggacaggggctgcaggtggaggcacgagaaagccacccgaccccttgaggtcatgctgtttgcgtcggagaagagggccggtgtcagggtgccccctcggcctctgtgtgcacctccccgctccccgccccgtgcaggtcagcagagcctcgatccctctgcacgttccggccccctcccatctccagcagcgtctcttcccggggtgcccctcggactctcagctccctagtctgatccagcccaggcagggagcgggcgtgccacgtggccaggagtggcctccaggcccagctgcggccccttcgaccctgcggagggatcatggcccagctgttctaacatggccagaccaggacactctggccggcccgagaactgaaccgggaaggaggcagggaaaggggagggaggaagacaatggggaagcagtgacatccgagatgtagccagagacggacatcctggactgtcgtgcagggcaaggcgggtgggcgggagccaggtggcctgagagcccctccctgcccaggggtctctggtcaggcagttccttcccgggctgctggatcgtgtgtgcagggaacccgtcagcctggctgcccaagggcccagatgtcctctcagtgcccgggggtccttgacagccccagcaggagccccacgtgcctgtggggcaggccccgcaggtccctctctgtaggactcagaataccttctccaatgccacgtgctctccctgagtgcccagtgccacagagggccccgctggcgaggttacttcaacgtcaggctagagggtgcagcgagcaggactcacagcccaggttcccaggcaggtgtggcaggagcccattccctgggtcggtgttctcatgtcaccccacggtgaccctgctgtacagaggcgggggcgcggggcccctggctggtgccctcctgctgccagacttgtgctctgctgaagaggggctggcgcggcaggtatgagcccggcacggaggtcgtgagcagtgagaagcctggcacctatgggttcggggcagggaggccctggaaggtcctgtcctccgggagccctgcacagcaccccctggtggtggctcctgttcgtgtcggggctgcagcctcccttcctggacggttttccattcctccttcccactctccctgctgctcacccctcccatcctccacaggtcagaggtcatagctgcagggtcagcaggtcagggcctggggcaaccctaggaaggtgtgagtgtgagaagctggtccaaacctgcctcggctgcctggtggtgcggacagggagtcctgggcatccgtgagggctgcttcacggtcaggcttagggatgtgcagggtgacttggacgtgggtcatgagtctttgctccagaaagagggggctgagtgtccaaggccaatcccgagtctgtcacctaacaccatttgtgctcaaaaaactgaacagagtggacacaggccctgagtttgcgccccgaggccgagagagggcagatgtggtggtgacattcaccaccttggacccagacccagatgctacctgtccctgagctccaggaagttgtgagaagggcctgagctgttctgcactttctcgtggccgggcgtggctggtggtgcaggagttgctgccccagggtgagggcccggaagctcctgccagcacgtgccggggtggaaagggaagctgtccacagccctgtcaggactcagaacccggtgggtcaaggactttggtccggacccctgctgaagggtgagctgtccacatgtgcgccgagagcagaggtgaagccagggctcctgagtgcccccagccacagggtgcgcccgcccagcccctgccctgcagccgaaggcctccctgctggggggctgagtccagtggggccacaggcagctgggagcaggacaaggctgccaggcaaccagatggtgctgccgcttcctgccaggtgtgggtgcacagagagagagaggatgccggtctggggcctgacctggtgcacagcaggtgcctgaaatgccagggtggccatggggactgggtaccatgcataggccatgcatcgggatgcaacttctccttgcagcccctcagccccagggaggcagctgcctgccccacttttctccagagccatcatggccctgctcccaccccagccacggctgctcaggggcgctccgcatgctctggtctccatccacctgcagcccccactggggtgaggtggagcttcttgcctctccttgtgtctatttcctctgcttccacaactgaacggtgacaggtatttgctggatgagggagcacaccccaggtggtttcctctgagcctgggaggccttttcctgcctgtgggccccaggcccatcctgctgccacccccaggaggatgcccggctccttgtgacaagagtgaccctcgggaggcgtggggagtggggctggccggcctgcctgatggggtcctgagtccatggcgggtttgcatctcaggcctctgggctctggccgggctgggggctattgtccggctgagcggcctgggctgcggcccctccccgtccccgggaccagcctcacccactcgctctgccgcaggtcacacaggccgcctcctgcgggcattgctgggcctcagcctgctcttcctggtggcccatctcgccctccagatctgcctgcatattgtgccccgcctggaccagctcctgggacccagctgtgagtcgctgagggggcggggtagggatagccatcctgggggtcagggagagggccctgcagtgaccccgagtctcctgggggggttgactcagcctgatttatgtctggcctggatggtccaggtgaaacgctccagggatgaccaggccacggtgctggctgggcagagcctgacctgggttcccccgtctttctctgcaggcagccgctgggagaccctctcgcgacacataggggtcacaaggtaagaccattcctcccacccccaaccagcaagcctcccttggggatttcaggccccaggaagtggggggacccaggagggacagagggggacctggagactcatccacactcccacccacacctggagacccatccacgctcccacccacacctggagacccatccacactctcacccgcacctggagacccatccacactcccacccgcacctggagacccatccacactcccacccgcacctgcagacccatccacactctcacccgcacctggagacccatccacactcccacccgcacctggagacccatccacactcccacccgcacctggagacccatccacactcccacccgcacctggagacccatccacactcacacccacacctgtcctcttggtctgaccgcggctgctccctgctctccgcaggctggacctgaaggacatccccaacgccatccggctggtggcccctgacctgggcatcttggtggtctcctctgtctgcctcggcatctgcgggcgccttgcaaggaacacccggcagagcccacatccacgggagctggtgagggcagctgcgtcacccgtgtgtcagggaggtcattcgagagctgtggtctcagccattttgagggttattttaatctttttaaaacagatgtagacgttttggttgtaagttggtgttaaagagaggaggaagttccaaatcccaccccggggcccagcctgcagttccatccgttcagacctgtttctactcgggctctgcctctagtcagaaacctccacgccccgacatggcatctgtgcccttaggaactcttcacaggggaattatttggggccacgcggtggtggaaacctgcagtgctgggcagtgggtctggctggagagccactgcagaagggctgagaaggggcggccccagcaggccccatgcacttacaggcaaacaggctgtcggcccagagccccagcaggggcctggaccccaggagcgacgggcctgaagcagggcctctgtcctcggagtgggaggcagagtgaactttagctgctacaagacttggaggtccggccccgggaatctctgagctatggccccctcacacggacagggcatgagctggggcctgcgacacccaacgttgactgctcagaccctctcgtccctgcctgggcccacacttgccatccccaggctcagccaggatttatggccacctgggtgtcctggcccacgaactccctgccccgggcacccagcctcctcccacctcaccctcccagtgcccactcgtcagatagctgatgctcgccacgcacatgggcctcaacttctagaggagtcccgagagggaaaggggttccccaagccacacggtaggtcgagtacacctgctcagcctgcagaggcggcctgccctcctgcctgtcctgtcgccaccccatggggcaaggctggtgcccgcatttcagaagtgggaacggggacctggaggtcaagtggcttttccgggcatttgagggggtccagcgctcatgaggactgtgctgctgcctgtcctccaagagacccgcccctccctcgtcagcccctccatgctgatgggggcatggggcatgagcttcctagagtttggctgctgggaaggggctgggtggctggactctggtcatcttcatcttggtgtgatgggagcctgagtgtgcagctgcgccctgggcggccagcgtggacagacttcagagtgagctgggggcaccaggcacctgcaggataccgttccgaaagatctagaagccccatgacccggcctggcagggcccccgggcgaccctcactcccacatatgggcaacacacacacgtgtttgcaggcatgcgacggagtgtgccagccagcccgaggggtgctggccgctagaggagggtgaggggcgtctgcagcatggctgcggcacgtggggcccaagtcagggccgggcctgcccctcggtggcctcacaccccaagtcagggccgggcctgcccctcggtggcctcataccccaagtcagggccgggcctgcccctcggtggcctcacacctgttctctcacagcagctgcctctgttctttccactcccagaaatgctcatttttcacggctctgctcagaagatttcccagggccagggccagggccaggctgtgggtggggtgagcgatggcgccctgcgttctgatccagcctcgcccccagctgctgtgtggtctctgagaggggtgggctcagggcctgggcctctcctgccctgatctccctgttaccctggcctcggagcctcccagcagcccctgcagccctggaggcctgtgaggacaggactggtctggttcatctgtcccgtcaagcccccatcccatttgacagaaggggaataggctccgagaggctgtgacttgcctcggcctgcagagcacggagactgtcagagctggggtggggcctatcgtccagccccacagcctgcggtctcagcccgggcacctggcgcctcagccaccaggagagcccccgagtcacagatggggacactgggccggtggcagccaggccagagccagggcatctgacgacagccgtgttctttccaccacacctggctttccctggtgtttgggaaatggctgtgttctgggaaatctttagtcaggtccagggaaaacaggcccggcaggtctctctccccagccccaggccggcctgtcacttcttctaggcagctggccacctctgtccccccagaggcttggggagggcaggagtaccacccctatcttccaggcagagccacccagaaggctggcaggggtacacaggagaccctggagcctggccatgtccctcaggcccccttggtctctagagaccccccggggtatagacagggccccgctgctccctgggtgcgtggttcggggagatgaggtggtataggacagtctgtggtctgtctgtacctggcaaggtcatcacgtgcctgggcttggcaggacagaccctgggtcttcggccagggtgggagctgctaccaggaaggcctgcaggaactgtgagcttgagtgaggaagtaggaaggtgtcaggcagacctcagggacggctggggcctgtgcccggggaggctgtcctgtggccctcagaggagcagctgtggatgtggccgcctcccacgctcctggctgggcaggtgtgggctggagaggtggcgtcagtgcgatacacctgaccttgcccctgtccgtgaccttggcaggatgatgatgagagggatgtggatgccagcccgacggcagggctgcaggaagcagcaacgctggcccctacacggaggtcacggctggccgctcgtttccgagtcacggcccactggctgctggtggcggctgggcgggtcctggccgtaacactgcttgcactggcaggtacgcaccgaggcagggggcactggcagtcacactgggaggggtcttgggagttccctgatgactgtggagacagcgggacacatggcactggccaggtaccaccctgtgtgcccctgccccgcaggcatcgcccacccctcggccctctccagtgtctacctgctgctcttcctggccctctgcacctggtgggcctgccactttcccatcagcactcggggcttcagcagactctgcgtcgcggtggggtgcttcggcgccggccatctcatctgcctctactgctaccagatgcccttggcacaggctctgctcccgcctgccggcatctgggctaggtaacggcttgccacacagcccctttttcctgccaccctggtcccgcccacctggctcgtctagcccctgtggccccactgcctctggggtggtaggctgtgacgggtcttctctggacagggtgctgggtctcaaggacttcgtgggtcccaccaactgctccagcccccacgcgctggtcctcaacaccggcctggactggcctgtgtatgccagccccggcgtcctcctgctgctgtgctacgccacggcctctctgcgcaagctccgcgcgtaccgcccctccggccaggtgagcacctgccacccatggtgggtgggctgaggccaggccatggggctggtctcaggacctcctgcctctgggtggggtgtggagctggtttgggctcaagacgctggtctctgcagaggaaggaggcggcaaaggggtatgaggctcgggagctggagctagcagagctggaccagtggccccaggaacgggagtctgaccaggtgagcagccaggcaggtggagacgccagcgtggggggcgcccggccagcccgtgcatggctcagcgctgcttgcccacagcacgtggtgcccacagcacccgacaccgaggctgataactgcatcgtgcacgagctgaccggccagagctccgtcctgcggcggcctggtgagtaccgcacactgcaaggtatggctgggtgcggggggcggggcggaggccggtgctgccccctggtggccgcctggcgctctcgcatgctcgcgccgcacctctgcctgccgccccctcgggggcccaggacatccacgggtcggtgtcagtgacccccgagacccccagggcagccgagtggccatgtcactgaccaacccccaagacccccagggcagctgagtggccgcgtcgttgatccccaagaccccatggggggcctccaggtcccccaacccctccccagagaatgtggctatgctgtcttgtgctgttagctctgggagctgctccaggtggcccagtggccccaggaggccgctcgtccagggcaggggctggcctgggaactctgtgttggccacgtcgccttgggagggcctgggggctctttctggctactttctttctttaccctaacccttgattttccattttgcaatgtgtttctgaatgaagcaaatgaagccacggccctggggtgggggtcctgagagtcttcaggtgcgcagagctggaaagggggtcagggccacctttcccaccctttcaaggaaagtgaggcccagagaacggcaggtgctggcagggccatccctgacgctcagggacggtgtcagcccaattgccggagccctcgtgttctgcccatagcccaccgggggcctgtctctcctgctgtgtgcttgcccagggcccagattttagggcatagtcagggtggggaggcctgcagatcaacctgccgaagctgaccgctgtccccacctgcagtgcggcccaagcgggctgagcccagggaggcgtctccgctccacagcctgggccacctcatcatggaccagagctatgtgtgcgcgctcattgccatgatggtaggcggctgtgggggttggggtgggcggccccctctgccgcgcaggtgtggggcatcgcctgggtggggtgcgctgggcagctgtgcagccccctctgccgcgcaggtgtggggcatcgcctgggtggggtgcgctgggcagctgtgcagccccctctgccgcgcaggtgtggggcatcgcctgggtggggtgcgctgggcagctgtgcagccctctctgccgcgcaggtatggagcatcacctaccacagctggctgaccttcgtactgctgctctgggcctgcctcatctggacggtgcgcagccgccaccaactggccatgctgtgctcgccctgcatcctgctgtatgggatgacgctgtgctgcctacgctacgtgtgggccatggacctgcgccctgagctgcccaccaccctgggccccgtcagcctgcgccagctggggctggagcacacccgctacccctgtctggaccttggtgccatggtgagtgtgcaccaccacatccgggggtgcctgggtgcgcagacccatcagggttgtcgtcctgttcaatgtccacttgcccgggggagtggcagcgccaagaaggcagatgtgtctgtctgtccccttctgcccacccagagccagcccagagtagcttctcagtgagcgtttgttgactgaataaacagacaaccttgtgttggcacgggcaccacccctgtgccctgacactgtgtgagcgtgggctctgttggcacgggcaccacccctgtgccccgacactgtgtgtgagcatgggctatgcccattggcacaggcaccacccctgcgcccctgacagtgtgtgtgagcgtgggctctgcccattggcaccatgcacagccctgggtctcagtgacaagctgtgcaggccatgtgttcacagggtgcctgcgtgtccatgtgaaacgggtgccagcatcgtgtctggacacctgtttgcaggccagtgggtctcatcttgtgaaacttgtgagcctgtgtgccaacatatgcacctgtgagcttgtgtgcatatgtgagcacgcatgtgggcaaacatgcactccagcatgtggacatgtgtgcaggtgcgtgcatggatctgtgcccacgtgaactagtgaacccgtgtgtgactctgcgtgtgagcgcaagtgaacccatgcactcatccatggatgtgagagtgtgtgctcgtgtgcctctgagtgggtgtgagcgagagggtgttcggtgcctgtggggaggctgcggtggatgggctggtgccagccgcctgagagctcttgccccctgctataggagggtgctgggtcccccggctgtgggaggggtgctgggccccccggctgactgtgacaccctgcgcttgtcacagttgctctacaccctgaccttctggctcctgctgcgccagtttgtgaaagagaagctgctgaagtgggcagagtctccagctgcgctgacggaggtcaccgtggcagacacaggtgagtggtgggccagaggcgggggttgccctcctgcctgcccgccctgatgccatcgcctgcccctggcttggcccacagagcccacgcggacgcagacgctgttgcagagcctgggggagctggtgaagggcgtgtacgccaagtactggatctatgtgtgtgctggcatgttcatcgtggtcagcttcgccggccgcctcgtggtctacaagattgtctacatgttcctcttcctgctctgcctcaccctcttccaggtggctggggggccgggatgggggctggggcacggaccctccccgcggtcctcaccacccccacctcacccggcaggtctactacagcctgtggcggaagctgctcaaggccttctggtggctcgtggtggcctacaccatgctggtcctcatcgccgtctacaccttccagttccaggacttccctgcctactggcgcaacctcactggcttcaccgacgagcagtgagtccaggctggggcggtggggcaggggcgccgaaaccccgtgcacttccccggggctgcagcggctctgccgggggccgggccggtgctgatgctgcccctccacaggctgggggacctgggcctggagcagttcagcgtgtccgagctcttctccagcatcctggtgcccggcttcttcctcctggcctgcatcctgcagctgcactacttccacaggcccttcatgcagctcaccgacatggagcacgtgtccctgcctggcacgcgcctcccgcgctgggctcacaggtgcggccccgccctccctgtccggccctggagaggtgtagcctcctgggccagggagggagccaggtgggagttggacaggagccacatcttccaccttcagatcccaaggggcatttgctcataccaaggggatggcagtagcgtggaggtcacagggacagtgggcatgagttgcgacacagctgtgcacctgaactggcagctgcagcagaagcggtgccgacagggcttcttccagccccaggaaatgaggggcaggaacccagttgggagatgacattttcggaccctctcccaggcaggatgcagtgagtgggaccccactgctgcgggaggagcagcaggagcatcagcagcagcagcaggaggaggaggaggaggaggaggactccagggacgaggggctgggcgtggccactccccaccaggccacgcaggtgcctgaaggtgggttgggcgggcagagcacagctgccacccagtctgctgtgccatgtcccagctcggggggcgttggcagagtcccctctgggctccagagcctcttcctcacaggggacccgggaatccccgtttgtgccccgcactgaccctcacaccatcacaggggcagccaagtggggcctggtggctgagcggctgctggagctggcagccggcttctcggacgtcctctcacgcgtgcaggtgttcctgcggcggctgctggagcttcacgttttcaagctggtggccctgtacaccgtctgggtggccctgaaggaggtgagtgtggcaggcaactcagcttcccatctggggtggggtcgctctggcctgcccagctggcctccccaagcccagccccacgtgcccactgccctccccaagcccagccccacgtgcccactgccctcaggtgtcggtgatgaacctgctgctggtggtgctgtgggccttcgccctgccctacccacgcttccggcccatggcctcctgcctgtccaccgtgtggacctgcgtcatcatcgtgtgtaagatgctgtaccagctcaaggttgtcaacccccaggagtattccagcaactgcaccgaggtaccggcccccgagggctgggacgggaggaagctccaggcaactctgtattcgcagcccgaccctcctggggcagctgcctcagtgcagtggggccagcaatggagatggaggactctcccctgggggcgccaagggggcttcctggaggcagcatccttcgacctcaactgtggaccaggggcgcactccctgcacacaagggtgtccagtaggggcggagtcccagggtctccggcagtgaggacgggagggccccacccctggacagggagagacagtcaggcatctctgcctgggaccttctcgcacatccctccttctccctggacctctcttcactcccccagcccctgcccgtggtctccctgtttctcaaacacctggtccccttccccgtgaaggtggctccaaggctggcagcccccgtgtccctggctggggagcagtggacctgccccagagctgtggctgtggtgggctccgggcagggccagggggcactgtggcctgggagggggcactgatgcctggcctcttgccagcccttccccaacagcaccaacttgctgcccacggagatcagccagtccctgctgtaccgggggcccgtggaccctgccaactggtttggggtgcggaaagggttccccaacctgggctacatccaggtgagttgaagggctggtgggcggctgggcgggcgagtacccggctgccccctgacccttgccctccgcagaaccacctgcaagtgctgctgctgctggtattcgaggccatcgtgtaccggcgccaggagcactaccgccggcagcaccagctggccccgctgcctgcccaggccgtgtttgccagcggcacccgccagcagctggaccaggatctgctcggctgcctcaagtacttcatcaacttcttcttctacaaattcgggctggaggtgaggcaaggacattgcctccccctggggcagggcttggccttcgggagggagggacggctgcaccgtgcaggcaccgcaagcctggccccacctgggtttgcctgggccacagagggtgggggactcagggccaggcacggcttccctggactcctgtggtgtgtcggtgctgacaacaggcagggggccaagttagatctgctctactgtacagcccacctcctggagcctcagtttcccctgcacgatggcaactgccagccactcctgccctcttgacagcgccgctggccctgtccttgcttgatgcccgcagcctccaggcagggctgctgcaagcctgaggcctgctgggtgggacaagaaagtccctccccccagactcagtgcatccccacaccccgccctctcccctccccagatctgcttcctgatggccgtgaacgtgatcgggcagcgcatgaactttctggtgaccctgcacggttgctggctggtggccatcctcacccgcaggcaccgccaggccattgcccgcctctggcccaactactgcctcttcctggcgctgttcctgctgtaccagtacctgctgtgcctggggatgcccccggccctgtgcattggtgaggggcacgtggcttgggtgggagtgggctttgtggctttgtggatgcccgtgggggtgtttcccgcctgccccagactcctgtccaccctcctagacttagccttggcctcctccagtccctcctctctgctccacatcctacccaggcgccatcacctgcatcctgtctctcgggggcggcctggccccctccaggctctgctgttctcttttccttttttgcccagtattctatcttgaaatatttcaaaccttcaggaaagttgtgagtcacacaaggagcactgcgtcctctccgggccccgtgcgggtcggccgtgatgcctcacacccaagtgctgcctcgagcatgcgtctcctgggtgagggtgtctgagcccacagccccacacccgtggtcccgtctcctggcagtgccatcgtcaaacgtgtcctctgcattcaaacagccccggcctcagcacccttctttgtggccatttggttttcagacgggatctggtctgatgtttgctctagtctcttgtcttgggtctaagccgcccccgcctctcctgtctcttgggaagttccggagggaggccggtagcgttgctgacgccgtgagactggatttgcgtggctgtcctggtgctgcaggtctgctcaaggcacacagcaccctgcggtctgagatggggagtcacatttgtgcacgtggccggctcaggggcgtcccacctgccccacagtggcacccagcctgttggcactggtgggctgtgtggggtcagccttgtggttttacgaaacagactttctctctgctgtctctgtgtgtctgtcagctgggattcccatcaaggacagctgccatttgttactggctgctttccaagaattctgtcatccgcagaccctgggcctcccctctgctgagtgggtcctggcccctccggccacacactgttacatcatctccccgtatttggctgggcatggtggttcacgcctgtgatgccagcactttgggaggctgaggcaggagcatcccttgaggccaggagtttgaaaccactctggtcaacatagcaagaaccctttttttttttttttttttttttgagatggagtcttgctctgtcacccaggctggagtacactggcacaatctcggctcactgcagcctccgcctcccgggttcaagcaattctcctgtctcagcctccctcgtggctaggattacaggcaagcaccaccaggcccggctaatttttgtatttttagtagagacggggtttcaccatcttggccaggctggtcttgaactcctgacctcaggtgatccacccgcctcccaaattacaggcctccctcctgggattacaggcgtgagctgccacgcccggccccgtcttgttttctgctcccaggcgctgctgcctcatcttctgctacccaggcccagccttgtgctcacagccattgctccagggagcccaatcgagttctaggagcgtgaggtttagagcccggggtctgggcgctgggtgtgcctgttgctacagggctgcctcagcctctgggccctccagctcttccttgttgaaacatctgctttcgagcatcaccgaggccagctccccgtctcctgtccacctcttccttgttgaaatacctgctatcaagcgtcacctaggccagctccccttcttctgcctccttccacgcggctgcgccatgcagtcgccatcctgtgagatcagcatgtcctgggttccccaacatcgagggtaactttgtttttgtatcgtgaggttccctctgtggcagatggggctgtgggttcagcatgtcctgggttccccagcatcgagggtcactttgtttttgggtcgcgaggttccctctgtggcagatggggctgtgggttcagcatgtcctgggttccccagcatcgagggtcactttgtttttgtgtcgcgaggttccctctgtggcagatggggctgtgggttcagcatgtcctgggttccccagcatcgagggtcactttgtttttgggtcgcgaggttccctctgtggcagatgggggctgtgggttcagaatgtcctgggttccccagcatcgagggtcactttgtttttgggtcacgaggttccctctgtggcagatggggctgtgggttcagcatgtcctgggttccccagcatcgagggtcactttgtttttgtgtcgcgaggttccctctgtggcagatggggctgtgagatcagcatgtcctgggttccccaacatcgagggtcactttgtttttgggtcgcgaggttccctctgtggcagatggggctgtgggttcagcatgtcctgggttccccagcatcgagggtcactttgtttttgggtcgcgaggttccctctgtggcagatggggctgtgggttcagcatgtcctgggttccccagcatcgagggtcactttgtttttgggtcgcgaggttccctctgtggcagatggggctgtgggttcagaatgtcctgggttccccagcatcgagggtcactttgtttttgggtcgcgaggttccctctgtggcagatggggctgtgggttcagcatgtcctgggttccccagcatcgagggtcactttgtttttgggtcacgaggttccctctgtggcagatggggctgtgggttcagcatgtcctgggttccccaacatcgagggtcactttgtttttgggtcgcgaggttccctctgtggcagatggggctgtgagttcagcatgtcctgggttccccagcatggagggtcactttgtttttgtgtcgcaaggttccctctgtggcagatggggctgtgagttcagcatgtcctgggttccccagcatggagggtcactttgtttttgtgtcgcgaggttccctctgtggcagatggggctgtgagttcagcatgtcctgggttccccagcatggagggtcactttgtttttgtgtcgcgaggttccctctgtggcagatggggctgtgagttcagaatgtcctgggttccccagcatcgagggtcactttgtttttgtgtcgcgaggttccctctgtggcagatggggctgtgagatcagcatgtcctgggttccccaacatcgagggtcactttgtttttgggtcgcgaggttccctctgtggcagatggggctgtgggttcagcatgtcctgggttccccagcatcgagggtcactttgcttttgggtcgcgaggttccctctgtggcagatggggctgtgagttcagcatgtcctgggttccccagcatggagggtcactttgtttttgtgtcgcgaggttccctctgtggcagatggggctgtgggtttcgcagatgcgtggagtcacatccatgccctcagtccttagggaccgaccctccctgcctcacacgcctcccaggaagtgtggccgggggccggcagtgccacggctccctccccagcaggccccggccgctcccatccccagcacgtggtcctatcagaacgccacgtcagcaggactcccagcaggtggcctttaggtctggcttctttcacttggcagagcacactgaggtctgtctaggctgtcgcatggatcccggtcccacgtgctgagcagcgcgttcccagctgtggttgctgcaggttgaacttttcctggctgcaggcgtccgtgcagcttctggccgttgttttcagagctgtcctatcacacgcactgtcctatcatggaatatgacgccgtgtgggccacaactcaggcccagcagcccccacccccgtgctctctggcctcctgctcagttcctttgcccccaggggcttggtgcagagttgaaggaatctgtgtgtgtgaacacacaggacactagagctgtcagttctcgagacaccaggtgtgcgcgaggtgattcccatggaccctgagggctggtgatagactcgggtcaacgggtggggaccgggtgtctcaggccccaggcaggcccggcccttcctgacatgacaccccttcccccagattatccctggcgctggagccgggccgtccccatgaactccgcactcatcaagtggctgtacctgcctgatttcttccgggcccccaactccaccaacctcatcagtgagtgccccccaccacccccgcctctgcagaggaccctcagagtacattcacgcccccaaatctgctcacaagtgtgcacacaggcgtgcacgggcggaggtgtggtcaggcacatggcggcctgcaggccctgacctcgcacgcacgcacgcagacctcagcctgtgtgcacggcagcccttgtgcagatgccctcacaccggggctcccccagggacacccggccactcacccaggcagacgtgtgtccgctcccagcggctgcacgccgacaggcctggggtgggaggtgggatttatgcgccgtgcccacctcgtgtgggtccccgtgtggcacagcggcggctcctgtgtcctgcaggcgactttctcctgctgctgtgcgcctcccagcagtggcaggtgttctcagctgagcgcacagaggagtggcagcgcatggctggcgtcaacaccgaccgcctggagccgctgcggggggagcccaaccccgtgcccaactttatccactgcaggtgggttccacgtcaccctccacggggaaccttctgggaggggtggccggggcgcccgccctgacgctccggcctggcaggtcctaccttgacatgctgaaggtggccgtcttccgatacctgttctggctggtgctggtggtggtgtttgtcacgggggccacccgcatcagcatcttcgggctgggctacctgctggcctgcttctacctgctgctcttcggcacggccctgctgcagagggacacacgggcccgcctcgtgctgtgggactgcctcattctgtacaacgtcaccgtcatcatctccaagaacatgctgtcggtgagcctccggcccccccgcacccaccgccctggggccccgctggccccgctgaccctgctctcccccagctcctggcctgcgtcttcgtggagcagatgcagaccggcttctgctgggtcatccagctcttcagccttgtatgcaccgtcaagggctactatgaccgtgagtggccaggacggtggcgggggagggcgtggggaagccccctgctcctgggccctgggcctgacccttgccggtgcctgccttgcagccaaggagatgatggacagagaccaggactgcctgctgcctgtggaggaggctggcatcatctgggacagcgtctgcttcttcttcctgctgctgcagcgccgcgtcttccttagccattactacctgcacgtcagggccgacctccaggccaccgccctgctagcctccaggcaagcttgggcccagacacagcccagagctcccgtcttggggctgggagggggcaatgggaggttcctcactgtctcaggccccggcccgtggagggcaggctctgccactctgtgacatgggcgtgtcatctagagggagaatgaaggccggcagatccccggcaccatcacactctgccccagtgctgggtctgtcagagaccacaggctgcagtgctgacggtggctggtgtctcacccccagccaactttcccactaagggctaagtttctccaccagcgggagggccactgtgtggtgtcacgactgccccagggaggggttctggcttggggccagctttgccttcttccctgcagctgtggtggggtgggtgccaccagacgcccctgcatctgtacggcagaagggcctgtcctcgccgcagacagcacggagggtgggggcagcagatgcctcccccgtgggtgcctcttgtccagcgtgggcagagaggagcaggctgagctgtcccgggctgagcggggagcggcggctgcccatgttgctggggtcgagtgcctggtgctcacaccccatccccgcctccctacaggggcttcgccctctacaacgctgccaacctcaagagcattgactttcaccgcaggatagaggagaagtccctggcccagctgaaaagacagtaggtgcctctggggcggggactccccggctcctccccccaatgctcagcataccccacctttccccaccacaggatggagcgtatccgtgccaagcaggagaagcacaggcagggccgggtggaccgcagtcgcccccaggacaccctgggccccaaggaccccggcctggagccaggtgagtgcagctggagtcgggcacccagggccccgtgtccagcatgtctgtgcctgctggcgtgtgctgcgtctgtgcccatgtgacgtcccacagggctcccagcccgcctgtcctgtccgcatgatcaccctctgtctggcaggccccatggccgccctgtgactgtccgtccacgcacatgggctctgagccccatggccccacacggcccccgtcactgtgggtgtccgtgtctgtctccacctatcctgtctccaagacgggagcactcacagccccgacccctcctggtggcttgactgctgcctcatgctcaccctgcccctccacagggcccgacagtccagggggctcctccccgccacggaggcagtggtggcggccctggctggaccacgccacaggtacccccaattaggccgcctgtggccaccctctcaggccctctgtgcccccatctgtcctctgcctggcctgctatcttcccctcccttcccccgactcccaggccctgagcgtcaggacgtgctcaggcctcctgggtcggggggtgcctcactggctgcagacccctgggctgactatgtcctctcctggctatgccccagcccttccaacagtgggagtctcggagcttgccccgatgacacatggtggtcgagcagcgatctcacctgggacccagcagcactgcgttattctgtttttgtttctttttgagatggagtctcgctctgtcactgcaggctggagtgcagtggcatgatctcagctcactgcaatctctacctcccgggttcaagtgatcctcctgcctcagcctcccaagtaactgagactacaagcatgtgccccactccaggccttttttttttttttttggagacggtgtcttgccctgtcgcccaggctacagtgcaatggcgtgattgcggctcactacaacccccacctcccaggttcaaaggattctcctgcctcagcctcccaagtagctgggattacaggtgcccgccaccacgcccagctaattttcgtatttttagtagagatggggtttcaccatattggccaggctggtctcgaacctctgacttcaggtgatccgcccgcctcagcctcccacagtgctgggattacaggcgtcagccaccgtgcccggcctgttctgtttttctaactctcacacagcctcctgggttttccccggtcctctgcagtcggcccactctgcaccccagcccgcgctggctctgctcctcagctgccctgcccacctctgtcttgtcccaccgcgctggcctgtgtcttgtgcctgcactgctcccggctactccgcatgggaagggtggctctcgggccttggcccatgcaggcggagggggtctggctgggagtctccctgcatggaaggctggctctcagtgctgcctgcccacagtcatccactccggggactacttcctgtttgagtccgacagtgaggaagaggaggaggctgttcctgaagacccgaggccgtcggcacagagtgccttccaggtgaggtgggagagccccgtcggccccactccaaccacagagcttgtggtcctggaccagggcagcatagagggtgtcagatgcccccagggcctgggagccgagctcctccacctccagttagcccaccccgccccatccaggcctcccaagtcccatgggaaaccaggctacagggacatgggtcatgtgtagcctgctgccccacggtcttggctctgaccacccaggttctggtggctgcccgtggcctgacctgtgagaccggcccaacacctttgtgctggccgcctggctgtcctgggtccatctttgggcccctggctcttggtgttagaccagcccacccaactcctgaatgggtgggagtcttcccccacagcccctcagggtccccatccgggaggggctcagggacacggaggtccctgggagacacagagcagggatctggatctggcgcccggcttgcccaaccccagcttcccgcctgggtctgatggctcgggaggccgggtcctaacccgggggctggccgacagctggcgtaccaggcatgggtgaccaacgcccaggcggtgctgaggcggcggcagcaggagcaggagcaggcaaggcaggaacaggcaggacagctacccacaggtgagctggggggcgtggggactctgaggggaagccgcgggactgccagtcactcaccagcatcctgtgcccaggaggtggtcccagccaggaggtggagccagcagagggccccgaggaggcagcggcaggtacgtgggcccggggctggggagtgggaggtctctcttggccccacaggctgcccctccagcgccccctcccgccctcccgcaggccggagccatgtggtgcagagggtgctgagcacggcgcagttcctgtggatgctggggcaggcgctagtggatgagctgacacgctggctgcaggagttcacccggcaccacggcaccatgagcgacgtgctgcgggcagagcgctacctcctcacacaggagctcctgcaggtgagcctgcccgtgcaccacgctcgtccctgctctgcctgactacgcccctgcctgcttaacagcctagtcccgcgcccactgcacgaaaccccgtgtggggacaagagctggacgcagccctgagccccctgctgtgccctgcagggcggcgaagtgcacaggggcgtgctggatcagctgtacacaagccaggccgaggccacgctgccaggccccaccgaggcccccaatgccccaagcaccgtgtccaggtaggtgcgggggtgacccgagccccagctgctgcccctggtgtgtgggcatcgcctagccatccccgaccctcgccattcccttgtaccccaaaggaccgtgggcactttccaccctgaccctccctgtagcctggggtcaggccatagagcaggattctctgtgactcggcttccctccccagtgggctgggcgcggaggagccactcagcagcatgacagacgacatgggcagccccctgagcaccggctaccacacgcgcagtggcagtgaggaggcagtcaccgaccccggggagcgtgaggctggtgcctctctgtaccagggactgatgcggacggccagcgagctgctcctggacaggtgggggcgggacgcgcacaacaccagcctcaccatggccctcggggagcagccgaacaggggcaggagactgactgtgaccggcaacagatcgggccgtcatgccttcgggcagtcccagactcccccaaacacgcgggtctccctgtaggcgcctgcgcatcccagagctggaggaggcagagctgtttgcggaggggcagggccgggcgctgcggctgctgcgggccgtgtaccagtgtgtggccgcccactcggagctgctctgctacttcatcatcatcctcaaccacatggtcacggcctccgccggctcgctggtgctgcccgtgctcgtcttcctgtgggccatgctgtcgatcccgaggcccagcaagcgcttctggatgacggccatcgtcttcaccgaggtgggccgaggccgcgggggagggggcgcccggcccaccgcgccgtgaccctccccgcgtgctgagccccctcccccacagatcgcggtggtcgtcaagtacctgttccagtttgggttcttcccctggaacagccacgtggtgctgcggcgctacgagaacaagccctacttcccgccccgcatcctgggcctggagaagactgacggctacatcaagtacgacctggtgcagctcatggcccttttcttccaccgctcccagctgctggtgagtgtgagccttggctggcaatgcggggctgggcaggccctctgggcacctgtgctctccaccagggaggcaaggccccctcaccacaccctcccgcccctcagtgctatggcctctgggaccatgaggaggactcaccatccaaggagcatgacaagagcggcgaggaggagcagggagccgaggaggggccaggggtgcctgcggccaccaccgaagaccacattcaggtggaagccagggtcggacccacggacgggaccccagaaccccaagtggagctcaggccccgtgatacgaggcgcatcagtctacgttttagaagaaggaagaaggagggcccagcacggaaaggagcggcagccatcggtataagcgccctgcctcacaacctcctgcctacccagttttctgagtggggctactgcagggagggtctttctcagatgagacggccaagcccagtgcgaggcccacctggatcccaggaaggtgccacttctgagccacagctcccggctctgcctacagagccgtccctgactgctgcccccggggatgctccccacgtgtagggtgactgttggcctgggctggcccctcacagttgccccagacagaggacacagccccagctgtctccttgccagtgacactgggagctttcctgtgctccgtctgcttgtctgtcaaacagggagaatgccagcctcttagggtggtcaggagccatgagccaggcccagtccccagggggcccaggcagaagtcagcttttccctacagaagctgaggacagggaggaagaagagggggaggaagagaaagaggcccccacggggagagagaagaggccaagccgctctggaggaagagtaagggcggccgggcggcggctgcagggcttctgcctgtccctgtgagtgatggcggccgggggcagctggggagtgggggtggggaggcgggtactgggcccaggctgagcgcccccttccgcagggcccagggcacatatcggccgctacggcgcttcttccacgacatcctgcacaccaagtaccgcgcagccaccgacgtctatgccctcatgttcctggctgatgttgtcgacttcatcatcatcatttttggcttctgggcctttggggtgagccaggcccgggacccaaacccagtgtacgcagagctcagcagccacccacatcccctgggcttggctccccctgacctgtgctctcctggccacagaagcactcggcggccacagacatcacgtcctccctatcagacgaccaggtacccgaggctttcctggtcatgctgctgatccagttcagtaccatggtggttgaccgcgccctctacctgcgcaagaccgtgctgggcaagctggccttccaggtggcgctggtgctggccatccacctatggatgttcttcatcctgcccgccgtcactgagaggtgggcccacgcgtgggggcgctcggtctccaggggcggggcagtgcaggctgggggccctgcggggctgtttctgatggggtccttgacctggccatcccgccccaggatgttcaaccagaatgtggtggcccagctctggtacttcgtgaagtgcatctacttcgccctgtccgcctaccagatccgctgcggctaccccacccgcatcctcggcaacttcctcaccaagaagtacaatcatctcaacctcttcctcttccaggggtgagtgcaggtccgccggggtgggggtcacggcccgggcatgagggagcccacctgacgggaaccctggctgtgggcaggttccggctggtgccgttcctggtggagctgcgggcagtgatggactgggtgtggacggacaccacgctgtccctgtccagctggatgtgtgtggaggacatctatgccaacatcttcatcatcaaatgcagccgagagacagagaaggtgcctgggcccagggcgggggccgggacaagggccagggatatgccctctccctaagacagaggcactgctgccacgagaacccgtggtgctggaggccctcccagggctcggagcccatggggacatgaggcgagcccacccactagctgatcacgaggccagtgatcttggcagctgcgagtgagtgctgggcgcagaagtgggcagcggagttggtcctgttccaggcaggctggcagcagagcagggcctggtgcagggaggaccggacagccactgtttgctgcattcttgtttaatggcctttctcagagagaattcgtgcgtcagaccactcccccacgtaaaaagtacaactcaggggtttctagtggattcacagttgagcatctgccttctcaccacttcaaaaagaaaccccgggccgggcacagcggctcacccctatcatcccagcactgtaagagacccaggagggaggtactgctcaaggccaggagttcaagatctgcacggccacaagcgagaccctgcctcagcaaatgtaaaaataaaacaattagctggcaatggcagctcatgcctatggtcccagcactttgggaggccaaaggaggaggatcagttgaggccaggagttttaagaccagcctgggcaacatagtgagtgagactctctctacaaaaaaataaacgttagctgggtgtgatggtgcacacttgtgctcccagctactctggaggctgaggtgggaggatggcttgaggccaggagtacagggctgcattaagcctgatcacaccactgcactccagcttgggcaacagagtgagaccctgtctctaaaaaagtaaaaagaagaaacccagtgcccattggctgtcactcggtttcccctcccctggcccctagtaaccccttgtctaggtcctggttctatgagatttgcctacagtggatatttcatgaaaacaggctcagacagcgtttgtccttttgtaatcagctttccttgctcagcatggtgtctctgaggtccacccacgtggcagtatggccttcctgtttatgggcaaatgatattctgtggcatggatggaccacaacatgctcatctgttgatgggctgttgcccgtgatgctgccaggcacgccggtgtacacgtctgtgtgcccgtgcccctggtcctgtggctgcacgccagggccggaagtgctggtggtggtttccatcatgaggagctgctggttttccatagcagctccaccatctaaggttcccaccaccaacacgaggttgcggtttctccacatcctcaacaacctgttactatgtcttttttgttctggccatgctgctgaacgggtggcttgctgtgggctcttctcagttccctgttgaccgacgctgagcgtcttttcatgtgcttggccatttgtgtatcttctctggggaaatgtctattcaaatcctttctccatagtttagttgggcttttgagataggatctcaggctgaagtgcagtggcatgaccttcactcactgtagcctctgcctcccaggttcaagcgattctcccacctcagcctcccgagtagctgggactacaggtgtgcaccaccaggcctggctagtcttttgtatttttggccaggttcgtctcaaactcctgacctcaaagtgctgggatgacaagtgtgagccgccacacccagcagttgggttgttttttattagagttttttctgtttttctgcagatacattggctagaaatgactgaattggaagaattctctgtattctctggttgctagaaccttatcaattaaaatttgcagaaaatttctccaattctatggactgtctcttaaattttcttggtgtctttggaagcacaaagtatttattttggtaatatctggtttactttgtttccttcgccaaatccaggttcatgaagatttgcccgttttcttctaaaagttctatagttttagctctgaagtttcgctctttgatccactttgaggtaaattttggcacatggtatgaggcaggagttgcgtttcattctcctgcctggggcggtgcctgcaccgtgttgaaaaaggttgtccgttcccactgaacggtcacagctcccttgtctaagatcaacgacccctgaacatgagggttccaactggactcttagttctactccactggcctgtgtctgcccaccattactaccgctgtgccatactgaggtcaggctggggcttttctgggctgctgggtgagctggagaatgcggttgtgtgacccgcaggaagggcagagctgagcgtatgacctgtgtcgttcccctccagaaatacccgcagcccaaagggcagaagaagaagaagatcgtcaagtacggcatgggtggcctcatcatcctcttcctcatcgccatcatctggttcccactgctcttcatgtcgctggtgcgctccgtggttggggttgtcaaccagcccatcgatgtcaccgtcaccctgaagctgggcggctatgaggtgagcatgtgtgggtccgcctgtccattcccatcccctgggggttctggccaaggtggtgcaccacccccagccgctcctccacgctcatcttcgtggccccgtgtccccgtgcctgccccagccgctgttcaccatgagcgcccagcagccgtccatcatccccttcacggcccaggcctatgaggagctgtcccggcagtttgacccccagccggtaagtggcctctgccctgtgaaagctggtgtggggaggcggctgcagtcactgagggtgtcacttgtacccagctggccatgcagttcatcagccagtacagccctgaggacatcgtcacggcgcagattgagggcagctccggggcgctgtggcgcatcagtccccccagccgtgcccagatgaagcgggagctctacaacggcacggccgacatcaccctgcgcttcacctggaacttccagaggttcgtcctggacttggggcagtgcctgggtgggtggacccactacagtgggtcacgctgtgttcccacccccagggacctggcgaagggaggcactgtggagtatgccaacgagaagcacatgctggccctggcccccaacagcactgcacggcggcagctggccagcctgctcgagggcacctcggaccagtctgtgtgagtgaagggcccgggtggtgggcaggagggctgtgccaggttggctgggccaggcctgacctgccagcacctccctgcagggtcatccctaatctcttccccaagtacatccgtgcccccaacgggcccgaagccaaccctgtgaagcagctgcagcccagtgagtatgggcgtgggggttgggggaggctagagaggggtgacctgcggcctcaacgatcttctccctccatcccagatgaggaggccgactacctcggcgtgcgtatccagctgcggagggagcagggtgcgggggccaccggcttcctcgaatggtgggtcatcgagctgcaggagtgccggaccgactgcaacctgctgcccatggtcattttcagtgacaaggtcagcccaccgagcctcggcttcctggctggctacgggtgagtgagtggctgggggggcaccccgcagctcggggggctccgggcggccccaggactcaccagcttcccccgcagcatcatggggctgtacgtgtccatcgtgctggtcatcggcaagttcgtgcgcggattcttcagcgagatctcgcactccattatgttcgaggagctgccgtgcgtggaccgcatcctcaagctctgccaggacatcttcctggtgcgggagactcgggagctggagctggaggaggagttgtacgccaagctcatcttcctctaccgctcaccggagaccatgatcaagtggactcgtgagaaggagtaggagctgctgctggcgcccgagagggaaggagccggcctgctgggcagcgtggccacaaggggcggcactcctcaggccgggggagccactgccccgtccaaggccgccagctgtgatgcatcctcccggcctgcctgagccctgatgctgctgtcagagaaggacactgcgtccccacggcctgcgtggcgctgccgtcccccacgtgtactgtagagttttttttttaattaaaaaatgttttatttatacaaatggacaatcaga

SEQ ID NO: 2 - DNA, 69883 bp, Homo sapiens
  source 1..69883: mol_type = genomic DNA
gggagccgccgtccggcccagctcggccccagtgagccgagcgctgcgctccgccgaggggcagggcggtcgcctgagcgagcgcgggcccgggacgtcggcaccggcggggcggccgaaggagaaggaggaagaggagaaggcggcgcgcgggtccccgcgggtcagccatggcgcgccggccccggggcccccgcaccgccccatagcgccgcggcgtccgctcggtctgggccgggccctgggccctccagccatggagccgcacgtgctcggcgcggtcctgtactggctgctgctgccctgcgcgctgctggctggtgagtggggggcgggcgcctgggggcgacgggagggggctgcgtctcggctccccacggcctggacaccggacgacgccggccggggcgagggctgcgggcgagcgggcgcggaaattcccagggacgcgcgacccgggcgcccgcattcctgaagcatgagcgcgccaggcggcggcggggctcctgtcccagggccgggctggaagggcggcggcggctgggggagacggcaccgcgtgcccacgggggcggtcgagcgagcgccgggcatagcgcggctggcgtctccgccggggcgctgcggagaggaggccgccgggcgaggcggtgtttgccccggtgggaagggccgcggcggtggtggggggagcacgaatctctttttctctttcgggtttaaaaaaaaaagcgcaaagttgcatcaggacttcctgacaatctgggagaaggcgggcttcctgcctggagctgtttaatttggagcttcccgagcccaacgaacgtccgtgcccagggcccagccccgctcaccgctgcacccccctctgccggactgaggcggtcccacactttggaaaaaaatagtgtgggttcctccctggtcctcccttgccctactgggctcagtttcgcaggggcgggggccggcctctgccctggtctgggggaggggacacccccggaggctgtggcctggtgtcagggcggggcaggggtccccagtcctggcatctgtgttccctgcttgccgggcagtggtgcccctttcgcgaagcacacccgggtggcttggtgctgcacggcctggcacccctacccttccccgaccctggcctagccgggacccagggtccgcgccctccgcccgggggctccccacgtgtgattgatctgggaagcagtcggatggaattaacccacggacaagtgggacggtttgcattgggagtccgccatggacacggcaggtggggccttttgattgtaaaagccctttcggagcccttgcctcgctccaggtgggagctcgcccagcgctagctttggggatctagagccgcctgcctgaggctcccagacagactgcgttttgatcggtcgcacagaaaggtggtgaaacttggggaagattttctagacaggaatcaatgaaaaccattgaggctggagaggagaggttttgagcaactctcttcagtgcggtcagccctgtgtggactgggcagcctgggacctgctcccagtgcagggtcagatgggccgtaaacagggcccggctgtgttccttcctgtgccttgaaaacaagcaggacagcctggcacagaggcagagtctagagctgacaggccttagagagggaaacaggaaagcttctgaaacgtcccgttcacactgatcgttccatttcctcttgtgtctgagtgggagcgggtgtcctccctgcagggaatgccccccctctcagatggcagctgctccttgggcagagttggcaatgtttttctttaaatgaccagatggtaaatattttcatgtcacaaaatcttcttcttctggtatttttccagccattaaatgtaaaagccctcctgagttcatgggctgtacctaaacaggtgttgagcccgattctgtggcacatgtggtttgctgccccctgggcattggtcagggggcctgggttctgccttctcgattgctatccgcgtgggggatctgggggagggatcactgttcttcttgcttttggcctccttggggaggatggggaggtagcccaggggtgctcacccaggccccgtgtcagtcttctatgaaacttttaaagaatagtgatgactgactgtctgtctgtatggtactttccttaaacctaaaactggtcccaaataaagtctcttaatttgaaagatgctgaagcccgggccataccccacactgattctgtgtctggggatggggcgtggggcctgggcctcactcagtgttttctctcagtcacctgggggagatggaagtggagccggccaagaaccctgcctgcctgcctgctggccgggactcctgagtcaggctctctggccctggggtgtgggcagctccagatggacccgcgatgtgcaggttcagctggcctggccggaggtgggacactggctttgctgtctttggagtgccccctccctctctggcgagctttggctggaagcagttctaccgtgttttggaaatgaatgaggccttcagaaggcattagtcagtgtgtgcctgcgctggctcagacagtgcctggtgagggtttgagtcatcctggggtgcccctggcccccacgccctccctctccagtgcaggatcattacccaaaaatctggcagggagctgccccacccacagggagcaggggcctccttcagcagtctcacctaatgttgctggagccttgggggatcagggcccatctcttctagagagatgtcagggcagggctgggcgcgatggctcacacctgtaatcccagagcttcgggaggccaaggtgggaggattgcttgagcgtagccattcgagagcagcctgggcaacgtagagatccccatctctatgaaaaatatttaaaaattagctgggcatggtggtagtgcacctgtagtcccagctactcaggaggccaaggtgggatgattgtttgagtccaggagttggaggctacagtgagccatgattgtatgactgcactccagcctgggtgacagaccctgtctcaaaaaagaaaaaggaaaaaaaagggcaggacatttgtgattctgataatatgggaccgcacctccagttctatcagtgggaaatctagacagggctgcggaaagccagctggtgcgagaggagccaccgtgtcactgactgtgggacacccacgtgggctgacaatatggcttctgcttttcagggtgcctcgtggcacagcctaggggcacacccacggccggcaagctgggcgtcacctccttcaggctgattgtcactgagaagtgtcaccatttagcatgagggatgctgcctcctttttcagaacattgtcaccatcagggtctcaccactcctgggaggcggccgagaagctggggaacagcaggcactcggctcacagttgctcagcagtgcagaccctctgagctgagcatggcagagtcacccttcggaggcctgtgcccgggtctcaggacctgcacagaaccctggcctgtcccatccgagggtgctgggaagagcatggccgtggcagagtagggtgggagctgctttcctctgtggcttgggggccccttctgagcatcagctccctggtgtgacagaggggcgcactctgtccccatgctgggcctggaggctggatgagtcagcaggagagcctggggcctgcctcacagcaccaagggctgcaggtgtgagtgtgcacatgtgtgcgtgtttggggaaggggccagggactgcccaggagctgaggatgggtcacagcgggtgctcgtcccgcagcgggtcactggtgcccaggacacagggagctccagccccagctgccagggtcccacagagaggaagtttcctctgggggtgggtgggggcgcacagtctctgatcctggccccaaggcagcttcctgggcggtgtctctcctgtgctgactcggcagtgcatttgctttcggtgctcaaagatgaaggggaaccaccgtgggccttgacggcctcatctgcccgctgcagcccactcctgagatgggaccaccgcagtcgtcagggtccagtgagagccgcatcttgcaggaagccattcctggcctctctggcctcagaaatccccttttcaattcaacaaaatgttagtcttctgtttagctattttagaaatagacagttaggcttttttctcttttttcttaaagacagaggctcactctgtcgcccaggctggagtgtagcggcgtgattttggctccctacaacttcagcctccagggctcaagccatcctcctgcctcagcctcctgagtagctgagattacaggtgtgtagcaccatacctaatttttgtattttttgtagagacggggtttcaccactttggccaggctggccttgaactcctgaccttaagcgatctgcccgcctcagcctccaaaagtgctggggttacaggcatgagccaccgcgcccggctgacagttagacttttgcttctttgtttatataagctttttcttctggttccaaaagcaagtttgcccttccttgtggtagagaatcctagctcacagagcagtttagaagccagcacagtatcccacacacacatctggcatggacagacccttccttgctgggtgtgggtcctgtgttcttctgaaaggcaagctgttcccagccaacccctgcccctctctgccttagcctgcctggaggcctgagtctcctgggtgactgtgaggtgggacccccccttcccctacccccacccacatcctctgtatctgccttctgtcctgctcttgacctttgagctcctctgctggcttcagggtgcggctgttgagcctattttttggattaggactctggggtgagggaagttaattcacacacccaagatcacactggtgggaagggacaggcccggggtgaaggcttctcctctccttggcggttgagtcccacacctgctggccgaggcacctgaaggggacttggggtccagggtcactgggaggacgggggcaggcagaggggtggccgacctggtggcggctcgtgggcagcagccgaccctatcttgctctgaacgtgtggggccctcacccccttctctgggtctgggtttcctcccctgtaagtgacaccgtaaaagcttcacagccgcttccaagtctcaggtctctcgggctttggatctcacacccaggctgggtgggggtaggggcgggacagccgtcccccccggaaggctcagaattcctcgcacaatcgtggggccaggagacccgcaacacaggctttcccagctgcgctgagtgccgcggtggccgggggtccgtcgggcctccatggagctgaggggaaggggccactcaccgcctggtcccggagcacacagggcagctcccaggagcacagaggctccttgggacctgtggggctgtcggcctccctcatgctgcacacacagcgcgtccccaggggtgtctgcagcccagcccatcccactgcagattcccgaccactcagattcattcatgcatcctctcacgccggtcctgtgggacagagctctgggcagcagccagaagtccaagttctggttcagggccagtggaggtgggtgttggggtggggctggagctccctgctctccctcccaagctagccaggaaaggaggttgggggccccgcacggtcattgctgtttattcacaaagcgcgatgctgagcacaggcggggaaagaaaagtgcgatcagtgccaggaaaatggggctcccccgacgccgtccaaaatgggatcccttgccgggcgcggtggctcacacctgtaatcccagcactttgggaagccgaggcaggtggatcacctgaggtcaggagttcgagaccagcctgaccaacatggcaaaactccatctcagctaaaaatacaaaaattagctgggcatggtggcgggcacctgtattcccagctgggaaggctgagacaggagaatcgccggaacccaggaggtagaggttgcagtgagccacagtcgtgccactgcactccagcttgggggcttgggggacagcaagactccctctcaaaaaaaaaaaaaaaaagtgaagtcgtaaatcagattaaattccctttttaaccctttgaacctctgtcctcccctgttcccagcgggaagcctctggtgaacgcgccatgcaccccacctgcccccgctctctgggtctctctcccagctggaacggccgcttccccaggtgccttccctgggccacagccttgtgcctcggcggctgctggatgcctgggtgtgtgggtggctcccagtgtgtgggatggcacacgagcctctcgcccttctgtgtggggtcgcacacccaccgcaggccgtatttttgctcacgttcatgtttctccacggtggacgctgggttgcagggacccttcctgtgtgcgggtgaggatctgggcagctgctggtgccgggcccatggggacgctgaccgtcccgggtgccggctctgaggtgtgcagtggacggctgtcctgccgggcgctgccagggccccttaggccgacgtgcgtggccacccgattccccgccgttgtctcaggaacctttgccgagtggggtggatcaatttttcgggtgtgttttaatagcataattacagggagtatttcaggctccctctgacgggccggcagggtttggctgccggctgtttaccaggctccaatctgcacactatttttctgtgggtatatatagctggggctgcttctccttcctcaggttcaggctaaagagggacagcagccgcctcagccaccccctgtggtttcctttgcctgtggatgggcggctaaaatgggcccaggaagagtcaagaacaaggccggctctcggtgccacagctctacccccaaaagcaggaagggggctcgggccatgcccatctgtgagctacaccggtccgggagcggcatcaggcaggggagtcctggacccccgcagtgctggggtgtgtttgtccgccctccctcccgtgtgtctagaagcctccagcctcggggaaaacaatgaaactcaactgtgacttaaacagattcccaggcccgcaggagctcccggaggcttgtggctgtggcgagacctggagggccatgcgggagggacagacgcaggtttgcggaggccgcctgcccaggaggggcgtcaaaggaggggacagatgtgggtttagggaggccacctgcccgggaggagcctcgaaggaaggcacaggcgtgggtttggggctgcctgccctggaggggcctcaaggaccccaggtctggtcttggtctcactcacctcctggaccccccaaggcctgcagtttgcaatctgtcgcctgggacccccactgtctgcctttacgcagctcagccaccacgcggccctcgctccctcatttacttgatttctgtttatggttaaagtaccgtttaaaacgacacatcattaaagcaacatgaaagggagttttgaaaagggaagccatcgtccatcccactgcccctgcctcagcggggatttacttttcctttcctgtctgcgggccagtgacaatgaggaccccgcaatgtgtctgcgggccagtgacaatgaggaccccgcaatgtgtctgcgggccagtgacagtgaggaccccgcaatgtgtctgcgggccagtgacagtgaggaccctgcaatgtgtctgcgggccagtgacaatgaggaccctgcaatgggcggcctgtaaggctctgccctggcctccgctgccttcgctttctccctccttggtgggtgcacgcccttgtgcttttcctaaaagagcaggtcctccgggcatggtggctcacgccggtaatcccagcactttgggaggccgagacgggtggatcccaaggccaggagttcaagaccagcctggccaacatggcaaaaccctgtccctactgaaaatacaaaaattagctgggtatggtgacaggcatctgtaatcctagctactcgggaggctgaggcaggagaatcacttgaatccgggagacggagtttgcagtgagccgagatcacaccattgcactccagcctgggcaacaagagcgaaactccatctcaaaaaagaagaagaagaaaatcctgacacttcagcctggtgcaggcccttccctccttctagtccctgccaagaagtgagccgggcccagatctcctgccgggcggggaatgagcacacacattcccctcttgggacagacagcagcagcagccctgttgcacacatgaggacgtacaggctcaggggccgtgggtggcagagaggctatcagcgccggactggcccgccccgagccagggtccagccccacagtcctgtccccaagccctggcccttctgcggtcactcccgtctgctgaatcccctactctgcccctggtgtgtggccccccagttccctcctgtgttcattccctgctaacctcccgtggcttcgcctccccagatgccctgagcacacagcctctccccttcctcccctcctagatgtgcacataggagccgcccaaaggctggggcagctagtggggcccctccaagggaagctgggccccgggcaatgccctgagccaccaggtcctggccctgcgtctcatcccttcttttttttttttgagacagagtctcgctctgtcgccctggctggagtgcagtggcccagtctgggctcactgcaagctctgcctcccaggttcacgccattctccttcctcagcctcccgagtagctgggactacaggcgcccaccaccacactccgctaattttttgtatttttagtagagacggggtttcaccgtgttaaccaggatggtctcaatctcctgacctcatgatccacccgcctcggcctcccagaatgctgggatgacaggcgtgagccaccgcgcccggcctcatttggttccttctgtgcaccacagtgtgggatctcgggtcctgggtggcacgtgtttaacctgaaaggaaactgcccctgcgctccccagcttgtagcccgtgggcctggctctgaggccccgactgccccacggcctgtgctgcgcatgctggagccaggctccggctctggttggcgcctcccgtggctttaatctgcagtgacctgggtgcttaacgagggcctttgcctctgcgtgtgcactgccttcttctgaggagtctcctcaagtctcccagctgtttaaacaatggggtcttttgtcttttgacctgttggtgccatcagccagcctcctgcacatactctccctacccttggctgccactcctgtcctgcccttcgccatgtcttttttcttttcttttttttttttttttttgacagcgtcttattctgccacccaggctgtagtggagtggcacgatcttggctcactgcaccctccgcctccctggttcaaccgattctcctgccttagcctcccaagtagctaggactacaggtgcccaccaccacacccggctaatttttgtattgttagtggagatggagtttcaccattttggccaggctggtctcaaactcctgaccttaggtgatccgcctgccttggcctcccaaagtgctgggattacaggcgtgagccactgcgccgggcccccgccatgtgtttttaagagcaggcatttcattcagatgaagcccggacactcttgggggttctgctcaggggctctacctgccactgaacgtcctctccctgtgtgggtggccatggcattcagcctgtgttgggccttgttctgcctggctactggccgctaggtctgccccggcatcctctgtgtcctgtgtgggcgcctctgcctcctgctccagcctgtgccaggcaatcctgctcaccttccaggagccaggcctctccccagggcctgcgtcctgtcagggtcagggacggcccctctgccatgctccggagtccctggtcccctcactccgttacgtcctgggtgtcacgggtacggccgggaactctgtcgtcttcactgtctgggccggggcctccctgggtgtctgctggatggagtgggtgcctttgggtccctgcaaagtgagcctgcctcccagcaccgccctgtcgttacatagccactatctttgcgcctgtttttccttcctttgactggttcctctggggttaattcccaggcctggtattaccatctctgaatgcctgggtggtttcagcgcccaggaggctgtgctgaggtatcttagaccatgtgggcaccgttcgctcctgcgcagccggctccggggtgccctgtttttgcggaatcctgcagggaaccccatgtaccctaagagtgctctccccagccactgtggcataagacaagcggtctctttgcccttgggccccatccttgtctggtcggcccttcttcatgggtccagcgcgggattgccggcttccttttcaggcttcctgggacccccactcagacctgcagctgggccagcgatgcccacccgtttctcctccacgtggtatacagaggtgcccaggctgctgctggggactctggagcccaggagtgagtctccttgaccctgagctgtcctggctatcacagctgggtcctgttttccccttcagcacccacgggtgtctttctccagtttatattgttatttttatttatttacttatcgagacagagtctcgctctgtcgcccaagctggtgtgcagtggcacaatctcgactcatcgcaaactccgcctcccgggttcaagcgattctcatgcctcagcctcctgagtagctgggattacaggcatgtatcaccaggctggacctctagtttatattattacagcctggtcagggagtcactgggcgtcgccatcctgtgggtgggaagggggcccaggcagaggccaggaggaggtgacagtcatgcgcttagaagctcaggccacgcccacccagccctgcctggtgaccggttcctgctgtgttgggagctgcatcccagaccttatcgccgggcacagaacttccaagccaggggagagggaggcctggaggggccccaatctctgaaggtcagcagtggcggggaggaggcttgcggtgctgaagggactcgggggacctgcagggaggtgttggtggtcagggatggtggcacctgaggggacttttggcagggccagaagtgcccagagcaagctccggtggggccctgcactggagggctggggtcgagtgaccctcttcctaagaccacccaggaggacactgggtgcagggtggccggagccctcacccccagtaggcagctgctgtccactccgccgacctgcctgtcacccagtaggcagctgctgtccactcccccgacctgcctgtcacccagtaggcagctgctgtccactcccccgacctgcctgtcacccaataggcagctgctgtccagtcccccgacctgcctgtcagccttttccctgtcaggccctgttcctaggagcctggagacctcaggggtggccttgagcccccagggtttttttgaggggaagcgccagctgctgtcttcacccttcccctagtgaggccaggctgtgcagggccacgtggaggcagtctgtgctgcgcccatcggtcgcctggcttcctgccgaccctcggcccccagccacctctggtctcgggcaaggcccctccccctgcccacctcctccctggcccccacgccaggtgggcagacctcctctgcggtttttattcagggggtccctcgttggctgcccactcttggagggctgtcctgactcaggcctcccctctactcagatcccctgagcgagggcctgggcgtgacgccgggagttactggggggccagagggggaggctcagcctgaatcaatgagacccaggaggaaggaggcacgtggacctgagggctggctggagccgctggtgacagatggaggagtaattgctgcttccagagcacagcgagctcgagctccctggagtgccagaagcttctgggtggacagacaggccgcctcacattccagaggctgacacagtcttccaggcacccctggggccagctggaagccatgtgcctccactcacgtgtcccgtgggtgtttggaggggaggctggccctgccatggcccaccccagcctgcggcctcaggagcttgcacatttgcaagggggtgacatgaagactgagccagggctgcggcgggcatccccctcagagaacagggaggagggggcacaggcctttttttttttttttttgacagagtctcactctttggccaggctggaatgcagtggcacgatctcagctcactgccaccttcgcctcccaggttcaagcgattctcctgcctcagcctccccagtagctgagattacaggcacccaccatcacgcccggctcatttttgtatttttagtagagacggggttttaccatgttgtccaggatggtcttgatctgttgacctcgtgatctgcttgcctcggcctcccaaagtgctgggattacaggcgtgagccactgtgccctgccaggcacgggcctttttatgagggaggaagccgtgccctctgccacctgcctgtgggctggggcctgcaggctcccagggccctcagctccagcgtggctgttggttattggcacctgggcctcatgtccacatggaaagtatattccgagctggtggctaagccacaagagcctgcttacctgtcccaacccccggctcacttgctgccatgatacgtctgggattctgggccctccttctgagcttggacacagacttgggacttcagcgctggacacaggtgctggatctggggtctcaatggcaccagggatggtcattctgacagccatgcccagagtctcaggagggcctggggccgcaggtggacaggcatcctgaccagagtctcaggagggcctggggccgcgggtggacaggcatagagtctcaggtgggcctggggccgcgggtggacgggcatagagtctcaggagggcctggggccgcgggtggatgggcatagagtctcaggagggcctggggccgcgggtggacgggcatagagtctcaggtgggcctggggccgcgggtggacgggcatagagtctcaggagggcctggggccgcgggtggacgggcatagagtctcaggagggcctggggccgcgggtggacgggcatagagtctcaggagggcctggggccgcgggtggacgggcatagagtctcaggagggcctggggccgcgggtggacgggcatagagtctcaggagggcctggggccgcgggtggacgggcatctggacgtcggctccactagtgtcacctctgcaaccttgtcccacccagcacctccctatggccctgctctgactgtgggctgagggggacccccatggctagcttgggacctccgctggacactgtcatagggcacttgtcgagtggccctgctgaggttcaggcttgggcgtgtgtggcccctgagcccagcagctgcgtgagccgtggggagccgagcccccagtcaagaaagctgtgtgctccttaccctgcctgctgggcttggggcctgcagggcaagggaggcggtgggatgggagccccgcctggcctggtttcttgttgtcgccgcactcctagctgtatgtagtcagatgaggctcccagtcctggctctgctgctgagcaccattgggccggtggttgggtgaatctctcagcctctctgagcctccagcacttcgtccttaagagtggaactggtctggacctcccaagggggggtcactgggtgcagagttcctgcaaggctgggcccggtgccagtagcggtcgctctgtggggcctttcgcacggttgatttttatctccctgtgagattagcgcctgccctgctctgcctcacggctctggctctccgggactgagaccacctgctaagtgacaggggcggaaggaccttgcaacacagagcaagacgtgatagggtggagctgctccgcccaggccccgcctgcagatgagagggcccagagcctcttaatcccctgcgctcagggcatgtgacggcagggagtctctccagtgccgcccaccggattcctatagttagagctcctcgcccaagcccccagggcacctcactggcctcccaggtgaggacatgaggcccagcgactctggtggtggctggtgtcacctatggcagggccgcgcggggcccagtggaggacgcctggcccggacctgactgctccgtgggaaccgtggggcgtgctcagggtgacttggtgcggctgggctgccagccggcctggctgagtcactggcagtgaaggcaccaggtggtggacgtgctcggcagctggcgggagagtacccccacccaggaccgtgttcaggctgagggggaagggagcggcccaggcaggcaccgcccatggcggggcccccgcactaacggccacctctcccgatggactgcagttctggagccgcccctgaggagaacgccctgctcagtgtcctggcagactctggactcttcaccgtgccatcctcgctacagcctggccaggcagccgctcccgtccccccaccccacagacaggaactgcagactcctaaaggtcgagccagctcccaagtccatagccagaaagtgatgcaggcggggcccggctggggggcaggtgtgaccgggtcatgggggagcgggctgtgcacaggcatcaggctgagaggtctcggggatacgtcctgggagccgcctgccaggcgtcctgtggggctgcctgcaggcctcctctggctttggcaggttctgtcccgacagaccgaggcttgggaagaccggattgtgcactctctgcctggagagagggtggggaggggctggggcgtgtgggcagggagaacaccctgggaggcatcagtcctcttccctggggacgggccatgcgtcaacagtccctcacttcctctctcaggggcagaggcgtgggggtcccgagcctccaggttggagtaggttaggagggcctcagagccccctgctgccccgccatgtggcattggaaggggtgtgaccccctaggggatctgttaaccacactccgccccatgccggcctctctgttccctctaacctgcgcaggagcagggccagctctgagctagcagaggtgacatgaggctcgtcctggccccgcccaccctggcccctccctgtttcccaccacgctccgcccaccagagctacatgttgtgtttgagcctcagccccagacctggctcaggtcctcaggaggaagcagtgacagccaggcccggggtcctctccctgctctgctccctgcgtgctgactggaggctgcaggtccccaggcttggccctgaccctcaggaatggagccggccgatgtgaggtgggggctccggtcatgtgcagtggtaggagaggaggcgggaccggtcccacagctccattgctgccgaggcgttccgcaggtctgtcttcatattggtaaggaaaatgcgaggatggtgtagcctgggcatcccagtccccaagtccgaaatctaaatccaagcggaaaattccaggcctgacttcatacgccaggtcacagtcagaagccagaattattgaaagtatgctctgggcgtggtggcctcacacctgtaatcccagcactttgggaggccatggtgggaggatcacttgaggccaggagtttgagagcagcctgggtaacatagtgagacccccgtcattatttacacaaaattatgtgactgcctttagggtatgtgtacgaggtatatattgaaatgtgagtagaattttgtgttgagtcccatccccaagatacctcattatgcgtgtgcaaatattccaaaatccagaaaatcctaaactggaaatacttctggcccctttggatgaggggcccccactctgcatagcaagtgtgggcgcgggtgctgctgtgtcctagcgtacttcagtgtgtggcctttgcagtgaatagggctggtgtcctcactgtacagatgaggaaactgaggcccagcttgctttgccaaggtgcatccggccccggccatggctattctggctccagatcccatggtctgcagccacaatactgctgtgccccggactgggccctgcagctcgcggtgtcgctggcctgcccattgtgggcaccgcccccaccccagactggccgaggcctagaagggagcagggcctggctgaggctgcaggggtggggacggtcagccagcccctcactgccaggaagggcgcatccatcctggcctctccccagggagaagggaggagcggctgagagggaagcgctcttgccctgtggacgagctcctgccccacggactagggagcccccgcccacaacctgcttgtcagggccacccgggacccccgggagttcggctgctcgctctgctgttaggaattggattagttttccataaaaacaggatgtggtgggtgagagggcagtgtgtccgtctttctcactcccctttttccaggaactgagcacgcgcataggttttagccagggccgtccagtcccctccccaccccccacagggaacaatccactctctgctcttaagtggccacttaatcagcttctcctcctggcccggggagcttcttggagccggcctgccgtggtgggaacagctatggggacaccctgccataaggtccagcagctaagctgggatgtggggggaggggctgcgaggcccaggcagtgtgccaggccgcacaagaggagcccagctcttgccccaccagctggcagccctggaccgaggttgggcccgtgaggttggctgggccctgggccctgggcccccctccccaggacacgactgtggtggcacatggctttgggggctcgtgggtcccactttgcagacctctgctttaaggggtctggtccacggggtcccctctggagggcctgggggaaatctcagggacctggggtctggacccgggggagggagcgggagaagcatgtgcgtgagtctcgtgctgtcagggagcccgggaagtctgcgagggcttgggggttgtgtcagggagtgtgggttttgcccttcagtggtggaagctggcttgagtcccctcgatccctcaaggctgtagtcctgactcggggctgtagggcagggcagggtggggtcaccctgaggcagaaggctcagcggagatgttctggcttggctctgcccctccctggctgggtggcctcaccctgtgacctttggggaccctggttcctctgagccaggggacagcagtgactccgccttcctaggtggctgaggatgacatgggctccccctgcaaatgtgggttctggctcagtggccaagtgtatgatggtatgtggctcttggggtcctgagagagatgggagaggagcaggggtttgtagggaagctggcggcttccaccccagccagtcacctgcagtgggggagttccaaagctgactgaagcttcgaccttgtggctggtccccttccttcctgcctcagtcattctggtctctgggggatcagggctggggggctctgggctgtgggggcctgtttttgtgacttaaagctctcccagcacagccccctgacctccttcctcatgggcaggacctggcccaggggtctcagcacagccacaggccagggatgcccttgcagatggccctggatggaattccagaactcagaaatgtctccttcccgtaaggatgtcccgagactcatgagaccgtttccttctgggaagggggaggaatggggagatgagtgaagagcgcactgcagctcaatccgggaagaagctaatcaatcaatcagggaagccaatcgatcggagaagctgatgggaaagctgtgcttggtagaataggctcgtgcgggcagagcagtcgcggcactcacaggctgactgtgaggaccttgggtgttactttgtgcttctctgcattttaaaaacttttgaaagtggagaagaggaaagtgaatgttctctgagatttcatggaaaggggaaactgaggcccactctagccagtttgggcccagggttccaacctggggtggccccggccctcgtggcctgaggtgatcgtccctgtggctctgagagcagctggggccgggtccccgttctggggctggtgatcctggggaagagccaggcagtgccctgcccacctagtggttatgagcccagaatgttgatttttttcccttggttgcttcatgactttgttgaatttccagagtatgtgtgggggcccccggcgtcccactcgccccagccctgtggcagcagagctggctgtcaagctcagtcagctgggcccagggccccggaggtaggtgggtgtgtgcctgagcttcccctttgggccctgccaggtgctgggagggaccacacaggcggcaggaactcggggtccccaggcctcggccacaccagcctggtgcttgttatatattgatatgtctctctacctgtgaaatgggtatttattttaaggagctgacgcacgcgattgtggggctgtcacgtctgaagttcgcagggcgcgctggctgtcaagagccgctgttgcagtctcgagtccaggctgtgcgctcaggccgggcttctctgtggcggtcctgggaattcctcctccgggcccttaggcgacactcccctgccttcagctgattggatgaggccctcatgccgtctgctgatttaaatcaatctcattttaaaaacacctgcacaccaaattgcgattggcgtttgactaaactgggcagcgtggcccagcagcgctgacacatgaatcaaccctcacaccccacccgcaggcgtgaccacggcacccacgctgccgactgagaacacagccgtgcgctgactcgcatgtgatgtctctggtcgcacctcctccatcctcagcaccctgttcagaatggagatagcggccgggtgcagtgctcacacctgtatccccagcaccttgggaggcctgggtgggcgggtcacctgaggtcaggagttcgagaccagcctggccaacatggtgaaaccccatctctactgaaaatagaaaaattagccgggtgtggtggcaggtgtctgtagtcccagctactcgggaggctgaggcaggagaatcgcttgaacccaggaggtagaggttgtagtgcgcgagattgtgccactgcactccagcctgggcgggcgacagagtgagactctgtctcaaaaaaaaaaaaaaaaaaaattagagataacgacttgggctctctagagaccagggagccaggctccaggcgctgctgtccagccccttgccagctatacgaggtgctcacctggccacgtgtccgggcagtgcttctggggtggcatccactgggagggcaagatggttctggtacaggtgcccaattctgtccccatttcacttactgggaactcaaggcacagagaggggagggctgagctaggaccagaccccagtctcctgcagaagtacacagacatgatgtacgaccatcctggacacctgccctgagattcccctccctctcctgccctgtcccagtggcctggggggaaggggaagcaaggttctgaagggagtgtgaccagacacctgcccgtgacaccccctctccaggctgcctccgagtggctggtgactcccctcctgcctgcgagggaggtggccaggttgcattcctctctgagtgccggggaagtccctagagagcaggccagcctgtgactgggccctggggcagtctagacaggccagactggacaggccagggggctgggtgccgctgggtaaatcacagggtgagggctctgagtcagcaccccatcttctgtcctgggtccagcaccgctgaggacacagtgggcagccgggtctgccagggccaggtagctgtgttgagaaggcagtgctcctgagaggcggctaccgggaggttttcaatggccaggcttcttaggaagcccttgttgcctctctggggtgagttgctggggccatggttggagtggtcgccagtgtctgcccctggtgccgagggcggagtcctcgttttgggaggtcacggcatgatgctgggagtcaaaggcaggccgtggcaggggcattccttttttctttcttttttttttttttttgagacggagtttcactcttgtcgcccaggctggagtgcagtggtgcgatcccagctcactgggatcccaggctcccaggttcaagcgattctcctgcctcagcctcccgagtagctgggattacaggtacgtgccaccatgcccggctaatttttgtatttttagtagcgacggggtttcgtcatgttggccaggctggtctcgaactcctgacctcatgatccacccgccttagcttcccaaagtgttggcattacaggtgtgagccaccgcgcccggcctggcaggggaatgtcgacgcgtgatctctgcctggagagcacgttcatgtttcccagaggacactttagaacatggcgcctgggtttggatgaacctcagcctaagaatctacctgctcaggatccagcgacgctggtggtgtggacttcagctctggagaatgggttatatggaacctgggcgccgggagggcattgccacgtgcttgctgctggggcttcaagaggaccccatctcctgtggccgagaccccgtgtctcaaggcacatcccctttcgtaccccgccccaccctccgcagcttcatgacctctgggtttcccccaggaccttcgcatctgatgttcccagatccttcctgccactgggtcctgctctggtgccccccggggaagccttccctgaggacccagctcagcatctggtgctaactgtggctcatcgtgcactttggcccccaggaggctgtgggctcctgaaacccctctgaaccacgggcttccaaccacacctgaccccctgcgtgccgaccctgtgtgcaggtgtacaggtgtgcctggggcaggatggctgtgacggcctcacagagcccggagagctgcctcctagcttccaaagccttcatttcaggaatcttaccctctcaattagtctggaatgctgggggcggggccagctccaggtcacagagcgaccttgtttacccagaccttaacatcggcccttccatgctaatcaatgtaatcattgtggtccatgcggcctctggaatgtgctgtccactccctgggtcagccagagagtgtcagggagcacctaccggctgttacccagtgctgccctgacctggttcttcactctgcacatttgtatcacgccagaccctggctggcagctccaggtgacgaggcatgtcagtgccttcctgttctgttctgttttgttttgttttttaaatcgggatgaggccttcctgtgttgcccaggttggtcttgaactctcagggtcaagcgaaccttctgccttggcctcataaactgctggattacaggcaggagtcaccatacctggcccactgctactttctagatgaagagacagaatcccagagaagaagcaggggtttggctgctggtctggagccggttctgctcacctccagcttctgccttgggccgccctgttcacacaggagctgctcacaggctgagacctcgagcagggccctcctagaggaactgggcccccgtaagtgccctgagccgccaggagccggccctgcgtctcatccctatctccggaggacattggctgctagctcaccagctggcccctgggcaggcttgaatcatgacctggaacgccaggtgtctctggctctacccctggacctgcaccctgtccaagtgccccagggccagactgttttggttgcaccctctggacgggctacccccatgatggctgctcatggaaagctgtggttcttagggagctgccaattcctagtcctgcagcctggagctcctgggtataaggtgggggctcgggcgtattggggactgggggtctcaggaaagagcctgtgggacctgtgaactcatagctgctggccgagggaccaccttgtggctgtccttctcagctaggcctggtcagggcttgtgtgcagggcggctgaagctgtgggaggccacactgtccacacagtgccctgtaagcccaccgtgcctcagtttccccgtctgacaagtggcacaacagaagccacttcctggcacacagctcagggtcagggccgacacagcacttgtgggctgccgggagacccgagaggctgccccttccttgccttggctgccacgggtgacctggcaaacccctctggccgtggcaccactgggggtctacccttggcagtcagggttggccgcttggctgggagccccttctcctccccagacacatcctctccttggggctggagggggtcctgccgtccccgggattgtcgagcagcaggaatccaggagggcagtgcctgcagctcagatgggggccagtgggcagggccgatccaagggtggcaggaaagtgcccatcactgacctcaggtgggggaggccatggtgtgtgaaggaaggagagctgagtgggaggtcttactttgtcaccgcccctctgagtgcctgctgtgtgcaaaggccagcgggggccctttcttcagctgggctctgcccagacccctgagctctgggtgggccgggagggagaccttgctgctcacagaggtggctctgcctgacccagctcccttcccagggcacagtgggggcatagggtcggctccgtcagacattccgggacctgcgtccttcctggggccacaccctctacccactgtcccccacctatttacctgtatctgcgccagagatggctgcccagataagccctggtttcctccctttctggagaggctgcgggggctggcgaggaacccacctgcgcagagagtcaggggattgctctgtgtggaacgcaggcctcacccatgccctggaatctgtccccttctctgttgctgagggatgagtcccagatccctgaccctgcaggggagcccagcacagaaaaactctgaggcctccacaccctggcagcgctgctggtcgtctgtggggaaggacaggccctgggagggagggggaggtgcggagggcagtggggagggtcaggaggaagtgggggaagggccacccaggccccgatgtgggggatgtctcacgcgtggggtggggcattctcatctctgcttggtctcctgccatgctgggggtcgttcactgcggaccccaagtaccatgaagatggggatgggatgctgagcagcatcggggagaacgcaaaggcacctcccagctcacccgcccccaccccgcaagcacaaccattgccatggtgtggggaccggaaggcggcggctttgggaccagactgcttctgcctcgggccgtgccgctgggcctttggtcagcaccatcgtgccctgcaagtcactcttagcctggtgccctcctgggcagggcagtgccacaagagctcaggcccagatgtgcaggtgccactgttccacccaccagcaggtcacctggggaaccctcccctcctgcagcctctgtgtgctcatctgtgaaatgggcgtggtggagtcacccctggcttgtgggagaatcccaggggctgatgcctgcaggaccctgtgggctttgccccgctccctgggcagagacagttcccccagtcccacccacgtggctgtgtgcagcaggtgcctgctgaccctctgttcctgcacagtcaccttcttcacagacggacccccaccctgccctgcaaacccctccaggggctgcgggctgagtgtgtccgggagggtgtcctgactctccacgccagcaggtctgagagcagatggctgtggcaggtgcggtgggtgcccagcccacagcagccaccaggcctgcaggaccctgccccgtgtaggtcagatgagccataaaactgagtttcctggacactgagctaattaaacctggacaccgagctaattaaatggtccagaagctcctcagtgccccaggctgctggccgggctccaagtaggtgaggaacattccgtttacctcctgctgcgtcgaaggcgggtggctcccctcgggcccctgcctgtcccgggccccctgggtgctgctgcgtcaaaggcgggtgtctccgcttgggaccctgcctgtcctgggcttcctgggtgctgctgcctcgaagacgggtggctcccctcgggcccctgcctgtcctgggctccctgggtcctgctgccttaaaggcgggtcgttcccctcggatccctgcctgtcttgagctccctgggtgctgcctgccctgtggccacgtcccactgttgcctttggacaaagctctcctagggcctggggttctcccatgagatgacaggggttggttcaaaggtctctgaggtttttagagctttgtgaagtgttctagaatccattttctctctcattgctggacagagagtatagactgggctttttcttgagtttctctcaactcttctttgtccatgaggcaggaagaggggcttcctcagtcctcagcttggggtggattgggatggacagagaaccgtgcaggatcccagcctgagacggtccagcgccccgggggagcggagcccgtggctcagccgtctgtagccacggccggggtcactgtcactgagggcacggaggccccgccggcgaggtccgaggtgggcgtgtggggtggtgggcgctggaggcaggacctctgcttgtggagggtgggccaggcgtggaccaggtgtcacgtccgctggggcctttgaggggcaggtggggattggcgcggagaaggagtgaagcagctggggttttgggagcctgtgcggagcaggcccggatgccagggtggctggtgaaggtgggcctggccgggctcggcctccattgggggagccccccaggtcccctccccacacagcttccccctctgtctgctgtccaaggctcctgtgctggcactctggggtggtaggccatgcagcccgtgtgaacctccatagaccttgctgtgaacgctgcacggggcgtctgggggcgggctggcttccccccgtcccctgggccaaccttgccagccttcttcttccataaaagtgggatccgtctgagccctcatggcccctctgggtctgggtttgctttttgtgctctgcctggggtcatggcagggaaggcccagcaggctcccttgcagagcagggcagagcagcggccgccaggagctgcctgtaccacgtctgctcttcttcctctcttcctctgcccggccccgcccaccgtcgaaagcactagcacgggagtgtttatcggtttctcttccaagccaaataaggcagagagcgctcttgcaggagttggagcaggccagggggagggcagctggggcttggaccaggaccccggctccctgtagctgccctggcctggcagctgtcatccgcgaactctgatcctcgcctgccctcacccggcccctttcagagcttcccatgctcagctggtagcaaaagaagtcaagacctagcaaggtgcctgccccggccaggaggcccaggttctagccctgcagctgccgctgtgagctctggggactcagacgactgatgtccttcccagcttctgtttcctttgatgagaggaaggttggaccaagcgacctgccctgcttctgtctgtttcctccagtccttttcctttttgaccagttcacttgttgataactccagcaacgcatgaaaacattccccttgtaaaaaagggaaatgccgtgggtaggggggaccaaggacctcttctgcaggagcagacgcaacagcttggtgtggatcttcctcagcctttttgtccatcctagacatgtggaatttcagttttacacaaaaggaatggtaggcgtctgactctctgtggcttgtatcacttggcagtgccacagggattggtgcatgtttgctctctcatgctatgtgtcaggcactgttgtctctgcgacaatcctgtgaggagggagttatggttcttgatttatagacgaacaaactaagacagaggagattgggctcatctgtggtcatctcagcagttgtgagaggtcagggccagagcccagtggtgtcattctgcagggcacatacagacacgacccacagggttctgccgcaccccctgccgcttggcctgctctgcaggtgggtgccgaggctgttcctgcatctgctgtgtcttgtgagtccgtgggtgcctgtggagtgggggcagtgggtttgcatttccaagcactgcctctgtcataggctcagggagaagcagttacgcatgagctgctatgagccacgggtatgactggcatcacagcaggaggagccctggggatggctgagtgacgatgggtggggacagagctcacagaagccagggttgcccccaggccaggccactgctggggccaaggctgggatgcagcacgatgttggtctgtcagggagtggcaggaagcccacagggtcagctgacacttaacccagagtggtgggagccttgaatgccagactgagggccacacagttggtctcggtgcacaaggacttgggctgagagggctgttgctcctcaatggccatctccttgtgggaggagagtccgggcagccgcttccaggaacccacccatgcttgaaatagtgggaccacagggctgaggaggctcctggggattcctcccaggattcagctgctctgtccccaaccccggagggcctattcgtctagccctatatcctctctgccagttaggtcagggcagagaagtagaggcacagagggtggcggaggggtgtccagccaggcctttctgtggcaccttctgaggctggctggagtctgtgaaatctgtgataactggcccagacccttcctgcctcctggggcatctgctggagctgaggctgctgggggagtctgcctgtgacttgagtctcttgctgggccatgctgggctcagttcgcctgtctgtggggtgggatggtgccacccttaggttgttgggaggacccaaggagagtgatgcctccagccatggcagctggcccagctccggcccgcagcctggctccttcagggccaggaacccccaggtcatggactccaaccctcgggctctcctgcttcccaggtgacgaagcttcaacccatgggctctccttcctgggtgtggaagccctggagaaggtggagatgggatctcagtccagcccattggctccacttcctttgagaagctctttcccccgtcttggcccccagcccttgttcaagcacctgtccctctcctgtctcctaggccccgtcagtccttttggagacctgttcccacccgcccctgctctcacaggctgccagtgtccacacactctcaggcactgtcattggagccttttaaccacacgggaaagcaggcaggttaggtaactaccccccacccccccgccagcaacgccccctgcatacccagggcctgcaccaggtgctgctgcgctgccagctctctcgaggtccctcctcctaacctcaatgcatcgcgtcttccagcccccggctccgagggctcagcctccaggtggtctaacctggagtctaaacctggattgccatctgcctgcctcagcccttcccttccccagggaccgagggccagtgcggctccccagccacccattttttgtttgttttgagacggagtctcgctctgtcgcccaggctggagtgcagtgtcctgatctcggctcactgcaagctccatctcctgggttcacgccattctcctgcctcagccccccaagtagctgggactacaggtgcccgccaccacacccggctacttttttgtatttttagtagagacagggtttcaccatgttggccaggatggtctcgatctcctgaccttctgatctgcccgcctcggcctcccaaagtgctgggattacaggcatgagccactgcacctggcttgtttgtttttatttttattgttttttgagacggaatcttgctctattgcccaggctggagtgcagtgacataatctcagctcactgcaacctccacctcccaggttcaagcgattctcttgcctcagcctcccaagtagctgggactacaggcacccaccaccacacccagatacattttgtatttttagtagagatggggtttcaccatgttggccaggctggtttcgaactcctgaccatgagatggcgggcttgagccatcttcccgcctcggcctcccaaagtgctgggattacaggcgtgagccacggtgcccggccccagccaccagttttgtacagcactcaggtgggtgtggttcctgaggacatgtcacacgagatccttccggtccattagcccaccccgccccatcagtggccagtgcggccattgtggggtccagagtcccgaggcagaacgactgactccaggttgtggaggggcctcttaggatccttctaggcacccaggttgggcaggtgcctccatgcctaagactgagctcctggtggacatcgggctgggtgtgggcgtcctgcacaccctccctgtggcctggtgcccgtgaggagtggacagtcaccccgagagcatagaccaccctcgaggctcccagatggcggccgcagagccccacttccctgcctggtgtgtgataccttccatggctgaggacacctctgactgctcactcctgtgaccctggacccaagtgctgggccacacctggactccaggccccagtccttccttcttctgctgtcctggctcctgggtggcccccgccctccccgctccgaggctctgccctgtgctgggaacccaggaattgccaccccctggccccggtttctcatgtcaggatctgctggccaggttgactcctgagcgccactgtctcctcagacttgctggtcccacccggtccccacctgtccaccgcccgaggctctcctgtgaggccgctctgcagctcttggcctgggggcccccggcctctcatggtgccttctgcactgtcccctttggagatgggagctttgagggtaggcgccaccgttcggtgaaaaggcggtataagcaaagcaacccccagatactgaaggagccgggaaataaaaggaggcagacagagcaagtgtgttggtacccgcctatttactggcaggaacttacagacggaaacatggcctcaggcagcctccaggcaggtagagctctgaacccaaacgcccgatctggggctcgtatcgtgccctgggaggaatgtgcaggtggctgggaacgtggcgggtggggtggccagattcctgctgcagcaccgggtttgtttaggaagaaacgtacgaggactagacactcttttttttctttcctgcacgtgaatgccagggagtaggtgctcctgcaggaagatgatgcatcaaccagccagtctggaggcattccgagacccggggttaatcagcagattagcatttaaataaagttactctgtccccacagccagctaggcccgagccacatggcgggggcggcgggggcggctgttcgccgcatcccaggcttgctccatctcctcgtctgcaatgtggaggtgacagtcgtggtcctcggctagtgccacaggactgtacgggacagatcacccagccccgtgcctgcactgggggcggcggtgacgtgatggccgcggcggtgacctgatggccgcaggtgtgtccaccctctggttaggcctctcctctccctggggcgcaacaggtgtcctcgggtgggaggttcagctgctgggcctggggccccagctttcagctcctgtgcaggcgtggagcgggacggcacgccggggcgttcgttcacacgatgcccccagctcgtctctgcctgctctgtgatgcagcctcagctcctgtgggtcccgggtcacccacccgtgtgggccgcagtctttctgcaccacgtcctgctctcggccagcgctggcctgcgggccccggcagtgtggggtcacacacaggccgcaccggctgtttcacggagcccccctgctcaaaatcctggccttgggtctctgtgtggctcctggagggagcatgtatgaggtgtccaggttgtgaaagccccatccggcagccctccctgtgacagtccttcaggcctcgtcaggcttggttcagcccgtgttagaggaggccagacagctcctcctcgatcaccacagggaacgagggttgggcagtttctcctctcccacccgggcgtgggtcagctgcaagggtctcatcccaggaactggatcctggcaggggggctcaggcatctgacatcccctggcaggtcttcctatgtgccagtgggagtgggtggtgtccagccctctttgtggaatgttccggttgagtggtgtgtggtcctggttaccaccatgtggcatttgtggctttcctggccagcacccactgaaagcagaggccagcccagcccttggacacccaggggcccagcagctaccgtgggggcagcctctcccctcctgactgtgctcctctcagctgtaggatgaaggtctgtggatgggtgatggctgggctctctgcttcccaaggagctggatgtgggccatagtcaggcgtgtgctggtgaggcgagggggttgggggttgggaaccacagctctctctgaccccatcccattgcagctgactcaggaaaaggcattacccccacggggtcacaggcccagcctgggcagctcctgatgggaacatgggagccacagggcagggcaggctcctgtccccgctccttggattgtctcaggccccgcctcccctttggctgaatttcttccttctctgcctgagcctgatctgcgaagcttccgacctgccctgagttggagccagtcttcagggccaaaagcaatgccgtctcctccaggcagcctgctctgaagtctccccctctggctttagcccacaaagtcacagctgaggccacatcaacacacttagaccaagagcactgccggctgcatagacagcacaggtagatccaaggagtgtttcgaaaagccgtggccctgcggccgggatgcccggacctgggtcctgaccactcttgggagctcctcccctctcagagcccatcccatctgaaaatgggggcattggagcattggaggagtgctcctcactcctgccctgctagccggctactccgaggaccccaagaccagatgatgggagaagtggtgggctgggctgtgaccctgagcccccttctcatcccacccgtgaggctgcccctgcttggtgtctgtggcaaggctgacagaagatggcgccagggaccagccagggccctcctgtctcagcccgtccttgggtgagtgggtgtgctctgggggtggcaggtgtgctccgggggcgggaggtgtgctgcggggggccgggtacgctctgggggggcgggtgagctctggggggctgggtgcgctctgggggggctggtgtgctctggggatggacaggtgtgctctggggggcggggtgtgctctggggatggataggtgtgctctgggggggcggttgtgctctgggggggcaggggtgctccgggggtgagtgtgctctggggggcggtgggtgtgctctgggggcggtgggtgggctttgggctaagtagccctcttcctctctgagccttttccttgtctgtgttgttggtgagtggccgccacctgggacaagccttagctgtctgtccaactgtccaccatgtccccggaccctagaccaagcccgctgcctggtggggcctgcttggtattgaatgaatgaatgaatggatgaatggatgaatggatgaatgaatgagtgaacaaactctggggggcgtgttgctgatcaccccagggcaggcccgtggtgggtcatggtctcaggcaggggacttgccctgccgggtgacgttggcctgccggggaacccctgccaaggtgcctcccatgcccccatcgtcctctgcctttgagtccactctgcccctcaaggttcccctctggctcggccaggggcagggcggtcagggaggcctccagctccagcccctccctcatctccatcctcagcccctgcacccgcctcatctccagccccagccccttgcagcctcaggctcgggcttctgagaatcctttgcagatttcagggctggttatttttttattctggcaaaatatacatccataaagctttgtttcctgagatgatctcaccctgtcgcccaggctggagtgcggcagcgtgatcatggctcactgcggcctcagcctcctgggctcaagtgatcctcctgctccagcctcctgagtagctgggaccacaggcatgcaccatcatccccggctaatttttgaatttttttgtagagatggggtctctgtgttgcctggtctggtctcgaactcctgggctcaagtgatccgcctgccttggcctcccaaagtgctgggattacaggcctcagccaccacacccggcctaaagtttgtcacgttagccgcattgaagggcacaggtcagcagcggtaagcatgttcgcattgttgtgcagccatcactgacacccatctctagaacttcttcgttttcccaaaggaaactgcacgcacggcaccacctccctggcctccccacagcctcagggtctggtgaccttcatccccaggacctacgcttctgactccaggagccctgcctggtgtccacaccacactaaggtgccctgccacgggcttcgaacccaaccctggtgcacttgttgagcattggacgttcctggcaggtcagaccgtgagctcctctgctctgacggatgcatggacggctgccacctggctggacaggtgcagaggggaggtgccaccttctctggggacaggactggggcagaactcacccggctgaaaagtgcagctgtggagaacttcagaacttcagaagcctttgctgtgaatgcattttcctcctgcctcttgggccagatcttagggattttttttttttttaatgaaaatgtataatcgtcaaaaaactttagatttaaaagcatggagccgatggctatgcttggtgcgttaatgggaggaagaggggatcttaacttcagggagggctcctgggggagctgggggaggctgctgtgtccagcagggcgggcggtgccccccggagcccggcactccgcgatgtgtgcgctaagccgaggccgccttgagcccgtagcagcgccgagcgcgattctttcgtgtctgcttccgggagggtggaagggtgaagctgttgagagtgggaagggaggggctgtgcttcttgagtttgccgtgtgcccctgaccctttcaggtgcaagcgtcagctccacggtgccaatggggagatgggtctggaggccccaccaggctgtgaaaggcccggcctcctgtgctgtccgtggaggtcggggtctccctctgccctgcgcctcctctaacttgccctgcgttttccgtgcccctggtctagggtgaggctcgggtcttggggactgaggggcaggtgggtgtcaaggtgagagctgggctgggctgggcctccctccagaagacttcccggggctcctttggcaccaaggatggggcagatggtggcacgcatggcctcccctgggcacagagccagactatggagggatctcgttggctacatcctggctggggctttggggtcatggtgggagcagcagccaggggtctcgcttggacacgggtctgagctcactgtggcctccttcctcagcctcctgaccggggtcctgaggctggagggtgggtggagggtggagggtgggtggatagtgggctggaggatgggctgctggcgccccttaggcagcatgaggcgcctttccagtcccagtgtcctccctggtagccccggaaaggctgagtggtccctcaggtcccctgcgaggagacgacatcaggccccacccctcatctcctgacaaacctcacattcctaggaggaagcaggcctggctagcacgtggcaggcacagcctccctgactccagaccttatcagatcagtgcaagtgccagggagaccccgggtgggagctgtgtggttgggactgatgcaggcactggccggactctcaggatgagggtgggaggccaggccccgggaccttctgggacaggcaggagggcagacacgggacatggtgtgggggctgtggcgagtgcggccacgcagagcatttgggctgaaggcgcgctgtgtcccactgccccatacgtgtgcgggggctcccagacacgtgctgggggcaggaatcatagcatttttttgtttgattgttttttgagatggagtctcgctctgtcgcccaggctggagtgcagtggctcaatctcggctcactgcaacctccacctcccgggttcaagcgattctcctgcctcagcctcctgagtagctggaattacaggtgcccgccaccacacccagctaatttttgtatttttttgtcaagacagggattcaccatattggccaggctggtcttgaactcctgacctcaggtgatcctcctgctgcagcctcccaaactgctgggattacaggcgtgagccaccacacccagccaagaatagcattttctttctttctctttttgaggcggagtcttgctctgttgcccaggctggagtgcagtggtgccatctcagtgtaacctccgcctcccggtttcaagcaattcttctgcctcagcctcccgagtagctgggattacaggtgcccgccaccatgcccggctaatttttgtattttcggtggagacagggtttcaccacgttggccaggctggtcttgaactcctgacctcaagtgatccgcccgccttggcctcccaaagtgctgggatgacaggtgtgaaccactgcacccagcccaagaatagcatttttaaaggaaaaaaagcaaggtagaaagatgggtcaaaaccccagcgccccgctgggaggctggtgcccgtgtcgtggcgtgtgtgagatatgtacggaagcgcgaggcggcttccacggctctggctcagggcagcactcacagggcacacggcctcggcggtgtgacgagctgggccgtgggtgcgctgtcctctggggtggggctggccatcctctgttggcgacctagttaacgcccatctccccgcagcctgcctgctccgcttcagcggactctcgctggtctacctgctcttcctgctgctgctgccctggttccccggccccacccgatgcggcctccaaggtaaggccaggggaccctgcccacgctctcagggtgggagggggtgggcattcgtttggggccaaaattcgtacagctttcctcacctttaacagcccggggtggaagatgaggggtcccgaaataaaatttttttttttttttgagacggagtctcgctcagtcgcccaggctggagtgcagtggcgcgatctctgctcactgcaggcttcgcctcccaggttcacgccattctcctgcctcagcctcccgggtagctgggactacaggcgcccgccaccacgcctggcttatttttttttgtatttttagtagagacggggtttcaccgtgatagccaggatggtctcgatctcctgaccttgtgatccgcccgcctcggcctcccaaagtgctgggattacagacgtgagccaccgcgcccggcttttttttttttttttttttttttgagaccgagtctcgctctatctcccaggctggagtgcagtggcacaatcttggctcactgcaacctccgcatcccaggttcaagtaatcctcgcatcccagcctcccaagtagctgcaattacaggtgtgagccaccacgcccagccatcaaagtttcttattttcattgtagaccccaagctaagctcagaatcttttctgatccgtgttgcaggggctgtgaggggcgaggagggtcatgggggtggagtctctgtgtcccttgggaggccacagggagtggatgggccccaactgcccactctctgcaggctccaggcacgtttcctgcctgctgaggctctggcagcctgggagttccagccgcctggaccgccctcccgctgcccgctgtggggttggacctcagttgactttgatccacttgtgcgggggggggggggagctgagaccccccttgggaaccaccttggggtctgagctgggccaggactgtgttgctctcccgtatcctccaaacagagggcggtgaggccacgtgccaagcctgcagcctgggtgacgagggtagcagcaaaccctccatcctgtgacatgtcacctacctgccctgtgggggccatgtccattttgtgcccacgttttaggagagcgcgttttggagcaaacactcagagacctgtccccaaaggccatgggccaaacgtgggtggggccgggccctccgctggtctttgggtttggagggtgaagagccactgaaaggaaggaaggtccccagggagggcaagtgtaggcagcaagagaccttccccagcagagggcagagaggaggccaagcaggaccagggggcgtgactgtggagacaggctcttgggtgccgcccttctgggaggtgctggacgcatcaggctggcagcgtcggggcctgtccatggctacgaggtgtccaccgggctgcgtttttccaggggactctggggaggatctgcttcctcttccagctgctccaggtgcctaccttcctgggcttggggcccctccctccatctccagagccggtggggtggggtcaggatcacaccctgtctctcctgctccctctcccctgcggtaaccctgtggcttcgtcccatctgcctccccggctaatccaggccgacttccctggtttaaggtcagctgttgggcagcccccatttcctctgctgcctggactcgcctctgcatgtgagcttctgcgttcacagattctggggactggggtgcaggcatcattggggtccatcgttctgccagctgcaggtggaaggtcgctctccggcctcgcgacatctggctggaagcatcccagaggctcctcctagctcgtggtgctgtggggtgggcaatggtcttttttatttattttttttgagatggagtctcactctgtcacccaggctggagtgcaatggcacgatctgggctcactgcaacctccgcctcgcgggttcaagcgattctcccgcctcggcctcccgagtagctgggattacaggcgcccaccatcatgcctggctaatttttgtgtttttgtagagacaggggttttaccatgttggccaggctggtcttgaactcctgacctcaggtgatctgcccacctcggccgagactacaggcatgagctaccgtgcttggctttttttttttttttcgagtgggggtcttgcttggttgcccaggctggagagcagtggcatgatcatggctcactgcagcctccacctcctgggctcaagcgatcctcctgccttagcctaccaaagtgctgagattacaggtgtgagtcactgtgcccagcgcaggcactgatcttggatcatgggtagaattcaggggtgcagagaggcattttgggaggggcccagtgcggccgagtgctgggagtcgctggggcccgaggcccaaacccctccaggatgcatggctggggtgggtgggcacccccactcccggtccctatcctgggcctcccctttgctctgtggagccggttctgtctgttcccaggcccctggcgtttcgccgtttgttccgtaaatatttcatgcccagggcgcaattggaaacactttcctttaatcagcagtgggggaaggcaggcgcccagccaggccaggggaggagctggggtgggaagatttggagaggacccgggaggacttccctgcctgagcctcgtcagaggcccttcagagacggagatgctgcccagttttccgggagggagagaggaaagtgtgaggcttgcccgagccgagtgcccggggctttatgatttgtgcagctgctgggcttggcgtggccctggtggaagctctgacgccatctgagccttggtccccttgtaggcgtcagcctgatccttggggggctggaggttgagctcagactgaacagggaggagaggggtaggggctggggctggggcagagggaacagaccctggtgctccaggcagggctgcaggcagagccacagggggtggctcccagagacctgcttgtcatttagggactcagaagccccatcctgccttaggataaaacccacctccaagactgaccctgctgtctggagagagatcaccccccacctcttaccatgccccgtgcagggaggccctgccaccctctccagcttcactggtcccccggctctgccagccttgtgtagtctccatcctctgtctcaaaaggcccctctcgtggccctaccccgcagctcagccctagctgtcgctgggtgggcctcatggctcagcctcaaggcaccctgggtcccttggctggtcccatgcacaccccatggatcaggttccagggggctcccatgcacaccccacggatcagactcagggcaactccacactccccacagaacaatcaggcccagcccttgtccggcaggtgtggggatggtggctatggaccacgcatcaggatgctgccgtggtggggcccctcagcctttgcaggccgtgccggggtccaagggctccggctctgggggaggtccatgctctgtcaaccatccggtctgcaataccctggggcctccccgccccttcctgtttcctcctgagagccctgcaagctggagggggagccccggcccctttttttttttttttttttgagacggagtcttgttctgttgcccaggctggagtgcagtggcgcgatctcaactcactgcaacctccgcctcctgggtttaagtgaaagtgattctcctgcctcagcctcctgagtagctgagattacaggtgcccatcaccacgcctggctaatttttgtattttcagtagaaacagggtttcaccatgttggccaggctggtctcaaactcctgacctgaggtgatccacccacctcggcctcccaaaggtgtgaaccaccacgccaggccccctgcaccttcttctgggtgggcaagagccatgctgagcccctcccctgccatggataggggtggcaagaatagtccattccccatgggctccctccggcaggcagcggatgggttggggcccacacagtggttcaggaaaggctgaagggcggttgccggccagagctgggcgggtccctggcctgagcctcctctcactccctttcccaggatggaaaggccactgtgagctgggtgtgtcctgccaggagggagttggagtcgggggcaggagacccactgggtctcctgggctcctggaagaggcagcagtgggtccctgcggaggtggcctgatccccggctcagcctggttttcccagctttgcatttgggggtgggacctgcagggggagagtgacggggtgggcggggacaggggctgcaggtggaggcacgagaaagccacccgaccccttgaggtcatgctgtttgcgtcggagaagagggccggtgtcagggtgccccctcggcctctgtgtgcacctccccgctccccgccccgtgcaggtcagcagagcctcgatccctctgcacgttccggccccctcccatctccagcagcgtctcttcccggggtgcccctcggactctcagctccctagtctgatccagcccaggcagggagcgggcgtgccacgtggccaggagtggcctccaggcccagctgcggccccttcgaccctgcggagggatcatggcccagctgttctaacatggccagaccaggacactctggccggcccgagaactgaaccgggaaggaggcagggaaaggggagggaggaagacaatggggaagcagtgacatccgagatgtagccagagacggacatcctggactgtcgtgcagggcaaggcgggtgggcgggagccaggtggcctgagagcccctccctgcccaggggtctctggtcaggcagttccttcccgggctgctggatcgtgtgtgcagggaacccgtcagcctggctgcccaagggcccagatgtcctctcagtgcccgggggtccttgacagccccagcaggagccccacgtgcctgtggggcaggccccgcaggtccctctctgtaggactcagaataccttctccaatgccacgtgctctccctgagtgcccagtgccacagagggccccgctggcgaggttacttcaacgtcaggctagagggtgcagcgagcaggactcacagcccaggttcccaggcaggtgtggcaggagcccattccctgggtcggtgttctcatgtcaccccacggtgaccctgctgtacagaggcgggggcgcggggcccctggctggtgccctcctgctgccagacttgtgctctgctgaagaggggctggcgcggcaggtatgagcccggcacggaggtcgtgagcagtgagaagcctggcacctatgggttcggggcagggaggccctggaaggtcctgtcctccgggagccctgcacagcaccccctggtggtggctcctgttcgtgtcggggctgcagcctcccttcctggacggttttccattcctccttcccactctccctgctgctcacccctcccatcctccacaggtcagaggtcatagctgcagggtcagcaggtcagggcctggggcaaccctaggaaggtgtgagtgtgagaagctggtccaaacctgcctcggctgcctggtggtgcggacagggagtcctgggcatccgtgagggctgcttcacggtcaggcttagggatgtgcagggtgacttggacgtgggtcatgagtctttgctccagaaagagggggctgagtgtccaaggccaatcccgagtctgtcacctaacaccatttgtgctcaaaaaactgaacagagtggacacaggccctgagtttgcgccccgaggccgagagagggcagatgtggtggtgacattcaccaccttggacccagacccagatgctacctgtccctgagctccaggaagttgtgagaagggcctgagctgttctgcactttctcgtggccgggcgtggctggtggtgcaggagttgctgccccagggtgagggcccggaagctcctgccagcacgtgccggggtggaaagggaagctgtccacagccctgtcaggactcagaacccggtgggtcaaggactttggtccggacccctgctgaagggtgagctgtccacatgtgcgccgagagcagaggtgaagccagggctcctgagtgcccccagccacagggtgcgcccgcccagcccctgccctgcagccgaaggcctccctgctggggggctgagtccagtggggccacaggcagctgggagcaggacaaggctgccaggcaaccagatggtgctgccgcttcctgccaggtgtgggtgcacagagagagagaggatgccggtctggggcctgacctggtgcacagcaggtgcctgaaatgccagggtggccatggggactgggtaccatgcataggccatgcatcgggatgcaacttctccttgcagcccctcagccccagggaggcagctgcctgccccacttttctccagagccatcatggccctgctcccaccccagccacggctgctcaggggcgctccgcatgctctggtctccatccacctgcagcccccactggggtgaggtggagcttcttgcctctccttgtgtctatttcctctgcttccacaactgaacggtgacaggtatttgctggatgagggagcacaccccaggtggtttcctctgagcctgggaggccttttcctgcctgtgggccccaggcccatcctgctgccacccccaggaggatgcccggctccttgtgacaagagtgaccctcgggaggcgtggggagtggggctggccggcctgcctgatggggtcctgagtccatggcgggtttgcatctcaggcctctgggctctggccgggctgggggctattgtccggctgagcggcctgggctgcggcccctccccgtccccgggaccagcctcacccactcgctctgccgcaggtcacacaggccgcctcctgcgggcattgctgggcctcagcctgctcttcctggtggcccatctcgccctccagatctgcctgcatattgtgccccgcctggaccagctcctgggacccagctgtgagtcgctgagggggcggggtagggatagccatcctgggggtcagggagagggccctgcagtgaccccgagtctcctgggggggttgactcagcctgatttatgtctggcctggatggtccaggtgaaacgctccagggatgaccaggccacggtgctggctgggcagagcctgacctgggttcccccgtctttctctgcaggcagccgctgggagaccctctcgcgacacataggggtcacaaggtaagaccattcctcccacccccaaccagcaagcctcccttggggatttcaggccccaggaagtggggggacccaggagggacagagggggacctggagactcatccacactcccacccacacctggagacccatccacgctcccacccacacctggagacccatccacactctcacccgcacctggagacccatccacactcccacccgcacctggagacccatccacactcccacccgcacctgcagacccatccacactctcacccgcacctggagacccatccacactcccacccgcacctggagacccatccacactcccacccgcacctggagacccatccacactcccacccgcacctggagacccatccacactcacacccacacctgtcctcttggtctgaccgcggctgctccctgctctccgcaggctggacctgaaggacatccccaacgccatccggctggtggcccctgacctgggcatcttggtggtctcctctgtctgcctcggcatctgcgggcgccttgcaaggaacacccggcagagcccacatccacgggagctggtgagggcagctgcgtcacccgtgtgtcagggaggtcattcgagagctgtggtctcagccattttgagggttattttaatctttttaaaacagatgtagacgttttggttgtaagttggtgttaaagagaggaggaagttccaaatcccaccccggggcccagcctgcagttccatccgttcagacctgtttctactcgggctctgcctctagtcagaaacctccacgccccgacatggcatctgtgcccttaggaactcttcacaggggaattatttggggccacgcggtggtggaaacctgcagtgctgggcagtgggtctggctggagagccactgcagaagggctgagaaggggcggccccagcaggccccatgcacttacaggcaaacaggctgtcggcccagagccccagcaggggcctggaccccaggagcgacgggcctgaagcagggcctctgtcctcggagtgggaggcagagtgaactttagctgctacaagacttggaggtccggccccgggaatctctgagctatggccccctcacacggacagggcatgagctggggcctgcgacacccaacgttgactgctcagaccctctcgtccctgcctgggcccacacttgccatccccaggctcagccaggatttatggccacctgggtgtcctggcccacgaactccctgccccgggcacccagcctcctcccacctcaccctcccagtgcccactcgtcagatagctgatgctcgccacgcacatgggcctcaacttctagaggagtcccgagagggaaaggggttccccaagccacacggtaggtcgagtacacctgctcagcctgcagaggcggcctgccctcctgcctgtcctgtcgccaccccatggggcaaggctggtgcccgcatttcagaagtgggaacggggacctggaggtcaagtggcttttccgggcatttgagggggtccagcgctcatgaggactgtgctgctgcctgtcctccaagagacccgcccctccctcgtcagcccctccatgctgatgggggcatggggcatgagcttcctagagtttggctgctgggaaggggctgggtggctggactctggtcatcttcatcttggtgtgatgggagcctgagtgtgcagctgcgccctgggcggccagcgtggacagacttcagagtgagctgggggcaccaggcacctgcaggataccgttccgaaagatctagaagccccatgacccggcctggcagggcccccgggcgaccctcactcccacatatgggcaacacacacacgtgtttgcaggcatgcgacggagtgtgccagccagcccgaggggtgctggccgctagaggagggtgaggggcgtctgcagcatggctgcggcacgtggggcccaagtcagggccgggcctgcccctcggtggcctcacaccccaagtcagggccgggcctgcccctcggtggcctcataccccaagtcagggccgggcctgcccctcggtggcctcacacctgttctctcacagcagctgcctctgttctttccactcccagaaatgctcatttttcacggctctgctcagaagatttcccagggccagggccagggccaggctgtgggtggggtgagcgatggcgccctgcgttctgatccagcctcgcccccagctgctgtgtggtctctgagaggggtgggctcagggcctgggcctctcctgccctgatctccctgttaccctggcctcggagcctcccagcagcccctgcagccctggaggcctgtgaggacaggactggtctggttcatctgtcccgtcaagcccccatcccatttgacagaaggggaataggctccgagaggctgtgacttgcctcggcctgcagagcacggagactgtcagagctggggtggggcctatcgtccagccccacagcctgcggtctcagcccgggcacctggcgcctcagccaccaggagagcccccgagtcacagatggggacactgggccggtggcagccaggccagagccagggcatctgacgacagccgtgttctttccaccacacctggctttccctggtgtttgggaaatggctgtgttctgggaaatctttagtcaggtccagggaaaacaggcccggcaggtctctctccccagccccaggccggcctgtcacttcttctaggcagctggccacctctgtccccccagaggcttggggagggcaggagtaccacccctatcttccaggcagagccacccagaaggctggcaggggtacacaggagaccctggagcctggccatgtccctcaggcccccttggtctctagagaccccccggggtatagacagggccccgctgctccctgggtgcgtggttcggggagatgaggtggtataggacagtctgtggtctgtctgtacctggcaaggtcatcacgtgcctgggcttggcaggacagaccctgggtcttcggccagggtgggagctgctaccaggaaggcctgcaggaactgtgagcttgagtgaggaagtaggaaggtgtcaggcagacctcagggacggctggggcctgtgcccggggaggctgtcctgtggccctcagaggagcagctgtggatgtggccgcctcccacgctcctggctgggcaggtgtgggctggagaggtggcgtcagtgcgatacacctgaccttgcccctgtccgtgaccttggcaggatgatgatgagagggatgtggatgccagcccgacggcagggctgcaggaagcagcaacgctggcccctacacggaggtcacggctggccgctcgtttccgagtcacggcccactggctgctggtggcggctgggcgggtcctggccgtaacactgcttgcactggcaggtacgcaccgaggcagggggcactggcagtcacactgggaggggtcttgggagttccctgatgactgtggagacagcgggacacatggcactggccaggtaccaccctgtgtgcccctgccccgcaggcatcgcccacccctcggccctctccagtgtctacctgctgctcttcctggccctctgcacctggtgggcctgccactttcccatcagcactcggggcttcagcagactctgcgtcgcggtggggtgcttcggcgccggccatctcatctgcctctactgctaccagatgcccttggcacaggctctgctcccgcctgccggcatctgggctaggtaacggcttgccacacagcccctttttcctgccaccctggtcccgcccacctggctcgtctagcccctgtggccccactgcctctggggtggtaggctgtgacgggtcttctctggacagggtgctgggtctcaaggacttcgtgggtcccaccaactgctccagcccccacgcgctggtcctcaacaccggcctggactggcctgtgtatgccagccccggcgtcctcctgctgctgtgctacgccacggcctctctgcgcaagctccgcgcgtaccgcccctccggccaggtgagcacctgccacccatggtgggtgggctgaggccaggccatggggctggtctcaggacctcctgcctctgggtggggtgtggagctggtttgggctcaagacgctggtctctgcagaggaaggaggcggcaaaggggtatgaggctcgggagctggagctagcagagctggaccagtggccccaggaacgggagtctgaccaggtgagcagccaggcaggtggagacgccagcgtggggggcgcccggccagcccgtgcatggctcagcgctgcttgcccacagcacgtggtgcccacagcacccgacaccgaggctgataactgcatcgtgcacgagctgaccggccagagctccgtcctgcggcggcctggtgagtaccgcacactgcaaggtatggctgggtgcggggggcggggcggaggccggtgctgccccctggtggccgcctggcgctctcgcatgctcgcgccgcacctctgcctgccgccccctcgggggcccaggacatccacgggtcggtgtcagtgacccccgagacccccagggcagccgagtggccatgtcactgaccaacccccaagacccccagggcagctgagtggccgcgtcgttgatccccaagaccccatggggggcctccaggtcccccaacccctccccagagaatgtggctatgctgtcttgtgctgttagctctgggagctgctccaggtggcccagtggccccaggaggccgctcgtccagggcaggggctggcctgggaactctgtgttggccacgtcgccttgggagggcctgggggctctttctggctactttctttctttaccctaacccttgattttccattttgcaatgtgtttctgaatgaagcaaatgaagccacggccctggggtgggggtcctgagagtcttcaggtgcgcagagctggaaagggggtcagggccacctttcccaccctttcaaggaaagtgaggcccagagaacggcaggtgctggcagggccatccctgacgctcagggacggtgtcagcccaattgccggagccctcgtgttctgcccatagcccaccgggggcctgtctctcctgctgtgtgcttgcccagggcccagattttagggcatagtcagggtggggaggcctgcagatcaacctgccgaagctgaccgctgtccccacctgcagtgcggcccaagcgggctgagcccagggaggcgtctccgctccacagcctgggccacctcatcatggaccagagctatgtgtgcgcgctcattgccatgatggtaggcggctgtgggggttggggtgggcggccccctctgccgcgcaggtgtggggcatcgcctgggtggggtgcgctgggcagctgtgcagccccctctgccgcgcaggtgtggggcatcgcctgggtggggtgcgctgggcagctgtgcagccccctctgccgcgcaggtgtggggcatcgcctgggtggggtgcgctgggcagctgtgcagccctctctgccgcgcaggtatggagcatcacctaccacagctggctgaccttcgtactgctgctctgggcctgcctcatctggacggtgcgcagccgccaccaactggccatgctgtgctcgccctgcatcctgctgtatgggatgacgctgtgctgcctacgctacgtgtgggccatggacctgcgccctgagctgcccaccaccctgggccccgtcagcctgcgccagctggggctggagcacacccgctacccctgtctggaccttggtgccatggtgagtgtgcaccaccacatccgggggtgcctgggtgcgcagacccatcagggttgtcgtcctgttcaatgtccacttgcccgggggagtggcagcgccaagaaggcagatgtgtctgtctgtccccttctgcccacccagagccagcccagagtagcttctcagtgagcgtttgttgactgaataaacagacaaccttgtgttggcacgggcaccacccctgtgccctgacactgtgtgagcgtgggctctgttggcacgggcaccacccctgtgccccgacactgtgtgtgagcatgggctatgcccattggcacaggcaccacccctgcgcccctgacagtgtgtgtgagcgtgggctctgcccattggcaccatgcacagccctgggtctcagtgacaagctgtgcaggccatgtgttcacagggtgcctgcgtgtccatgtgaaacgggtgccagcatcgtgtctggacacctgtttgcaggccagtgggtctcatcttgtgaaacttgtgagcctgtgtgccaacatatgcacctgtgagcttgtgtgcatatgtgagcacgcatgtgggcaaacatgcactccagcatgtggacatgtgtgcaggtgcgtgcatggatctgtgcccacgtgaactagtgaacccgtgtgtgactctgcgtgtgagcgcaagtgaacccatgcactcatccatggatgtgagagtgtgtgctcgtgtgcctctgagtgggtgtgagcgagagggtgttcggtgcctgtggggaggctgcggtggatgggctggtgccagccgcctgagagctcttgccccctgctataggagggtgctgggtcccccggctgtgggaggggtgctgggccccccggctgactgtgacaccctgcgcttgtcacagttgctctacaccctgaccttctggctcctgctgcgccagtttgtgaaagagaagctgctgaagtgggcagagtctccagctgcgctgacggaggtcaccgtggcagacacaggtgagtggtgggccagaggcgggggttgccctcctgcctgcccgccctgatgccatcgcctgcccctggcttggcccacagagcccacgcggacgcagacgctgttgcagagcctgggggagctggtgaagggcgtgtacgccaagtactggatctatgtgtgtgctggcatgttcatcgtggtcagcttcgccggccgcctcgtggtctacaagattgtctacatgttcctcttcctgctctgcctcaccctcttccaggtggctggggggccgggatgggggctggggcacggaccctccccgcggtcctcaccacccccacctcacccggcaggtctactacagcctgtggcggaagctgctcaaggccttctggtggctcgtggtggcctacaccatgctggtcctcatcgccgtctacaccttccagttccaggacttccctgcctactggcgcaacctcactggcttcaccgacgagcagtgagtccaggctggggcggtggggcaggggcgccgaaaccccgtgcacttccccggggctgcagcggctctgccgggggccgggccggtgctgatgctgcccctccacaggctgggggacctgggcctggagcagttcagcgtgtccgagctcttctccagcatcctggtgcccggcttcttcctcctggcctgcatcctgcagctgcactacttccacaggcccttcatgcagctcaccgacatggagcacgtgtccctgcctggcacgcgcctcccgcgctgggctcacaggtgcggccccgccctccctgtccggccctggagaggtgtagcctcctgggccagggagggagccaggtgggagttggacaggagccacatcttccaccttcagatcccaaggggcatttgctcataccaaggggatggcagtagcgtggaggtcacagggacagtgggcatgagttgcgacacagctgtgcacctgaactggcagctgcagcagaagcggtgccgacagggcttcttccagccccaggaaatgaggggcaggaacccagttgggagatgacattttcggaccctctcccaggcaggatgcagtgagtgggaccccactgctgcgggaggagcagcaggagcatcagcagcagcagcaggaggaggaggaggaggaggaggactccagggacgaggggctgggcgtggccactccccaccaggccacgcaggtgcctgaaggtgggttgggcgggcagagcacagctgccacccagtctgctgtgccatgtcccagctcggggggcgttggcagagtcccctctgggctccagagcctcttcctcacaggggacccgggaatccccgtttgtgccccgcactgaccctcacaccatcacaggggcagccaagtggggcctggtggctgagcggctgctggagctggcagccggcttctcggacgtcctctcacgcgtgcaggtgttcctgcggcggctgctggagcttcacgttttcaagctggtggccctgtacaccgtctgggtggccctgaaggaggtgagtgtggcaggcaactcagcttcccatctggggtggggtcgctctggcctgcccagctggcctccccaagcccagccccacgtgcccactgccctccccaagcccagccccacgtgcccactgccctcaggtgtcggtgatgaacctgctgctggtggtgctgtgggccttcgccctgccctacccacgcttccggcccatggcctcctgcctgtccaccgtgtggacctgcgtcatcatcgtgtgtaagatgctgtaccagctcaaggttgtcaacccccaggagtattccagcaactgcaccgaggtaccggcccccgagggctgggacgggaggaagctccaggcaactctgtattcgcagcccgaccctcctggggcagctgcctcagtgcagtggggccagcaatggagatggaggactctcccctgggggcgccaagggggcttcctggaggcagcatccttcgacctcaactgtggaccaggggcgcactccctgcacacaagggtgtccagtaggggcggagtcccagggtctccggcagtgaggacgggagggccccacccctggacagggagagacagtcaggcatctctgcctgggaccttctcgcacatccctccttctccctggacctctcttcactcccccagcccctgcccgtggtctccctgtttctcaaacacctggtccccttccccgtgaaggtggctccaaggctggcagcccccgtgtccctggctggggagcagtggacctgccccagagctgtggctgtggtgggctccgggcagggccagggggcactgtggcctgggagggggcactgatgcctggcctcttgccagcccttccccaacagcaccaacttgctgcccacggagatcagccagtccctgctgtaccgggggcccgtggaccctgccaactggtttggggtgcggaaagggttccccaacctgggctacatccaggtgagttgaagggctggtgggcggctgggcgggcgagtacccggctgccccctgacccttgccctccgcagaaccacctgcaagtgctgctgctgctggtattcgaggccatcgtgtaccggcgccaggagcactaccgccggcagcaccagctggccccgctgcctgcccaggccgtgtttgccagcggcacccgccagcagctggaccaggatctgctcggctgcctcaagtacttcatcaacttcttcttctacaaattcgggctggaggtgaggcaaggacattgcctccccctggggcagggcttggccttcgggagggagggacggctgcaccgtgcaggcaccgcaagcctggccccacctgggtttgcctgggccacagagggtgggggactcagggccaggcacggcttccctggactcctgtggtgtgtcggtgctgacaacaggcagggggccaagttagatctgctctactgtacagcccacctcctggagcctcagtttcccctgcacgatggcaactgccagccactcctgccctcttgacagcgccgctggccctgtccttgcttgatgcccgcagcctccaggcagggctgctgcaagcctgaggcctgctgggtgggacaagaaagtccctccccccagactcagtgcatccccacaccccgccctctcccctccccagatctgcttcctgatggccgtgaacgtgatcgggcagcgcatgaactttctggtgaccctgcacggttgctggctggtggccatcctcacccgcaggcaccgccaggccattgcccgcctctggcccaactactgcctcttcctggcgctgttcctgctgtaccagtacctgctgtgcctggggatgcccccggccctgtgcattggtgaggggcacgtggcttgggtgggagtgggctttgtggctttgtggatgcccgtgggggtgtttcccgcctgccccagactcctgtccaccctcctagacttagccttggcctcctccagtccctcctctctgctccacatcctacccaggcgccatcacctgcatcctgtctctcgggggcggcctggccccctccaggctctgctgttctcttttccttttttgcccagtattctatcttgaaatatttcaaaccttcaggaaagttgtgagtcacacaaggagcactgcgtcctctccgggccccgtgcgggtcggccgtgatgcctcacacccaagtgctgcctcgagcatgcgtctcctgggtgagggtgtctgagcccacagccccacacccgtggtcccgtctcctggcagtgccatcgtcaaacgtgtcctctgcattcaaacagccccggcctcagcacccttctttgtggccatttggttttcagacgggatctggtctgatgtttgctctagtctcttgtcttgggtctaagccgcccccgcctctcctgtctcttgggaagttccggagggaggccggtagcgttgctgacgccgtgagactggatttgcgtggctgtcctggtgctgcaggtctgctcaaggcacacagcaccctgcggtctgagatggggagtcacatttgtgcacgtggccggctcaggggcgtcccacctgccccacagtggcacccagcctgttggcactggtgggctgtgtggggtcagccttgtggttttacgaaacagactttctctctgctgtctctgtgtgtctgtcagctgggattcccatcaaggacagctgccatttgttactggctgctttccaagaattctgtcatccgcagaccctgggcctcccctctgctgagtgggtcctggcccctccggccacacactgttacatcatctccccgtatttggctgggcatggtggttcacgcctgtgatgccagcactttgggaggctgaggcaggagcatcccttgaggccaggagtttgaaaccactctggtcaacatagcaagaaccctttttttttttttttttttttttgagatggagtcttgctctgtcacccaggctggagtacactggcacaatctcggctcactgcagcctccgcctcccgggttcaagcaattctcctgtctcagcctccctcgtggctaggattacaggcaagcaccaccaggcccggctaatttttgtatttttagtagagacggggtttcaccatcttggccaggctggtcttgaactcctgacctcaggtgatccacccgcctcccaaattacaggcctccctcctgggattacaggcgtgagctgccacgcccggccccgtcttgttttctgctcccaggcgctgctgcctcatcttctgctacccaggcccagccttgtgctcacagccattgctccagggagcccaatcgagttctaggagcgtgaggtttagagcccggggtctgggcgctgggtgtgcctgttgctacagggctgcctcagcctctgggccctccagctcttccttgttgaaacatctgctttcgagcatcaccgaggccagctccccgtctcctgtccacctcttccttgttgaaatacctgctatcaagcgtcacctaggccagctccccttcttctgcctccttccacgcggctgcgccatgcagtcgccatcctgtgagatcagcatgtcctgggttccccaacatcgagggtaactttgtttttgtatcgtgaggttccctctgtggcagatggggctgtgggttcagcatgtcctgggttccccagcatcgagggtcactttgtttttgggtcgcgaggttccctctgtggcagatggggctgtgggttcagcatgtcctgggttccccagcatcgagggtcactttgtttttgtgtcgcgaggttccctctgtggcagatggggctgtgggttcagcatgtcctgggttccccagcatcgagggtcactttgtttttgggtcgcgaggttccctctgtggcagatgggggctgtgggttcagaatgtcctgggttccccagcatcgagggtcactttgtttttgggtcacgaggttccctctgtggcagatggggctgtgggttcagcatgtcctgggttccccagcatcgagggtcactttgtttttgtgtcgcgaggttccctctgtggcagatggggctgtgagatcagcatgtcctgggttccccaacatcgagggtcactttgtttttgggtcgcgaggttccctctgtggcagatggggctgtgggttcagcatgtcctgggttccccagcatcgagggtcactttgtttttgggtcgcgaggttccctctgtggcagatggggctgtgggttcagcatgtcctgggttccccagcatcgagggtcactttgtttttgggtcgcgaggttccctctgtggcagatggggctgtgggttcagaatgtcctgggttccccagcatcgagggtcactttgtttttgggtcgcgaggttccctctgtggcagatggggctgtgggttcagcatgtcctgggttccccagcatcgagggtcactttgtttttgggtcacgaggttccctctgtggcagatggggctgtgggttcagcatgtcctgggttccccaacatcgagggtcactttgtttttgggtcgcgaggttccctctgtggcagatggggctgtgagttcagcatgtcctgggttccccagcatggagggtcactttgtttttgtgtcgcaaggttccctctgtggcagatggggctgtgagttcagcatgtcctgggttccccagcatggagggtcactttgtttttgtgtcgcgaggttccctctgtggcagatggggctgtgagttcagcatgtcctgggttccccagcatggagggtcactttgtttttgtgtcgcgaggttccctctgtggcagatggggctgtgagttcagaatgtcctgggttccccagcatcgagggtcactttgtttttgtgtcgcgaggttccctctgtggcagatggggctgtgagatcagcatgtcctgggttccccaacatcgagggtcactttgtttttgggtcgcgaggttccctctgtggcagatggggctgtgggttcagcatgtcctgggttccccagcatcgagggtcactttgcttttgggtcgcgaggttccctctgtggcagatggggctgtgagttcagcatgtcctgggttccccagcatggagggtcactttgtttttgtgtcgcgaggttccctctgtggcagatggggctgtgggtttcgcagatgcgtggagtcacatccatgccctcagtccttagggaccgaccctccctgcctcacacgcctcccaggaagtgtggccgggggccggcagtgccacggctccctccccagcaggccccggccgctcccatccccagcacgtggtcctatcagaacgccacgtcagcaggactcccagcaggtggcctttaggtctggcttctttcacttggcagagcacactgaggtctgtctaggctgtcgcatggatcccggtcccacgtgctgagcagcgcgttcccagctgtggttgctgcaggttgaacttttcctggctgcaggcgtccgtgcagcttctggccgttgttttcagagctgtcctatcacacgcactgtcctatcatggaatatgacgccgtgtgggccacaactcaggcccagcagcccccacccccgtgctctctggcctcctgctcagttcctttgcccccaggggcttggtgcagagttgaaggaatctgtgtgtgtgaacacacaggacactagagctgtcagttctcgagacaccaggtgtgcgcgaggtgattcccatggaccctgagggctggtgatagactcgggtcaacgggtggggaccgggtgtctcaggccccaggcaggcccggcccttcctgacatgacaccccttcccccagattatccctggcgctggagccgggccgtccccatgaactccgcactcatcaagtggctgtacctgcctgatttcttccgggcccccaactccaccaacctcatcagtgagtgccccccaccacccccgcctctgcagaggaccctcagagtacattcacgcccccaaatctgctcacaagtgtgcacacaggcgtgcacgggcggaggtgtggtcaggcacatggcggcctgcaggccctgacctcgcacgcacgcacgcagacctcagcctgtgtgcacggcagcccttgtgcagatgccctcacaccggggctcccccagggacacccggccactcacccaggcagacgtgtgtccgctcccagcggctgcacgccgacaggcctggggtgggaggtgggatttatgcgccgtgcccacctcgtgtgggtccccgtgtggcacagcggcggctcctgtgtcctgcaggcgactttctcctgctgctgtgcgcctcccagcagtggcaggtgttctcagctgagcgcacagaggagtggcagcgcatggctggcgtcaacaccgaccgcctggagccgctgcggggggagcccaaccccgtgcccaactttatccactgcaggtgggttccacgtcaccctccacggggaaccttctgggaggggtggccggggcgcccgccctgacgctccggcctggcaggtcctaccttgacatgctgaaggtggccgtcttccgatacctgttctggctggtgctggtggtggtgtttgtcacgggggccacccgcatcagcatcttcgggctgggctacctgctggcctgcttctacctgctgctcttcggcacggccctgctgcagagggacacacgggcccgcctcgtgctgtgggactgcctcattctgtacaacgtcaccgtcatcatctccaagaacatgctgtcggtgagcctccggcccccccgcacccaccgccctggggccccgctggccccgctgaccctgctctcccccagctcctggcctgcgtcttcgtggagcagatgcagaccggcttctgctgggtcatccagctcttcagccttgtatgcaccgtcaagggctactatgaccgtgagtggccaggacggtggcgggggagggcgtggggaagccccctgctcctgggccctgggcctgacccttgccggtgcctgccttgcagccaaggagatgatggacagagaccaggactgcctgctgcctgtggaggaggctggcatcatctgggacagcgtctgcttcttcttcctgctgctgcagcgccgcgtcttccttagccattactacctgcacgtcagggccgacctccaggccaccgccctgctagcctccaggcaagcttgggcccagacacagcccagagctcccgtcttggggctgggagggggcaatgggaggttcctcactgtctcaggccccggcccgtggagggcaggctctgccactctgtgacatgggcgtgtcatctagagggagaatgaaggccggcagatccccggcaccatcacactctgccccagtgctgggtctgtcagagaccacaggctgcagtgctgacggtggctggtgtctcacccccagccaactttcccactaagggctaagtttctccaccagcgggagggccactgtgtggtgtcacgactgccccagggaggggttctggcttggggccagctttgccttcttccctgcagctgtggtggggtgggtgccaccagacgcccctgcatctgtacggcagaagggcctgtcctcgccgcagacagcacggagggtgggggcagcagatgcctcccccgtgggtgcctcttgtccagcgtgggcagagaggagcaggctgagctgtcccgggctgagcggggagcggcggctgcccatgttgctggggtcgagtgcctggtgctcacaccccatccccgcctccctacaggggcttcgccctctacaacgctgccaacctcaagagcattgactttcaccgcaggatagaggagaagtccctggcccagctgaaaagacagtaggtgcctctggggcggggactccccggctcctccccccaatgctcagcataccccacctttccccaccacaggatggagcgtatccgtgccaagcaggagaagcacaggcagggccgggtggaccgcagtcgcccccaggacaccctgggccccaaggaccccggcctggagccaggtgagtgcagctggagtcgggcacccagggccccgtgtccagcatgtctgtgcctgctggcgtgtgctgcgtctgtgcccatgtgacgtcccacagggctcccagcccgcctgtcctgtccgcatgatcaccctctgtctggcaggccccatggccgccctgtgactgtccgtccacgcacatgggctctgagccccatggccccacacggcccccgtcactgtgggtgtccgtgtctgtctccacctatcctgtctccaagacgggagcactcacagccccgacccctcctggtggcttgactgctgcctcatgctcaccctgcccctccacagggcccgacagtccagggggctcctccccgccacggaggcagtggtggcggccctggctggaccacgccacaggtacccccaattaggccgcctgtggccaccctctcaggccctctgtgcccccatctgtcctctgcctggcctgctatcttcccctcccttcccccgactcccaggccctgagcgtcaggacgtgctcaggcctcctgggtcggggggtgcctcactggctgcagacccctgggctgactatgtcctctcctggctatgccccagcccttccaacagtgggagtctcggagcttgccccgatgacacatggtggtcgagcagcgatctcacctgggacccagcagcactgcgttattctgtttttgtttctttttgagatggagtctcgctctgtcactgcaggctggagtgcagtggcatgatctcagctcactgcaatctctacctcccgggttcaagtgatcctcctgcctcagcctcccaagtaactgagactacaagcatgtgccccactccaggccttttttttttttttttggagacggtgtcttgccctgtcgcccaggctacagtgcaatggcgtgattgcggctcactacaacccccacctcccaggttcaaaggattctcctgcctcagcctcccaagtagctgggattacaggtgcccgccaccacgcccagctaattttcgtatttttagtagagatggggtttcaccatattggccaggctggtctcgaacctctgacttcaggtgatccgcccgcctcagcctcccacagtgctgggattacaggcgtcagccaccgtgcccggcctgttctgtttttctaactctcacacagcctcctgggttttccccggtcctctgcagtcggcccactctgcaccccagcccgcgctggctctgctcctcagctgccctgcccacctctgtcttgtcccaccgcgctggcctgtgtcttgtgcctgcactgctcccggctactccgcatgggaagggtggctctcgggccttggcccatgcaggcggagggggtctggctgggagtctccctgcatggaaggctggctctcagtgctgcctgcccacagtcatccactccggggactacttcctgtttgagtccgacagtgaggaagaggaggaggctgttcctgaagacccgaggccgtcggcacagagtgccttccaggtgaggtgggagagccccgtcggccccactccaaccacagagcttgtggtcctggaccagggcagcatagagggtgtcagatgcccccagggcctgggagccgagctcctccacctccagttagcccaccccgccccatccaggcctcccaagtcccatgggaaaccaggctacagggacatgggtcatgtgtagcctgctgccccacggtcttggctctgaccacccaggttctggtggctgcccgtggcctgacctgtgagaccggcccaacacctttgtgctggccgcctggctgtcctgggtccatctttgggcccctggctcttggtgttagaccagcccacccaactcctgaatgggtgggagtcttcccccacagcccctcagggtccccatccgggaggggctcagggacacggaggtccctgggagacacagagcagggatctggatctggcgcccggcttgcccaaccccagcttcccgcctgggtctgatggctcgggaggccgggtcctaacccgggggctggccgacagctggcgtaccaggcatgggtgaccaacgcccaggcggtgctgaggcggcggcagcaggagcaggagcaggcaaggcaggaacaggcaggacagctacccacaggtgagctggggggcgtggggactctgaggggaagccgcgggactgccagtcactcaccagcatcctgtgcccaggaggtggtcccagccaggaggtggagccagcagagggccccgaggaggcagcggcaggtacgtgggcccggggctggggagtgggaggtctctcttggccccacaggctgcccctccagcgccccctcccgccctcccgcaggccggagccatgtggtgcagagggtgctgagcacggcgcagttcctgtggatgctggggcaggcgctagtggatgagctgacacgctggctgcaggagttcacccggcaccacggcaccatgagcgacgtgctgcgggcagagcgctacctcctcacacaggagctcctgcaggtgagcctgcccgtgcaccacgctcgtccctgctctgcctgactacgcccctgcctgcttaacagcctagtcccgcgcccactgcacgaaaccccgtgtggggacaagagctggacgcagccctgagccccctgctgtgccctgcagggcggcgaagtgcacaggggcgtgctggatcagctgtacacaagccaggccgaggccacgctgccaggccccaccgaggcccccaatgccccaagcaccgtgtccaggtaggtgcgggggtgacccgagccccagctgctgcccctggtgtgtgggcatcgcctagccatccccgaccctcgccattcccttgtaccccaaaggaccgtgggcactttccaccctgaccctccctgtagcctggggtcaggccatagagcaggattctctgtgactcggcttccctccccagtgggctgggcgcggaggagccactcagcagcatgacagacgacatgggcagccccctgagcaccggctaccacacgcgcagtggcagtgaggaggcagtcaccgaccccggggagcgtgaggctggtgcctctctgtaccagggactgatgcggacggccagcgagctgctcctggacaggtgggggcgggacgcgcacaacaccagcctcaccatggccctcggggagcagccgaacaggggcaggagactgactgtgaccggcaacagatcgggccgtcatgccttcgggcagtcccagactcccccaaacacgcgggtctccctgtaggcgcctgcgcatcccagagctggaggaggcagagctgtttgcggaggggcagggccgggcgctgcggctgctgcgggccgtgtaccagtgtgtggccgcccactcggagctgctctgctacttcatcatcatcctcaaccacatggtcacggcctccgccggctcgctggtgctgcccgtgctcgtcttcctgtgggccatgctgtcgatcccgaggcccagcaagcgcttctggatgacggccatcgtcttcaccgaggtgggccgaggccgcgggggagggggcgcccggcccaccgcgccgtgaccctccccgcgtgctgagccccctcccccacagatcgcggtggtcgtcaagtacctgttccagtttgggttcttcccctggaacagccacgtggtgctgcggcgctacgagaacaagccctacttcccgccccgcatcctgggcctggagaagactgacggctacatcaagtacgacctggtgcagctcatggcccttttcttccaccgctcccagctgctggtgagtgtgagccttggctggcaatgcggggctgggcaggccctctgggcacctgtgctctccaccagggaggcaaggccccctcaccacaccctcccgcccctcagtgctatggcctctgggaccatgaggaggactcaccatccaaggagcatgacaagagcggcgaggaggagcagggagccgaggaggggccaggggtgcctgcggccaccaccgaagaccacattcaggtggaagccagggtcggacccacggacgggaccccagaaccccaagtggagctcaggccccgtgatacgaggcgcatcagtctacgttttagaagaaggaagaaggagggcccagcacggaaaggagcggcagccatcggtataagcgccctgcctcacaacctcctgcctacccagttttctgagtggggctactgcagggagggtctttctcagatgagacggccaagcccagtgcgaggcccacctggatcccaggaaggtgccacttctgagccacagctcccggctctgcctacagagccgtccctgactgctgcccccggggatgctccccacgtgtagggtgactgttggcctgggctggcccctcacagttgccccagacagaggacacagccccagctgtctccttgccagtgacactgggagctttcctgtgctccgtctgcttgtctgtcaaacagggagaatgccagcctcttagggtggtcaggagccatgagccaggcccagtccccagggggcccaggcagaagtcagcttttccctacagaagctgaggacagggaggaagaagagggggaggaagagaaagaggcccccacggggagagagaagaggccaagccgctctggaggaagagtaagggcggccgggcggcggctgcagggcttctgcctgtccctgtgagtgatggcggccgggggcagctggggagtgggggtggggaggcgggtactgggcccaggctgagcgcccccttccgcagggcccagggcacatatcggccgctacggcgcttcttccacgacatcctgcacaccaagtaccgcgcagccaccgacgtctatgccctcatgttcctggctgatgttgtcgacttcatcatcatcatttttggcttctgggcctttggggtgagccaggcccgggacccaaacccagtgtacgcagagctcagcagccacccacatcccctgggcttggctccccctgacctgtgctctcctggccacagaagcactcggcggccacagacatcacgtcctccctatcagacgaccaggtacccgaggctttcctggtcatgctgctgatccagttcagtaccatggtggttgaccgcgccctctacctgcgcaagaccgtgctgggcaagctggccttccaggtggcgctggtgctggccatccacctatggatgttcttcatcctgcccgccgtcactgagaggtgggcccacgcgtgggggcgctcggtctccaggggcggggcagtgcaggctgggggccctgcggggctgtttctgatggggtccttgacctggccatcccgccccaggatgttcaaccagaatgtggtggcccagctctggtacttcgtgaagtgcatctacttcgccctgtccgcctaccagatccgctgcggctaccccacccgcatcctcggcaacttcctcaccaagaagtacaatcatctcaacctcttcctcttccaggggtgagtgcaggtccgccggggtgggggtcacggcccgggcatgagggagcccacctgacgggaaccctggctgtgggcaggttccggctggtgccgttcctggtggagctgcgggcagtgatggactgggtgtggacggacaccacgctgtccctgtccagctggatgtgtgtggaggacatctatgccaacatcttcatcatcaaatgcagccgagagacagagaaggtgcctgggcccagggcgggggccgggacaagggccagggatatgccctctccctaagacagaggcactgctgccacgagaacccgtggtgctggaggccctcccagggctcggagcccatggggacatgaggcgagcccacccactagctgatcacgaggccagtgatcttggcagctgcgagtgagtgctgggcgcagaagtgggcagcggagttggtcctgttccaggcaggctggcagcagagcagggcctggtgcagggaggaccggacagccactgtttgctgcattcttgtttaatggcctttctcagagagaattcgtgcgtcagaccactcccccacgtaaaaagtacaactcaggggtttctagtggattcacagttgagcatctgccttctcaccacttcaaaaagaaaccccgggccgggcacagcggctcacccctatcatcccagcactgtaagagacccaggagggaggtactgctcaaggccaggagttcaagatctgcacggccacaagcgagaccctgcctcagcaaatgtaaaaataaaacaattagctggcaatggcagctcatgcctatggtcccagcactttgggaggccaaaggaggaggatcagttgaggccaggagttttaagaccagcctgggcaacatagtgagtgagactctctctacaaaaaaataaacgttagctgggtgtgatggtgcacacttgtgctcccagctactctggaggctgaggtgggaggatggcttgaggccaggagtacagggctgcattaagcctgatcacaccactgcactccagcttgggcaacagagtgagaccctgtctctaaaaaagtaaaaagaagaaacccagtgcccattggctgtcactcggtttcccctcccctggcccctagtaaccccttgtctaggtcctggttctatgagatttgcctacagtggatatttcatgaaaacaggctcagacagcgtttgtccttttgtaatcagctttccttgctcagcatggtgtctctgaggtccacccacgtggcagtatggccttcctgtttatgggcaaatgatattctgtggcatggatggaccacaacatgctcatctgttgatgggctgttgcccgtgatgctgccaggcacgccggtgtacacgtctgtgtgcccgtgcccctggtcctgtggctgcacgccagggccggaagtgctggtggtggtttccatcatgaggagctgctggttttccatagcagctccaccatctaaggttcccaccaccaacacgaggttgcggtttctccacatcctcaacaacctgttactatgtcttttttgttctggccatgctgctgaacgggtggcttgctgtgggctcttctcagttccctgttgaccgacgctgagcgtcttttcatgtgcttggccatttgtgtatcttctctggggaaatgtctattcaaatcctttctccatagtttagttgggcttttgagataggatctcaggctgaagtgcagtggcatgaccttcactcactgtagcctctgcctcccaggttcaagcgattctcccacctcagcctcccgagtagctgggactacaggtgtgcaccaccaggcctggctagtcttttgtatttttggccaggttcgtctcaaactcctgacctcaaagtgctgggatgacaagtgtgagccgccacacccagcagttgggttgttttttattagagttttttctgtttttctgcagatacattggctagaaatgactgaattggaagaattctctgtattctctggttgctagaaccttatcaattaaaatttgcagaaaatttctccaattctatggactgtctcttaaattttcttggtgtctttggaagcacaaagtatttattttggtaatatctggtttactttgtttccttcgccaaatccaggttcatgaagatttgcccgttttcttctaaaagttctatagttttagctctgaagtttcgctctttgatccactttgaggtaaattttggcacatggtatgaggcaggagttgcgtttcattctcctgcctggggcggtgcctgcaccgtgttgaaaaaggttgtccgttcccactgaacggtcacagctcccttgtctaagatcaacgacccctgaacatgagggttccaactggactcttagttctactccactggcctgtgtctgcccaccattactaccgctgtgccatactgaggtcaggctggggcttttctgggctgctgggtgagctggagaatgcggttgtgtgacccgcaggaagggcagagctgagcgtatgacctgtgtcgttcccctccagaaatacccgcagcccaaagggcagaagaagaagaagatcgtcaagtacggcatgggtggcctcatcatcctcttcctcatcgccatcatctggttcccactgctcttcatgtcgctggtgcgctccgtggttggggttgtcaaccagcccatcgatgtcaccgtcaccctgaagctgggcggctatgaggtgagcatgtgtgggtccgcctgtccattcccatcccctgggggttctggccaaggtggtgcaccacccccagccgctcctccacgctcatcttcgtggccccgtgtccccgtgcctgccccagccgctgttcaccatgagcgcccagcagccgtccatcatccccttcacggcccaggcctatgaggagctgtcccggcagtttgacccccagccggtaagtggcctctgccctgtgaaagctggtgtggggaggcggctgcagtcactgagggtgtcacttgtacccagctggccatgcagttcatcagccagtacagccctgaggacatcgtcacggcgcagattgagggcagctccggggcgctgtggcgcatcagtccccccagccgtgcccagatgaagcgggagctctacaacggcacggccgacatcaccctgcgcttcacctggaacttccagaggttcgtcctggacttggggcagtgcctgggtgggtggacccactacagtgggtcacgctgtgttcccacccccagggacctggcgaagggaggcactgtggagtatgccaacgagaagcacatgctggccctggcccccaacagcactgcacggcggcagctggccagcctgctcgagggcacctcggaccagtctgtgtgagtgaagggcccgggtggtgggcaggagggctgtgccaggttggctgggccaggcctgacctgccagcacctccctgcagggtcatccctaatctcttccccaagtacatccgtgcccccaacgggcccgaagccaaccctgtgaagcagctgcagcccagtgagtatgggcgtgggggttgggggaggctagagaggggtgacctgcggcctcaacgatcttctccctccatcccagatgaggaggccgactacctcggcgtgcgtatccagctgcggagggagcagggtgcgggggccaccggcttcctcgaatggtgggtcatcgagctgcaggagtgccggaccgactgcaacctgctgcccatggtcattttcagtgacaaggtcagcccaccgagcctcggcttcctggctggctacgggtgagtgagtggctgggggggcaccccgcagctcggggggctccgggcggccccaggactcaccagcttcccccgcagcatcatggggctgtacgtgtccatcgtgctggtcatcggcaagttcgtgcgcggattcttcagcgagatctcgcactccattatgttcgaggagctgccgtgcgtggaccgcatcctcaagctctgccaggacatcttcctggtgcgggagactcgggagctggagctggaggaggagttgtacgccaagctcatcttcctctaccgctcactggagaccatgatcaagtggactcgtgagaaggagtaggagctgctgctggcgcccgagagggaaggagccggcctgctgggcagcgtggccacaaggggcggcactcctcaggccgggggagccactgccccgtccaaggccgccagctgtgatgcatcctcccggcctgcctgagccctgatgctgctgtcagagaaggacactgcgtccccacggcctgcgtggcgctgccgtcccccacgtgtactgtagagttttttttttaattaaaaaatgttttatttatacaaatggacaatcaga

SEQ ID NO: 3 - RNA, 8089 nt, Homo sapiens
  source 1..8089: mol_type = genomic RNA
gggagccgccgtccggcccagctcggccccagtgagccgagcgctgcgctccgccgaggggcagggcggtcgcctgagcgagcgcgggcccgggacgtcggcaccggcggggcggccgaaggagaaggaggaagaggagaaggcggcgcgcgggtccccgcgggtcagccatggcgcgccggccccggggcccccgcaccgccccatagcgccgcggcgtccgctcggtctgggccgggccctgggccctccagccatggagccgcacgtgctcggcgcggtcctgtactggctgctgctgccctgcgcgctgctggctgcctgcctgctccgcttcagcggactctcgctggtctacctgctcttcctgctgctgctgccctggttccccggccccacccgatgcggcctccaaggtcacacaggccgcctcctgcgggcattgctgggcctcagcctgctcttcctggtggcccatctcgccctccagatctgcctgcatattgtgccccgcctggaccagctcctgggacccagctgcagccgctgggagaccctctcgcgacacataggggtcacaaggctggacctgaaggacatccccaacgccatccggctggtggcccctgacctgggcatcttggtggtctcctctgtctgcctcggcatctgcgggcgccttgcaaggaacacccggcagagcccacatccacgggagctggatgatgatgagagggatgtggatgccagcccgacggcagggctgcaggaagcagcaacgctggcccctacacggaggtcacggctggccgctcgtttccgagtcacggcccactggctgctggtggcggctgggcgggtcctggccgtaacactgcttgcactggcaggcatcgcccacccctcggccctctccagtgtctacctgctgctcttcctggccctctgcacctggtgggcctgccactttcccatcagcactcggggcttcagcagactctgcgtcgcggtggggtgcttcggcgccggccatctcatctgcctctactgctaccagatgcccttggcacaggctctgctcccgcctgccggcatctgggctagggtgctgggtctcaaggacttcgtgggtcccaccaactgctccagcccccacgcgctggtcctcaacaccggcctggactggcctgtgtatgccagccccggcgtcctcctgctgctgtgctacgccacggcctctctgcgcaagctccgcgcgtaccgcccctccggccagaggaaggaggcggcaaaggggtatgaggctcgggagctggagctagcagagctggaccagtggccccaggaacgggagtctgaccagcacgtggtgcccacagcacccgacaccgaggctgataactgcatcgtgcacgagctgaccggccagagctccgtcctgcggcggcctgtgcggcccaagcgggctgagcccagggaggcgtctccgctccacagcctgggccacctcatcatggaccagagctatgtgtgcgcgctcattgccatgatggtatggagcatcacctaccacagctggctgaccttcgtactgctgctctgggcctgcctcatctggacggtgcgcagccgccaccaactggccatgctgtgctcgccctgcatcctgctgtatgggatgacgctgtgctgcctacgctacgtgtgggccatggacctgcgccctgagctgcccaccaccctgggccccgtcagcctgcgccagctggggctggagcacacccgctacccctgtctggaccttggtgccatgttgctctacaccctgaccttctggctcctgctgcgccagtttgtgaaagagaagctgctgaagtgggcagagtctccagctgcgctgacggaggtcaccgtggcagacacagagcccacgcggacgcagacgctgttgcagagcctgggggagctggtgaagggcgtgtacgccaagtactggatctatgtgtgtgctggcatgttcatcgtggtcagcttcgccggccgcctcgtggtctacaagattgtctacatgttcctcttcctgctctgcctcaccctcttccaggtctactacagcctgtggcggaagctgctcaaggccttctggtggctcgtggtggcctacaccatgctggtcctcatcgccgtctacaccttccagttccaggacttccctgcctactggcgcaacctcactggcttcaccgacgagcagctgggggacctgggcctggagcagttcagcgtgtccgagctcttctccagcatcctggtgcccggcttcttcctcctggcctgcatcctgcagctgcactacttccacaggcccttcatgcagctcaccgacatggagcacgtgtccctgcctggcacgcgcctcccgcgctgggctcacaggcaggatgcagtgagtgggaccccactgctgcgggaggagcagcaggagcatcagcagcagcagcaggaggaggaggaggaggaggaggactccagggacgaggggctgggcgtggccactccccaccaggccacgcaggtgcctgaaggggcagccaagtggggcctggtggctgagcggctgctggagctggcagccggcttctcggacgtcctctcacgcgtgcaggtgttcctgcggcggctgctggagcttcacgttttcaagctggtggccctgtacaccgtctgggtggccctgaaggaggtgtcggtgatgaacctgctgctggtggtgctgtgggccttcgccctgccctacccacgcttccggcccatggcctcctgcctgtccaccgtgtggacctgcgtcatcatcgtgtgtaagatgctgtaccagctcaaggttgtcaacccccaggagtattccagcaactgcaccgagcccttccccaacagcaccaacttgctgcccacggagatcagccagtccctgctgtaccgggggcccgtggaccctgccaactggtttggggtgcggaaagggttccccaacctgggctacatccagaaccacctgcaagtgctgctgctgctggtattcgaggccatcgtgtaccggcgccaggagcactaccgccggcagcaccagctggccccgctgcctgcccaggccgtgtttgccagcggcacccgccagcagctggaccaggatctgctcggctgcctcaagtacttcatcaacttcttcttctacaaattcgggctggagatctgcttcctgatggccgtgaacgtgatcgggcagcgcatgaactttctggtgaccctgcacggttgctggctggtggccatcctcacccgcaggcaccgccaggccattgcccgcctctggcccaactactgcctcttcctggcgctgttcctgctgtaccagtacctgctgtgcctggggatgcccccggccctgtgcattgattatccctggcgctggagccgggccgtccccatgaactccgcactcatcaagtggctgtacctgcctgatttcttccgggcccccaactccaccaacctcatcagcgactttctcctgctgctgtgcgcctcccagcagtggcaggtgttctcagctgagcgcacagaggagtggcagcgcatggctggcgtcaacaccgaccgcctggagccgctgcggggggagcccaaccccgtgcccaactttatccactgcaggtcctaccttgacatgctgaaggtggccgtcttccgatacctgttctggctggtgctggtggtggtgtttgtcacgggggccacccgcatcagcatcttcgggctgggctacctgctggcctgcttctacctgctgctcttcggcacggccctgctgcagagggacacacgggcccgcctcgtgctgtgggactgcctcattctgtacaacgtcaccgtcatcatctccaagaacatgctgtcgctcctggcctgcgtcttcgtggagcagatgcagaccggcttctgctgggtcatccagctcttcagccttgtatgcaccgtcaagggctactatgaccccaaggagatgatggacagagaccaggactgcctgctgcctgtggaggaggctggcatcatctgggacagcgtctgcttcttcttcctgctgctgcagcgccgcgtcttccttagccattactacctgcacgtcagggccgacctccaggccaccgccctgctagcctccaggggcttcgccctctacaacgctgccaacctcaagagcattgactttcaccgcaggatagaggagaagtccctggcccagctgaaaagacagatggagcgtatccgtgccaagcaggagaagcacaggcagggccgggtggaccgcagtcgcccccaggacaccctgggccccaaggaccccggcctggagccagggcccgacagtccagggggctcctccccgccacggaggcagtggtggcggccctggctggaccacgccacagtcatccactccggggactacttcctgtttgagtccgacagtgaggaagaggaggaggctgttcctgaagacccgaggccgtcggcacagagtgccttccagctggcgtaccaggcatgggtgaccaacgcccaggcggtgctgaggcggcggcagcaggagcaggagcaggcaaggcaggaacaggcaggacagctacccacaggaggtggtcccagccaggaggtggagccagcagagggccccgaggaggcagcggcaggccggagccatgtggtgcagagggtgctgagcacggcgcagttcctgtggatgctggggcaggcgctagtggatgagctgacacgctggctgcaggagttcacccggcaccacggcaccatgagcgacgtgctgcgggcagagcgctacctcctcacacaggagctcctgcagggcggcgaagtgcacaggggcgtgctggatcagctgtacacaagccaggccgaggccacgctgccaggccccaccgaggcccccaatgccccaagcaccgtgtccagtgggctgggcgcggaggagccactcagcagcatgacagacgacatgggcagccccctgagcaccggctaccacacgcgcagtggcagtgaggaggcagtcaccgaccccggggagcgtgaggctggtgcctctctgtaccagggactgatgcggacggccagcgagctgctcctggacaggcgcctgcgcatcccagagctggaggaggcagagctgtttgcggaggggcagggccgggcgctgcggctgctgcgggccgtgtaccagtgtgtggccgcccactcggagctgctctgctacttcatcatcatcctcaaccacatggtcacggcctccgccggctcgctggtgctgcccgtgctcgtcttcctgtgggccatgctgtcgatcccgaggcccagcaagcgcttctggatgacggccatcgtcttcaccgagatcgcggtggtcgtcaagtacctgttccagtttgggttcttcccctggaacagccacgtggtgctgcggcgctacgagaacaagccctacttcccgccccgcatcctgggcctggagaagactgacggctacatcaagtacgacctggtgcagctcatggcccttttcttccaccgctcccagctgctgtgctatggcctctgggaccatgaggaggactcaccatccaaggagcatgacaagagcggcgaggaggagcagggagccgaggaggggccaggggtgcctgcggccaccaccgaagaccacattcaggtggaagccagggtcggacccacggacgggaccccagaaccccaagtggagctcaggccccgtgatacgaggcgcatcagtctacgttttagaagaaggaagaaggagggcccagcacggaaaggagcggcagccatcgaagctgaggacagggaggaagaagagggggaggaagagaaagaggcccccacggggagagagaagaggccaagccgctctggaggaagagtaagggcggccgggcggcggctgcagggcttctgcctgtccctggcccagggcacatatcggccgctacggcgcttcttccacgacatcctgcacaccaagtaccgcgcagccaccgacgtctatgccctcatgttcctggctgatgttgtcgacttcatcatcatcatttttggcttctgggcctttgggaagcactcggcggccacagacatcacgtcctccctatcagacgaccaggtacccgaggctttcctggtcatgctgctgatccagttcagtaccatggtggttgaccgcgccctctacctgcgcaagaccgtgctgggcaagctggccttccaggtggcgctggtgctggccatccacctatggatgttcttcatcctgcccgccgtcactgagaggatgttcaaccagaatgtggtggcccagctctggtacttcgtgaagtgcatctacttcgccctgtccgcctaccagatccgctgcggctaccccacccgcatcctcggcaacttcctcaccaagaagtacaatcatctcaacctcttcctcttccaggggttccggctggtgccgttcctggtggagctgcgggcagtgatggactgggtgtggacggacaccacgctgtccctgtccagctggatgtgtgtggaggacatctatgccaacatcttcatcatcaaatgcagccgagagacagagaagaaatacccgcagcccaaagggcagaagaagaagaagatcgtcaagtacggcatgggtggcctcatcatcctcttcctcatcgccatcatctggttcccactgctcttcatgtcgctggtgcgctccgtggttggggttgtcaaccagcccatcgatgtcaccgtcaccctgaagctgggcggctatgagccgctgttcaccatgagcgcccagcagccgtccatcatccccttcacggcccaggcctatgaggagctgtcccggcagtttgacccccagccgctggccatgcagttcatcagccagtacagccctgaggacatcgtcacggcgcagattgagggcagctccggggcgctgtggcgcatcagtccccccagccgtgcccagatgaagcgggagctctacaacggcacggccgacatcaccctgcgcttcacctggaacttccagagggacctggcgaagggaggcactgtggagtatgccaacgagaagcacatgctggccctggcccccaacagcactgcacggcggcagctggccagcctgctcgagggcacctcggaccagtctgtggtcatccctaatctcttccccaagtacatccgtgcccccaacgggcccgaagccaaccctgtgaagcagctgcagcccaatgaggaggccgactacctcggcgtgcgtatccagctgcggagggagcagggtgcgggggccaccggcttcctcgaatggtgggtcatcgagctgcaggagtgccggaccgactgcaacctgctgcccatggtcattttcagtgacaaggtcagcccaccgagcctcggcttcctggctggctacggcatcatggggctgtacgtgtccatcgtgctggtcatcggcaagttcgtgcgcggattcttcagcgagatctcgcactccattatgttcgaggagctgccgtgcgtggaccgcatcctcaagctctgccaggacatcttcctggtgcgggagactcgggagctggagctggaggaggagttgtacgccaagctcatcttcctctaccgctcaccggagaccatgatcaagtggactcgtgagaaggagtaggagctgctgctggcgcccgagagggaaggagccggcctgctgggcagcgtggccacaaggggcggcactcctcaggccgggggagccactgccccgtccaaggccgccagctgtgatgcatcctcccggcctgcctgagccctgatgctgctgtcagagaaggacactgcgtccccacggcctgcgtggcgctgccgtcccccacgtgtactgtagagttttttttttaattaaaaaatgttttatttatacaaatggacaatcaga

SEQ ID NO: 4 - RNA, 1246 nt, Homo sapiens
  source 1..1246: mol_type = genomic RNA
atgaagatttgcccgttttcttctaaaagttctatagttttagctctgaagtttcgctctttgatccactttgagaaatacccgcagcccaaagggcagaagaagaagaagatcgtcaagtacggcatgggtggcctcatcatcctcttcctcatcgccatcatctggttcccactgctcttcatgtcgctggtgcgctccgtggttggggttgtcaaccagcccatcgatgtcaccgtcaccctgaagctgggcggctatgagccgctgttcaccatgagcgcccagcagccgtccatcatccccttcacggcccaggcctatgaggagctgtcccggcagtttgacccccagccgctggccatgcagttcatcagccagtacagccctgaggacatcgtcacggcgcagattgagggcagctccggggcgctgtggcgcatcagtccccccagccgtgcccagatgaagcgggagctctacaacggcacggccgacatcaccctgcgcttcacctggaacttccagagggacctggcgaagggaggcactgtggagtatgccaacgagaagcacatgctggccctggcccccaacagcactgcacggcggcagctggccagcctgctcgagggcacctcggaccagtctgtggtcatccctaatctcttccccaagtacatccgtgcccccaacgggcccgaagccaaccctgtgaagcagctgcagcccaatgaggaggccgactacctcggcgtgcgtatccagctgcggagggagcagggtgcgggggccaccggcttcctcgaatggtgggtcatcgagctgcaggagtgccggaccgactgcaacctgctgcccatggtcattttcagtgacaagcatcatggggctgtacgtgtccatcgtgctggtcatcggcaagttcgtgcgcggattcttcagcgagatctcgcactccattatgttcgaggagctgccgtgcgtggaccgcatcctcaagctctgccaggacatcttcctggtgcgggagactcgggagctggagctggaggaggagttgtacgccaagctcatcttcctctaccgctcaccggagaccatgatcaagtggactcgtgagaaggagtaggagctgctgctggcgcccgagagggaaggagccggcctgctgggcagcgtggccacaaggggcggcactcctcaggccgggggagccactgccccgtccaaggccgccagctgt

SEQ ID NO: 5 - RNA, 6401 nt, Homo sapiens
  source 1..6401: mol_type = genomic RNA
ccatggacctgcgccctgagctgcccaccaccctgggccccgtcagcctgcgccagctggggctggagcacacccgctacccctgtctggaccttggtgccatgttgctctacaccctgaccttctggctcctgctgcgccagtttgtgaaagagaagctgctgaagtgggcagagtctccagctgcgctgacggaggtcaccgtggcagacacagagcctacgcggacgcagacgctgttgcagagcctgggggagctggtgaagggcgtgtacgccaagtactggatctatgtgtgtgctggcatgttcatcgtggtcagcttcgccggccgcctcgtggtctacaagattgtctacatgttcctcttcctgctctgcctcaccctcttccaggtctactacagcctgtggcggaagctgctcaaggccttctggtggctcgtggtggcctacaccatgctggtcctcatcgccgtctacaccttccagttccaggacttccctgcctactggcgcaacctcactggcttcaccgacgagcagctgggggacctgggcctggagcagttcagcgtatccgagctcttctccagcatcctggtgcccggcttcttcctcctggcctgcatcctgcagctgcactacttccacaggcccttcatgcagctcaccgacatggagcacgtgtccctgcctggcacgcgcctcccgcgctgggctcacaggcaggatgcagtgagtgggaccccactgctgcgggaggagcagcaggagcatcagcagcagcagcaggaggaggaggaggaggaggactccagggacgaggggctgggcgtggccactccccaccaggccacgcaggtgcctgaaggggcagccaagtggggcctggtggctgagcgcctgctggagctggcagccggcttctcggacgtcctctcacgcgtgcaggtgttcctgcggcggctgctggagcttcacgttttcaagctggtggccctgtacaccgtctgggtggccctgaaggaggtgtcggtgatgaacctgctgctggtggtgctgtgggccttcgccctgccctacccacgcttccggcccatggcctcctgcctgtccaccgtgtggacctgcgtcatcatcgtgtgtaagatgctgtaccagctcaaggttgtcaacccccaggagtattccagcaactgcaccgagcccttccccaacagcaccaacttgctgcccacggagatcagccagtccctgctgtaccgggggcccgtggaccctgccaactggtttggggtgcggaaagggttccccaacctgggctacatccagaaccacctgcaagtgctgctgctgctggtattcgaggccatcgtgtaccggcgccaggagcactaccgccggcagcaccagctggccccgctgcctgcccaggccgtgtttgccagcggcacccgccagcagctggaccaggatctgctcggctgcctcaagtacttcatcaacttcttcttctacaaattcgggctggagatctgcttcctgatggccgtgaacgtgatcgggcagcgcatgaactttctggtgaccctgcacggttgctggctggtggccatcctcacccgcaggcaccgccaggccattgcccgcctctggcccaactactgcctcttcctggcgctgttcctgctgtaccagtacctgctgtgcctggggatgcccccggccctgtgcattgattatccctggcgctggagccgggccgtccccatgaactccgcactcatcaagtggctgtacctgcctgatttcttccgggcccccaactccaccaacctcatcagcgactttctcctgctgctgtgcgcctcccagcagtggcaggtgttctcagctgagcgcacagaggagtggcagcgcatggctggcgtcaacaccgaccgcctggagccgctgcggggggagcccaaccccgtgcccaactttatccactgcaggtcctaccttgacatgctgaaggtggccgtcttccgatacctgttctggctggtgctggtggtggtgtttgtcacgggggccacccgcatcagcatcttcgggctgggctacctgctggcctgcttctacctgctgctcttcggcacggccctgctgcagagggacacacgggcccgcctcgtgctgtgggactgcctcattctgtacaacgtcaccgtcatcatctccaagaacatgctgtcgctcctggcctgcgtcttcgtggagcagatgcagaccggcttctgctgggtcatccagctcttcagccttgtatgcaccgtcaagggctactatgaccccaaggagatgatggacagagaccaggactgcctgctgcctgtggaggaggctggcatcatctgggacagcgtctgcttcttcttcctgctgctgcagcgccgcgtcttccttagccattactacctgcacgtcagggccgacctccaggccaccgccctgctagcctccaggggcttcgccctctacaacgctgccaacctcaagagcattgacttccaccgcaggatagaggagaagtccctggcccagctgaaaagacagatggagcgtatccgtgccaagcaggagaagcacaggcagggccgggtggaccgcagtcgcccccaggacaccctgggccccaaggaccccggcctggagccagggcccgacagtccagggggctcctccccgccacggaggcagtggtggcggccctggctggaccacgccacagtcatccactccggggactacttcctgtttgagtccgacagtgaggaagaggaggaggctgttcctgaagacccgaggccgtcggcacagagtgccttccagctggcgtaccaggcatgggtgaccaacgcccaggcggtgctgaggcggcggcagcaggagcaggagcaggcaaggcaggaacaggcaggacagctacccacaggaggtggtcccagccaggaggtggagccagcagagggccccgaggaggcagcggcaggccggagccatgtggtgcagagggtgctgagcacggcgcagttcctgtggatgctggggcaggcgctagtggatgagctgacacgctggctgcaggagttcacccggcaccacggcaccatgagcgacgtgctgcgggcagagcgctacctcctcacacaggagctcctgcagggcggcgaagtgcacaggggcgtgctggatcagctgtacacaagccaggccgaggccacgctgccaggccccaccgaggcccccaatgccccaagcaccgtgtccagtgggctgggcgcggaggagccactcagcagcatgacagacgacatgggcagccccctgagcaccggctaccacacgcgcagtggcagtgaggaggcagtcaccgaccccggggagcgtgaggctggtgcctctctgtaccagggactgatgcggacggccagcgagctgctcctggacaggcgcctgcgcatcccagagctggaggaggcagagctgtttgcggaggggcagggccgggcgctgcggctgctgcgggccgtgtaccagtgtgtggccgcccactcggagctgctctgctacttcatcatcatcctcaaccacatggtcacggcctccgccggctccctggtgctgcccgtgctcgtcttcctgtgggccatgctgtcgatcccgaggcccagcaagcgcttctggatgacggccatcgtcttcaccgagatcgcggtggtcgtcaagtacctgttccagtttgggttcttcccctggaacagccacgtggtgctgcggcgctacgagaacaagccctacttcccgccccgcatcctgggcctggagaagactgacggctacatcaagtacgacctggtgcagctcatggcccttttcttccaccgctcccagctgctgtgctatggcctctgggaccatgaggaggactcaccatccaaggagcatgacaagagcggcgaggaggagcagggagccgaggaggggccaggggtgcctgcggccaccaccgaagaccacattcaggtggaagcgagggtcggacccacggacgggaccccagaaccccaagtggagctcaggccccgtgatacgaggcgcatcagtctacgttttagaagaaggaagaaggagggcccagcacggaaaggagcggcagccatcgaagctgaggacagggaggaagaagagggggaggaagagaaagaggcccccacggggagagagaagaggccaagccgctctggaggaagagtaagggcggccgggcggcggctgcagggcttctgcctgtccctggcccagggcacatatcggccgctacggcgcttcttccacgacatcctgcacaccaagtaccgcgcagccaccgacgtctatgccctcatgttcctggctgatgttgtcgacttcatcatcatcatttttggcttctgggcctttgggaagcactcggcggccacagacatcacgtcctccctatcagacgaccaggtacccgaggctttcctggtcatgctgctgatccagttcagtaccatggtggttgaccgcgccctctacctgcgcaagaccgtgctgggcaagctggccttccaggtggcgctggtgctggccatccacctatggatgttcttcatcctgcccgccgtcactgagaggatgttcaaccagaatgtggtggcccagctctggtacttcgtgaagtgcatctacttcgccctgtccgcctaccagatccgctgcggctaccccacccgcatcctcggcaacttcctcaccaagaagtacaatcatctcaacctcttcctcttccaggggttccggctggtgccgttcctggtggagctgcgggcagtgatggactgggtgtggacggacaccacgctgtccctgtccagctggatgtgtgtggaggacatctatgccaacatcttcatcatcaaatgcagccgagagacagagaagaaatacccgcagcccaaagggcagaagaagaagaagatcgtcaagtacggcatgggtggcctcatcatcctcttcctcatcgccatcatctggttcccgctgctcttcatgtcgctggtgcgctccgtggttggggttgtcaaccagcccatcgatgtcaccgtcaccctcaagctgggcggctatgagccgctgttcaccatgagcgcccagcagccgtccatcatccccttcacggcccaggcctatgaggagctgtcccggcagtttgacccccagccgctggccatgcagttcatcagccagtacagccctgaggacatcgtcacggcgcagattgagggcagctccggggcgctgtggcgcatcagtccccccagccgtgcccagatgaagcgggagctctacaacggcacggccgacatcaccctgcgcttcacctggaacttccagagggacctggcgaagggaggcactgtggagtatgccaacgagaagcacatgctggccctggcccccaacagcactgcacggcggcagctggccagcctgctcgagggcacctcggaccagtctgtggtcatccccaatctcttccccaagtacatccgtgcccccaacgggcccgaagccaaccctgtgaagcagctgcagcccaatgaggaggccgactacctcggcgtgcgtatccagctgcggagggagcagggtgcgggggccaccggcttcctcgaatggtgggtcatcgagctgcaggagtgccggaccgactgcaacctgctgcccatggtcattttcagtgacaaggtcagcccaccgagcctcggcttcctggctggctacggcatcatggggctgtacgtgtccatcgtgctggtcatcggcaagttcgtgcgcggattcttcagcgagatctcgcactccattatgttcgaggagctgccgtgcgtggaccgcatcctcaagctctgccaggacatcttcctggtgcgggagactcgggagctggagctggaggaggagttgtacgccaagctcatcttcctctaccgctcaccggagaccatgatcaagtggactcgtgagaaggagtaggagctgctgctggcgcccgagagggaaggagccggcctgctgggcagcgtggccacaaggggcggcactcctcaggccgggggagccactgccccgtccaaggccgccagctgtgatgcatcctcccggcctgcctgagccctgatgctgctgtcagagaaggacactgcgtccccacggcctgcgtggcgctgccgtcccccacgtgtactgtagagttttttttttaattaaaaaatgttttatttatacaaatggaaaaaaaaaaaaaaaaaaaaaaaaaaaaaaaaa

SEQ ID NO: 6 - RNA, 1655 nt, Homo sapiens
  source 1..1655: mol_type = genomic RNA
taccagatccgctgcggctaccccacccgcatcctcggcaacttcctcaccaagaagtacaatcatctcaacctcttcctcttccaggggttccggctggtgccgttcctggtggagctgcgggcagtgatggactgggtgtggacggacaccacgctgtccctgtccagctggatgtgtgtggaggacatctatgccaacatcttcatcatcaaatgcagccgagagacagagaaggttcatgaagatttgcccgttttcttctaaaagttctatagttttagctctgaagtttcgctctttgatccactttgagaaatacccgcagcccaaagggcagaagaagaagaagatcgtcaagtacggcatgggtggcctcatcatcctcttcctcatcgccatcatctggttcccgctgctcttcatgtcgctggtgcgctccgtggttggggttgtcaaccagcccatcgatgtcaccgtcaccctcaagctgggcggctatgagccgctgttcaccatgagcgcccagcagccgtccatcatccccttcacggcccaggcctatgaggagctgtcccggcagtttgacccccagccgctggccatgcagttcatcagccagtacagccctgaggacatcgtcacggcgcagattgagggcagctccggggcgctgtggcgcatcagtccccccagccgtgcccagatgaagcgggagctctacaacggcacggccgacatcaccctgcgcttcacctggaacttccagagggacctggcgaagggaggcactgtggagtatgccaacgagaagcacatgctggccctggcccccaacagcactgcacggcggcagctggccagcctgctcgagggcacctcggatcagtctgtggtcatccccaatctcttccccaagtacatccgtgcccccaacgggcccgaagccaaccctgtgaagcagctgcagcccaatgaggaggccgactacctcggcgtgcgtatccagctgcggagggagcagggtgcgggggccaccggcttcctcgaatggtgggtcatcgagctgcaggagtgccggaccgactgcaacctgctgcccatggtcattttcagtgacaagcatcatggggctgtacgtgtccatcgtgctggtcatcggcaagttcgtgcgcggattcttcagcgagatctcgcactccattatgttcgaggagctgccgtgcgtggaccgcatcctcaagctctgccaggacatcttcctggtgcgggagactcgggagctggagctggaggaggagttgtatgccaagctcatcttcctctaccgctcaccggagaccatgatcaagtggactcgtgagaaggagtaggagctgctgctggcgcccgagagggaaggagccggcctgctgggcagcgtggccacaaggggcggcactcctcaggccgggggagccactgccccgtccaaggccgccagctgtgatgcatcctcccggcctgcctgagccctgatgctgctgtcagagaaggacactgcgtccccacggcctgcgtggcgctgccgtcccccacgtgtactgtagagtttttttttttaattaaaaaatgttttatttatacaaatggaaaaaaaaaaaaaaaaaaaaaaa

SEQ ID NO: 7 - RNA, 6368 nt, Homo sapiens
  source 1..6368: mol_type = genomic RNA
ccatggacctgcgccctgagctgcccaccaccctgggccccgtcagcctgcgccagctggggctggagcacacccgctacccctgtctggaccttggtgccatgttgctctacaccctgaccttctggctcctgctgcgccagtttgtgaaagagaagctgctgaagtgggcagagtctccagctgcgctgacggaggtcaccgtggcagacacagagcctacgcggacgcagacgctgttgcagagcctgggggagctggtgaagggcgtgtacgccaagtactggatctatgtgtgtgctggcatgttcatcgtggtcagcttcgccggccgcctcgtggtctacaagattgtctacatgttcctcttcctgctctgcctcaccctcttccaggtctactacagcctgtggcggaagctgctcaaggccttctggtggctcgtggtggcctacaccatgctggtcctcatcgccgtctacaccttccagttccaggacttccctgcctactggcgcaacctcactggcttcaccgacgagcagctgggggacctgggcctggagcagttcagcgtatccgagctcttctccagcatcctggtgcccggcttcttcctcctggcctgcatcctgcagctgcactacttccacaggcccttcatgcagctcaccgacatggagcacgtgtccctgcctggcacgcgcctcccgcgctgggctcacaggcaggatgcagtgagtgggaccccactgctgcgggaggagcagcaggagcatcagcagcagcagcaggaggaggaggaggaggaggactccagggacgaggggctgggcgtggccactccccaccaggccacgcaggtgcctgaaggggcagccaagtggggcctggtggctgagcgcctgctggagctggcagccggcttctcggacgtcctctcacgcgtgcaggtgttcctgcggcggctgctggagcttcacgttttcaagctggtggccctgtacaccgtctgggtggccctgaaggaggtgtcggtgatgaacctgctgctggtggtgctgtgggccttcgccctgccctacccacgcttccggcccatggcctcctgcctgtccaccgtgtggacctgcgtcatcatcgtgtgtaagatgctgtaccagctcaaggttgtcaacccccaggagtattccagcaactgcaccgagcccttccccaacagcaccaacttgctgcccacggagatcagccagtccctgctgtaccgggggcccgtggaccctgccaactggtttggggtgcggaaagggttccccaacctgggctacatccagaaccacctgcaagtgctgctgctgctggtattcgaggccatcgtgtaccggcgccaggagcactaccgccggcagcaccagctggccccgctgcctgcccaggccgtgtttgccagcggcacccgccagcagctggaccaggatctgctcggctgcctcaagtacttcatcaacttcttcttctacaaattcgggctggagatctgcttcctgatggccgtgaacgtgatcgggcagcgcatgaactttctggtgaccctgcacggttgctggctggtggccatcctcacccgcaggcaccgccaggccattgcccgcctctggcccaactactgcctcttcctggcgctgttcctgctgtaccagtacctgctgtgcctggggatgcccccggccctgtgcattgattatccctggcgctggagccgggccgtccccatgaactccgcactcatcaagtggctgtacctgcctgatttcttccgggcccccaactccaccaacctcatcagcgactttctcctgctgctgtgcgcctcccagcagtggcaggtgttctcagctgagcgcacagaggagtggcagcgcatggctggcgtcaacaccgaccgcctggagccgctgcggggggagcccaaccccgtgcccaactttatccactgcaggtcctaccttgacatgctgaaggtggccgtcttccgatacctgttctggctggtgctggtggtggtgtttgtcacgggggccacccgcatcagcatcttcgggctgggctacctgctggcctgcttctacctgctgctcttcggcacggccctgctgcagagggacacacgggcccgcctcgtgctgtgggactgcctcattctgtacaacgtcaccgtcatcatctccaagaacatgctgtcgctcctggcctgcgtcttcgtggagcagatgcagaccggcttctgctgggtcatccagctcttcagccttgtatgcaccgtcaagggctactatgaccccaaggagatgatggacagagaccaggactgcctgctgcctgtggaggaggctggcatcatctgggacagcgtctgcttcttcttcctgctgctgcagcgccgcgtcttccttagccattactacctgcacgtcagggccgacctccaggccaccgccctgctagcctccaggggcttcgccctctacaacgctgccaacctcaagagcattgacttccaccgcaggatagaggagaagtccctggcccagctgaaaagacagatggagcgtatccgtgccaagcaggagaagcacaggcagggccgggtggaccgcagtcgcccccaggacaccctgggccccaaggaccccggcctggagccagggcccgacagtccagggggctcctccccgccacggaggcagtggtggcggccctggctggaccacgccacagtcatccactccggggactacttcctgtttgagtccgacagtgaggaagaggaggaggctgttcctgaagacccgaggccgtcggcacagagtgccttccagctggcgtaccaggcatgggtgaccaacgcccaggcggtgctgaggcggcggcagcaggagcaggagcaggcaaggcaggaacaggcaggacagctacccacaggaggtggtcccagccaggaggtggagccagcagagggccccgaggaggcagcggcaggccggagccatgtggtgcagagggtgctgagcacggcgcagttcctgtggatgctggggcaggcgctagtggatgagctgacacgctggctgcaggagttcacccggcaccacggcaccatgagcgacgtgctgcgggcagagcgctacctcctcacacaggagctcctgcagggcggcgaagtgcacaggggcgtgctggatcagctgtacacaagccaggccgaggccacgctgccaggccccaccgaggcccccaatgccccaagcaccgtgtccagtgggctgggcgcggaggagccactcagcagcatgacagacgacatgggcagccccctgagcaccggctaccacacgcgcagtggcagtgaggaggcagtcaccgaccccggggagcgtgaggctggtgcctctctgtaccagggactgatgcggacggccagcgagctgctcctggacaggcgcctgcgcatcccagagctggaggaggcagagctgtttgcggaggggcagggccgggcgctgcggctgctgcgggccgtgtaccagtgtgtggccgcccactcggagctgctctgctacttcatcatcatcctcaaccacatggtcacggcctccgccggctccctggtgctgcccgtgctcgtcttcctgtgggccatgctgtcgatcccgaggcccagcaagcgcttctggatgacggccatcgtcttcaccgagatcgcggtggtcgtcaagtacctgttccagtttgggttcttcccctggaacagccacgtggtgctgcggcgctacgagaacaagccctacttcccgccccgcatcctgggcctggagaagactgacggctacatcaagtacgacctggtgcagctcatggcccttttcttccaccgctcccagctgctgtgctatggcctctgggaccatgaggaggactcaccatccaaggagcatgacaagagcggcgaggaggagcagggagccgaggaggggccaggggtgcctgcggccaccaccgaagaccacattcaggtggaagcgagggtcggacccacggacgggaccccagaaccccaagtggagctcaggccccgtgatacgaggcgcatcagtctacgttttagaagaaggaagaaggagggcccagcacggaaaggagcggcagccatcgaagctgaggacagggaggaagaagagggggaggaagagaaagaggcccccacggggagagagaagaggccaagccgctctggaggaagagtaagggcggccgggcggcggctgcagggcttctgcctgtccctggcccagggcacatatcggccgctacggcgcttcttccacgacatcctgcacaccaagtaccgcgcagccaccgacgtctatgccctcatgttcctggctgatgttgtcgacttcatcatcatcatttttggcttctgggcctttgggaagcactcggcggccacagacatcacgtcctccctatcagacgaccaggtacccgaggctttcctggtcatgctgctgatccagttcagtaccatggtggttgaccgcgccctctacctgcgcaagaccgtgctgggcaagctggccttccaggtggcgctggtgctggccatccacctatggatgttcttcatcctgcccgccgtcactgagaggatgttcaaccagaatgtggtggcccagctctggtacttcgtgaagtgcatctacttcgccctgtccgcctaccagatccgctgcggctaccccacccgcatcctcggcaacttcctcaccaagaagtacaatcatctcaacctcttcctcttccaggggttccggctggtgccgttcctggtggagctgcgggcagtgatggactgggtgtggacggacaccacgctgtccctgtccagctggatgtgtgtggaggacatctatgccaacatcttcatcatcaaatgcagccgagagacagagaagaaatacccgcagcccaaagggcagaagaagaagaagatcgtcaagtacggcatgggtggcctcatcatcctcttcctcatcgccatcatctggttcccgctgctcttcatgtcgctggtgcgctccgtggttggggttgtcaaccagcccatcgatgtcaccgtcaccctcaagctgggcggctatgagccgctgttcaccatgagcgcccagcagccgtccatcatccccttcacggcccaggcctatgaggagctgtcccggcagtttgacccccagccgctggccatgcagttcatcagccagtacagccctgaggacatcgtcacggcgcagattgagggcagctccggggcgctgtggcgcatcagtccccccagccgtgcccagatgaagcgggagctctacaacggcacggccgacatcaccctgcgcttcacctggaacttccagagggacctggcgaagggaggcactgtggagtatgccaacgagaagcacatgctggccctggcccccaacagcactgcacggcggcagctggccagcctgctcgagggcacctcggaccagtctgtggtcatccccaatctcttccccaagtacatccgtgcccccaacgggcccgaagccaaccctgtgaagcagctgcagcccaatgaggaggccgactacctcggcgtgcgtatccagctgcggagggagcagggtgcgggggccaccggcttcctcgaatggtgggtcatcgagctgcaggagtgccggaccgactgcaacctgctgcccatggtcattttcagtgacaaggtcagcccaccgagcctcggcttcctggctggctacggcatcatggggctgtacgtgtccatcgtgctggtcatcggcaagttcgtgcgcggattcttcagcgagatctcgcactccattatgttcgaggagctgccgtgcgtggaccgcatcctcaagctctgccaggacatcttcctggtgcgggagactcgggagctggagctggaggaggagttgtacgccaagctcatcttcctctaccgctcaccggagaccatgatcaagtggactcgtgagaaggagtaggagctgctgctggcgcccgagagggaaggagccggcctgctgggcagcgtggccacaaggggcggcactcctcaggccgggggagccactgccccgtccaaggccgccagctgtgatgcatcctcccggcctgcctgagccctgatgctgctgtcagagaaggacactgcgtccccacggcctgcgtggcgctgccgtcccccacgtgtactgtagagttttttttttaattaaaaaatgttttatttatacaaatgg

SEQ ID NO: 8 - RNA, 6273 nt, Homo sapiens
  source 1..6273: mol_type = genomic RNA
atggtatggagcatcacctaccacagctggctgaccttcgtactgctgctctgggcctgcctcatctggacggtgcgcagccgccaccaactggccatgctgtgctcgccctgcatcctgctgtatgggatgacgctgtgctgcctacgctacgtgtgggccatggacctgcgccctgagctgcccaccaccctgggccccgtcagcctgcgccagctggggctggagcacacccgctacccctgtctggaccttggtgccatgttgctctacaccctgaccttctggctcctgctgcgccagtttgtgaaagagaagctgctgaagtgggcagagtctccagctgcgctgacggaggtcaccgtggcagacacagagcccacgcggacgcagacgctgttgcagagcctgggggagctggtgaagggcgtgtacgccaagtactggatctatgtgtgtgctggcatgttcatcgtggtcagcttcgccggccgcctcgtggtctacaagattgtctacatgttcctcttcctgctctgcctcaccctcttccaggtctactacagcctgtggcggaagctgctcaaggccttctggtggctcgtggtggcctacaccatgctggtcctcatcgccgtctacaccttccagttccaggacttccctgcctactggcgcaacctcactggcttcaccgacgagcagctgggggacctgggcctggagcagttcagcgtgtccgagctcttctccagcatcctggtgcccggcttcttcctcctggcctgcatcctgcagctgcactacttccacaggcccttcatgcagctcaccgacatggagcacgtgtccctgcctggcacgcgcctcccgcgctgggctcacaggcaggatgcagtgagtgggaccccactgctgcgggaggagcagcaggagcatcagcagcagcagcaggaggaggaggaggaggaggaggactccagggacgaggggctgggcgtggccactccccaccaggccacgcaggtgcctgaaggggcagccaagtggggcctggtggctgagcggctgctggagctggcagccggcttctcggacgtcctctcacgcgtgcaggtgttcctgcggcggctgctggagcttcacgttttcaagctggtggccctgtacaccgtctgggtggccctgaaggaggtgtcggtgatgaacctgctgctggtggtgctgtgggccttcgccctgccctacccacgcttccggcccatggcctcctgcctgtccaccgtgtggacctgcgtcatcatcgtgtgtaagatgctgtaccagctcaaggttgtcaacccccaggagtattccagcaactgcaccgagcccttccccaacagcaccaacttgctgcccacggagatcagccagtccctgctgtaccgggggcccgtggaccctgccaactggtttggggtgcggaaagggttccccaacctgggctacatccagaaccacctgcaagtgctgctgctgctggtattcgaggccatcgtgtaccggcgccaggagcactaccgccggcagcaccagctggccccgctgcctgcccaggccgtgtttgccagcggcacccgccagcagctggaccaggatctgctcggctgcctcaagtacttcatcaacttcttcttctacaaattcgggctggagatctgcttcctgatggccgtgaacgtgatcgggcagcgcatgaactttctggtgaccctgcacggttgctggctggtggccatcctcacccgcaggcaccgccaggccattgcccgcctctggcccaactactgcctcttcctggcgctgttcctgctgtaccagtacctgctgtgcctggggatgcccccggccctgtgcattgattatccctggcgctggagccgggccgtccccatgaactccgcactcatcaagtggctgtacctgcctgatttcttccgggcccccaactccaccaacctcatcagcgactttctcctgctgctgtgcgcctcccagcagtggcaggtgttctcagctgagcgcacagaggagtggcagcgcatggctggcgtcaacaccgaccgcctggagccgctgcggggggagcccaaccccgtgcccaactttatccactgcaggtcctaccttgacatgctgaaggtggccgtcttccgatacctgttctggctggtgctggtggtggtgtttgtcacgggggccacccgcatcagcatcttcgggctgggctacctgctggcctgcttctacctgctgctcttcggcacggccctgctgcagagggacacacgggcccgcctcgtgctgtgggactgcctcattctgtacaacgtcaccgtcatcatctccaagaacatgctgtcgctcctggcctgcgtcttcgtggagcagatgcagaccggcttctgctgggtcatccagctcttcagccttgtatgcaccgtcaagggctactatgaccccaaggagatgatggacagagaccaggactgcctgctgcctgtggaggaggctggcatcatctgggacagcgtctgcttcttcttcctgctgctgcagcgccgcgtcttccttagccattactacctgcacgtcagggccgacctccaggccaccgccctgctagcctccaggggcttcgccctctacaacgctgccaacctcaagagcattgacttccaccgcaggatagaggagaagtccctggcccagctgaaaagacagatggagcgtatccgtgccaagcaggagaagcacaggcagggccgggtggaccgcagtcgcccccaggacaccctgggccccaaggaccccggcctggagccagggcccgacagtccagggggctcctccccgccacggaggcagtggtggcggccctggctggaccacgccacagtcatccactccggggactacttcctgtttgagtccgacagtgaggaagaggaggaggctgttcctgaagacccgaggccgtcggcacagagtgccttccagctggcgtaccaggcatgggtgaccaacgcccaggcggtgctgaggcggcggcagcaggagcaggagcaggcaaggcaggaacaggcaggacagctacccacaggaggtggtcccagccaggaggtggagccagcagagggccccgaggaggcagcggcaggccggagccatgtggtgcagagggtgctgagcacggcgcagttcctgtggatgctggggcaggcgctagtggatgagctgacacgctggctgcaggagttcacccggcaccacggcaccatgagcgacgtgctgcgggcagagcgctacctcctcacacaggagctcctgcagggcggcgaagtgcacaggggcgtgctggatcagctgtacacaagccaggccgaggccacgctgccaggccccaccgaggcccccaatgccccaagcaccgtgtccagtgggctgggcgcggaggagccactcagcagcatgacagacgacatgggcagccccctgagcaccggctaccacacgcgcagtggcagtgaggaggcagtcaccgaccccggggagcgtgaggctggtgcctctctgtaccagggactgatgcggacggccagcgagctgctcctggacaggcgcctgcgcatcccagagctggaggaggcagagctgtttgcggaggggcagggccgggcgctgcggctgctgcgggccgtgtaccagtgtgtggccgcccactcggagctgctctgctacttcatcatcatcctcaaccacatggtcacggcctccgccggctcgctggtgctgcccgtgctcgtcttcctgtgggccatgctgtcgatcccgaggcccagcaagcgcttctggatgacggccatcgtcttcaccgagatcgcggtggtcgtcaagtacctgttccagtttgggttcttcccctggaacagccacgtggtgctgcggcgctacgagaacaagccctacttcccgccccgcatcctgggcctggagaagactgacggctacatcaagtacgacctggtgcagctcatggcccttttcttccaccgctcccagctgctgtgctatggcctctgggaccatgaggaggactcaccatccaaggagcatgacaagagcggcgaggaggagcagggagccgaggaggggccaggggtgcctgcggccaccaccgaagaccacattcaggtggaagcgagggtcggacccacggacgggaccccagaaccccaagtggagctcaggccccgtgatacgaggcgcatcagtctacgttttagaagaaggaagaaggagggcccagcacggaaaggagcggcagccatcgaagctgaggacagggaggaagaagagggggaggaagagaaagaggcccccacggggagagagaagaggccaagccgctctggaggaagagtaagggcggccgggcggcggctgcagggcttctgcctgtccctggcccagggcacatatcggccgctacggcgcttcttccacgacatcctgcacaccaagtaccgcgcagccaccgacgtctatgccctcatgttcctggctgatgttgtcgacttcatcatcatcatttttggcttctgggcctttgggaagcactcggcggccacagacatcacgtcctccctatcagacgaccaggtacccgaggctttcctggtcatgctgctgatccagttcagtaccatggtggttgaccgcgccctctacctgcgcaagaccgtgctgggcaagctggccttccaggtggcgctggtgctggccatccacctatggatgttcttcatcctgcccgccgtcactgagaggatgttcaaccagaatgtggtggcccagctctggtacttcgtgaagtgcatctacttcgccctgtccgcctaccagatccgctgcggctaccccacccgcatcctcggcaacttcctcaccaagaagtacaatcatctcaacctcttcctcttccaggggttccggctggtgccgttcctggtggagctgcgggcagtgatggactgggtgtggacggacaccacgctgtccctgtccagctggatgtgtgtggaggacatctatgccaacatcttcatcatcaaatgcagccgagagacagagaagaaatacccgcagcccaaagggcagaagaagaagaagatcgtcaagtacggcatgggtggcctcatcatcctcttcctcatcgccatcatctggttcccgctgctcttcatgtcgctggtgcgctccgtggttggggttgtcaaccagcccatcgatgtcaccgtcaccctgaagctgggcggctatgagccgctgttcaccatgagcgcccagcagccgtccatcatccccttcacggcccaggcctatgaggagctgtcccggcagtttgacccccagccgctggccatgcagttcatcagccagtacagccctgaggacatcgtcacggcgcagattgagggcagctccggggcgctgtggcgcatcagtccccccagccgtgcccagatgaagcgggagctctacaacggcacggccgacatcaccctgcgcttcacctggaacttccagagggacctggcgaagggaggcactgtggagtatgccaacgagaagcacatgctggccctggcccccaacagcactgcacggcggcagctggccagcctgctcgagggcacctcggaccagtctgtggtcatccccaatctcttccccaagtacatccgtgcccccaacgggcccgaagccaaccctgtgaagcagctgcagcccaatgaggaggccgactacctcggcgtgcgtatccagctgcggagggagcagggtgcgggggccaccggcttcctcgaatggtgggtcatcgagctgcaggagtgccggaccgactgcaacctgctgcccatggtcattttcagtgacaaggtcagcccaccgagcctcggcttcctggctggctacggcatcatggggctgtacgtgtccatcgtgctggtcatcggcaagttcgtgcgcggattcttcagcgagatctcgcactccattatgttcgaggagctgccgtgcgtggaccgcatcctcaagctctgccaggacatcttcctggtgcgggagactcgggagctggagctggaggaggagttgtacgccaagctcatcttcctctaccgctcaccggagaccatgatcaagtggactcgtgagaaggagtag

SEQ ID NO: 9 - RNA, 7563 nt, Homo sapiens
  source 1..7563: mol_type = genomic RNA
atggagccgcacgtgttcggcgcggtcctgtactggctgctgctgccctgcgcgctgctggctgcctgcctgctccgcttcagcggactctcgctggtctacctgctcttcctgctgctgctgccctggttccccggccccacccgatgcggcctccaaggtcacacaggccgcctcctgcgggcattgctgggcctcagcctgctcttcctggtggcccatctcgccctccagatctgcctgcatactgtgccccgcctggaccagctcctgggacccagctgcagccgctgggagaccctctcgcgacacataggggtcacaaggctggacctgaaggacatccccaacgccatccggctggtggcccctgacctgggcatcttggtggtctcctctgtctgcctcggcatctgcgggcgccttgcaaggaacacccggcagagcccacatccacgggagctggatgatgatgagagggatgtggatgccagcccgacggcagggctgcaggaagcagcaacgctggcccctacacggaggtcacggctggccgctcgtttccgagtcacggcccactggctgctggtggcggctgggcgggtcctggccgtaacactgcttgcactggcaggcatcgcccacccctcggccctctccagtgtctacctgctgctcttcctggccctctgcacctggtgggcctgccactttcccatcagcactcggggcttcagcagactctgcgccgcggtggggtgcttcggcgccggccatctcatctgcctctactgctaccagatgcccttggcacaggctctgctcccgcctgccggcatctgggctagggtgctgggtctcaaggacttcgtgggtcccaccaactgctccagcccccacgcgctggtcctcaacaccggcctggactggcctgtgtatgccagccccggcgtcctcctgctgctgtgctacgccacggcctctctgcgcaagctccgcgcgtaccgcccctccggccagaggaaggaggcggcaaaggggtatgaggctcgggagctggagctagcagagctggaccagtggccccaggaacgggagtctgaccagcacgtggtgcccacagcacccgacaccgaggctgataactgcatcgtgcacgagctgaccggccagagctccgtcctgcggcggcctgtgcggcccaagcgggctgagcccagggaggcgtctccgctccacagcctgggccacctcatcatggaccagagctatgtgtgcgcgctcattgccatgatggtatggagcatcacctaccacagctggctgaccttcgtactgctgctctgggcctgcctcatctggacggtgcgcagccgccaccaactggccatgctgtgctcgccctgcatcctgctgtatgggatgacgctgtgctgcctacgctacgtgtgggccatggacctgcgccctgagctgcccaccaccctgggccccgtcagcctgcgccagctggggctggagcacacccgctacccctgtctggaccttggtgccatgttgctctacaccctgaccttctggctcctgctgcgccagtttgtgaaagagaagctgctgaagtgggcagagtctccagctgcgctgacggaggtcaccgtggcagacacagagcccacgcggacgcagacgctgttgcagagcctgggggagctggtgaagggcgtgtacgccaagtactggatctatgtgtgtgctggcatgttcatcgtggtcagcttcgccggccgcctcgtggtctacaagattgtctacatgttcctcttcctgctctgcctcaccctcttccaggtctactacagcctgtggcggaagctgctcaaggccttctggtggctcgtggtggcctacaccatgctggtcctcatcgccgtctacaccttccagttccaggacttccctgcctactggcgcaacctcactggcttcaccgacgagcagctgggggacctgggcctggagcagttcagcgtgtccgagctcttctccagcatcctggtgcccggcttcttcctcctggcctgcatcctgcagctgcactacttccacaggcccttcatgcagctcaccgacatggagcacgtgtccctgcctggcacgcgcctcccgcgctgggctcacaggcaggatgcagtgagtgggaccccactgctgcgggaggagcagcaggagcatcagcagcagcagcaggaggaggaggaggaggaggaggactccagggacgaggggctgggcgtggccactccccaccaggccacgcaggtgcctgaaggggcagccaagtggggcctggtggctgagcggctgctggagctggcagccggcttctcggacgtcctctcacgcgtgcaggtgttcctgcggcggctgctggagcttcacgttttcaagctggtggccctgtacaccgtctgggtggccctgaaggaggtgtcggtgatgaacctgctgctggtggtgctgtgggccttcgccctgccctacccacgcttccggcccatggcctcctgcctgtccaccgtgtggacctgcgtcatcatcgtgtgtaagatgctgtaccagctcaaggttgtcaacccccaggagtattccagcaactgcaccgagcccttccccaacagcaccaacttgctgcccacggagatcagccagtccctgctgtaccgggggcccgtggaccctgccaactggtttggggtgcggaaagggttccccaacctgggctacatccagaaccacctgcaagtgctgctgctgctggtattcgaggccatcgtgtaccggcgccaggagcactaccgccggcagcaccagctggccccgctgcctgcccaggccgtgtttgccagcggcacccgccagcagctggaccaggatctgctcggctgcctcaagtacttcatcaacttcttcttctacaaattcgggctggagatctgcttcctgatggccgtgaacgtgatcgggcagcgcatgaactttctggtgaccctgcacggttgctggctggtggccatcctcacccgcaggcaccgccaggccattgcccgcctctggcccaactactgcctcttcctggcgctgttcctgctgtaccagtacctgctgtgcctggggatgcccccggccctgtgcattgattatccctggcgctggagccgggccgtccccatgaactccgcacccatcaagtggctgtacctgcctgatttcttccgggcccccaactccaccaacctcatcagcgactttctcctgctgctgtgcgcctcccagcagtggcaggtgttctcagctgagcgcacagaggagtggcagcgcatggctggcgtcaacaccgaccgcctggagccgctgcggggggagcccaaccccgtgcccaactttatccactgcaggtcctaccttgacatgctgaagctggccgtcttccgatacctgttctggctggtgctggtggtggtgtttgtcacgggggccacccgcatcagcatcttcgggctgggctacctgctggcctgcttctacctgctgctcttcggcacggccctgctgcagagggacacacgggcccgcctcgtgctgtgggactgcctcattctgtacaacgtcaccgtcatcatctccaagaacatgctgtcgctcctggcctgcgtcttcgtggagcagatgcagaccggcttctgctgggtcatccagctcttcagccttgtatgcaccgtcaagggctactatgaccccaaggagatgatggacagagaccaggactgcctgctgcctgtggaggaggctggcatcatctgggacagcgtctgcttcttcttcctgctgctgcagcgccgcgtcttccttagccattactacctgcacgtcagggccgacctccaggccaccgccctgctagcctccaggggcttcgccctctacaacgctgccaacctcaagagcattgacttccaccgcaggatagaggagaagtccctggcccagctgaaaagacagatggagcgtatccgtgccaagcaggagaagcacaggcagggccgggtggaccgcagtcgcccccaggacaccctgggccccaaggaccccggcctggagccagggcccgacagtccagggggctcctccccgctacggaggcagtggtggcggccctggctggaccacgccacagtcatccactccggggactacttcctgtttgagtccgacagtgaggaagaggaggaggctgttcctgaagacccgaggccgtcggcacagagtgccttccagctggcgtaccaggcatgggtgaccaacgcccaggcggtgctgaggcggcggcagcaggagcaggagcaggcaaggcaggaacaggcaggacagctacccacaggaggtggtcccagccaggaggtggagccagcagagggccccgaggaggcagcggcaggccggagccatgtggtgcagagggtgctgagcacggcgcagttcctgtggatgctggggcaggcgctagtggatgagctgacacgctggctgcaggagttcacccggcaccacggcaccatgagcgacgtgctgcgggcagagcgctacctcctcacacaggagctcctgcagggcggcgaagtgcacaggggcgtgctggatcagctgtacacaagccaggccgaggccacgctgccaggccccaccgaggcccccaatgccccaagcaccgtgtccagtgggctgggcgcggaggagccactcagcagcatgacagacgacatgggcagccccctgagcaccggctaccacacgcgcagtggcagtgaggaggcagtcaccgaccccggggagcgtgaggctggtgcctctctgtaccagggactgatgcggacggccagcgagctgctcctggacaggcgcctgcgcatcccagagctggaggaggcagagctgtttgcggaggggcagggccgggcgctgcggctgctgcgggccgtgtaccagtgtgtggccgcccactcggagctgctctgctacttcatcatcatcctcaaccacatggtcacggcctccgccggctcgctggtgctgcccgtgctcgtcttcctgtgggccatgctgtcgatcccgaggcccagcaagcgcttctggatgacggccatcgtcttcaccgagatcgcggtggtcgtcaagtacctgttccagtttgggttcttcccctggaacagccacgtggtgctgcggcgctacgagaacaagccctacttcccgccccgcatcctgggcctggagaagactgacggctacatcaagtacgacctggtgcagctcatggcccttttcttccaccgctcccagctgctgtgctatggcctctgggaccatgaggaggactcaccatccaaggagcatgacaagagcggcgaggaggagcagggagccgaggaggggccaggggtgcctgcggccaccaccgaagaccacattcaggtggaagccagggtcggacccacggacgggaccccagaatcccaagtggagctcaggccccgtgatacgaggcgcatcagtctacgttttagaagaaggaaggagggcccagcacggaaaggagcggcagccatcgaagctgaggacagggaggaagaagagggggaggaagagaaagaggcccccacggggagagagaagaggccaagccgctctggaggaagagtaagggcggccgggcggcggctgcagggcttctgcctgtccctggcccagggcacatatcggccgctacggcgcttcttccacgacatcctgcacaccaagtaccgcgcagccaccgacgtctatgccctcatgttcctggctgatgttgtcgacttcatcatcatcatttttggcttctgggcctttgggaagcactcggcggccacagacatcacgtcctccctatcagacgaccaggtacccgaggctttcctggtcatgctgctgatccagttcagtaccatggtggttgaccgcgccctctacctgcgcaagaccgtgctgggcaagctggccttccaggtggcgctggtgctggccatccacctatggatgttcttcatcctgcccgccgtcactgagaggatgttcaaccagaatgtggtggcccagctctggtacttcgtgaagtgcatctacttcgccctgtccgcctaccagatccgctgcggctaccccacccgcatcctcggcaacttcctcaccaagaagtacaatcatctcaacctcttcctcttccaggggttccggctggtgccgttcctggtggagctgcgggcagtgatggactgggtgtggacggacaccacgctgtccctgtccagctggatgtgtgtggaggacatctatgccaacatcttcatcatcaaatgcagccgagagacagagaagaaatacccgcagcccaaagggcagaagaagaagaagatcgtcaagtacggcatgggtggcctcatcatcctcttcctcatcgccatcatctggttcccgctgctcttcatgtcgctggtgcgctccgtggttggggttgtcaaccagcccatcgatgtcaccgtcaccctgaagctgggcggctatgagccgctgttcaccatgagcgcccagcagccgtccatcatccccttcacggcccaggcctatgaggagctgtcccggcagtttgacccccagccgctggccatgcagttcatcagccagtacagccctgaggacgtcgtcacggcgcagattgagggcagctccggggcgctgtggcgcatcagtccccccagccgtgcccagatgaagcgggagctctacaacggcacggccgacatcaccctgcgcttcacctggaacttccagagggacctggcgaagggaggcactgtggagtatgccaacgagaagcacatgctggccctggcccccaacagcactgcacggcggcagctggccagcctgctcgagggcacctcggaccagtctgtggtcatccccaatctcttccccaagtacatccgtgcccccaacgggcccgaagccaaccctgtgaagcagctgcagcccaatgaggaggccgactacctcggcgtgcgtatccagctgcggagggagcagggtgcgggggccaccggcttcctcgaatggtgggtcatcgaactgcaggagtgccggaccgactgcaacctgctgcccatggtcattttcagtgacaaggtcagcccaccgagcctcggcttcctggctggctacggcatcatggggctgtacgtgtccatcgtgctggtcatcggcaagttcgtgcgcggattcttcagcgagatctcgcactccattatgttcgaggagctgccgtgcgtggaccgcatcctcaagctctgccaggacatcttcctggtgcgggagactcgggagctggagctggaggaggagttgtacgccaagctcatcttcctctaccgctcaccggagaccatgatcaagtggactcgtgagaaggagtag

SEQ ID NO: 10 - RNA, 1635 nt, Homo sapiens
  source 1..1635: mol_type = genomic RNA
taccagatccgctgcggctaccccacccgcatcctcggcaacttcctcaccaagaagtacaatcatctcaacctcttcctcttccaggggttccggctggtgccgttcctggtggagctgcgggcagtgatggactgggtgtggacggacaccacgctgtccctgtccagctggatgtgtgtggaggacatctatgccaacatcttcatcatcaaatgcagccgagagacagagaaggttcatgaagatttgcccgttttcttctaaaagttctatagttttagctctgaagtttcgctctttgatccactttgagaaatacccgcagcccaaagggcagaagaagaagaagatcgtcaagtacggcatgggtggcctcatcatcctcttcctcatcgccatcatctggttcccactgctcttcatgtcgctggtgcgctccgtggttggggttgtcaaccagcccatcgatgtcaccgtcaccctgaagctgggcggctatgagccgctgttcaccatgagcgcccagcagccgtccatcatccccttcacggcccaggcctatgaggagctgtcccggcagtttgacccccagccgctggccatgcagttcatcagccagtacagccctgaggacatcgtcacggcgcagattgagggcagctccggggcgctgtggcgcatcagtccccccagccgtgcccagatgaagcgggagctctacaacggcacggccgacatcaccctgcgcttcacctggaacttccagagggacctggcgaagggaggcactgtggagtatgccaacgagaagcacatgctggccctggcccccaacagcactgcacggcggcagctggccagcctgctcgagggcacctcggaccagtctgtggtcatccctaatctcttccccaagtacatccgtgcccccaacgggcccgaagccaaccctgtgaagcagctgcagcccaatgaggaggccgactacctcggcgtgcgtatccagctgcggagggagcagggtgcgggggccaccggcttcctcgaatggtgggtcatcgagctgcaggagtgccggaccgactgcaacctgctgcccatggtcattttcagtgacaagcatcatggggctgtacgtgtccatcgtgctggtcatcggcaagttcgtgcgcggattcttcagcgagatctcgcactccattatgttcgaggagctgccgtgcgtggaccgcatcctcaagctctgccaggacatcttcctggtgcgggagactcgggagctggagctggaggaggagttgtacgccaagctcatcttcctctaccgctcaccggagaccatgatcaagtggactcgtgagaaggagtaggagctgctgctggcgcccgagagggaaggagccggcctgctgggcagcgtggccacaaggggcggcactcctcaggccgggggagccactgccccgtccaaggccgccagctgtgatgcatcctcccggcctgcctgagccctgatgctgctgtcagagaaggacactgcgtccccacggcctgcgtggcgctgccgtcccccacgtgtactgtagagttttttttttaattaaaaaatgttttatttatacaaatggacaa

SEQ ID NO: 11 - RNA, 660 nt, Homo sapiens
  source 1..660: mol_type = genomic RNA
ggtcatccctaatctcttccccaagtacatccgtgcccccaacgggcccgaagccaaccctgtgaagcagctgcagcccaatgaggaggccgactacctcggctgacaaggtcagcccaccgagcctcggcttcctggctggctacggcatcatggggctgtacgtgtccatcgtgctggtcatcggcaagttcgtgcgcggattcttcagcgagatctcgcactccattatgttcgaggagctgccgtgcgtggaccgcatcctcaagctctgccaggacatcttcctggtgcgggagactcgggagctggagctggaggaggagttgtacgccaagctcatcttcctctaccgctcaccggagaccatgatcaagtggactcgtgagaaggagtaggagctgctgctggcgcccgagagggaaggagccggcctgctgggcagcgtggccacaaggggcggcactcctcaggccgggggagccactgccccgtccaaggccgccagctgtgatgcatcctcccggcctgcctgagccctgatgctgctgtcagagaaggacactgcgtccccacggcctgcgtggcgctgccgtcccccacgtgtactgtagagttttttttttaattaaaaaatgttttatttatacaaatggacaa

SEQ ID NO: 12 - RNA, 8072 nt, Homo sapiens
  source 1..8072: mol_type = genomic RNA
cgtccggcccagctcggccccagtgagccgagcgctgcgctccgccgaggggcagggcggtcgcctgagcgagcgcgggcccgggacgtcggcaccggcggggcggccgaaggagaaggaggaagaggagaaggcggcgcgcgggtccccgcgggtcagccatggcgcgccggccccggggcccccgcaccgccccatagcgccgcggcgtccgctcggtctgggccgggccctgggccctccagccatggagccgcacgtgctcggcgcggtcctgtactggctgctgctgccctgcgcgctgctggctgcctgcctgctccgcttcagcggactctcgctggtctacctgctcttcctgctgctgctgccctggttccccggccccacccgatgcggcctccaaggtcacacaggccgcctcctgcgggcattgctgggcctcagcctgctcttcctggtggcccatctcgccctccagatctgcctgcatattgtgccccgcctggaccagctcctgggacccagctgcagccgctgggagaccctctcgcgacacataggggtcacaaggctggacctgaaggacatccccaacgccatccggctggtggcccctgacctgggcatcttggtggtctcctctgtctgcctcggcatctgcgggcgccttgcaaggaacacccggcagagcccacatccacgggagctggatgatgatgagagggatgtggatgccagcccgacggcagggctgcaggaagcagcaacgctggcccctacacggaggtcacggctggccgctcgtttccgagtcacggcccactggctgctggtggcggctgggcgggtcctggccgtaacactgcttgcactggcaggcatcgcccacccctcggccctctccagtgtctacctgctgctcttcctggccctctgcacctggtgggcctgccactttcccatcagcactcggggcttcagcagactctgcgtcgcggtggggtgcttcggcgccggccatctcatctgcctctactgctaccagatgcccttggcacaggctctgctcccgcctgccggcatctgggctagggtgctgggtctcaaggacttcgtgggtcccaccaactgctccagcccccacgcgctggtcctcaacaccggcctggactggcctgtgtatgccagccccggcgtcctcctgctgctgtgctacgccacggcctctctgcgcaagctccgcgcgtaccgcccctccggccagaggaaggaggcggcaaaggggtatgaggctcgggagctggagctagcagagctggaccagtggccccaggaacgggagtctgaccagcacgtggtgcccacagcacccgacaccgaggctgataactgcatcgtgcacgagctgaccggccagagctccgtcctgcggcggcctgtgcggcccaagcgggctgagcccagggaggcgtctccgctccacagcctgggccacctcatcatggaccagagctatgtgtgcgcgctcattgccatgatggtatggagcatcacctaccacagctggctgaccttcgtactgctgctctgggcctgcctcatctggacggtgcgcagccgccaccaactggccatgctgtgctcgccctgcatcctgctgtatgggatgacgctgtgctgcctacgctacgtgtgggccatggacctgcgccctgagctgcccaccaccctgggccccgtcagcctgcgccagctggggctggagcacacccgctacccctgtctggaccttggtgccatgttgctctacaccctgaccttctggctcctgctgcgccagtttgtgaaagagaagctgctgaagtgggcagagtctccagctgcgctgacggaggtcaccgtggcagacacagagcccacgcggacgcagacgctgttgcagagcctgggggagctggtgaagggcgtgtacgccaagtactggatctatgtgtgtgctggcatgttcatcgtggtcagcttcgccggccgcctcgtggtctacaagattgtctacatgttcctcttcctgctctgcctcaccctcttccaggtctactacagcctgtggcggaagctgctcaaggccttctggtggctcgtggtggcctacaccatgctggtcctcatcgccgtctacaccttccagttccaggacttccctgcctactggcgcaacctcactggcttcaccgacgagcagctgggggacctgggcctggagcagttcagcgtgtccgagctcttctccagcatcctggtgcccggcttcttcctcctggcctgcatcctgcagctgcactacttccacaggcccttcatgcagctcaccgacatggagcacgtgtccctgcctggcacgcgcctcccgcgctgggctcacaggcaggatgcagtgagtgggaccccactgctgcgggaggagcagcaggagcatcagcagcagcagcaggaggaggaggaggaggaggaggactccagggacgaggggctgggcgtggccactccccaccaggccacgcaggtgcctgaaggggcagccaagtggggcctggtggctgagcggctgctggagctggcagccggcttctcggacgtcctctcacgcgtgcaggtgttcctgcggcggctgctggagcttcacgttttcaagctggtggccctgtacaccgtctgggtggccctgaaggaggtgtcggtgatgaacctgctgctggtggtgctgtgggccttcgccctgccctacccacgcttccggcccatggcctcctgcctgtccaccgtgtggacctgcgtcatcatcgtgtgtaagatgctgtaccagctcaaggttgtcaacccccaggagtattccagcaactgcaccgagcccttccccaacagcaccaacttgctgcccacggagatcagccagtccctgctgtaccgggggcccgtggaccctgccaactggtttggggtgcggaaagggttccccaacctgggctacatccagaaccacctgcaagtgctgctgctgctggtattcgaggccatcgtgtaccggcgccaggagcactaccgccggcagcaccagctggccccgctgcctgcccaggccgtgtttgccagcggcacccgccagcagctggaccaggatctgctcggctgcctcaagtacttcatcaacttcttcttctacaaattcgggctggagatctgcttcctgatggccgtgaacgtgatcgggcagcgcatgaactttctggtgaccctgcacggttgctggctggtggccatcctcacccgcaggcaccgccaggccattgcccgcctctggcccaactactgcctcttcctggcgctgttcctgctgtaccagtacctgctgtgcctggggatgcccccggccctgtgcattgattatccctggcgctggagccgggccgtccccatgaactccgcactcatcaagtggctgtacctgcctgatttcttccgggcccccaactccaccaacctcatcagcgactttctcctgctgctgtgcgcctcccagcagtggcaggtgttctcagctgagcgcacagaggagtggcagcgcatggctggcgtcaacaccgaccgcctggagccgctgcggggggagcccaaccccgtgcccaactttatccactgcaggtcctaccttgacatgctgaaggtggccgtcttccgatacctgttctggctggtgctggtggtggtgtttgtcacgggggccacccgcatcagcatcttcgggctgggctacctgctggcctgcttctacctgctgctcttcggcacggccctgctgcagagggacacacgggcccgcctcgtgctgtgggactgcctcattctgtacaacgtcaccgtcatcatctccaagaacatgctgtcgctcctggcctgcgtcttcgtggagcagatgcagaccggcttctgctgggtcatccagctcttcagccttgtatgcaccgtcaagggctactatgaccccaaggagatgatggacagagaccaggactgcctgctgcctgtggaggaggctggcatcatctgggacagcgtctgcttcttcttcctgctgctgcagcgccgcgtcttccttagccattactacctgcacgtcagggccgacctccaggccaccgccctgctagcctccaggggcttcgccctctacaacgctgccaacctcaagagcattgactttcaccgcaggatagaggagaagtccctggcccagctgaaaagacagatggagcgtatccgtgccaagcaggagaagcacaggcagggccgggtggaccgcagtcgcccccaggacaccctgggccccaaggaccccggcctggagccagggcccgacagtccagggggctcctccccgccacggaggcagtggtggcggccctggctggaccacgccacagtcatccactccggggactacttcctgtttgagtccgacagtgaggaagaggaggaggctgttcctgaagacccgaggccgtcggcacagagtgccttccagctggcgtaccaggcatgggtgaccaacgcccaggcggtgctgaggcggcggcagcaggagcaggagcaggcaaggcaggaacaggcaggacagctacccacaggaggtggtcccagccaggaggtggagccagcagagggccccgaggaggcagcggcaggccggagccatgtggtgcagagggtgctgagcacggcgcagttcctgtggatgctggggcaggcgctagtggatgagctgacacgctggctgcaggagttcacccggcaccacggcaccatgagcgacgtgctgcgggcagagcgctacctcctcacacaggagctcctgcagggcggcgaagtgcacaggggcgtgctggatcagctgtacacaagccaggccgaggccacgctgccaggccccaccgaggcccccaatgccccaagcaccgtgtccagtgggctgggcgcggaggagccactcagcagcatgacagacgacatgggcagccccctgagcaccggctaccacacgcgcagtggcagtgaggaggcagtcaccgaccccggggagcgtgaggctggtgcctctctgtaccagggactgatgcggacggccagcgagctgctcctggacaggcgcctgcgcatcccagagctggaggaggcagagctgtttgcggaggggcagggccgggcgctgcggctgctgcgggccgtgtaccagtgtgtggccgcccactcggagctgctctgctacttcatcatcatcctcaaccacatggtcacggcctccgccggctcgctggtgctgcccgtgctcgtcttcctgtgggccatgctgtcgatcccgaggcccagcaagcgcttctggatgacggccatcgtcttcaccgagatcgcggtggtcgtcaagtacctgttccagtttgggttcttcccctggaacagccacgtggtgctgcggcgctacgagaacaagccctacttcccgccccgcatcctgggcctggagaagactgacggctacatcaagtacgacctggtgcagctcatggcccttttcttccaccgctcccagctgctgtgctatggcctctgggaccatgaggaggactcaccatccaaggagcatgacaagagcggcgaggaggagcagggagccgaggaggggccaggggtgcctgcggccaccaccgaagaccacattcaggtggaagccagggtcggacccacggacgggaccccagaaccccaagtggagctcaggccccgtgatacgaggcgcatcagtctacgttttagaagaaggaagaaggagggcccagcacggaaaggagcggcagccatcgaagctgaggacagggaggaagaagagggggaggaagagaaagaggcccccacggggagagagaagaggccaagccgctctggaggaagagtaagggcggccgggcggcggctgcagggcttctgcctgtccctggcccagggcacatatcggccgctacggcgcttcttccacgacatcctgcacaccaagtaccgcgcagccaccgacgtctatgccctcatgttcctggctgatgttgtcgacttcatcatcatcatttttggcttctgggcctttgggaagcactcggcggccacagacatcacgtcctccctatcagacgaccaggtacccgaggctttcctggtcatgctgctgatccagttcagtaccatggtggttgaccgcgccctctacctgcgcaagaccgtgctgggcaagctggccttccaggtggcgctggtgctggccatccacctatggatgttcttcatcctgcccgccgtcactgagaggatgttcaaccagaatgtggtggcccagctctggtacttcgtgaagtgcatctacttcgccctgtccgcctaccagatccgctgcggctaccccacccgcatcctcggcaacttcctcaccaagaagtacaatcatctcaacctcttcctcttccaggggttccggctggtgccgttcctggtggagctgcgggcagtgatggactgggtgtggacggacaccacgctgtccctgtccagctggatgtgtgtggaggacatctatgccaacatcttcatcatcaaatgcagccgagagacagagaagaaatacccgcagcccaaagggcagaagaagaagaagatcgtcaagtacggcatgggtggcctcatcatcctcttcctcatcgccatcatctggttcccactgctcttcatgtcgctggtgcgctccgtggttggggttgtcaaccagcccatcgatgtcaccgtcaccctgaagctgggcggctatgagccgctgttcaccatgagcgcccagcagccgtccatcatccccttcacggcccaggcctatgaggagctgtcccggcagtttgacccccagccgctggccatgcagttcatcagccagtacagccctgaggacatcgtcacggcgcagattgagggcagctccggggcgctgtggcgcatcagtccccccagccgtgcccagatgaagcgggagctctacaacggcacggccgacatcaccctgcgcttcacctggaacttccagagggacctggcgaagggaggcactgtggagtatgccaacgagaagcacatgctggccctggcccccaacagcactgcacggcggcagctggccagcctgctcgagggcacctcggaccagtctgtggtcatccctaatctcttccccaagtacatccgtgcccccaacgggcccgaagccaaccctgtgaagcagctgcagcccaatgaggaggccgactacctcggcgtgcgtatccagctgcggagggagcagggtgcgggggccaccggcttcctcgaatggtgggtcatcgagctgcaggagtgccggaccgactgcaacctgctgcccatggtcattttcagtgacaaggtcagcccaccgagcctcggcttcctggctggctacggcatcatggggctgtacgtgtccatcgtgctggtcatcggcaagttcgtgcgcggattcttcagcgagatctcgcactccattatgttcgaggagctgccgtgcgtggaccgcatcctcaagctctgccaggacatcttcctggtgcgggagactcgggagctggagctggaggaggagttgtacgccaagctcatcttcctctaccgctcaccggagaccatgatcaagtggactcgtgagaaggagtaggagctgctgctggcgcccgagagggaaggagccggcctgctgggcagcgtggccacaaggggcggcactcctcaggccgggggagccactgccccgtccaaggccgccagctgtgatgcatcctcccggcctgcctgagccctgatgctgctgtcagagaaggacactgcgtccccacggcctgcgtggcgctgccgtcccccacgtgtactgtagagttttttttttaattaaaaaatgttttatttatacaaatgga

SEQ ID NO: 13 - RNA, 8089 nt, Homo sapiens
  source 1..8089: mol_type = genomic RNA
gggagccgccgtccggcccagctcggccccagtgagccgagcgctgcgctccgccgaggggcagggcggtcgcctgagcgagcgcgggcccgggacgtcggcaccggcggggcggccgaaggagaaggaggaagaggagaaggcggcgcgcgggtccccgcgggtcagccatggcgcgccggccccggggcccccgcaccgccccatagcgccgcggcgtccgctcggtctgggccgggccctgggccctccagccatggagccgcacgtgctcggcgcggtcctgtactggctgctgctgccctgcgcgctgctggctgcctgcctgctccgcttcagcggactctcgctggtctacctgctcttcctgctgctgctgccctggttccccggccccacccgatgcggcctccaaggtcacacaggccgcctcctgcgggcattgctgggcctcagcctgctcttcctggtggcccatctcgccctccagatctgcctgcatattgtgccccgcctggaccagctcctgggacccagctgcagccgctgggagaccctctcgcgacacataggggtcacaaggctggacctgaaggacatccccaacgccatccggctggtggcccctgacctgggcatcttggtggtctcctctgtctgcctcggcatctgcgggcgccttgcaaggaacacccggcagagcccacatccacgggagctggatgatgatgagagggatgtggatgccagcccgacggcagggctgcaggaagcagcaacgctggcccctacacggaggtcacggctggccgctcgtttccgagtcacggcccactggctgctggtggcggctgggcgggtcctggccgtaacactgcttgcactggcaggcatcgcccacccctcggccctctccagtgtctacctgctgctcttcctggccctctgcacctggtgggcctgccactttcccatcagcactcggggcttcagcagactctgcgtcgcggtggggtgcttcggcgccggccatctcatctgcctctactgctaccagatgcccttggcacaggctctgctcccgcctgccggcatctgggctagggtgctgggtctcaaggacttcgtgggtcccaccaactgctccagcccccacgcgctggtcctcaacaccggcctggactggcctgtgtatgccagccccggcgtcctcctgctgctgtgctacgccacggcctctctgcgcaagctccgcgcgtaccgcccctccggccagaggaaggaggcggcaaaggggtatgaggctcgggagctggagctagcagagctggaccagtggccccaggaacgggagtctgaccagcacgtggtgcccacagcacccgacaccgaggctgataactgcatcgtgcacgagctgaccggccagagctccgtcctgcggcggcctgtgcggcccaagcgggctgagcccagggaggcgtctccgctccacagcctgggccacctcatcatggaccagagctatgtgtgcgcgctcattgccatgatggtatggagcatcacctaccacagctggctgaccttcgtactgctgctctgggcctgcctcatctggacggtgcgcagccgccaccaactggccatgctgtgctcgccctgcatcctgctgtatgggatgacgctgtgctgcctacgctacgtgtgggccatggacctgcgccctgagctgcccaccaccctgggccccgtcagcctgcgccagctggggctggagcacacccgctacccctgtctggaccttggtgccatgttgctctacaccctgaccttctggctcctgctgcgccagtttgtgaaagagaagctgctgaagtgggcagagtctccagctgcgctgacggaggtcaccgtggcagacacagagcccacgcggacgcagacgctgttgcagagcctgggggagctggtgaagggcgtgtacgccaagtactggatctatgtgtgtgctggcatgttcatcgtggtcagcttcgccggccgcctcgtggtctacaagattgtctacatgttcctcttcctgctctgcctcaccctcttccaggtctactacagcctgtggcggaagctgctcaaggccttctggtggctcgtggtggcctacaccatgctggtcctcatcgccgtctacaccttccagttccaggacttccctgcctactggcgcaacctcactggcttcaccgacgagcagctgggggacctgggcctggagcagttcagcgtgtccgagctcttctccagcatcctggtgcccggcttcttcctcctggcctgcatcctgcagctgcactacttccacaggcccttcatgcagctcaccgacatggagcacgtgtccctgcctggcacgcgcctcccgcgctgggctcacaggcaggatgcagtgagtgggaccccactgctgcgggaggagcagcaggagcatcagcagcagcagcaggaggaggaggaggaggaggaggactccagggacgaggggctgggcgtggccactccccaccaggccacgcaggtgcctgaaggggcagccaagtggggcctggtggctgagcggctgctggagctggcagccggcttctcggacgtcctctcacgcgtgcaggtgttcctgcggcggctgctggagcttcacgttttcaagctggtggccctgtacaccgtctgggtggccctgaaggaggtgtcggtgatgaacctgctgctggtggtgctgtgggccttcgccctgccctacccacgcttccggcccatggcctcctgcctgtccaccgtgtggacctgcgtcatcatcgtgtgtaagatgctgtaccagctcaaggttgtcaacccccaggagtattccagcaactgcaccgagcccttccccaacagcaccaacttgctgcccacggagatcagccagtccctgctgtaccgggggcccgtggaccctgccaactggtttggggtgcggaaagggttccccaacctgggctacatccagaaccacctgcaagtgctgctgctgctggtattcgaggccatcgtgtaccggcgccaggagcactaccgccggcagcaccagctggccccgctgcctgcccaggccgtgtttgccagcggcacccgccagcagctggaccaggatctgctcggctgcctcaagtacttcatcaacttcttcttctacaaattcgggctggagatctgcttcctgatggccgtgaacgtgatcgggcagcgcatgaactttctggtgaccctgcacggttgctggctggtggccatcctcacccgcaggcaccgccaggccattgcccgcctctggcccaactactgcctcttcctggcgctgttcctgctgtaccagtacctgctgtgcctggggatgcccccggccctgtgcattgattatccctggcgctggagccgggccgtccccatgaactccgcactcatcaagtggctgtacctgcctgatttcttccgggcccccaactccaccaacctcatcagcgactttctcctgctgctgtgcgcctcccagcagtggcaggtgttctcagctgagcgcacagaggagtggcagcgcatggctggcgtcaacaccgaccgcctggagccgctgcggggggagcccaaccccgtgcccaactttatccactgcaggtcctaccttgacatgctgaaggtggccgtcttccgatacctgttctggctggtgctggtggtggtgtttgtcacgggggccacccgcatcagcatcttcgggctgggctacctgctggcctgcttctacctgctgctcttcggcacggccctgctgcagagggacacacgggcccgcctcgtgctgtgggactgcctcattctgtacaacgtcaccgtcatcatctccaagaacatgctgtcgctcctggcctgcgtcttcgtggagcagatgcagaccggcttctgctgggtcatccagctcttcagccttgtatgcaccgtcaagggctactatgaccccaaggagatgatggacagagaccaggactgcctgctgcctgtggaggaggctggcatcatctgggacagcgtctgcttcttcttcctgctgctgcagcgccgcgtcttccttagccattactacctgcacgtcagggccgacctccaggccaccgccctgctagcctccaggggcttcgccctctacaacgctgccaacctcaagagcattgactttcaccgcaggatagaggagaagtccctggcccagctgaaaagacagatggagcgtatccgtgccaagcaggagaagcacaggcagggccgggtggaccgcagtcgcccccaggacaccctgggccccaaggaccccggcctggagccagggcccgacagtccagggggctcctccccgccacggaggcagtggtggcggccctggctggaccacgccacagtcatccactccggggactacttcctgtttgagtccgacagtgaggaagaggaggaggctgttcctgaagacccgaggccgtcggcacagagtgccttccagctggcgtaccaggcatgggtgaccaacgcccaggcggtgctgaggcggcggcagcaggagcaggagcaggcaaggcaggaacaggcaggacagctacccacaggaggtggtcccagccaggaggtggagccagcagagggccccgaggaggcagcggcaggccggagccatgtggtgcagagggtgctgagcacggcgcagttcctgtggatgctggggcaggcgctagtggatgagctgacacgctggctgcaggagttcacccggcaccacggcaccatgagcgacgtgctgcgggcagagcgctacctcctcacacaggagctcctgcagggcggcgaagtgcacaggggcgtgctggatcagctgtacacaagccaggccgaggccacgctgccaggccccaccgaggcccccaatgccccaagcaccgtgtccagtgggctgggcgcggaggagccactcagcagcatgacagacgacatgggcagccccctgagcaccggctaccacacgcgcagtggcagtgaggaggcagtcaccgaccccggggagcgtgaggctggtgcctctctgtaccagggactgatgcggacggccagcgagctgctcctggacaggcgcctgcgcatcccagagctggaggaggcagagctgtttgcggaggggcagggccgggcgctgcggctgctgcgggccgtgtaccagtgtgtggccgcccactcggagctgctctgctacttcatcatcatcctcaaccacatggtcacggcctccgccggctcgctggtgctgcccgtgctcgtcttcctgtgggccatgctgtcgatcccgaggcccagcaagcgcttctggatgacggccatcgtcttcaccgagatcgcggtggtcgtcaagtacctgttccagtttgggttcttcccctggaacagccacgtggtgctgcggcgctacgagaacaagccctacttcccgccccgcatcctgggcctggagaagactgacggctacatcaagtacgacctggtgcagctcatggcccttttcttccaccgctcccagctgctgtgctatggcctctgggaccatgaggaggactcaccatccaaggagcatgacaagagcggcgaggaggagcagggagccgaggaggggccaggggtgcctgcggccaccaccgaagaccacattcaggtggaagccagggtcggacccacggacgggaccccagaaccccaagtggagctcaggccccgtgatacgaggcgcatcagtctacgttttagaagaaggaagaaggagggcccagcacggaaaggagcggcagccatcgaagctgaggacagggaggaagaagagggggaggaagagaaagaggcccccacggggagagagaagaggccaagccgctctggaggaagagtaagggcggccgggcggcggctgcagggcttctgcctgtccctggcccagggcacatatcggccgctacggcgcttcttccacgacatcctgcacaccaagtaccgcgcagccaccgacgtctatgccctcatgttcctggctgatgttgtcgacttcatcatcatcatttttggcttctgggcctttgggaagcactcggcggccacagacatcacgtcctccctatcagacgaccaggtacccgaggctttcctggtcatgctgctgatccagttcagtaccatggtggttgaccgcgccctctacctgcgcaagaccgtgctgggcaagctggccttccaggtggcgctggtgctggccatccacctatggatgttcttcatcctgcccgccgtcactgagaggatgttcaaccagaatgtggtggcccagctctggtacttcgtgaagtgcatctacttcgccctgtccgcctaccagatccgctgcggctaccccacccgcatcctcggcaacttcctcaccaagaagtacaatcatctcaacctcttcctcttccaggggttccggctggtgccgttcctggtggagctgcgggcagtgatggactgggtgtggacggacaccacgctgtccctgtccagctggatgtgtgtggaggacatctatgccaacatcttcatcatcaaatgcagccgagagacagagaagaaatacccgcagcccaaagggcagaagaagaagaagatcgtcaagtacggcatgggtggcctcatcatcctcttcctcatcgccatcatctggttcccactgctcttcatgtcgctggtgcgctccgtggttggggttgtcaaccagcccatcgatgtcaccgtcaccctgaagctgggcggctatgagccgctgttcaccatgagcgcccagcagccgtccatcatccccttcacggcccaggcctatgaggagctgtcccggcagtttgacccccagccgctggccatgcagttcatcagccagtacagccctgaggacatcgtcacggcgcagattgagggcagctccggggcgctgtggcgcatcagtccccccagccgtgcccagatgaagcgggagctctacaacggcacggccgacatcaccctgcgcttcacctggaacttccagagggacctggcgaagggaggcactgtggagtatgccaacgagaagcacatgctggccctggcccccaacagcactgcacggcggcagctggccagcctgctcgagggcacctcggaccagtctgtggtcatccctaatctcttccccaagtacatccgtgcccccaacgggcccgaagccaaccctgtgaagcagctgcagcccaatgaggaggccgactacctcggcgtgcgtatccagctgcggagggagcagggtgcgggggccaccggcttcctcgaatggtgggtcatcgagctgcaggagtgccggaccgactgcaacctgctgcccatggtcattttcagtgacaaggtcagcccaccgagcctcggcttcctggctggctacggcatcatggggctgtacgtgtccatcgtgctggtcatcggcaagttcgtgcgcggattcttcagcgagatctcgcactccattatgttcgaggagctgccgtgcgtggaccgcatcctcaagctctgccaggacatcttcctggtgcgggagactcgggagctggagctggaggaggagttgtacgccaagctcatcttcctctaccgctcactggagaccatgatcaagtggactcgtgagaaggagtaggagctgctgctggcgcccgagagggaaggagccggcctgctgggcagcgtggccacaaggggcggcactcctcaggccgggggagccactgccccgtccaaggccgccagctgtgatgcatcctcccggcctgcctgagccctgatgctgctgtcagagaaggacactgcgtccccacggcctgcgtggcgctgccgtcccccacgtgtactgtagagttttttttttaattaaaaaatgttttatttatacaaatggacaatcaga

SEQ ID NO: 14 - RNA, 1246 nt, Homo sapiens
  source 1..1246: mol_type = genomic RNA
atgaagatttgcccgttttcttctaaaagttctatagttttagctctgaagtttcgctctttgatccactttgagaaatacccgcagcccaaagggcagaagaagaagaagatcgtcaagtacggcatgggtggcctcatcatcctcttcctcatcgccatcatctggttcccactgctcttcatgtcgctggtgcgctccgtggttggggttgtcaaccagcccatcgatgtcaccgtcaccctgaagctgggcggctatgagccgctgttcaccatgagcgcccagcagccgtccatcatccccttcacggcccaggcctatgaggagctgtcccggcagtttgacccccagccgctggccatgcagttcatcagccagtacagccctgaggacatcgtcacggcgcagattgagggcagctccggggcgctgtggcgcatcagtccccccagccgtgcccagatgaagcgggagctctacaacggcacggccgacatcaccctgcgcttcacctggaacttccagagggacctggcgaagggaggcactgtggagtatgccaacgagaagcacatgctggccctggcccccaacagcactgcacggcggcagctggccagcctgctcgagggcacctcggaccagtctgtggtcatccctaatctcttccccaagtacatccgtgcccccaacgggcccgaagccaaccctgtgaagcagctgcagcccaatgaggaggccgactacctcggcgtgcgtatccagctgcggagggagcagggtgcgggggccaccggcttcctcgaatggtgggtcatcgagctgcaggagtgccggaccgactgcaacctgctgcccatggtcattttcagtgacaagcatcatggggctgtacgtgtccatcgtgctggtcatcggcaagttcgtgcgcggattcttcagcgagatctcgcactccattatgttcgaggagctgccgtgcgtggaccgcatcctcaagctctgccaggacatcttcctggtgcgggagactcgggagctggagctggaggaggagttgtacgccaagctcatcttcctctaccgctcactggagaccatgatcaagtggactcgtgagaaggagtaggagctgctgctggcgcccgagagggaaggagccggcctgctgggcagcgtggccacaaggggcggcactcctcaggccgggggagccactgccccgtccaaggccgccagctgt

SEQ ID NO: 15 - RNA, 6401 nt, Homo sapiens
  source 1..6401: mol_type = genomic RNA
ccatggacctgcgccctgagctgcccaccaccctgggccccgtcagcctgcgccagctggggctggagcacacccgctacccctgtctggaccttggtgccatgttgctctacaccctgaccttctggctcctgctgcgccagtttgtgaaagagaagctgctgaagtgggcagagtctccagctgcgctgacggaggtcaccgtggcagacacagagcctacgcggacgcagacgctgttgcagagcctgggggagctggtgaagggcgtgtacgccaagtactggatctatgtgtgtgctggcatgttcatcgtggtcagcttcgccggccgcctcgtggtctacaagattgtctacatgttcctcttcctgctctgcctcaccctcttccaggtctactacagcctgtggcggaagctgctcaaggccttctggtggctcgtggtggcctacaccatgctggtcctcatcgccgtctacaccttccagttccaggacttccctgcctactggcgcaacctcactggcttcaccgacgagcagctgggggacctgggcctggagcagttcagcgtatccgagctcttctccagcatcctggtgcccggcttcttcctcctggcctgcatcctgcagctgcactacttccacaggcccttcatgcagctcaccgacatggagcacgtgtccctgcctggcacgcgcctcccgcgctgggctcacaggcaggatgcagtgagtgggaccccactgctgcgggaggagcagcaggagcatcagcagcagcagcaggaggaggaggaggaggaggactccagggacgaggggctgggcgtggccactccccaccaggccacgcaggtgcctgaaggggcagccaagtggggcctggtggctgagcgcctgctggagctggcagccggcttctcggacgtcctctcacgcgtgcaggtgttcctgcggcggctgctggagcttcacgttttcaagctggtggccctgtacaccgtctgggtggccctgaaggaggtgtcggtgatgaacctgctgctggtggtgctgtgggccttcgccctgccctacccacgcttccggcccatggcctcctgcctgtccaccgtgtggacctgcgtcatcatcgtgtgtaagatgctgtaccagctcaaggttgtcaacccccaggagtattccagcaactgcaccgagcccttccccaacagcaccaacttgctgcccacggagatcagccagtccctgctgtaccgggggcccgtggaccctgccaactggtttggggtgcggaaagggttccccaacctgggctacatccagaaccacctgcaagtgctgctgctgctggtattcgaggccatcgtgtaccggcgccaggagcactaccgccggcagcaccagctggccccgctgcctgcccaggccgtgtttgccagcggcacccgccagcagctggaccaggatctgctcggctgcctcaagtacttcatcaacttcttcttctacaaattcgggctggagatctgcttcctgatggccgtgaacgtgatcgggcagcgcatgaactttctggtgaccctgcacggttgctggctggtggccatcctcacccgcaggcaccgccaggccattgcccgcctctggcccaactactgcctcttcctggcgctgttcctgctgtaccagtacctgctgtgcctggggatgcccccggccctgtgcattgattatccctggcgctggagccgggccgtccccatgaactccgcactcatcaagtggctgtacctgcctgatttcttccgggcccccaactccaccaacctcatcagcgactttctcctgctgctgtgcgcctcccagcagtggcaggtgttctcagctgagcgcacagaggagtggcagcgcatggctggcgtcaacaccgaccgcctggagccgctgcggggggagcccaaccccgtgcccaactttatccactgcaggtcctaccttgacatgctgaaggtggccgtcttccgatacctgttctggctggtgctggtggtggtgtttgtcacgggggccacccgcatcagcatcttcgggctgggctacctgctggcctgcttctacctgctgctcttcggcacggccctgctgcagagggacacacgggcccgcctcgtgctgtgggactgcctcattctgtacaacgtcaccgtcatcatctccaagaacatgctgtcgctcctggcctgcgtcttcgtggagcagatgcagaccggcttctgctgggtcatccagctcttcagccttgtatgcaccgtcaagggctactatgaccccaaggagatgatggacagagaccaggactgcctgctgcctgtggaggaggctggcatcatctgggacagcgtctgcttcttcttcctgctgctgcagcgccgcgtcttccttagccattactacctgcacgtcagggccgacctccaggccaccgccctgctagcctccaggggcttcgccctctacaacgctgccaacctcaagagcattgacttccaccgcaggatagaggagaagtccctggcccagctgaaaagacagatggagcgtatccgtgccaagcaggagaagcacaggcagggccgggtggaccgcagtcgcccccaggacaccctgggccccaaggaccccggcctggagccagggcccgacagtccagggggctcctccccgccacggaggcagtggtggcggccctggctggaccacgccacagtcatccactccggggactacttcctgtttgagtccgacagtgaggaagaggaggaggctgttcctgaagacccgaggccgtcggcacagagtgccttccagctggcgtaccaggcatgggtgaccaacgcccaggcggtgctgaggcggcggcagcaggagcaggagcaggcaaggcaggaacaggcaggacagctacccacaggaggtggtcccagccaggaggtggagccagcagagggccccgaggaggcagcggcaggccggagccatgtggtgcagagggtgctgagcacggcgcagttcctgtggatgctggggcaggcgctagtggatgagctgacacgctggctgcaggagttcacccggcaccacggcaccatgagcgacgtgctgcgggcagagcgctacctcctcacacaggagctcctgcagggcggcgaagtgcacaggggcgtgctggatcagctgtacacaagccaggccgaggccacgctgccaggccccaccgaggcccccaatgccccaagcaccgtgtccagtgggctgggcgcggaggagccactcagcagcatgacagacgacatgggcagccccctgagcaccggctaccacacgcgcagtggcagtgaggaggcagtcaccgaccccggggagcgtgaggctggtgcctctctgtaccagggactgatgcggacggccagcgagctgctcctggacaggcgcctgcgcatcccagagctggaggaggcagagctgtttgcggaggggcagggccgggcgctgcggctgctgcgggccgtgtaccagtgtgtggccgcccactcggagctgctctgctacttcatcatcatcctcaaccacatggtcacggcctccgccggctccctggtgctgcccgtgctcgtcttcctgtgggccatgctgtcgatcccgaggcccagcaagcgcttctggatgacggccatcgtcttcaccgagatcgcggtggtcgtcaagtacctgttccagtttgggttcttcccctggaacagccacgtggtgctgcggcgctacgagaacaagccctacttcccgccccgcatcctgggcctggagaagactgacggctacatcaagtacgacctggtgcagctcatggcccttttcttccaccgctcccagctgctgtgctatggcctctgggaccatgaggaggactcaccatccaaggagcatgacaagagcggcgaggaggagcagggagccgaggaggggccaggggtgcctgcggccaccaccgaagaccacattcaggtggaagcgagggtcggacccacggacgggaccccagaaccccaagtggagctcaggccccgtgatacgaggcgcatcagtctacgttttagaagaaggaagaaggagggcccagcacggaaaggagcggcagccatcgaagctgaggacagggaggaagaagagggggaggaagagaaagaggcccccacggggagagagaagaggccaagccgctctggaggaagagtaagggcggccgggcggcggctgcagggcttctgcctgtccctggcccagggcacatatcggccgctacggcgcttcttccacgacatcctgcacaccaagtaccgcgcagccaccgacgtctatgccctcatgttcctggctgatgttgtcgacttcatcatcatcatttttggcttctgggcctttgggaagcactcggcggccacagacatcacgtcctccctatcagacgaccaggtacccgaggctttcctggtcatgctgctgatccagttcagtaccatggtggttgaccgcgccctctacctgcgcaagaccgtgctgggcaagctggccttccaggtggcgctggtgctggccatccacctatggatgttcttcatcctgcccgccgtcactgagaggatgttcaaccagaatgtggtggcccagctctggtacttcgtgaagtgcatctacttcgccctgtccgcctaccagatccgctgcggctaccccacccgcatcctcggcaacttcctcaccaagaagtacaatcatctcaacctcttcctcttccaggggttccggctggtgccgttcctggtggagctgcgggcagtgatggactgggtgtggacggacaccacgctgtccctgtccagctggatgtgtgtggaggacatctatgccaacatcttcatcatcaaatgcagccgagagacagagaagaaatacccgcagcccaaagggcagaagaagaagaagatcgtcaagtacggcatgggtggcctcatcatcctcttcctcatcgccatcatctggttcccgctgctcttcatgtcgctggtgcgctccgtggttggggttgtcaaccagcccatcgatgtcaccgtcaccctcaagctgggcggctatgagccgctgttcaccatgagcgcccagcagccgtccatcatccccttcacggcccaggcctatgaggagctgtcccggcagtttgacccccagccgctggccatgcagttcatcagccagtacagccctgaggacatcgtcacggcgcagattgagggcagctccggggcgctgtggcgcatcagtccccccagccgtgcccagatgaagcgggagctctacaacggcacggccgacatcaccctgcgcttcacctggaacttccagagggacctggcgaagggaggcactgtggagtatgccaacgagaagcacatgctggccctggcccccaacagcactgcacggcggcagctggccagcctgctcgagggcacctcggaccagtctgtggtcatccccaatctcttccccaagtacatccgtgcccccaacgggcccgaagccaaccctgtgaagcagctgcagcccaatgaggaggccgactacctcggcgtgcgtatccagctgcggagggagcagggtgcgggggccaccggcttcctcgaatggtgggtcatcgagctgcaggagtgccggaccgactgcaacctgctgcccatggtcattttcagtgacaaggtcagcccaccgagcctcggcttcctggctggctacggcatcatggggctgtacgtgtccatcgtgctggtcatcggcaagttcgtgcgcggattcttcagcgagatctcgcactccattatgttcgaggagctgccgtgcgtggaccgcatcctcaagctctgccaggacatcttcctggtgcgggagactcgggagctggagctggaggaggagttgtacgccaagctcatcttcctctaccgctcactggagaccatgatcaagtggactcgtgagaaggagtaggagctgctgctggcgcccgagagggaaggagccggcctgctgggcagcgtggccacaaggggcggcactcctcaggccgggggagccactgccccgtccaaggccgccagctgtgatgcatcctcccggcctgcctgagccctgatgctgctgtcagagaaggacactgcgtccccacggcctgcgtggcgctgccgtcccccacgtgtactgtagagttttttttttaattaaaaaatgttttatttatacaaatggaaaaaaaaaaaaaaaaaaaaaaaaaaaaaaaaa

SEQ ID NO: 16 - RNA, 1655 nt, Homo sapiens
  source 1..1655: mol_type = genomic RNA
taccagatccgctgcggctaccccacccgcatcctcggcaacttcctcaccaagaagtacaatcatctcaacctcttcctcttccaggggttccggctggtgccgttcctggtggagctgcgggcagtgatggactgggtgtggacggacaccacgctgtccctgtccagctggatgtgtgtggaggacatctatgccaacatcttcatcatcaaatgcagccgagagacagagaaggttcatgaagatttgcccgttttcttctaaaagttctatagttttagctctgaagtttcgctctttgatccactttgagaaatacccgcagcccaaagggcagaagaagaagaagatcgtcaagtacggcatgggtggcctcatcatcctcttcctcatcgccatcatctggttcccgctgctcttcatgtcgctggtgcgctccgtggttggggttgtcaaccagcccatcgatgtcaccgtcaccctcaagctgggcggctatgagccgctgttcaccatgagcgcccagcagccgtccatcatccccttcacggcccaggcctatgaggagctgtcccggcagtttgacccccagccgctggccatgcagttcatcagccagtacagccctgaggacatcgtcacggcgcagattgagggcagctccggggcgctgtggcgcatcagtccccccagccgtgcccagatgaagcgggagctctacaacggcacggccgacatcaccctgcgcttcacctggaacttccagagggacctggcgaagggaggcactgtggagtatgccaacgagaagcacatgctggccctggcccccaacagcactgcacggcggcagctggccagcctgctcgagggcacctcggatcagtctgtggtcatccccaatctcttccccaagtacatccgtgcccccaacgggcccgaagccaaccctgtgaagcagctgcagcccaatgaggaggccgactacctcggcgtgcgtatccagctgcggagggagcagggtgcgggggccaccggcttcctcgaatggtgggtcatcgagctgcaggagtgccggaccgactgcaacctgctgcccatggtcattttcagtgacaagcatcatggggctgtacgtgtccatcgtgctggtcatcggcaagttcgtgcgcggattcttcagcgagatctcgcactccattatgttcgaggagctgccgtgcgtggaccgcatcctcaagctctgccaggacatcttcctggtgcgggagactcgggagctggagctggaggaggagttgtatgccaagctcatcttcctctaccgctcactggagaccatgatcaagtggactcgtgagaaggagtaggagctgctgctggcgcccgagagggaaggagccggcctgctgggcagcgtggccacaaggggcggcactcctcaggccgggggagccactgccccgtccaaggccgccagctgtgatgcatcctcccggcctgcctgagccctgatgctgctgtcagagaaggacactgcgtccccacggcctgcgtggcgctgccgtcccccacgtgtactgtagagtttttttttttaattaaaaaatgttttatttatacaaatggaaaaaaaaaaaaaaaaaaaaaaa

SEQ ID NO: 17 - RNA, 6368 nt, Homo sapiens
  source 1..6368: mol_type = genomic RNA
ccatggacctgcgccctgagctgcccaccaccctgggccccgtcagcctgcgccagctggggctggagcacacccgctacccctgtctggaccttggtgccatgttgctctacaccctgaccttctggctcctgctgcgccagtttgtgaaagagaagctgctgaagtgggcagagtctccagctgcgctgacggaggtcaccgtggcagacacagagcctacgcggacgcagacgctgttgcagagcctgggggagctggtgaagggcgtgtacgccaagtactggatctatgtgtgtgctggcatgttcatcgtggtcagcttcgccggccgcctcgtggtctacaagattgtctacatgttcctcttcctgctctgcctcaccctcttccaggtctactacagcctgtggcggaagctgctcaaggccttctggtggctcgtggtggcctacaccatgctggtcctcatcgccgtctacaccttccagttccaggacttccctgcctactggcgcaacctcactggcttcaccgacgagcagctgggggacctgggcctggagcagttcagcgtatccgagctcttctccagcatcctggtgcccggcttcttcctcctggcctgcatcctgcagctgcactacttccacaggcccttcatgcagctcaccgacatggagcacgtgtccctgcctggcacgcgcctcccgcgctgggctcacaggcaggatgcagtgagtgggaccccactgctgcgggaggagcagcaggagcatcagcagcagcagcaggaggaggaggaggaggaggactccagggacgaggggctgggcgtggccactccccaccaggccacgcaggtgcctgaaggggcagccaagtggggcctggtggctgagcgcctgctggagctggcagccggcttctcggacgtcctctcacgcgtgcaggtgttcctgcggcggctgctggagcttcacgttttcaagctggtggccctgtacaccgtctgggtggccctgaaggaggtgtcggtgatgaacctgctgctggtggtgctgtgggccttcgccctgccctacccacgcttccggcccatggcctcctgcctgtccaccgtgtggacctgcgtcatcatcgtgtgtaagatgctgtaccagctcaaggttgtcaacccccaggagtattccagcaactgcaccgagcccttccccaacagcaccaacttgctgcccacggagatcagccagtccctgctgtaccgggggcccgtggaccctgccaactggtttggggtgcggaaagggttccccaacctgggctacatccagaaccacctgcaagtgctgctgctgctggtattcgaggccatcgtgtaccggcgccaggagcactaccgccggcagcaccagctggccccgctgcctgcccaggccgtgtttgccagcggcacccgccagcagctggaccaggatctgctcggctgcctcaagtacttcatcaacttcttcttctacaaattcgggctggagatctgcttcctgatggccgtgaacgtgatcgggcagcgcatgaactttctggtgaccctgcacggttgctggctggtggccatcctcacccgcaggcaccgccaggccattgcccgcctctggcccaactactgcctcttcctggcgctgttcctgctgtaccagtacctgctgtgcctggggatgcccccggccctgtgcattgattatccctggcgctggagccgggccgtccccatgaactccgcactcatcaagtggctgtacctgcctgatttcttccgggcccccaactccaccaacctcatcagcgactttctcctgctgctgtgcgcctcccagcagtggcaggtgttctcagctgagcgcacagaggagtggcagcgcatggctggcgtcaacaccgaccgcctggagccgctgcggggggagcccaaccccgtgcccaactttatccactgcaggtcctaccttgacatgctgaaggtggccgtcttccgatacctgttctggctggtgctggtggtggtgtttgtcacgggggccacccgcatcagcatcttcgggctgggctacctgctggcctgcttctacctgctgctcttcggcacggccctgctgcagagggacacacgggcccgcctcgtgctgtgggactgcctcattctgtacaacgtcaccgtcatcatctccaagaacatgctgtcgctcctggcctgcgtcttcgtggagcagatgcagaccggcttctgctgggtcatccagctcttcagccttgtatgcaccgtcaagggctactatgaccccaaggagatgatggacagagaccaggactgcctgctgcctgtggaggaggctggcatcatctgggacagcgtctgcttcttcttcctgctgctgcagcgccgcgtcttccttagccattactacctgcacgtcagggccgacctccaggccaccgccctgctagcctccaggggcttcgccctctacaacgctgccaacctcaagagcattgacttccaccgcaggatagaggagaagtccctggcccagctgaaaagacagatggagcgtatccgtgccaagcaggagaagcacaggcagggccgggtggaccgcagtcgcccccaggacaccctgggccccaaggaccccggcctggagccagggcccgacagtccagggggctcctccccgccacggaggcagtggtggcggccctggctggaccacgccacagtcatccactccggggactacttcctgtttgagtccgacagtgaggaagaggaggaggctgttcctgaagacccgaggccgtcggcacagagtgccttccagctggcgtaccaggcatgggtgaccaacgcccaggcggtgctgaggcggcggcagcaggagcaggagcaggcaaggcaggaacaggcaggacagctacccacaggaggtggtcccagccaggaggtggagccagcagagggccccgaggaggcagcggcaggccggagccatgtggtgcagagggtgctgagcacggcgcagttcctgtggatgctggggcaggcgctagtggatgagctgacacgctggctgcaggagttcacccggcaccacggcaccatgagcgacgtgctgcgggcagagcgctacctcctcacacaggagctcctgcagggcggcgaagtgcacaggggcgtgctggatcagctgtacacaagccaggccgaggccacgctgccaggccccaccgaggcccccaatgccccaagcaccgtgtccagtgggctgggcgcggaggagccactcagcagcatgacagacgacatgggcagccccctgagcaccggctaccacacgcgcagtggcagtgaggaggcagtcaccgaccccggggagcgtgaggctggtgcctctctgtaccagggactgatgcggacggccagcgagctgctcctggacaggcgcctgcgcatcccagagctggaggaggcagagctgtttgcggaggggcagggccgggcgctgcggctgctgcgggccgtgtaccagtgtgtggccgcccactcggagctgctctgctacttcatcatcatcctcaaccacatggtcacggcctccgccggctccctggtgctgcccgtgctcgtcttcctgtgggccatgctgtcgatcccgaggcccagcaagcgcttctggatgacggccatcgtcttcaccgagatcgcggtggtcgtcaagtacctgttccagtttgggttcttcccctggaacagccacgtggtgctgcggcgctacgagaacaagccctacttcccgccccgcatcctgggcctggagaagactgacggctacatcaagtacgacctggtgcagctcatggcccttttcttccaccgctcccagctgctgtgctatggcctctgggaccatgaggaggactcaccatccaaggagcatgacaagagcggcgaggaggagcagggagccgaggaggggccaggggtgcctgcggccaccaccgaagaccacattcaggtggaagcgagggtcggacccacggacgggaccccagaaccccaagtggagctcaggccccgtgatacgaggcgcatcagtctacgttttagaagaaggaagaaggagggcccagcacggaaaggagcggcagccatcgaagctgaggacagggaggaagaagagggggaggaagagaaagaggcccccacggggagagagaagaggccaagccgctctggaggaagagtaagggcggccgggcggcggctgcagggcttctgcctgtccctggcccagggcacatatcggccgctacggcgcttcttccacgacatcctgcacaccaagtaccgcgcagccaccgacgtctatgccctcatgttcctggctgatgttgtcgacttcatcatcatcatttttggcttctgggcctttgggaagcactcggcggccacagacatcacgtcctccctatcagacgaccaggtacccgaggctttcctggtcatgctgctgatccagttcagtaccatggtggttgaccgcgccctctacctgcgcaagaccgtgctgggcaagctggccttccaggtggcgctggtgctggccatccacctatggatgttcttcatcctgcccgccgtcactgagaggatgttcaaccagaatgtggtggcccagctctggtacttcgtgaagtgcatctacttcgccctgtccgcctaccagatccgctgcggctaccccacccgcatcctcggcaacttcctcaccaagaagtacaatcatctcaacctcttcctcttccaggggttccggctggtgccgttcctggtggagctgcgggcagtgatggactgggtgtggacggacaccacgctgtccctgtccagctggatgtgtgtggaggacatctatgccaacatcttcatcatcaaatgcagccgagagacagagaagaaatacccgcagcccaaagggcagaagaagaagaagatcgtcaagtacggcatgggtggcctcatcatcctcttcctcatcgccatcatctggttcccgctgctcttcatgtcgctggtgcgctccgtggttggggttgtcaaccagcccatcgatgtcaccgtcaccctcaagctgggcggctatgagccgctgttcaccatgagcgcccagcagccgtccatcatccccttcacggcccaggcctatgaggagctgtcccggcagtttgacccccagccgctggccatgcagttcatcagccagtacagccctgaggacatcgtcacggcgcagattgagggcagctccggggcgctgtggcgcatcagtccccccagccgtgcccagatgaagcgggagctctacaacggcacggccgacatcaccctgcgcttcacctggaacttccagagggacctggcgaagggaggcactgtggagtatgccaacgagaagcacatgctggccctggcccccaacagcactgcacggcggcagctggccagcctgctcgagggcacctcggaccagtctgtggtcatccccaatctcttccccaagtacatccgtgcccccaacgggcccgaagccaaccctgtgaagcagctgcagcccaatgaggaggccgactacctcggcgtgcgtatccagctgcggagggagcagggtgcgggggccaccggcttcctcgaatggtgggtcatcgagctgcaggagtgccggaccgactgcaacctgctgcccatggtcattttcagtgacaaggtcagcccaccgagcctcggcttcctggctggctacggcatcatggggctgtacgtgtccatcgtgctggtcatcggcaagttcgtgcgcggattcttcagcgagatctcgcactccattatgttcgaggagctgccgtgcgtggaccgcatcctcaagctctgccaggacatcttcctggtgcgggagactcgggagctggagctggaggaggagttgtacgccaagctcatcttcctctaccgctcactggagaccatgatcaagtggactcgtgagaaggagtaggagctgctgctggcgcccgagagggaaggagccggcctgctgggcagcgtggccacaaggggcggcactcctcaggccgggggagccactgccccgtccaaggccgccagctgtgatgcatcctcccggcctgcctgagccctgatgctgctgtcagagaaggacactgcgtccccacggcctgcgtggcgctgccgtcccccacgtgtactgtagagttttttttttaattaaaaaatgttttatttatacaaatgg

SEQ ID NO: 18 - RNA, 6273 nt, Homo sapiens
  source 1..6273: mol_type = genomic RNA
atggtatggagcatcacctaccacagctggctgaccttcgtactgctgctctgggcctgcctcatctggacggtgcgcagccgccaccaactggccatgctgtgctcgccctgcatcctgctgtatgggatgacgctgtgctgcctacgctacgtgtgggccatggacctgcgccctgagctgcccaccaccctgggccccgtcagcctgcgccagctggggctggagcacacccgctacccctgtctggaccttggtgccatgttgctctacaccctgaccttctggctcctgctgcgccagtttgtgaaagagaagctgctgaagtgggcagagtctccagctgcgctgacggaggtcaccgtggcagacacagagcccacgcggacgcagacgctgttgcagagcctgggggagctggtgaagggcgtgtacgccaagtactggatctatgtgtgtgctggcatgttcatcgtggtcagcttcgccggccgcctcgtggtctacaagattgtctacatgttcctcttcctgctctgcctcaccctcttccaggtctactacagcctgtggcggaagctgctcaaggccttctggtggctcgtggtggcctacaccatgctggtcctcatcgccgtctacaccttccagttccaggacttccctgcctactggcgcaacctcactggcttcaccgacgagcagctgggggacctgggcctggagcagttcagcgtgtccgagctcttctccagcatcctggtgcccggcttcttcctcctggcctgcatcctgcagctgcactacttccacaggcccttcatgcagctcaccgacatggagcacgtgtccctgcctggcacgcgcctcccgcgctgggctcacaggcaggatgcagtgagtgggaccccactgctgcgggaggagcagcaggagcatcagcagcagcagcaggaggaggaggaggaggaggaggactccagggacgaggggctgggcgtggccactccccaccaggccacgcaggtgcctgaaggggcagccaagtggggcctggtggctgagcggctgctggagctggcagccggcttctcggacgtcctctcacgcgtgcaggtgttcctgcggcggctgctggagcttcacgttttcaagctggtggccctgtacaccgtctgggtggccctgaaggaggtgtcggtgatgaacctgctgctggtggtgctgtgggccttcgccctgccctacccacgcttccggcccatggcctcctgcctgtccaccgtgtggacctgcgtcatcatcgtgtgtaagatgctgtaccagctcaaggttgtcaacccccaggagtattccagcaactgcaccgagcccttccccaacagcaccaacttgctgcccacggagatcagccagtccctgctgtaccgggggcccgtggaccctgccaactggtttggggtgcggaaagggttccccaacctgggctacatccagaaccacctgcaagtgctgctgctgctggtattcgaggccatcgtgtaccggcgccaggagcactaccgccggcagcaccagctggccccgctgcctgcccaggccgtgtttgccagcggcacccgccagcagctggaccaggatctgctcggctgcctcaagtacttcatcaacttcttcttctacaaattcgggctggagatctgcttcctgatggccgtgaacgtgatcgggcagcgcatgaactttctggtgaccctgcacggttgctggctggtggccatcctcacccgcaggcaccgccaggccattgcccgcctctggcccaactactgcctcttcctggcgctgttcctgctgtaccagtacctgctgtgcctggggatgcccccggccctgtgcattgattatccctggcgctggagccgggccgtccccatgaactccgcactcatcaagtggctgtacctgcctgatttcttccgggcccccaactccaccaacctcatcagcgactttctcctgctgctgtgcgcctcccagcagtggcaggtgttctcagctgagcgcacagaggagtggcagcgcatggctggcgtcaacaccgaccgcctggagccgctgcggggggagcccaaccccgtgcccaactttatccactgcaggtcctaccttgacatgctgaaggtggccgtcttccgatacctgttctggctggtgctggtggtggtgtttgtcacgggggccacccgcatcagcatcttcgggctgggctacctgctggcctgcttctacctgctgctcttcggcacggccctgctgcagagggacacacgggcccgcctcgtgctgtgggactgcctcattctgtacaacgtcaccgtcatcatctccaagaacatgctgtcgctcctggcctgcgtcttcgtggagcagatgcagaccggcttctgctgggtcatccagctcttcagccttgtatgcaccgtcaagggctactatgaccccaaggagatgatggacagagaccaggactgcctgctgcctgtggaggaggctggcatcatctgggacagcgtctgcttcttcttcctgctgctgcagcgccgcgtcttccttagccattactacctgcacgtcagggccgacctccaggccaccgccctgctagcctccaggggcttcgccctctacaacgctgccaacctcaagagcattgacttccaccgcaggatagaggagaagtccctggcccagctgaaaagacagatggagcgtatccgtgccaagcaggagaagcacaggcagggccgggtggaccgcagtcgcccccaggacaccctgggccccaaggaccccggcctggagccagggcccgacagtccagggggctcctccccgccacggaggcagtggtggcggccctggctggaccacgccacagtcatccactccggggactacttcctgtttgagtccgacagtgaggaagaggaggaggctgttcctgaagacccgaggccgtcggcacagagtgccttccagctggcgtaccaggcatgggtgaccaacgcccaggcggtgctgaggcggcggcagcaggagcaggagcaggcaaggcaggaacaggcaggacagctacccacaggaggtggtcccagccaggaggtggagccagcagagggccccgaggaggcagcggcaggccggagccatgtggtgcagagggtgctgagcacggcgcagttcctgtggatgctggggcaggcgctagtggatgagctgacacgctggctgcaggagttcacccggcaccacggcaccatgagcgacgtgctgcgggcagagcgctacctcctcacacaggagctcctgcagggcggcgaagtgcacaggggcgtgctggatcagctgtacacaagccaggccgaggccacgctgccaggccccaccgaggcccccaatgccccaagcaccgtgtccagtgggctgggcgcggaggagccactcagcagcatgacagacgacatgggcagccccctgagcaccggctaccacacgcgcagtggcagtgaggaggcagtcaccgaccccggggagcgtgaggctggtgcctctctgtaccagggactgatgcggacggccagcgagctgctcctggacaggcgcctgcgcatcccagagctggaggaggcagagctgtttgcggaggggcagggccgggcgctgcggctgctgcgggccgtgtaccagtgtgtggccgcccactcggagctgctctgctacttcatcatcatcctcaaccacatggtcacggcctccgccggctcgctggtgctgcccgtgctcgtcttcctgtgggccatgctgtcgatcccgaggcccagcaagcgcttctggatgacggccatcgtcttcaccgagatcgcggtggtcgtcaagtacctgttccagtttgggttcttcccctggaacagccacgtggtgctgcggcgctacgagaacaagccctacttcccgccccgcatcctgggcctggagaagactgacggctacatcaagtacgacctggtgcagctcatggcccttttcttccaccgctcccagctgctgtgctatggcctctgggaccatgaggaggactcaccatccaaggagcatgacaagagcggcgaggaggagcagggagccgaggaggggccaggggtgcctgcggccaccaccgaagaccacattcaggtggaagcgagggtcggacccacggacgggaccccagaaccccaagtggagctcaggccccgtgatacgaggcgcatcagtctacgttttagaagaaggaagaaggagggcccagcacggaaaggagcggcagccatcgaagctgaggacagggaggaagaagagggggaggaagagaaagaggcccccacggggagagagaagaggccaagccgctctggaggaagagtaagggcggccgggcggcggctgcagggcttctgcctgtccctggcccagggcacatatcggccgctacggcgcttcttccacgacatcctgcacaccaagtaccgcgcagccaccgacgtctatgccctcatgttcctggctgatgttgtcgacttcatcatcatcatttttggcttctgggcctttgggaagcactcggcggccacagacatcacgtcctccctatcagacgaccaggtacccgaggctttcctggtcatgctgctgatccagttcagtaccatggtggttgaccgcgccctctacctgcgcaagaccgtgctgggcaagctggccttccaggtggcgctggtgctggccatccacctatggatgttcttcatcctgcccgccgtcactgagaggatgttcaaccagaatgtggtggcccagctctggtacttcgtgaagtgcatctacttcgccctgtccgcctaccagatccgctgcggctaccccacccgcatcctcggcaacttcctcaccaagaagtacaatcatctcaacctcttcctcttccaggggttccggctggtgccgttcctggtggagctgcgggcagtgatggactgggtgtggacggacaccacgctgtccctgtccagctggatgtgtgtggaggacatctatgccaacatcttcatcatcaaatgcagccgagagacagagaagaaatacccgcagcccaaagggcagaagaagaagaagatcgtcaagtacggcatgggtggcctcatcatcctcttcctcatcgccatcatctggttcccgctgctcttcatgtcgctggtgcgctccgtggttggggttgtcaaccagcccatcgatgtcaccgtcaccctgaagctgggcggctatgagccgctgttcaccatgagcgcccagcagccgtccatcatccccttcacggcccaggcctatgaggagctgtcccggcagtttgacccccagccgctggccatgcagttcatcagccagtacagccctgaggacatcgtcacggcgcagattgagggcagctccggggcgctgtggcgcatcagtccccccagccgtgcccagatgaagcgggagctctacaacggcacggccgacatcaccctgcgcttcacctggaacttccagagggacctggcgaagggaggcactgtggagtatgccaacgagaagcacatgctggccctggcccccaacagcactgcacggcggcagctggccagcctgctcgagggcacctcggaccagtctgtggtcatccccaatctcttccccaagtacatccgtgcccccaacgggcccgaagccaaccctgtgaagcagctgcagcccaatgaggaggccgactacctcggcgtgcgtatccagctgcggagggagcagggtgcgggggccaccggcttcctcgaatggtgggtcatcgagctgcaggagtgccggaccgactgcaacctgctgcccatggtcattttcagtgacaaggtcagcccaccgagcctcggcttcctggctggctacggcatcatggggctgtacgtgtccatcgtgctggtcatcggcaagttcgtgcgcggattcttcagcgagatctcgcactccattatgttcgaggagctgccgtgcgtggaccgcatcctcaagctctgccaggacatcttcctggtgcgggagactcgggagctggagctggaggaggagttgtacgccaagctcatcttcctctaccgctcactggagaccatgatcaagtggactcgtgagaaggagtag

SEQ ID NO: 19 - RNA, 7563 nt, Homo sapiens
  source 1..7563: mol_type = genomic RNA
atggagccgcacgtgttcggcgcggtcctgtactggctgctgctgccctgcgcgctgctggctgcctgcctgctccgcttcagcggactctcgctggtctacctgctcttcctgctgctgctgccctggttccccggccccacccgatgcggcctccaaggtcacacaggccgcctcctgcgggcattgctgggcctcagcctgctcttcctggtggcccatctcgccctccagatctgcctgcatactgtgccccgcctggaccagctcctgggacccagctgcagccgctgggagaccctctcgcgacacataggggtcacaaggctggacctgaaggacatccccaacgccatccggctggtggcccctgacctgggcatcttggtggtctcctctgtctgcctcggcatctgcgggcgccttgcaaggaacacccggcagagcccacatccacgggagctggatgatgatgagagggatgtggatgccagcccgacggcagggctgcaggaagcagcaacgctggcccctacacggaggtcacggctggccgctcgtttccgagtcacggcccactggctgctggtggcggctgggcgggtcctggccgtaacactgcttgcactggcaggcatcgcccacccctcggccctctccagtgtctacctgctgctcttcctggccctctgcacctggtgggcctgccactttcccatcagcactcggggcttcagcagactctgcgccgcggtggggtgcttcggcgccggccatctcatctgcctctactgctaccagatgcccttggcacaggctctgctcccgcctgccggcatctgggctagggtgctgggtctcaaggacttcgtgggtcccaccaactgctccagcccccacgcgctggtcctcaacaccggcctggactggcctgtgtatgccagccccggcgtcctcctgctgctgtgctacgccacggcctctctgcgcaagctccgcgcgtaccgcccctccggccagaggaaggaggcggcaaaggggtatgaggctcgggagctggagctagcagagctggaccagtggccccaggaacgggagtctgaccagcacgtggtgcccacagcacccgacaccgaggctgataactgcatcgtgcacgagctgaccggccagagctccgtcctgcggcggcctgtgcggcccaagcgggctgagcccagggaggcgtctccgctccacagcctgggccacctcatcatggaccagagctatgtgtgcgcgctcattgccatgatggtatggagcatcacctaccacagctggctgaccttcgtactgctgctctgggcctgcctcatctggacggtgcgcagccgccaccaactggccatgctgtgctcgccctgcatcctgctgtatgggatgacgctgtgctgcctacgctacgtgtgggccatggacctgcgccctgagctgcccaccaccctgggccccgtcagcctgcgccagctggggctggagcacacccgctacccctgtctggaccttggtgccatgttgctctacaccctgaccttctggctcctgctgcgccagtttgtgaaagagaagctgctgaagtgggcagagtctccagctgcgctgacggaggtcaccgtggcagacacagagcccacgcggacgcagacgctgttgcagagcctgggggagctggtgaagggcgtgtacgccaagtactggatctatgtgtgtgctggcatgttcatcgtggtcagcttcgccggccgcctcgtggtctacaagattgtctacatgttcctcttcctgctctgcctcaccctcttccaggtctactacagcctgtggcggaagctgctcaaggccttctggtggctcgtggtggcctacaccatgctggtcctcatcgccgtctacaccttccagttccaggacttccctgcctactggcgcaacctcactggcttcaccgacgagcagctgggggacctgggcctggagcagttcagcgtgtccgagctcttctccagcatcctggtgcccggcttcttcctcctggcctgcatcctgcagctgcactacttccacaggcccttcatgcagctcaccgacatggagcacgtgtccctgcctggcacgcgcctcccgcgctgggctcacaggcaggatgcagtgagtgggaccccactgctgcgggaggagcagcaggagcatcagcagcagcagcaggaggaggaggaggaggaggaggactccagggacgaggggctgggcgtggccactccccaccaggccacgcaggtgcctgaaggggcagccaagtggggcctggtggctgagcggctgctggagctggcagccggcttctcggacgtcctctcacgcgtgcaggtgttcctgcggcggctgctggagcttcacgttttcaagctggtggccctgtacaccgtctgggtggccctgaaggaggtgtcggtgatgaacctgctgctggtggtgctgtgggccttcgccctgccctacccacgcttccggcccatggcctcctgcctgtccaccgtgtggacctgcgtcatcatcgtgtgtaagatgctgtaccagctcaaggttgtcaacccccaggagtattccagcaactgcaccgagcccttccccaacagcaccaacttgctgcccacggagatcagccagtccctgctgtaccgggggcccgtggaccctgccaactggtttggggtgcggaaagggttccccaacctgggctacatccagaaccacctgcaagtgctgctgctgctggtattcgaggccatcgtgtaccggcgccaggagcactaccgccggcagcaccagctggccccgctgcctgcccaggccgtgtttgccagcggcacccgccagcagctggaccaggatctgctcggctgcctcaagtacttcatcaacttcttcttctacaaattcgggctggagatctgcttcctgatggccgtgaacgtgatcgggcagcgcatgaactttctggtgaccctgcacggttgctggctggtggccatcctcacccgcaggcaccgccaggccattgcccgcctctggcccaactactgcctcttcctggcgctgttcctgctgtaccagtacctgctgtgcctggggatgcccccggccctgtgcattgattatccctggcgctggagccgggccgtccccatgaactccgcacccatcaagtggctgtacctgcctgatttcttccgggcccccaactccaccaacctcatcagcgactttctcctgctgctgtgcgcctcccagcagtggcaggtgttctcagctgagcgcacagaggagtggcagcgcatggctggcgtcaacaccgaccgcctggagccgctgcggggggagcccaaccccgtgcccaactttatccactgcaggtcctaccttgacatgctgaagctggccgtcttccgatacctgttctggctggtgctggtggtggtgtttgtcacgggggccacccgcatcagcatcttcgggctgggctacctgctggcctgcttctacctgctgctcttcggcacggccctgctgcagagggacacacgggcccgcctcgtgctgtgggactgcctcattctgtacaacgtcaccgtcatcatctccaagaacatgctgtcgctcctggcctgcgtcttcgtggagcagatgcagaccggcttctgctgggtcatccagctcttcagccttgtatgcaccgtcaagggctactatgaccccaaggagatgatggacagagaccaggactgcctgctgcctgtggaggaggctggcatcatctgggacagcgtctgcttcttcttcctgctgctgcagcgccgcgtcttccttagccattactacctgcacgtcagggccgacctccaggccaccgccctgctagcctccaggggcttcgccctctacaacgctgccaacctcaagagcattgacttccaccgcaggatagaggagaagtccctggcccagctgaaaagacagatggagcgtatccgtgccaagcaggagaagcacaggcagggccgggtggaccgcagtcgcccccaggacaccctgggccccaaggaccccggcctggagccagggcccgacagtccagggggctcctccccgctacggaggcagtggtggcggccctggctggaccacgccacagtcatccactccggggactacttcctgtttgagtccgacagtgaggaagaggaggaggctgttcctgaagacccgaggccgtcggcacagagtgccttccagctggcgtaccaggcatgggtgaccaacgcccaggcggtgctgaggcggcggcagcaggagcaggagcaggcaaggcaggaacaggcaggacagctacccacaggaggtggtcccagccaggaggtggagccagcagagggccccgaggaggcagcggcaggccggagccatgtggtgcagagggtgctgagcacggcgcagttcctgtggatgctggggcaggcgctagtggatgagctgacacgctggctgcaggagttcacccggcaccacggcaccatgagcgacgtgctgcgggcagagcgctacctcctcacacaggagctcctgcagggcggcgaagtgcacaggggcgtgctggatcagctgtacacaagccaggccgaggccacgctgccaggccccaccgaggcccccaatgccccaagcaccgtgtccagtgggctgggcgcggaggagccactcagcagcatgacagacgacatgggcagccccctgagcaccggctaccacacgcgcagtggcagtgaggaggcagtcaccgaccccggggagcgtgaggctggtgcctctctgtaccagggactgatgcggacggccagcgagctgctcctggacaggcgcctgcgcatcccagagctggaggaggcagagctgtttgcggaggggcagggccgggcgctgcggctgctgcgggccgtgtaccagtgtgtggccgcccactcggagctgctctgctacttcatcatcatcctcaaccacatggtcacggcctccgccggctcgctggtgctgcccgtgctcgtcttcctgtgggccatgctgtcgatcccgaggcccagcaagcgcttctggatgacggccatcgtcttcaccgagatcgcggtggtcgtcaagtacctgttccagtttgggttcttcccctggaacagccacgtggtgctgcggcgctacgagaacaagccctacttcccgccccgcatcctgggcctggagaagactgacggctacatcaagtacgacctggtgcagctcatggcccttttcttccaccgctcccagctgctgtgctatggcctctgggaccatgaggaggactcaccatccaaggagcatgacaagagcggcgaggaggagcagggagccgaggaggggccaggggtgcctgcggccaccaccgaagaccacattcaggtggaagccagggtcggacccacggacgggaccccagaatcccaagtggagctcaggccccgtgatacgaggcgcatcagtctacgttttagaagaaggaaggagggcccagcacggaaaggagcggcagccatcgaagctgaggacagggaggaagaagagggggaggaagagaaagaggcccccacggggagagagaagaggccaagccgctctggaggaagagtaagggcggccgggcggcggctgcagggcttctgcctgtccctggcccagggcacatatcggccgctacggcgcttcttccacgacatcctgcacaccaagtaccgcgcagccaccgacgtctatgccctcatgttcctggctgatgttgtcgacttcatcatcatcatttttggcttctgggcctttgggaagcactcggcggccacagacatcacgtcctccctatcagacgaccaggtacccgaggctttcctggtcatgctgctgatccagttcagtaccatggtggttgaccgcgccctctacctgcgcaagaccgtgctgggcaagctggccttccaggtggcgctggtgctggccatccacctatggatgttcttcatcctgcccgccgtcactgagaggatgttcaaccagaatgtggtggcccagctctggtacttcgtgaagtgcatctacttcgccctgtccgcctaccagatccgctgcggctaccccacccgcatcctcggcaacttcctcaccaagaagtacaatcatctcaacctcttcctcttccaggggttccggctggtgccgttcctggtggagctgcgggcagtgatggactgggtgtggacggacaccacgctgtccctgtccagctggatgtgtgtggaggacatctatgccaacatcttcatcatcaaatgcagccgagagacagagaagaaatacccgcagcccaaagggcagaagaagaagaagatcgtcaagtacggcatgggtggcctcatcatcctcttcctcatcgccatcatctggttcccgctgctcttcatgtcgctggtgcgctccgtggttggggttgtcaaccagcccatcgatgtcaccgtcaccctgaagctgggcggctatgagccgctgttcaccatgagcgcccagcagccgtccatcatccccttcacggcccaggcctatgaggagctgtcccggcagtttgacccccagccgctggccatgcagttcatcagccagtacagccctgaggacgtcgtcacggcgcagattgagggcagctccggggcgctgtggcgcatcagtccccccagccgtgcccagatgaagcgggagctctacaacggcacggccgacatcaccctgcgcttcacctggaacttccagagggacctggcgaagggaggcactgtggagtatgccaacgagaagcacatgctggccctggcccccaacagcactgcacggcggcagctggccagcctgctcgagggcacctcggaccagtctgtggtcatccccaatctcttccccaagtacatccgtgcccccaacgggcccgaagccaaccctgtgaagcagctgcagcccaatgaggaggccgactacctcggcgtgcgtatccagctgcggagggagcagggtgcgggggccaccggcttcctcgaatggtgggtcatcgaactgcaggagtgccggaccgactgcaacctgctgcccatggtcattttcagtgacaaggtcagcccaccgagcctcggcttcctggctggctacggcatcatggggctgtacgtgtccatcgtgctggtcatcggcaagttcgtgcgcggattcttcagcgagatctcgcactccattatgttcgaggagctgccgtgcgtggaccgcatcctcaagctctgccaggacatcttcctggtgcgggagactcgggagctggagctggaggaggagttgtacgccaagctcatcttcctctaccgctcactggagaccatgatcaagtggactcgtgagaaggagtag

SEQ ID NO: 20 - RNA, 1635 nt, Homo sapiens
  source 1..1635: mol_type = genomic RNA
taccagatccgctgcggctaccccacccgcatcctcggcaacttcctcaccaagaagtacaatcatctcaacctcttcctcttccaggggttccggctggtgccgttcctggtggagctgcgggcagtgatggactgggtgtggacggacaccacgctgtccctgtccagctggatgtgtgtggaggacatctatgccaacatcttcatcatcaaatgcagccgagagacagagaaggttcatgaagatttgcccgttttcttctaaaagttctatagttttagctctgaagtttcgctctttgatccactttgagaaatacccgcagcccaaagggcagaagaagaagaagatcgtcaagtacggcatgggtggcctcatcatcctcttcctcatcgccatcatctggttcccactgctcttcatgtcgctggtgcgctccgtggttggggttgtcaaccagcccatcgatgtcaccgtcaccctgaagctgggcggctatgagccgctgttcaccatgagcgcccagcagccgtccatcatccccttcacggcccaggcctatgaggagctgtcccggcagtttgacccccagccgctggccatgcagttcatcagccagtacagccctgaggacatcgtcacggcgcagattgagggcagctccggggcgctgtggcgcatcagtccccccagccgtgcccagatgaagcgggagctctacaacggcacggccgacatcaccctgcgcttcacctggaacttccagagggacctggcgaagggaggcactgtggagtatgccaacgagaagcacatgctggccctggcccccaacagcactgcacggcggcagctggccagcctgctcgagggcacctcggaccagtctgtggtcatccctaatctcttccccaagtacatccgtgcccccaacgggcccgaagccaaccctgtgaagcagctgcagcccaatgaggaggccgactacctcggcgtgcgtatccagctgcggagggagcagggtgcgggggccaccggcttcctcgaatggtgggtcatcgagctgcaggagtgccggaccgactgcaacctgctgcccatggtcattttcagtgacaagcatcatggggctgtacgtgtccatcgtgctggtcatcggcaagttcgtgcgcggattcttcagcgagatctcgcactccattatgttcgaggagctgccgtgcgtggaccgcatcctcaagctctgccaggacatcttcctggtgcgggagactcgggagctggagctggaggaggagttgtacgccaagctcatcttcctctaccgctcactggagaccatgatcaagtggactcgtgagaaggagtaggagctgctgctggcgcccgagagggaaggagccggcctgctgggcagcgtggccacaaggggcggcactcctcaggccgggggagccactgccccgtccaaggccgccagctgtgatgcatcctcccggcctgcctgagccctgatgctgctgtcagagaaggacactgcgtccccacggcctgcgtggcgctgccgtcccccacgtgtactgtagagttttttttttaattaaaaaatgttttatttatacaaatggacaa

SEQ ID NO: 21 - RNA, 660 nt, Homo sapiens
  source 1..660: mol_type = genomic RNA
ggtcatccctaatctcttccccaagtacatccgtgcccccaacgggcccgaagccaaccctgtgaagcagctgcagcccaatgaggaggccgactacctcggctgacaaggtcagcccaccgagcctcggcttcctggctggctacggcatcatggggctgtacgtgtccatcgtgctggtcatcggcaagttcgtgcgcggattcttcagcgagatctcgcactccattatgttcgaggagctgccgtgcgtggaccgcatcctcaagctctgccaggacatcttcctggtgcgggagactcgggagctggagctggaggaggagttgtacgccaagctcatcttcctctaccgctcactggagaccatgatcaagtggactcgtgagaaggagtaggagctgctgctggcgcccgagagggaaggagccggcctgctgggcagcgtggccacaaggggcggcactcctcaggccgggggagccactgccccgtccaaggccgccagctgtgatgcatcctcccggcctgcctgagccctgatgctgctgtcagagaaggacactgcgtccccacggcctgcgtggcgctgccgtcccccacgtgtactgtagagttttttttttaattaaaaaatgttttatttatacaaatggacaa

SEQ ID NO: 22 - RNA, 8072 nt, Homo sapiens
  source 1..8072: mol_type = genomic RNA
cgtccggcccagctcggccccagtgagccgagcgctgcgctccgccgaggggcagggcggtcgcctgagcgagcgcgggcccgggacgtcggcaccggcggggcggccgaaggagaaggaggaagaggagaaggcggcgcgcgggtccccgcgggtcagccatggcgcgccggccccggggcccccgcaccgccccatagcgccgcggcgtccgctcggtctgggccgggccctgggccctccagccatggagccgcacgtgctcggcgcggtcctgtactggctgctgctgccctgcgcgctgctggctgcctgcctgctccgcttcagcggactctcgctggtctacctgctcttcctgctgctgctgccctggttccccggccccacccgatgcggcctccaaggtcacacaggccgcctcctgcgggcattgctgggcctcagcctgctcttcctggtggcccatctcgccctccagatctgcctgcatattgtgccccgcctggaccagctcctgggacccagctgcagccgctgggagaccctctcgcgacacataggggtcacaaggctggacctgaaggacatccccaacgccatccggctggtggcccctgacctgggcatcttggtggtctcctctgtctgcctcggcatctgcgggcgccttgcaaggaacacccggcagagcccacatccacgggagctggatgatgatgagagggatgtggatgccagcccgacggcagggctgcaggaagcagcaacgctggcccctacacggaggtcacggctggccgctcgtttccgagtcacggcccactggctgctggtggcggctgggcgggtcctggccgtaacactgcttgcactggcaggcatcgcccacccctcggccctctccagtgtctacctgctgctcttcctggccctctgcacctggtgggcctgccactttcccatcagcactcggggcttcagcagactctgcgtcgcggtggggtgcttcggcgccggccatctcatctgcctctactgctaccagatgcccttggcacaggctctgctcccgcctgccggcatctgggctagggtgctgggtctcaaggacttcgtgggtcccaccaactgctccagcccccacgcgctggtcctcaacaccggcctggactggcctgtgtatgccagccccggcgtcctcctgctgctgtgctacgccacggcctctctgcgcaagctccgcgcgtaccgcccctccggccagaggaaggaggcggcaaaggggtatgaggctcgggagctggagctagcagagctggaccagtggccccaggaacgggagtctgaccagcacgtggtgcccacagcacccgacaccgaggctgataactgcatcgtgcacgagctgaccggccagagctccgtcctgcggcggcctgtgcggcccaagcgggctgagcccagggaggcgtctccgctccacagcctgggccacctcatcatggaccagagctatgtgtgcgcgctcattgccatgatggtatggagcatcacctaccacagctggctgaccttcgtactgctgctctgggcctgcctcatctggacggtgcgcagccgccaccaactggccatgctgtgctcgccctgcatcctgctgtatgggatgacgctgtgctgcctacgctacgtgtgggccatggacctgcgccctgagctgcccaccaccctgggccccgtcagcctgcgccagctggggctggagcacacccgctacccctgtctggaccttggtgccatgttgctctacaccctgaccttctggctcctgctgcgccagtttgtgaaagagaagctgctgaagtgggcagagtctccagctgcgctgacggaggtcaccgtggcagacacagagcccacgcggacgcagacgctgttgcagagcctgggggagctggtgaagggcgtgtacgccaagtactggatctatgtgtgtgctggcatgttcatcgtggtcagcttcgccggccgcctcgtggtctacaagattgtctacatgttcctcttcctgctctgcctcaccctcttccaggtctactacagcctgtggcggaagctgctcaaggccttctggtggctcgtggtggcctacaccatgctggtcctcatcgccgtctacaccttccagttccaggacttccctgcctactggcgcaacctcactggcttcaccgacgagcagctgggggacctgggcctggagcagttcagcgtgtccgagctcttctccagcatcctggtgcccggcttcttcctcctggcctgcatcctgcagctgcactacttccacaggcccttcatgcagctcaccgacatggagcacgtgtccctgcctggcacgcgcctcccgcgctgggctcacaggcaggatgcagtgagtgggaccccactgctgcgggaggagcagcaggagcatcagcagcagcagcaggaggaggaggaggaggaggaggactccagggacgaggggctgggcgtggccactccccaccaggccacgcaggtgcctgaaggggcagccaagtggggcctggtggctgagcggctgctggagctggcagccggcttctcggacgtcctctcacgcgtgcaggtgttcctgcggcggctgctggagcttcacgttttcaagctggtggccctgtacaccgtctgggtggccctgaaggaggtgtcggtgatgaacctgctgctggtggtgctgtgggccttcgccctgccctacccacgcttccggcccatggcctcctgcctgtccaccgtgtggacctgcgtcatcatcgtgtgtaagatgctgtaccagctcaaggttgtcaacccccaggagtattccagcaactgcaccgagcccttccccaacagcaccaacttgctgcccacggagatcagccagtccctgctgtaccgggggcccgtggaccctgccaactggtttggggtgcggaaagggttccccaacctgggctacatccagaaccacctgcaagtgctgctgctgctggtattcgaggccatcgtgtaccggcgccaggagcactaccgccggcagcaccagctggccccgctgcctgcccaggccgtgtttgccagcggcacccgccagcagctggaccaggatctgctcggctgcctcaagtacttcatcaacttcttcttctacaaattcgggctggagatctgcttcctgatggccgtgaacgtgatcgggcagcgcatgaactttctggtgaccctgcacggttgctggctggtggccatcctcacccgcaggcaccgccaggccattgcccgcctctggcccaactactgcctcttcctggcgctgttcctgctgtaccagtacctgctgtgcctggggatgcccccggccctgtgcattgattatccctggcgctggagccgggccgtccccatgaactccgcactcatcaagtggctgtacctgcctgatttcttccgggcccccaactccaccaacctcatcagcgactttctcctgctgctgtgcgcctcccagcagtggcaggtgttctcagctgagcgcacagaggagtggcagcgcatggctggcgtcaacaccgaccgcctggagccgctgcggggggagcccaaccccgtgcccaactttatccactgcaggtcctaccttgacatgctgaaggtggccgtcttccgatacctgttctggctggtgctggtggtggtgtttgtcacgggggccacccgcatcagcatcttcgggctgggctacctgctggcctgcttctacctgctgctcttcggcacggccctgctgcagagggacacacgggcccgcctcgtgctgtgggactgcctcattctgtacaacgtcaccgtcatcatctccaagaacatgctgtcgctcctggcctgcgtcttcgtggagcagatgcagaccggcttctgctgggtcatccagctcttcagccttgtatgcaccgtcaagggctactatgaccccaaggagatgatggacagagaccaggactgcctgctgcctgtggaggaggctggcatcatctgggacagcgtctgcttcttcttcctgctgctgcagcgccgcgtcttccttagccattactacctgcacgtcagggccgacctccaggccaccgccctgctagcctccaggggcttcgccctctacaacgctgccaacctcaagagcattgactttcaccgcaggatagaggagaagtccctggcccagctgaaaagacagatggagcgtatccgtgccaagcaggagaagcacaggcagggccgggtggaccgcagtcgcccccaggacaccctgggccccaaggaccccggcctggagccagggcccgacagtccagggggctcctccccgccacggaggcagtggtggcggccctggctggaccacgccacagtcatccactccggggactacttcctgtttgagtccgacagtgaggaagaggaggaggctgttcctgaagacccgaggccgtcggcacagagtgccttccagctggcgtaccaggcatgggtgaccaacgcccaggcggtgctgaggcggcggcagcaggagcaggagcaggcaaggcaggaacaggcaggacagctacccacaggaggtggtcccagccaggaggtggagccagcagagggccccgaggaggcagcggcaggccggagccatgtggtgcagagggtgctgagcacggcgcagttcctgtggatgctggggcaggcgctagtggatgagctgacacgctggctgcaggagttcacccggcaccacggcaccatgagcgacgtgctgcgggcagagcgctacctcctcacacaggagctcctgcagggcggcgaagtgcacaggggcgtgctggatcagctgtacacaagccaggccgaggccacgctgccaggccccaccgaggcccccaatgccccaagcaccgtgtccagtgggctgggcgcggaggagccactcagcagcatgacagacgacatgggcagccccctgagcaccggctaccacacgcgcagtggcagtgaggaggcagtcaccgaccccggggagcgtgaggctggtgcctctctgtaccagggactgatgcggacggccagcgagctgctcctggacaggcgcctgcgcatcccagagctggaggaggcagagctgtttgcggaggggcagggccgggcgctgcggctgctgcgggccgtgtaccagtgtgtggccgcccactcggagctgctctgctacttcatcatcatcctcaaccacatggtcacggcctccgccggctcgctggtgctgcccgtgctcgtcttcctgtgggccatgctgtcgatcccgaggcccagcaagcgcttctggatgacggccatcgtcttcaccgagatcgcggtggtcgtcaagtacctgttccagtttgggttcttcccctggaacagccacgtggtgctgcggcgctacgagaacaagccctacttcccgccccgcatcctgggcctggagaagactgacggctacatcaagtacgacctggtgcagctcatggcccttttcttccaccgctcccagctgctgtgctatggcctctgggaccatgaggaggactcaccatccaaggagcatgacaagagcggcgaggaggagcagggagccgaggaggggccaggggtgcctgcggccaccaccgaagaccacattcaggtggaagccagggtcggacccacggacgggaccccagaaccccaagtggagctcaggccccgtgatacgaggcgcatcagtctacgttttagaagaaggaagaaggagggcccagcacggaaaggagcggcagccatcgaagctgaggacagggaggaagaagagggggaggaagagaaagaggcccccacggggagagagaagaggccaagccgctctggaggaagagtaagggcggccgggcggcggctgcagggcttctgcctgtccctggcccagggcacatatcggccgctacggcgcttcttccacgacatcctgcacaccaagtaccgcgcagccaccgacgtctatgccctcatgttcctggctgatgttgtcgacttcatcatcatcatttttggcttctgggcctttgggaagcactcggcggccacagacatcacgtcctccctatcagacgaccaggtacccgaggctttcctggtcatgctgctgatccagttcagtaccatggtggttgaccgcgccctctacctgcgcaagaccgtgctgggcaagctggccttccaggtggcgctggtgctggccatccacctatggatgttcttcatcctgcccgccgtcactgagaggatgttcaaccagaatgtggtggcccagctctggtacttcgtgaagtgcatctacttcgccctgtccgcctaccagatccgctgcggctaccccacccgcatcctcggcaacttcctcaccaagaagtacaatcatctcaacctcttcctcttccaggggttccggctggtgccgttcctggtggagctgcgggcagtgatggactgggtgtggacggacaccacgctgtccctgtccagctggatgtgtgtggaggacatctatgccaacatcttcatcatcaaatgcagccgagagacagagaagaaatacccgcagcccaaagggcagaagaagaagaagatcgtcaagtacggcatgggtggcctcatcatcctcttcctcatcgccatcatctggttcccactgctcttcatgtcgctggtgcgctccgtggttggggttgtcaaccagcccatcgatgtcaccgtcaccctgaagctgggcggctatgagccgctgttcaccatgagcgcccagcagccgtccatcatccccttcacggcccaggcctatgaggagctgtcccggcagtttgacccccagccgctggccatgcagttcatcagccagtacagccctgaggacatcgtcacggcgcagattgagggcagctccggggcgctgtggcgcatcagtccccccagccgtgcccagatgaagcgggagctctacaacggcacggccgacatcaccctgcgcttcacctggaacttccagagggacctggcgaagggaggcactgtggagtatgccaacgagaagcacatgctggccctggcccccaacagcactgcacggcggcagctggccagcctgctcgagggcacctcggaccagtctgtggtcatccctaatctcttccccaagtacatccgtgcccccaacgggcccgaagccaaccctgtgaagcagctgcagcccaatgaggaggccgactacctcggcgtgcgtatccagctgcggagggagcagggtgcgggggccaccggcttcctcgaatggtgggtcatcgagctgcaggagtgccggaccgactgcaacctgctgcccatggtcattttcagtgacaaggtcagcccaccgagcctcggcttcctggctggctacggcatcatggggctgtacgtgtccatcgtgctggtcatcggcaagttcgtgcgcggattcttcagcgagatctcgcactccattatgttcgaggagctgccgtgcgtggaccgcatcctcaagctctgccaggacatcttcctggtgcgggagactcgggagctggagctggaggaggagttgtacgccaagctcatcttcctctaccgctcactggagaccatgatcaagtggactcgtgagaaggagtaggagctgctgctggcgcccgagagggaaggagccggcctgctgggcagcgtggccacaaggggcggcactcctcaggccgggggagccactgccccgtccaaggccgccagctgtgatgcatcctcccggcctgcctgagccctgatgctgctgtcagagaaggacactgcgtccccacggcctgcgtggcgctgccgtcccccacgtgtactgtagagttttttttttaattaaaaaatgttttatttatacaaatgga

SEQ ID NO: 23 - DNA, 8089 bp, Homo sapiens
  source 1..8089: mol_type = genomic DNA
gggagccgccgtccggcccagctcggccccagtgagccgagcgctgcgctccgccgaggggcagggcggtcgcctgagcgagcgcgggcccgggacgtcggcaccggcggggcggccgaaggagaaggaggaagaggagaaggcggcgcgcgggtccccgcgggtcagccatggcgcgccggccccggggcccccgcaccgccccatagcgccgcggcgtccgctcggtctgggccgggccctgggccctccagccatggagccgcacgtgctcggcgcggtcctgtactggctgctgctgccctgcgcgctgctggctgcctgcctgctccgcttcagcggactctcgctggtctacctgctcttcctgctgctgctgccctggttccccggccccacccgatgcggcctccaaggtcacacaggccgcctcctgcgggcattgctgggcctcagcctgctcttcctggtggcccatctcgccctccagatctgcctgcatattgtgccccgcctggaccagctcctgggacccagctgcagccgctgggagaccctctcgcgacacataggggtcacaaggctggacctgaaggacatccccaacgccatccggctggtggcccctgacctgggcatcttggtggtctcctctgtctgcctcggcatctgcgggcgccttgcaaggaacacccggcagagcccacatccacgggagctggatgatgatgagagggatgtggatgccagcccgacggcagggctgcaggaagcagcaacgctggcccctacacggaggtcacggctggccgctcgtttccgagtcacggcccactggctgctggtggcggctgggcgggtcctggccgtaacactgcttgcactggcaggcatcgcccacccctcggccctctccagtgtctacctgctgctcttcctggccctctgcacctggtgggcctgccactttcccatcagcactcggggcttcagcagactctgcgtcgcggtggggtgcttcggcgccggccatctcatctgcctctactgctaccagatgcccttggcacaggctctgctcccgcctgccggcatctgggctagggtgctgggtctcaaggacttcgtgggtcccaccaactgctccagcccccacgcgctggtcctcaacaccggcctggactggcctgtgtatgccagccccggcgtcctcctgctgctgtgctacgccacggcctctctgcgcaagctccgcgcgtaccgcccctccggccagaggaaggaggcggcaaaggggtatgaggctcgggagctggagctagcagagctggaccagtggccccaggaacgggagtctgaccagcacgtggtgcccacagcacccgacaccgaggctgataactgcatcgtgcacgagctgaccggccagagctccgtcctgcggcggcctgtgcggcccaagcgggctgagcccagggaggcgtctccgctccacagcctgggccacctcatcatggaccagagctatgtgtgcgcgctcattgccatgatggtatggagcatcacctaccacagctggctgaccttcgtactgctgctctgggcctgcctcatctggacggtgcgcagccgccaccaactggccatgctgtgctcgccctgcatcctgctgtatgggatgacgctgtgctgcctacgctacgtgtgggccatggacctgcgccctgagctgcccaccaccctgggccccgtcagcctgcgccagctggggctggagcacacccgctacccctgtctggaccttggtgccatgttgctctacaccctgaccttctggctcctgctgcgccagtttgtgaaagagaagctgctgaagtgggcagagtctccagctgcgctgacggaggtcaccgtggcagacacagagcccacgcggacgcagacgctgttgcagagcctgggggagctggtgaagggcgtgtacgccaagtactggatctatgtgtgtgctggcatgttcatcgtggtcagcttcgccggccgcctcgtggtctacaagattgtctacatgttcctcttcctgctctgcctcaccctcttccaggtctactacagcctgtggcggaagctgctcaaggccttctggtggctcgtggtggcctacaccatgctggtcctcatcgccgtctacaccttccagttccaggacttccctgcctactggcgcaacctcactggcttcaccgacgagcagctgggggacctgggcctggagcagttcagcgtgtccgagctcttctccagcatcctggtgcccggcttcttcctcctggcctgcatcctgcagctgcactacttccacaggcccttcatgcagctcaccgacatggagcacgtgtccctgcctggcacgcgcctcccgcgctgggctcacaggcaggatgcagtgagtgggaccccactgctgcgggaggagcagcaggagcatcagcagcagcagcaggaggaggaggaggaggaggaggactccagggacgaggggctgggcgtggccactccccaccaggccacgcaggtgcctgaaggggcagccaagtggggcctggtggctgagcggctgctggagctggcagccggcttctcggacgtcctctcacgcgtgcaggtgttcctgcggcggctgctggagcttcacgttttcaagctggtggccctgtacaccgtctgggtggccctgaaggaggtgtcggtgatgaacctgctgctggtggtgctgtgggccttcgccctgccctacccacgcttccggcccatggcctcctgcctgtccaccgtgtggacctgcgtcatcatcgtgtgtaagatgctgtaccagctcaaggttgtcaacccccaggagtattccagcaactgcaccgagcccttccccaacagcaccaacttgctgcccacggagatcagccagtccctgctgtaccgggggcccgtggaccctgccaactggtttggggtgcggaaagggttccccaacctgggctacatccagaaccacctgcaagtgctgctgctgctggtattcgaggccatcgtgtaccggcgccaggagcactaccgccggcagcaccagctggccccgctgcctgcccaggccgtgtttgccagcggcacccgccagcagctggaccaggatctgctcggctgcctcaagtacttcatcaacttcttcttctacaaattcgggctggagatctgcttcctgatggccgtgaacgtgatcgggcagcgcatgaactttctggtgaccctgcacggttgctggctggtggccatcctcacccgcaggcaccgccaggccattgcccgcctctggcccaactactgcctcttcctggcgctgttcctgctgtaccagtacctgctgtgcctggggatgcccccggccctgtgcattgattatccctggcgctggagccgggccgtccccatgaactccgcactcatcaagtggctgtacctgcctgatttcttccgggcccccaactccaccaacctcatcagcgactttctcctgctgctgtgcgcctcccagcagtggcaggtgttctcagctgagcgcacagaggagtggcagcgcatggctggcgtcaacaccgaccgcctggagccgctgcggggggagcccaaccccgtgcccaactttatccactgcaggtcctaccttgacatgctgaaggtggccgtcttccgatacctgttctggctggtgctggtggtggtgtttgtcacgggggccacccgcatcagcatcttcgggctgggctacctgctggcctgcttctacctgctgctcttcggcacggccctgctgcagagggacacacgggcccgcctcgtgctgtgggactgcctcattctgtacaacgtcaccgtcatcatctccaagaacatgctgtcgctcctggcctgcgtcttcgtggagcagatgcagaccggcttctgctgggtcatccagctcttcagccttgtatgcaccgtcaagggctactatgaccccaaggagatgatggacagagaccaggactgcctgctgcctgtggaggaggctggcatcatctgggacagcgtctgcttcttcttcctgctgctgcagcgccgcgtcttccttagccattactacctgcacgtcagggccgacctccaggccaccgccctgctagcctccaggggcttcgccctctacaacgctgccaacctcaagagcattgactttcaccgcaggatagaggagaagtccctggcccagctgaaaagacagatggagcgtatccgtgccaagcaggagaagcacaggcagggccgggtggaccgcagtcgcccccaggacaccctgggccccaaggaccccggcctggagccagggcccgacagtccagggggctcctccccgccacggaggcagtggtggcggccctggctggaccacgccacagtcatccactccggggactacttcctgtttgagtccgacagtgaggaagaggaggaggctgttcctgaagacccgaggccgtcggcacagagtgccttccagctggcgtaccaggcatgggtgaccaacgcccaggcggtgctgaggcggcggcagcaggagcaggagcaggcaaggcaggaacaggcaggacagctacccacaggaggtggtcccagccaggaggtggagccagcagagggccccgaggaggcagcggcaggccggagccatgtggtgcagagggtgctgagcacggcgcagttcctgtggatgctggggcaggcgctagtggatgagctgacacgctggctgcaggagttcacccggcaccacggcaccatgagcgacgtgctgcgggcagagcgctacctcctcacacaggagctcctgcagggcggcgaagtgcacaggggcgtgctggatcagctgtacacaagccaggccgaggccacgctgccaggccccaccgaggcccccaatgccccaagcaccgtgtccagtgggctgggcgcggaggagccactcagcagcatgacagacgacatgggcagccccctgagcaccggctaccacacgcgcagtggcagtgaggaggcagtcaccgaccccggggagcgtgaggctggtgcctctctgtaccagggactgatgcggacggccagcgagctgctcctggacaggcgcctgcgcatcccagagctggaggaggcagagctgtttgcggaggggcagggccgggcgctgcggctgctgcgggccgtgtaccagtgtgtggccgcccactcggagctgctctgctacttcatcatcatcctcaaccacatggtcacggcctccgccggctcgctggtgctgcccgtgctcgtcttcctgtgggccatgctgtcgatcccgaggcccagcaagcgcttctggatgacggccatcgtcttcaccgagatcgcggtggtcgtcaagtacctgttccagtttgggttcttcccctggaacagccacgtggtgctgcggcgctacgagaacaagccctacttcccgccccgcatcctgggcctggagaagactgacggctacatcaagtacgacctggtgcagctcatggcccttttcttccaccgctcccagctgctgtgctatggcctctgggaccatgaggaggactcaccatccaaggagcatgacaagagcggcgaggaggagcagggagccgaggaggggccaggggtgcctgcggccaccaccgaagaccacattcaggtggaagccagggtcggacccacggacgggaccccagaaccccaagtggagctcaggccccgtgatacgaggcgcatcagtctacgttttagaagaaggaagaaggagggcccagcacggaaaggagcggcagccatcgaagctgaggacagggaggaagaagagggggaggaagagaaagaggcccccacggggagagagaagaggccaagccgctctggaggaagagtaagggcggccgggcggcggctgcagggcttctgcctgtccctggcccagggcacatatcggccgctacggcgcttcttccacgacatcctgcacaccaagtaccgcgcagccaccgacgtctatgccctcatgttcctggctgatgttgtcgacttcatcatcatcatttttggcttctgggcctttgggaagcactcggcggccacagacatcacgtcctccctatcagacgaccaggtacccgaggctttcctggtcatgctgctgatccagttcagtaccatggtggttgaccgcgccctctacctgcgcaagaccgtgctgggcaagctggccttccaggtggcgctggtgctggccatccacctatggatgttcttcatcctgcccgccgtcactgagaggatgttcaaccagaatgtggtggcccagctctggtacttcgtgaagtgcatctacttcgccctgtccgcctaccagatccgctgcggctaccccacccgcatcctcggcaacttcctcaccaagaagtacaatcatctcaacctcttcctcttccaggggttccggctggtgccgttcctggtggagctgcgggcagtgatggactgggtgtggacggacaccacgctgtccctgtccagctggatgtgtgtggaggacatctatgccaacatcttcatcatcaaatgcagccgagagacagagaagaaatacccgcagcccaaagggcagaagaagaagaagatcgtcaagtacggcatgggtggcctcatcatcctcttcctcatcgccatcatctggttcccactgctcttcatgtcgctggtgcgctccgtggttggggttgtcaaccagcccatcgatgtcaccgtcaccctgaagctgggcggctatgagccgctgttcaccatgagcgcccagcagccgtccatcatccccttcacggcccaggcctatgaggagctgtcccggcagtttgacccccagccgctggccatgcagttcatcagccagtacagccctgaggacatcgtcacggcgcagattgagggcagctccggggcgctgtggcgcatcagtccccccagccgtgcccagatgaagcgggagctctacaacggcacggccgacatcaccctgcgcttcacctggaacttccagagggacctggcgaagggaggcactgtggagtatgccaacgagaagcacatgctggccctggcccccaacagcactgcacggcggcagctggccagcctgctcgagggcacctcggaccagtctgtggtcatccctaatctcttccccaagtacatccgtgcccccaacgggcccgaagccaaccctgtgaagcagctgcagcccaatgaggaggccgactacctcggcgtgcgtatccagctgcggagggagcagggtgcgggggccaccggcttcctcgaatggtgggtcatcgagctgcaggagtgccggaccgactgcaacctgctgcccatggtcattttcagtgacaaggtcagcccaccgagcctcggcttcctggctggctacggcatcatggggctgtacgtgtccatcgtgctggtcatcggcaagttcgtgcgcggattcttcagcgagatctcgcactccattatgttcgaggagctgccgtgcgtggaccgcatcctcaagctctgccaggacatcttcctggtgcgggagactcgggagctggagctggaggaggagttgtacgccaagctcatcttcctctaccgctcaccggagaccatgatcaagtggactcgtgagaaggagtaggagctgctgctggcgcccgagagggaaggagccggcctgctgggcagcgtggccacaaggggcggcactcctcaggccgggggagccactgccccgtccaaggccgccagctgtgatgcatcctcccggcctgcctgagccctgatgctgctgtcagagaaggacactgcgtccccacggcctgcgtggcgctgccgtcccccacgtgtactgtagagttttttttttaattaaaaaatgttttatttatacaaatggacaatcaga

SEQ ID NO: 24 - DNA, 1246 bp, Homo sapiens
  source 1..1246: mol_type = genomic DNA
atgaagatttgcccgttttcttctaaaagttctatagttttagctctgaagtttcgctctttgatccactttgagaaatacccgcagcccaaagggcagaagaagaagaagatcgtcaagtacggcatgggtggcctcatcatcctcttcctcatcgccatcatctggttcccactgctcttcatgtcgctggtgcgctccgtggttggggttgtcaaccagcccatcgatgtcaccgtcaccctgaagctgggcggctatgagccgctgttcaccatgagcgcccagcagccgtccatcatccccttcacggcccaggcctatgaggagctgtcccggcagtttgacccccagccgctggccatgcagttcatcagccagtacagccctgaggacatcgtcacggcgcagattgagggcagctccggggcgctgtggcgcatcagtccccccagccgtgcccagatgaagcgggagctctacaacggcacggccgacatcaccctgcgcttcacctggaacttccagagggacctggcgaagggaggcactgtggagtatgccaacgagaagcacatgctggccctggcccccaacagcactgcacggcggcagctggccagcctgctcgagggcacctcggaccagtctgtggtcatccctaatctcttccccaagtacatccgtgcccccaacgggcccgaagccaaccctgtgaagcagctgcagcccaatgaggaggccgactacctcggcgtgcgtatccagctgcggagggagcagggtgcgggggccaccggcttcctcgaatggtgggtcatcgagctgcaggagtgccggaccgactgcaacctgctgcccatggtcattttcagtgacaagcatcatggggctgtacgtgtccatcgtgctggtcatcggcaagttcgtgcgcggattcttcagcgagatctcgcactccattatgttcgaggagctgccgtgcgtggaccgcatcctcaagctctgccaggacatcttcctggtgcgggagactcgggagctggagctggaggaggagttgtacgccaagctcatcttcctctaccgctcaccggagaccatgatcaagtggactcgtgagaaggagtaggagctgctgctggcgcccgagagggaaggagccggcctgctgggcagcgtggccacaaggggcggcactcctcaggccgggggagccactgccccgtccaaggccgccagctgt

SEQ ID NO: 25 - DNA, 6401 bp, Homo sapiens
  source 1..6401: mol_type = genomic DNA
ccatggacctgcgccctgagctgcccaccaccctgggccccgtcagcctgcgccagctggggctggagcacacccgctacccctgtctggaccttggtgccatgttgctctacaccctgaccttctggctcctgctgcgccagtttgtgaaagagaagctgctgaagtgggcagagtctccagctgcgctgacggaggtcaccgtggcagacacagagcctacgcggacgcagacgctgttgcagagcctgggggagctggtgaagggcgtgtacgccaagtactggatctatgtgtgtgctggcatgttcatcgtggtcagcttcgccggccgcctcgtggtctacaagattgtctacatgttcctcttcctgctctgcctcaccctcttccaggtctactacagcctgtggcggaagctgctcaaggccttctggtggctcgtggtggcctacaccatgctggtcctcatcgccgtctacaccttccagttccaggacttccctgcctactggcgcaacctcactggcttcaccgacgagcagctgggggacctgggcctggagcagttcagcgtatccgagctcttctccagcatcctggtgcccggcttcttcctcctggcctgcatcctgcagctgcactacttccacaggcccttcatgcagctcaccgacatggagcacgtgtccctgcctggcacgcgcctcccgcgctgggctcacaggcaggatgcagtgagtgggaccccactgctgcgggaggagcagcaggagcatcagcagcagcagcaggaggaggaggaggaggaggactccagggacgaggggctgggcgtggccactccccaccaggccacgcaggtgcctgaaggggcagccaagtggggcctggtggctgagcgcctgctggagctggcagccggcttctcggacgtcctctcacgcgtgcaggtgttcctgcggcggctgctggagcttcacgttttcaagctggtggccctgtacaccgtctgggtggccctgaaggaggtgtcggtgatgaacctgctgctggtggtgctgtgggccttcgccctgccctacccacgcttccggcccatggcctcctgcctgtccaccgtgtggacctgcgtcatcatcgtgtgtaagatgctgtaccagctcaaggttgtcaacccccaggagtattccagcaactgcaccgagcccttccccaacagcaccaacttgctgcccacggagatcagccagtccctgctgtaccgggggcccgtggaccctgccaactggtttggggtgcggaaagggttccccaacctgggctacatccagaaccacctgcaagtgctgctgctgctggtattcgaggccatcgtgtaccggcgccaggagcactaccgccggcagcaccagctggccccgctgcctgcccaggccgtgtttgccagcggcacccgccagcagctggaccaggatctgctcggctgcctcaagtacttcatcaacttcttcttctacaaattcgggctggagatctgcttcctgatggccgtgaacgtgatcgggcagcgcatgaactttctggtgaccctgcacggttgctggctggtggccatcctcacccgcaggcaccgccaggccattgcccgcctctggcccaactactgcctcttcctggcgctgttcctgctgtaccagtacctgctgtgcctggggatgcccccggccctgtgcattgattatccctggcgctggagccgggccgtccccatgaactccgcactcatcaagtggctgtacctgcctgatttcttccgggcccccaactccaccaacctcatcagcgactttctcctgctgctgtgcgcctcccagcagtggcaggtgttctcagctgagcgcacagaggagtggcagcgcatggctggcgtcaacaccgaccgcctggagccgctgcggggggagcccaaccccgtgcccaactttatccactgcaggtcctaccttgacatgctgaaggtggccgtcttccgatacctgttctggctggtgctggtggtggtgtttgtcacgggggccacccgcatcagcatcttcgggctgggctacctgctggcctgcttctacctgctgctcttcggcacggccctgctgcagagggacacacgggcccgcctcgtgctgtgggactgcctcattctgtacaacgtcaccgtcatcatctccaagaacatgctgtcgctcctggcctgcgtcttcgtggagcagatgcagaccggcttctgctgggtcatccagctcttcagccttgtatgcaccgtcaagggctactatgaccccaaggagatgatggacagagaccaggactgcctgctgcctgtggaggaggctggcatcatctgggacagcgtctgcttcttcttcctgctgctgcagcgccgcgtcttccttagccattactacctgcacgtcagggccgacctccaggccaccgccctgctagcctccaggggcttcgccctctacaacgctgccaacctcaagagcattgacttccaccgcaggatagaggagaagtccctggcccagctgaaaagacagatggagcgtatccgtgccaagcaggagaagcacaggcagggccgggtggaccgcagtcgcccccaggacaccctgggccccaaggaccccggcctggagccagggcccgacagtccagggggctcctccccgccacggaggcagtggtggcggccctggctggaccacgccacagtcatccactccggggactacttcctgtttgagtccgacagtgaggaagaggaggaggctgttcctgaagacccgaggccgtcggcacagagtgccttccagctggcgtaccaggcatgggtgaccaacgcccaggcggtgctgaggcggcggcagcaggagcaggagcaggcaaggcaggaacaggcaggacagctacccacaggaggtggtcccagccaggaggtggagccagcagagggccccgaggaggcagcggcaggccggagccatgtggtgcagagggtgctgagcacggcgcagttcctgtggatgctggggcaggcgctagtggatgagctgacacgctggctgcaggagttcacccggcaccacggcaccatgagcgacgtgctgcgggcagagcgctacctcctcacacaggagctcctgcagggcggcgaagtgcacaggggcgtgctggatcagctgtacacaagccaggccgaggccacgctgccaggccccaccgaggcccccaatgccccaagcaccgtgtccagtgggctgggcgcggaggagccactcagcagcatgacagacgacatgggcagccccctgagcaccggctaccacacgcgcagtggcagtgaggaggcagtcaccgaccccggggagcgtgaggctggtgcctctctgtaccagggactgatgcggacggccagcgagctgctcctggacaggcgcctgcgcatcccagagctggaggaggcagagctgtttgcggaggggcagggccgggcgctgcggctgctgcgggccgtgtaccagtgtgtggccgcccactcggagctgctctgctacttcatcatcatcctcaaccacatggtcacggcctccgccggctccctggtgctgcccgtgctcgtcttcctgtgggccatgctgtcgatcccgaggcccagcaagcgcttctggatgacggccatcgtcttcaccgagatcgcggtggtcgtcaagtacctgttccagtttgggttcttcccctggaacagccacgtggtgctgcggcgctacgagaacaagccctacttcccgccccgcatcctgggcctggagaagactgacggctacatcaagtacgacctggtgcagctcatggcccttttcttccaccgctcccagctgctgtgctatggcctctgggaccatgaggaggactcaccatccaaggagcatgacaagagcggcgaggaggagcagggagccgaggaggggccaggggtgcctgcggccaccaccgaagaccacattcaggtggaagcgagggtcggacccacggacgggaccccagaaccccaagtggagctcaggccccgtgatacgaggcgcatcagtctacgttttagaagaaggaagaaggagggcccagcacggaaaggagcggcagccatcgaagctgaggacagggaggaagaagagggggaggaagagaaagaggcccccacggggagagagaagaggccaagccgctctggaggaagagtaagggcggccgggcggcggctgcagggcttctgcctgtccctggcccagggcacatatcggccgctacggcgcttcttccacgacatcctgcacaccaagtaccgcgcagccaccgacgtctatgccctcatgttcctggctgatgttgtcgacttcatcatcatcatttttggcttctgggcctttgggaagcactcggcggccacagacatcacgtcctccctatcagacgaccaggtacccgaggctttcctggtcatgctgctgatccagttcagtaccatggtggttgaccgcgccctctacctgcgcaagaccgtgctgggcaagctggccttccaggtggcgctggtgctggccatccacctatggatgttcttcatcctgcccgccgtcactgagaggatgttcaaccagaatgtggtggcccagctctggtacttcgtgaagtgcatctacttcgccctgtccgcctaccagatccgctgcggctaccccacccgcatcctcggcaacttcctcaccaagaagtacaatcatctcaacctcttcctcttccaggggttccggctggtgccgttcctggtggagctgcgggcagtgatggactgggtgtggacggacaccacgctgtccctgtccagctggatgtgtgtggaggacatctatgccaacatcttcatcatcaaatgcagccgagagacagagaagaaatacccgcagcccaaagggcagaagaagaagaagatcgtcaagtacggcatgggtggcctcatcatcctcttcctcatcgccatcatctggttcccgctgctcttcatgtcgctggtgcgctccgtggttggggttgtcaaccagcccatcgatgtcaccgtcaccctcaagctgggcggctatgagccgctgttcaccatgagcgcccagcagccgtccatcatccccttcacggcccaggcctatgaggagctgtcccggcagtttgacccccagccgctggccatgcagttcatcagccagtacagccctgaggacatcgtcacggcgcagattgagggcagctccggggcgctgtggcgcatcagtccccccagccgtgcccagatgaagcgggagctctacaacggcacggccgacatcaccctgcgcttcacctggaacttccagagggacctggcgaagggaggcactgtggagtatgccaacgagaagcacatgctggccctggcccccaacagcactgcacggcggcagctggccagcctgctcgagggcacctcggaccagtctgtggtcatccccaatctcttccccaagtacatccgtgcccccaacgggcccgaagccaaccctgtgaagcagctgcagcccaatgaggaggccgactacctcggcgtgcgtatccagctgcggagggagcagggtgcgggggccaccggcttcctcgaatggtgggtcatcgagctgcaggagtgccggaccgactgcaacctgctgcccatggtcattttcagtgacaaggtcagcccaccgagcctcggcttcctggctggctacggcatcatggggctgtacgtgtccatcgtgctggtcatcggcaagttcgtgcgcggattcttcagcgagatctcgcactccattatgttcgaggagctgccgtgcgtggaccgcatcctcaagctctgccaggacatcttcctggtgcgggagactcgggagctggagctggaggaggagttgtacgccaagctcatcttcctctaccgctcaccggagaccatgatcaagtggactcgtgagaaggagtaggagctgctgctggcgcccgagagggaaggagccggcctgctgggcagcgtggccacaaggggcggcactcctcaggccgggggagccactgccccgtccaaggccgccagctgtgatgcatcctcccggcctgcctgagccctgatgctgctgtcagagaaggacactgcgtccccacggcctgcgtggcgctgccgtcccccacgtgtactgtagagttttttttttaattaaaaaatgttttatttatacaaatggaaaaaaaaaaaaaaaaaaaaaaaaaaaaaaaaa

SEQ ID NO: 26 - DNA, 1655 bp, Homo sapiens
  source 1..1655: mol_type = genomic DNA
taccagatccgctgcggctaccccacccgcatcctcggcaacttcctcaccaagaagtacaatcatctcaacctcttcctcttccaggggttccggctggtgccgttcctggtggagctgcgggcagtgatggactgggtgtggacggacaccacgctgtccctgtccagctggatgtgtgtggaggacatctatgccaacatcttcatcatcaaatgcagccgagagacagagaaggttcatgaagatttgcccgttttcttctaaaagttctatagttttagctctgaagtttcgctctttgatccactttgagaaatacccgcagcccaaagggcagaagaagaagaagatcgtcaagtacggcatgggtggcctcatcatcctcttcctcatcgccatcatctggttcccgctgctcttcatgtcgctggtgcgctccgtggttggggttgtcaaccagcccatcgatgtcaccgtcaccctcaagctgggcggctatgagccgctgttcaccatgagcgcccagcagccgtccatcatccccttcacggcccaggcctatgaggagctgtcccggcagtttgacccccagccgctggccatgcagttcatcagccagtacagccctgaggacatcgtcacggcgcagattgagggcagctccggggcgctgtggcgcatcagtccccccagccgtgcccagatgaagcgggagctctacaacggcacggccgacatcaccctgcgcttcacctggaacttccagagggacctggcgaagggaggcactgtggagtatgccaacgagaagcacatgctggccctggcccccaacagcactgcacggcggcagctggccagcctgctcgagggcacctcggatcagtctgtggtcatccccaatctcttccccaagtacatccgtgcccccaacgggcccgaagccaaccctgtgaagcagctgcagcccaatgaggaggccgactacctcggcgtgcgtatccagctgcggagggagcagggtgcgggggccaccggcttcctcgaatggtgggtcatcgagctgcaggagtgccggaccgactgcaacctgctgcccatggtcattttcagtgacaagcatcatggggctgtacgtgtccatcgtgctggtcatcggcaagttcgtgcgcggattcttcagcgagatctcgcactccattatgttcgaggagctgccgtgcgtggaccgcatcctcaagctctgccaggacatcttcctggtgcgggagactcgggagctggagctggaggaggagttgtatgccaagctcatcttcctctaccgctcaccggagaccatgatcaagtggactcgtgagaaggagtaggagctgctgctggcgcccgagagggaaggagccggcctgctgggcagcgtggccacaaggggcggcactcctcaggccgggggagccactgccccgtccaaggccgccagctgtgatgcatcctcccggcctgcctgagccctgatgctgctgtcagagaaggacactgcgtccccacggcctgcgtggcgctgccgtcccccacgtgtactgtagagtttttttttttaattaaaaaatgttttatttatacaaatggaaaaaaaaaaaaaaaaaaaaaaa

SEQ ID NO: 27 - DNA, 6368 bp, Homo sapiens
  source 1..6368: mol_type = genomic DNA
ccatggacctgcgccctgagctgcccaccaccctgggccccgtcagcctgcgccagctggggctggagcacacccgctacccctgtctggaccttggtgccatgttgctctacaccctgaccttctggctcctgctgcgccagtttgtgaaagagaagctgctgaagtgggcagagtctccagctgcgctgacggaggtcaccgtggcagacacagagcctacgcggacgcagacgctgttgcagagcctgggggagctggtgaagggcgtgtacgccaagtactggatctatgtgtgtgctggcatgttcatcgtggtcagcttcgccggccgcctcgtggtctacaagattgtctacatgttcctcttcctgctctgcctcaccctcttccaggtctactacagcctgtggcggaagctgctcaaggccttctggtggctcgtggtggcctacaccatgctggtcctcatcgccgtctacaccttccagttccaggacttccctgcctactggcgcaacctcactggcttcaccgacgagcagctgggggacctgggcctggagcagttcagcgtatccgagctcttctccagcatcctggtgcccggcttcttcctcctggcctgcatcctgcagctgcactacttccacaggcccttcatgcagctcaccgacatggagcacgtgtccctgcctggcacgcgcctcccgcgctgggctcacaggcaggatgcagtgagtgggaccccactgctgcgggaggagcagcaggagcatcagcagcagcagcaggaggaggaggaggaggaggactccagggacgaggggctgggcgtggccactccccaccaggccacgcaggtgcctgaaggggcagccaagtggggcctggtggctgagcgcctgctggagctggcagccggcttctcggacgtcctctcacgcgtgcaggtgttcctgcggcggctgctggagcttcacgttttcaagctggtggccctgtacaccgtctgggtggccctgaaggaggtgtcggtgatgaacctgctgctggtggtgctgtgggccttcgccctgccctacccacgcttccggcccatggcctcctgcctgtccaccgtgtggacctgcgtcatcatcgtgtgtaagatgctgtaccagctcaaggttgtcaacccccaggagtattccagcaactgcaccgagcccttccccaacagcaccaacttgctgcccacggagatcagccagtccctgctgtaccgggggcccgtggaccctgccaactggtttggggtgcggaaagggttccccaacctgggctacatccagaaccacctgcaagtgctgctgctgctggtattcgaggccatcgtgtaccggcgccaggagcactaccgccggcagcaccagctggccccgctgcctgcccaggccgtgtttgccagcggcacccgccagcagctggaccaggatctgctcggctgcctcaagtacttcatcaacttcttcttctacaaattcgggctggagatctgcttcctgatggccgtgaacgtgatcgggcagcgcatgaactttctggtgaccctgcacggttgctggctggtggccatcctcacccgcaggcaccgccaggccattgcccgcctctggcccaactactgcctcttcctggcgctgttcctgctgtaccagtacctgctgtgcctggggatgcccccggccctgtgcattgattatccctggcgctggagccgggccgtccccatgaactccgcactcatcaagtggctgtacctgcctgatttcttccgggcccccaactccaccaacctcatcagcgactttctcctgctgctgtgcgcctcccagcagtggcaggtgttctcagctgagcgcacagaggagtggcagcgcatggctggcgtcaacaccgaccgcctggagccgctgcggggggagcccaaccccgtgcccaactttatccactgcaggtcctaccttgacatgctgaaggtggccgtcttccgatacctgttctggctggtgctggtggtggtgtttgtcacgggggccacccgcatcagcatcttcgggctgggctacctgctggcctgcttctacctgctgctcttcggcacggccctgctgcagagggacacacgggcccgcctcgtgctgtgggactgcctcattctgtacaacgtcaccgtcatcatctccaagaacatgctgtcgctcctggcctgcgtcttcgtggagcagatgcagaccggcttctgctgggtcatccagctcttcagccttgtatgcaccgtcaagggctactatgaccccaaggagatgatggacagagaccaggactgcctgctgcctgtggaggaggctggcatcatctgggacagcgtctgcttcttcttcctgctgctgcagcgccgcgtcttccttagccattactacctgcacgtcagggccgacctccaggccaccgccctgctagcctccaggggcttcgccctctacaacgctgccaacctcaagagcattgacttccaccgcaggatagaggagaagtccctggcccagctgaaaagacagatggagcgtatccgtgccaagcaggagaagcacaggcagggccgggtggaccgcagtcgcccccaggacaccctgggccccaaggaccccggcctggagccagggcccgacagtccagggggctcctccccgccacggaggcagtggtggcggccctggctggaccacgccacagtcatccactccggggactacttcctgtttgagtccgacagtgaggaagaggaggaggctgttcctgaagacccgaggccgtcggcacagagtgccttccagctggcgtaccaggcatgggtgaccaacgcccaggcggtgctgaggcggcggcagcaggagcaggagcaggcaaggcaggaacaggcaggacagctacccacaggaggtggtcccagccaggaggtggagccagcagagggccccgaggaggcagcggcaggccggagccatgtggtgcagagggtgctgagcacggcgcagttcctgtggatgctggggcaggcgctagtggatgagctgacacgctggctgcaggagttcacccggcaccacggcaccatgagcgacgtgctgcgggcagagcgctacctcctcacacaggagctcctgcagggcggcgaagtgcacaggggcgtgctggatcagctgtacacaagccaggccgaggccacgctgccaggccccaccgaggcccccaatgccccaagcaccgtgtccagtgggctgggcgcggaggagccactcagcagcatgacagacgacatgggcagccccctgagcaccggctaccacacgcgcagtggcagtgaggaggcagtcaccgaccccggggagcgtgaggctggtgcctctctgtaccagggactgatgcggacggccagcgagctgctcctggacaggcgcctgcgcatcccagagctggaggaggcagagctgtttgcggaggggcagggccgggcgctgcggctgctgcgggccgtgtaccagtgtgtggccgcccactcggagctgctctgctacttcatcatcatcctcaaccacatggtcacggcctccgccggctccctggtgctgcccgtgctcgtcttcctgtgggccatgctgtcgatcccgaggcccagcaagcgcttctggatgacggccatcgtcttcaccgagatcgcggtggtcgtcaagtacctgttccagtttgggttcttcccctggaacagccacgtggtgctgcggcgctacgagaacaagccctacttcccgccccgcatcctgggcctggagaagactgacggctacatcaagtacgacctggtgcagctcatggcccttttcttccaccgctcccagctgctgtgctatggcctctgggaccatgaggaggactcaccatccaaggagcatgacaagagcggcgaggaggagcagggagccgaggaggggccaggggtgcctgcggccaccaccgaagaccacattcaggtggaagcgagggtcggacccacggacgggaccccagaaccccaagtggagctcaggccccgtgatacgaggcgcatcagtctacgttttagaagaaggaagaaggagggcccagcacggaaaggagcggcagccatcgaagctgaggacagggaggaagaagagggggaggaagagaaagaggcccccacggggagagagaagaggccaagccgctctggaggaagagtaagggcggccgggcggcggctgcagggcttctgcctgtccctggcccagggcacatatcggccgctacggcgcttcttccacgacatcctgcacaccaagtaccgcgcagccaccgacgtctatgccctcatgttcctggctgatgttgtcgacttcatcatcatcatttttggcttctgggcctttgggaagcactcggcggccacagacatcacgtcctccctatcagacgaccaggtacccgaggctttcctggtcatgctgctgatccagttcagtaccatggtggttgaccgcgccctctacctgcgcaagaccgtgctgggcaagctggccttccaggtggcgctggtgctggccatccacctatggatgttcttcatcctgcccgccgtcactgagaggatgttcaaccagaatgtggtggcccagctctggtacttcgtgaagtgcatctacttcgccctgtccgcctaccagatccgctgcggctaccccacccgcatcctcggcaacttcctcaccaagaagtacaatcatctcaacctcttcctcttccaggggttccggctggtgccgttcctggtggagctgcgggcagtgatggactgggtgtggacggacaccacgctgtccctgtccagctggatgtgtgtggaggacatctatgccaacatcttcatcatcaaatgcagccgagagacagagaagaaatacccgcagcccaaagggcagaagaagaagaagatcgtcaagtacggcatgggtggcctcatcatcctcttcctcatcgccatcatctggttcccgctgctcttcatgtcgctggtgcgctccgtggttggggttgtcaaccagcccatcgatgtcaccgtcaccctcaagctgggcggctatgagccgctgttcaccatgagcgcccagcagccgtccatcatccccttcacggcccaggcctatgaggagctgtcccggcagtttgacccccagccgctggccatgcagttcatcagccagtacagccctgaggacatcgtcacggcgcagattgagggcagctccggggcgctgtggcgcatcagtccccccagccgtgcccagatgaagcgggagctctacaacggcacggccgacatcaccctgcgcttcacctggaacttccagagggacctggcgaagggaggcactgtggagtatgccaacgagaagcacatgctggccctggcccccaacagcactgcacggcggcagctggccagcctgctcgagggcacctcggaccagtctgtggtcatccccaatctcttccccaagtacatccgtgcccccaacgggcccgaagccaaccctgtgaagcagctgcagcccaatgaggaggccgactacctcggcgtgcgtatccagctgcggagggagcagggtgcgggggccaccggcttcctcgaatggtgggtcatcgagctgcaggagtgccggaccgactgcaacctgctgcccatggtcattttcagtgacaaggtcagcccaccgagcctcggcttcctggctggctacggcatcatggggctgtacgtgtccatcgtgctggtcatcggcaagttcgtgcgcggattcttcagcgagatctcgcactccattatgttcgaggagctgccgtgcgtggaccgcatcctcaagctctgccaggacatcttcctggtgcgggagactcgggagctggagctggaggaggagttgtacgccaagctcatcttcctctaccgctcaccggagaccatgatcaagtggactcgtgagaaggagtaggagctgctgctggcgcccgagagggaaggagccggcctgctgggcagcgtggccacaaggggcggcactcctcaggccgggggagccactgccccgtccaaggccgccagctgtgatgcatcctcccggcctgcctgagccctgatgctgctgtcagagaaggacactgcgtccccacggcctgcgtggcgctgccgtcccccacgtgtactgtagagttttttttttaattaaaaaatgttttatttatacaaatgg

SEQ ID NO: 28 - DNA, 6273 bp, Homo sapiens
  source 1..6273: mol_type = genomic DNA
atggtatggagcatcacctaccacagctggctgaccttcgtactgctgctctgggcctgcctcatctggacggtgcgcagccgccaccaactggccatgctgtgctcgccctgcatcctgctgtatgggatgacgctgtgctgcctacgctacgtgtgggccatggacctgcgccctgagctgcccaccaccctgggccccgtcagcctgcgccagctggggctggagcacacccgctacccctgtctggaccttggtgccatgttgctctacaccctgaccttctggctcctgctgcgccagtttgtgaaagagaagctgctgaagtgggcagagtctccagctgcgctgacggaggtcaccgtggcagacacagagcccacgcggacgcagacgctgttgcagagcctgggggagctggtgaagggcgtgtacgccaagtactggatctatgtgtgtgctggcatgttcatcgtggtcagcttcgccggccgcctcgtggtctacaagattgtctacatgttcctcttcctgctctgcctcaccctcttccaggtctactacagcctgtggcggaagctgctcaaggccttctggtggctcgtggtggcctacaccatgctggtcctcatcgccgtctacaccttccagttccaggacttccctgcctactggcgcaacctcactggcttcaccgacgagcagctgggggacctgggcctggagcagttcagcgtgtccgagctcttctccagcatcctggtgcccggcttcttcctcctggcctgcatcctgcagctgcactacttccacaggcccttcatgcagctcaccgacatggagcacgtgtccctgcctggcacgcgcctcccgcgctgggctcacaggcaggatgcagtgagtgggaccccactgctgcgggaggagcagcaggagcatcagcagcagcagcaggaggaggaggaggaggaggaggactccagggacgaggggctgggcgtggccactccccaccaggccacgcaggtgcctgaaggggcagccaagtggggcctggtggctgagcggctgctggagctggcagccggcttctcggacgtcctctcacgcgtgcaggtgttcctgcggcggctgctggagcttcacgttttcaagctggtggccctgtacaccgtctgggtggccctgaaggaggtgtcggtgatgaacctgctgctggtggtgctgtgggccttcgccctgccctacccacgcttccggcccatggcctcctgcctgtccaccgtgtggacctgcgtcatcatcgtgtgtaagatgctgtaccagctcaaggttgtcaacccccaggagtattccagcaactgcaccgagcccttccccaacagcaccaacttgctgcccacggagatcagccagtccctgctgtaccgggggcccgtggaccctgccaactggtttggggtgcggaaagggttccccaacctgggctacatccagaaccacctgcaagtgctgctgctgctggtattcgaggccatcgtgtaccggcgccaggagcactaccgccggcagcaccagctggccccgctgcctgcccaggccgtgtttgccagcggcacccgccagcagctggaccaggatctgctcggctgcctcaagtacttcatcaacttcttcttctacaaattcgggctggagatctgcttcctgatggccgtgaacgtgatcgggcagcgcatgaactttctggtgaccctgcacggttgctggctggtggccatcctcacccgcaggcaccgccaggccattgcccgcctctggcccaactactgcctcttcctggcgctgttcctgctgtaccagtacctgctgtgcctggggatgcccccggccctgtgcattgattatccctggcgctggagccgggccgtccccatgaactccgcactcatcaagtggctgtacctgcctgatttcttccgggcccccaactccaccaacctcatcagcgactttctcctgctgctgtgcgcctcccagcagtggcaggtgttctcagctgagcgcacagaggagtggcagcgcatggctggcgtcaacaccgaccgcctggagccgctgcggggggagcccaaccccgtgcccaactttatccactgcaggtcctaccttgacatgctgaaggtggccgtcttccgatacctgttctggctggtgctggtggtggtgtttgtcacgggggccacccgcatcagcatcttcgggctgggctacctgctggcctgcttctacctgctgctcttcggcacggccctgctgcagagggacacacgggcccgcctcgtgctgtgggactgcctcattctgtacaacgtcaccgtcatcatctccaagaacatgctgtcgctcctggcctgcgtcttcgtggagcagatgcagaccggcttctgctgggtcatccagctcttcagccttgtatgcaccgtcaagggctactatgaccccaaggagatgatggacagagaccaggactgcctgctgcctgtggaggaggctggcatcatctgggacagcgtctgcttcttcttcctgctgctgcagcgccgcgtcttccttagccattactacctgcacgtcagggccgacctccaggccaccgccctgctagcctccaggggcttcgccctctacaacgctgccaacctcaagagcattgacttccaccgcaggatagaggagaagtccctggcccagctgaaaagacagatggagcgtatccgtgccaagcaggagaagcacaggcagggccgggtggaccgcagtcgcccccaggacaccctgggccccaaggaccccggcctggagccagggcccgacagtccagggggctcctccccgccacggaggcagtggtggcggccctggctggaccacgccacagtcatccactccggggactacttcctgtttgagtccgacagtgaggaagaggaggaggctgttcctgaagacccgaggccgtcggcacagagtgccttccagctggcgtaccaggcatgggtgaccaacgcccaggcggtgctgaggcggcggcagcaggagcaggagcaggcaaggcaggaacaggcaggacagctacccacaggaggtggtcccagccaggaggtggagccagcagagggccccgaggaggcagcggcaggccggagccatgtggtgcagagggtgctgagcacggcgcagttcctgtggatgctggggcaggcgctagtggatgagctgacacgctggctgcaggagttcacccggcaccacggcaccatgagcgacgtgctgcgggcagagcgctacctcctcacacaggagctcctgcagggcggcgaagtgcacaggggcgtgctggatcagctgtacacaagccaggccgaggccacgctgccaggccccaccgaggcccccaatgccccaagcaccgtgtccagtgggctgggcgcggaggagccactcagcagcatgacagacgacatgggcagccccctgagcaccggctaccacacgcgcagtggcagtgaggaggcagtcaccgaccccggggagcgtgaggctggtgcctctctgtaccagggactgatgcggacggccagcgagctgctcctggacaggcgcctgcgcatcccagagctggaggaggcagagctgtttgcggaggggcagggccgggcgctgcggctgctgcgggccgtgtaccagtgtgtggccgcccactcggagctgctctgctacttcatcatcatcctcaaccacatggtcacggcctccgccggctcgctggtgctgcccgtgctcgtcttcctgtgggccatgctgtcgatcccgaggcccagcaagcgcttctggatgacggccatcgtcttcaccgagatcgcggtggtcgtcaagtacctgttccagtttgggttcttcccctggaacagccacgtggtgctgcggcgctacgagaacaagccctacttcccgccccgcatcctgggcctggagaagactgacggctacatcaagtacgacctggtgcagctcatggcccttttcttccaccgctcccagctgctgtgctatggcctctgggaccatgaggaggactcaccatccaaggagcatgacaagagcggcgaggaggagcagggagccgaggaggggccaggggtgcctgcggccaccaccgaagaccacattcaggtggaagcgagggtcggacccacggacgggaccccagaaccccaagtggagctcaggccccgtgatacgaggcgcatcagtctacgttttagaagaaggaagaaggagggcccagcacggaaaggagcggcagccatcgaagctgaggacagggaggaagaagagggggaggaagagaaagaggcccccacggggagagagaagaggccaagccgctctggaggaagagtaagggcggccgggcggcggctgcagggcttctgcctgtccctggcccagggcacatatcggccgctacggcgcttcttccacgacatcctgcacaccaagtaccgcgcagccaccgacgtctatgccctcatgttcctggctgatgttgtcgacttcatcatcatcatttttggcttctgggcctttgggaagcactcggcggccacagacatcacgtcctccctatcagacgaccaggtacccgaggctttcctggtcatgctgctgatccagttcagtaccatggtggttgaccgcgccctctacctgcgcaagaccgtgctgggcaagctggccttccaggtggcgctggtgctggccatccacctatggatgttcttcatcctgcccgccgtcactgagaggatgttcaaccagaatgtggtggcccagctctggtacttcgtgaagtgcatctacttcgccctgtccgcctaccagatccgctgcggctaccccacccgcatcctcggcaacttcctcaccaagaagtacaatcatctcaacctcttcctcttccaggggttccggctggtgccgttcctggtggagctgcgggcagtgatggactgggtgtggacggacaccacgctgtccctgtccagctggatgtgtgtggaggacatctatgccaacatcttcatcatcaaatgcagccgagagacagagaagaaatacccgcagcccaaagggcagaagaagaagaagatcgtcaagtacggcatgggtggcctcatcatcctcttcctcatcgccatcatctggttcccgctgctcttcatgtcgctggtgcgctccgtggttggggttgtcaaccagcccatcgatgtcaccgtcaccctgaagctgggcggctatgagccgctgttcaccatgagcgcccagcagccgtccatcatccccttcacggcccaggcctatgaggagctgtcccggcagtttgacccccagccgctggccatgcagttcatcagccagtacagccctgaggacatcgtcacggcgcagattgagggcagctccggggcgctgtggcgcatcagtccccccagccgtgcccagatgaagcgggagctctacaacggcacggccgacatcaccctgcgcttcacctggaacttccagagggacctggcgaagggaggcactgtggagtatgccaacgagaagcacatgctggccctggcccccaacagcactgcacggcggcagctggccagcctgctcgagggcacctcggaccagtctgtggtcatccccaatctcttccccaagtacatccgtgcccccaacgggcccgaagccaaccctgtgaagcagctgcagcccaatgaggaggccgactacctcggcgtgcgtatccagctgcggagggagcagggtgcgggggccaccggcttcctcgaatggtgggtcatcgagctgcaggagtgccggaccgactgcaacctgctgcccatggtcattttcagtgacaaggtcagcccaccgagcctcggcttcctggctggctacggcatcatggggctgtacgtgtccatcgtgctggtcatcggcaagttcgtgcgcggattcttcagcgagatctcgcactccattatgttcgaggagctgccgtgcgtggaccgcatcctcaagctctgccaggacatcttcctggtgcgggagactcgggagctggagctggaggaggagttgtacgccaagctcatcttcctctaccgctcaccggagaccatgatcaagtggactcgtgagaaggagtag

SEQ ID NO: 29 - DNA, 7563 bp, Homo sapiens
  source 1..7563: mol_type = genomic DNA
atggagccgcacgtgttcggcgcggtcctgtactggctgctgctgccctgcgcgctgctggctgcctgcctgctccgcttcagcggactctcgctggtctacctgctcttcctgctgctgctgccctggttccccggccccacccgatgcggcctccaaggtcacacaggccgcctcctgcgggcattgctgggcctcagcctgctcttcctggtggcccatctcgccctccagatctgcctgcatactgtgccccgcctggaccagctcctgggacccagctgcagccgctgggagaccctctcgcgacacataggggtcacaaggctggacctgaaggacatccccaacgccatccggctggtggcccctgacctgggcatcttggtggtctcctctgtctgcctcggcatctgcgggcgccttgcaaggaacacccggcagagcccacatccacgggagctggatgatgatgagagggatgtggatgccagcccgacggcagggctgcaggaagcagcaacgctggcccctacacggaggtcacggctggccgctcgtttccgagtcacggcccactggctgctggtggcggctgggcgggtcctggccgtaacactgcttgcactggcaggcatcgcccacccctcggccctctccagtgtctacctgctgctcttcctggccctctgcacctggtgggcctgccactttcccatcagcactcggggcttcagcagactctgcgccgcggtggggtgcttcggcgccggccatctcatctgcctctactgctaccagatgcccttggcacaggctctgctcccgcctgccggcatctgggctagggtgctgggtctcaaggacttcgtgggtcccaccaactgctccagcccccacgcgctggtcctcaacaccggcctggactggcctgtgtatgccagccccggcgtcctcctgctgctgtgctacgccacggcctctctgcgcaagctccgcgcgtaccgcccctccggccagaggaaggaggcggcaaaggggtatgaggctcgggagctggagctagcagagctggaccagtggccccaggaacgggagtctgaccagcacgtggtgcccacagcacccgacaccgaggctgataactgcatcgtgcacgagctgaccggccagagctccgtcctgcggcggcctgtgcggcccaagcgggctgagcccagggaggcgtctccgctccacagcctgggccacctcatcatggaccagagctatgtgtgcgcgctcattgccatgatggtatggagcatcacctaccacagctggctgaccttcgtactgctgctctgggcctgcctcatctggacggtgcgcagccgccaccaactggccatgctgtgctcgccctgcatcctgctgtatgggatgacgctgtgctgcctacgctacgtgtgggccatggacctgcgccctgagctgcccaccaccctgggccccgtcagcctgcgccagctggggctggagcacacccgctacccctgtctggaccttggtgccatgttgctctacaccctgaccttctggctcctgctgcgccagtttgtgaaagagaagctgctgaagtgggcagagtctccagctgcgctgacggaggtcaccgtggcagacacagagcccacgcggacgcagacgctgttgcagagcctgggggagctggtgaagggcgtgtacgccaagtactggatctatgtgtgtgctggcatgttcatcgtggtcagcttcgccggccgcctcgtggtctacaagattgtctacatgttcctcttcctgctctgcctcaccctcttccaggtctactacagcctgtggcggaagctgctcaaggccttctggtggctcgtggtggcctacaccatgctggtcctcatcgccgtctacaccttccagttccaggacttccctgcctactggcgcaacctcactggcttcaccgacgagcagctgggggacctgggcctggagcagttcagcgtgtccgagctcttctccagcatcctggtgcccggcttcttcctcctggcctgcatcctgcagctgcactacttccacaggcccttcatgcagctcaccgacatggagcacgtgtccctgcctggcacgcgcctcccgcgctgggctcacaggcaggatgcagtgagtgggaccccactgctgcgggaggagcagcaggagcatcagcagcagcagcaggaggaggaggaggaggaggaggactccagggacgaggggctgggcgtggccactccccaccaggccacgcaggtgcctgaaggggcagccaagtggggcctggtggctgagcggctgctggagctggcagccggcttctcggacgtcctctcacgcgtgcaggtgttcctgcggcggctgctggagcttcacgttttcaagctggtggccctgtacaccgtctgggtggccctgaaggaggtgtcggtgatgaacctgctgctggtggtgctgtgggccttcgccctgccctacccacgcttccggcccatggcctcctgcctgtccaccgtgtggacctgcgtcatcatcgtgtgtaagatgctgtaccagctcaaggttgtcaacccccaggagtattccagcaactgcaccgagcccttccccaacagcaccaacttgctgcccacggagatcagccagtccctgctgtaccgggggcccgtggaccctgccaactggtttggggtgcggaaagggttccccaacctgggctacatccagaaccacctgcaagtgctgctgctgctggtattcgaggccatcgtgtaccggcgccaggagcactaccgccggcagcaccagctggccccgctgcctgcccaggccgtgtttgccagcggcacccgccagcagctggaccaggatctgctcggctgcctcaagtacttcatcaacttcttcttctacaaattcgggctggagatctgcttcctgatggccgtgaacgtgatcgggcagcgcatgaactttctggtgaccctgcacggttgctggctggtggccatcctcacccgcaggcaccgccaggccattgcccgcctctggcccaactactgcctcttcctggcgctgttcctgctgtaccagtacctgctgtgcctggggatgcccccggccctgtgcattgattatccctggcgctggagccgggccgtccccatgaactccgcacccatcaagtggctgtacctgcctgatttcttccgggcccccaactccaccaacctcatcagcgactttctcctgctgctgtgcgcctcccagcagtggcaggtgttctcagctgagcgcacagaggagtggcagcgcatggctggcgtcaacaccgaccgcctggagccgctgcggggggagcccaaccccgtgcccaactttatccactgcaggtcctaccttgacatgctgaagctggccgtcttccgatacctgttctggctggtgctggtggtggtgtttgtcacgggggccacccgcatcagcatcttcgggctgggctacctgctggcctgcttctacctgctgctcttcggcacggccctgctgcagagggacacacgggcccgcctcgtgctgtgggactgcctcattctgtacaacgtcaccgtcatcatctccaagaacatgctgtcgctcctggcctgcgtcttcgtggagcagatgcagaccggcttctgctgggtcatccagctcttcagccttgtatgcaccgtcaagggctactatgaccccaaggagatgatggacagagaccaggactgcctgctgcctgtggaggaggctggcatcatctgggacagcgtctgcttcttcttcctgctgctgcagcgccgcgtcttccttagccattactacctgcacgtcagggccgacctccaggccaccgccctgctagcctccaggggcttcgccctctacaacgctgccaacctcaagagcattgacttccaccgcaggatagaggagaagtccctggcccagctgaaaagacagatggagcgtatccgtgccaagcaggagaagcacaggcagggccgggtggaccgcagtcgcccccaggacaccctgggccccaaggaccccggcctggagccagggcccgacagtccagggggctcctccccgctacggaggcagtggtggcggccctggctggaccacgccacagtcatccactccggggactacttcctgtttgagtccgacagtgaggaagaggaggaggctgttcctgaagacccgaggccgtcggcacagagtgccttccagctggcgtaccaggcatgggtgaccaacgcccaggcggtgctgaggcggcggcagcaggagcaggagcaggcaaggcaggaacaggcaggacagctacccacaggaggtggtcccagccaggaggtggagccagcagagggccccgaggaggcagcggcaggccggagccatgtggtgcagagggtgctgagcacggcgcagttcctgtggatgctggggcaggcgctagtggatgagctgacacgctggctgcaggagttcacccggcaccacggcaccatgagcgacgtgctgcgggcagagcgctacctcctcacacaggagctcctgcagggcggcgaagtgcacaggggcgtgctggatcagctgtacacaagccaggccgaggccacgctgccaggccccaccgaggcccccaatgccccaagcaccgtgtccagtgggctgggcgcggaggagccactcagcagcatgacagacgacatgggcagccccctgagcaccggctaccacacgcgcagtggcagtgaggaggcagtcaccgaccccggggagcgtgaggctggtgcctctctgtaccagggactgatgcggacggccagcgagctgctcctggacaggcgcctgcgcatcccagagctggaggaggcagagctgtttgcggaggggcagggccgggcgctgcggctgctgcgggccgtgtaccagtgtgtggccgcccactcggagctgctctgctacttcatcatcatcctcaaccacatggtcacggcctccgccggctcgctggtgctgcccgtgctcgtcttcctgtgggccatgctgtcgatcccgaggcccagcaagcgcttctggatgacggccatcgtcttcaccgagatcgcggtggtcgtcaagtacctgttccagtttgggttcttcccctggaacagccacgtggtgctgcggcgctacgagaacaagccctacttcccgccccgcatcctgggcctggagaagactgacggctacatcaagtacgacctggtgcagctcatggcccttttcttccaccgctcccagctgctgtgctatggcctctgggaccatgaggaggactcaccatccaaggagcatgacaagagcggcgaggaggagcagggagccgaggaggggccaggggtgcctgcggccaccaccgaagaccacattcaggtggaagccagggtcggacccacggacgggaccccagaatcccaagtggagctcaggccccgtgatacgaggcgcatcagtctacgttttagaagaaggaaggagggcccagcacggaaaggagcggcagccatcgaagctgaggacagggaggaagaagagggggaggaagagaaagaggcccccacggggagagagaagaggccaagccgctctggaggaagagtaagggcggccgggcggcggctgcagggcttctgcctgtccctggcccagggcacatatcggccgctacggcgcttcttccacgacatcctgcacaccaagtaccgcgcagccaccgacgtctatgccctcatgttcctggctgatgttgtcgacttcatcatcatcatttttggcttctgggcctttgggaagcactcggcggccacagacatcacgtcctccctatcagacgaccaggtacccgaggctttcctggtcatgctgctgatccagttcagtaccatggtggttgaccgcgccctctacctgcgcaagaccgtgctgggcaagctggccttccaggtggcgctggtgctggccatccacctatggatgttcttcatcctgcccgccgtcactgagaggatgttcaaccagaatgtggtggcccagctctggtacttcgtgaagtgcatctacttcgccctgtccgcctaccagatccgctgcggctaccccacccgcatcctcggcaacttcctcaccaagaagtacaatcatctcaacctcttcctcttccaggggttccggctggtgccgttcctggtggagctgcgggcagtgatggactgggtgtggacggacaccacgctgtccctgtccagctggatgtgtgtggaggacatctatgccaacatcttcatcatcaaatgcagccgagagacagagaagaaatacccgcagcccaaagggcagaagaagaagaagatcgtcaagtacggcatgggtggcctcatcatcctcttcctcatcgccatcatctggttcccgctgctcttcatgtcgctggtgcgctccgtggttggggttgtcaaccagcccatcgatgtcaccgtcaccctgaagctgggcggctatgagccgctgttcaccatgagcgcccagcagccgtccatcatccccttcacggcccaggcctatgaggagctgtcccggcagtttgacccccagccgctggccatgcagttcatcagccagtacagccctgaggacgtcgtcacggcgcagattgagggcagctccggggcgctgtggcgcatcagtccccccagccgtgcccagatgaagcgggagctctacaacggcacggccgacatcaccctgcgcttcacctggaacttccagagggacctggcgaagggaggcactgtggagtatgccaacgagaagcacatgctggccctggcccccaacagcactgcacggcggcagctggccagcctgctcgagggcacctcggaccagtctgtggtcatccccaatctcttccccaagtacatccgtgcccccaacgggcccgaagccaaccctgtgaagcagctgcagcccaatgaggaggccgactacctcggcgtgcgtatccagctgcggagggagcagggtgcgggggccaccggcttcctcgaatggtgggtcatcgaactgcaggagtgccggaccgactgcaacctgctgcccatggtcattttcagtgacaaggtcagcccaccgagcctcggcttcctggctggctacggcatcatggggctgtacgtgtccatcgtgctggtcatcggcaagttcgtgcgcggattcttcagcgagatctcgcactccattatgttcgaggagctgccgtgcgtggaccgcatcctcaagctctgccaggacatcttcctggtgcgggagactcgggagctggagctggaggaggagttgtacgccaagctcatcttcctctaccgctcaccggagaccatgatcaagtggactcgtgagaaggagtag

SEQ ID NO: 30 - DNA, 1635 bp, Homo sapiens
  source 1..1635: mol_type = genomic DNA
taccagatccgctgcggctaccccacccgcatcctcggcaacttcctcaccaagaagtacaatcatctcaacctcttcctcttccaggggttccggctggtgccgttcctggtggagctgcgggcagtgatggactgggtgtggacggacaccacgctgtccctgtccagctggatgtgtgtggaggacatctatgccaacatcttcatcatcaaatgcagccgagagacagagaaggttcatgaagatttgcccgttttcttctaaaagttctatagttttagctctgaagtttcgctctttgatccactttgagaaatacccgcagcccaaagggcagaagaagaagaagatcgtcaagtacggcatgggtggcctcatcatcctcttcctcatcgccatcatctggttcccactgctcttcatgtcgctggtgcgctccgtggttggggttgtcaaccagcccatcgatgtcaccgtcaccctgaagctgggcggctatgagccgctgttcaccatgagcgcccagcagccgtccatcatccccttcacggcccaggcctatgaggagctgtcccggcagtttgacccccagccgctggccatgcagttcatcagccagtacagccctgaggacatcgtcacggcgcagattgagggcagctccggggcgctgtggcgcatcagtccccccagccgtgcccagatgaagcgggagctctacaacggcacggccgacatcaccctgcgcttcacctggaacttccagagggacctggcgaagggaggcactgtggagtatgccaacgagaagcacatgctggccctggcccccaacagcactgcacggcggcagctggccagcctgctcgagggcacctcggaccagtctgtggtcatccctaatctcttccccaagtacatccgtgcccccaacgggcccgaagccaaccctgtgaagcagctgcagcccaatgaggaggccgactacctcggcgtgcgtatccagctgcggagggagcagggtgcgggggccaccggcttcctcgaatggtgggtcatcgagctgcaggagtgccggaccgactgcaacctgctgcccatggtcattttcagtgacaagcatcatggggctgtacgtgtccatcgtgctggtcatcggcaagttcgtgcgcggattcttcagcgagatctcgcactccattatgttcgaggagctgccgtgcgtggaccgcatcctcaagctctgccaggacatcttcctggtgcgggagactcgggagctggagctggaggaggagttgtacgccaagctcatcttcctctaccgctcaccggagaccatgatcaagtggactcgtgagaaggagtaggagctgctgctggcgcccgagagggaaggagccggcctgctgggcagcgtggccacaaggggcggcactcctcaggccgggggagccactgccccgtccaaggccgccagctgtgatgcatcctcccggcctgcctgagccctgatgctgctgtcagagaaggacactgcgtccccacggcctgcgtggcgctgccgtcccccacgtgtactgtagagttttttttttaattaaaaaatgttttatttatacaaatggacaa

SEQ ID NO: 31 - DNA, 660 bp, Homo sapiens
  source 1..660: mol_type = genomic DNA
ggtcatccctaatctcttccccaagtacatccgtgcccccaacgggcccgaagccaaccctgtgaagcagctgcagcccaatgaggaggccgactacctcggctgacaaggtcagcccaccgagcctcggcttcctggctggctacggcatcatggggctgtacgtgtccatcgtgctggtcatcggcaagttcgtgcgcggattcttcagcgagatctcgcactccattatgttcgaggagctgccgtgcgtggaccgcatcctcaagctctgccaggacatcttcctggtgcgggagactcgggagctggagctggaggaggagttgtacgccaagctcatcttcctctaccgctcaccggagaccatgatcaagtggactcgtgagaaggagtaggagctgctgctggcgcccgagagggaaggagccggcctgctgggcagcgtggccacaaggggcggcactcctcaggccgggggagccactgccccgtccaaggccgccagctgtgatgcatcctcccggcctgcctgagccctgatgctgctgtcagagaaggacactgcgtccccacggcctgcgtggcgctgccgtcccccacgtgtactgtagagttttttttttaattaaaaaatgttttatttatacaaatggacaa

SEQ ID NO: 32 - DNA, 8072 bp, Homo sapiens
  source 1..8072: mol_type = genomic DNA
cgtccggcccagctcggccccagtgagccgagcgctgcgctccgccgaggggcagggcggtcgcctgagcgagcgcgggcccgggacgtcggcaccggcggggcggccgaaggagaaggaggaagaggagaaggcggcgcgcgggtccccgcgggtcagccatggcgcgccggccccggggcccccgcaccgccccatagcgccgcggcgtccgctcggtctgggccgggccctgggccctccagccatggagccgcacgtgctcggcgcggtcctgtactggctgctgctgccctgcgcgctgctggctgcctgcctgctccgcttcagcggactctcgctggtctacctgctcttcctgctgctgctgccctggttccccggccccacccgatgcggcctccaaggtcacacaggccgcctcctgcgggcattgctgggcctcagcctgctcttcctggtggcccatctcgccctccagatctgcctgcatattgtgccccgcctggaccagctcctgggacccagctgcagccgctgggagaccctctcgcgacacataggggtcacaaggctggacctgaaggacatccccaacgccatccggctggtggcccctgacctgggcatcttggtggtctcctctgtctgcctcggcatctgcgggcgccttgcaaggaacacccggcagagcccacatccacgggagctggatgatgatgagagggatgtggatgccagcccgacggcagggctgcaggaagcagcaacgctggcccctacacggaggtcacggctggccgctcgtttccgagtcacggcccactggctgctggtggcggctgggcgggtcctggccgtaacactgcttgcactggcaggcatcgcccacccctcggccctctccagtgtctacctgctgctcttcctggccctctgcacctggtgggcctgccactttcccatcagcactcggggcttcagcagactctgcgtcgcggtggggtgcttcggcgccggccatctcatctgcctctactgctaccagatgcccttggcacaggctctgctcccgcctgccggcatctgggctagggtgctgggtctcaaggacttcgtgggtcccaccaactgctccagcccccacgcgctggtcctcaacaccggcctggactggcctgtgtatgccagccccggcgtcctcctgctgctgtgctacgccacggcctctctgcgcaagctccgcgcgtaccgcccctccggccagaggaaggaggcggcaaaggggtatgaggctcgggagctggagctagcagagctggaccagtggccccaggaacgggagtctgaccagcacgtggtgcccacagcacccgacaccgaggctgataactgcatcgtgcacgagctgaccggccagagctccgtcctgcggcggcctgtgcggcccaagcgggctgagcccagggaggcgtctccgctccacagcctgggccacctcatcatggaccagagctatgtgtgcgcgctcattgccatgatggtatggagcatcacctaccacagctggctgaccttcgtactgctgctctgggcctgcctcatctggacggtgcgcagccgccaccaactggccatgctgtgctcgccctgcatcctgctgtatgggatgacgctgtgctgcctacgctacgtgtgggccatggacctgcgccctgagctgcccaccaccctgggccccgtcagcctgcgccagctggggctggagcacacccgctacccctgtctggaccttggtgccatgttgctctacaccctgaccttctggctcctgctgcgccagtttgtgaaagagaagctgctgaagtgggcagagtctccagctgcgctgacggaggtcaccgtggcagacacagagcccacgcggacgcagacgctgttgcagagcctgggggagctggtgaagggcgtgtacgccaagtactggatctatgtgtgtgctggcatgttcatcgtggtcagcttcgccggccgcctcgtggtctacaagattgtctacatgttcctcttcctgctctgcctcaccctcttccaggtctactacagcctgtggcggaagctgctcaaggccttctggtggctcgtggtggcctacaccatgctggtcctcatcgccgtctacaccttccagttccaggacttccctgcctactggcgcaacctcactggcttcaccgacgagcagctgggggacctgggcctggagcagttcagcgtgtccgagctcttctccagcatcctggtgcccggcttcttcctcctggcctgcatcctgcagctgcactacttccacaggcccttcatgcagctcaccgacatggagcacgtgtccctgcctggcacgcgcctcccgcgctgggctcacaggcaggatgcagtgagtgggaccccactgctgcgggaggagcagcaggagcatcagcagcagcagcaggaggaggaggaggaggaggaggactccagggacgaggggctgggcgtggccactccccaccaggccacgcaggtgcctgaaggggcagccaagtggggcctggtggctgagcggctgctggagctggcagccggcttctcggacgtcctctcacgcgtgcaggtgttcctgcggcggctgctggagcttcacgttttcaagctggtggccctgtacaccgtctgggtggccctgaaggaggtgtcggtgatgaacctgctgctggtggtgctgtgggccttcgccctgccctacccacgcttccggcccatggcctcctgcctgtccaccgtgtggacctgcgtcatcatcgtgtgtaagatgctgtaccagctcaaggttgtcaacccccaggagtattccagcaactgcaccgagcccttccccaacagcaccaacttgctgcccacggagatcagccagtccctgctgtaccgggggcccgtggaccctgccaactggtttggggtgcggaaagggttccccaacctgggctacatccagaaccacctgcaagtgctgctgctgctggtattcgaggccatcgtgtaccggcgccaggagcactaccgccggcagcaccagctggccccgctgcctgcccaggccgtgtttgccagcggcacccgccagcagctggaccaggatctgctcggctgcctcaagtacttcatcaacttcttcttctacaaattcgggctggagatctgcttcctgatggccgtgaacgtgatcgggcagcgcatgaactttctggtgaccctgcacggttgctggctggtggccatcctcacccgcaggcaccgccaggccattgcccgcctctggcccaactactgcctcttcctggcgctgttcctgctgtaccagtacctgctgtgcctggggatgcccccggccctgtgcattgattatccctggcgctggagccgggccgtccccatgaactccgcactcatcaagtggctgtacctgcctgatttcttccgggcccccaactccaccaacctcatcagcgactttctcctgctgctgtgcgcctcccagcagtggcaggtgttctcagctgagcgcacagaggagtggcagcgcatggctggcgtcaacaccgaccgcctggagccgctgcggggggagcccaaccccgtgcccaactttatccactgcaggtcctaccttgacatgctgaaggtggccgtcttccgatacctgttctggctggtgctggtggtggtgtttgtcacgggggccacccgcatcagcatcttcgggctgggctacctgctggcctgcttctacctgctgctcttcggcacggccctgctgcagagggacacacgggcccgcctcgtgctgtgggactgcctcattctgtacaacgtcaccgtcatcatctccaagaacatgctgtcgctcctggcctgcgtcttcgtggagcagatgcagaccggcttctgctgggtcatccagctcttcagccttgtatgcaccgtcaagggctactatgaccccaaggagatgatggacagagaccaggactgcctgctgcctgtggaggaggctggcatcatctgggacagcgtctgcttcttcttcctgctgctgcagcgccgcgtcttccttagccattactacctgcacgtcagggccgacctccaggccaccgccctgctagcctccaggggcttcgccctctacaacgctgccaacctcaagagcattgactttcaccgcaggatagaggagaagtccctggcccagctgaaaagacagatggagcgtatccgtgccaagcaggagaagcacaggcagggccgggtggaccgcagtcgcccccaggacaccctgggccccaaggaccccggcctggagccagggcccgacagtccagggggctcctccccgccacggaggcagtggtggcggccctggctggaccacgccacagtcatccactccggggactacttcctgtttgagtccgacagtgaggaagaggaggaggctgttcctgaagacccgaggccgtcggcacagagtgccttccagctggcgtaccaggcatgggtgaccaacgcccaggcggtgctgaggcggcggcagcaggagcaggagcaggcaaggcaggaacaggcaggacagctacccacaggaggtggtcccagccaggaggtggagccagcagagggccccgaggaggcagcggcaggccggagccatgtggtgcagagggtgctgagcacggcgcagttcctgtggatgctggggcaggcgctagtggatgagctgacacgctggctgcaggagttcacccggcaccacggcaccatgagcgacgtgctgcgggcagagcgctacctcctcacacaggagctcctgcagggcggcgaagtgcacaggggcgtgctggatcagctgtacacaagccaggccgaggccacgctgccaggccccaccgaggcccccaatgccccaagcaccgtgtccagtgggctgggcgcggaggagccactcagcagcatgacagacgacatgggcagccccctgagcaccggctaccacacgcgcagtggcagtgaggaggcagtcaccgaccccggggagcgtgaggctggtgcctctctgtaccagggactgatgcggacggccagcgagctgctcctggacaggcgcctgcgcatcccagagctggaggaggcagagctgtttgcggaggggcagggccgggcgctgcggctgctgcgggccgtgtaccagtgtgtggccgcccactcggagctgctctgctacttcatcatcatcctcaaccacatggtcacggcctccgccggctcgctggtgctgcccgtgctcgtcttcctgtgggccatgctgtcgatcccgaggcccagcaagcgcttctggatgacggccatcgtcttcaccgagatcgcggtggtcgtcaagtacctgttccagtttgggttcttcccctggaacagccacgtggtgctgcggcgctacgagaacaagccctacttcccgccccgcatcctgggcctggagaagactgacggctacatcaagtacgacctggtgcagctcatggcccttttcttccaccgctcccagctgctgtgctatggcctctgggaccatgaggaggactcaccatccaaggagcatgacaagagcggcgaggaggagcagggagccgaggaggggccaggggtgcctgcggccaccaccgaagaccacattcaggtggaagccagggtcggacccacggacgggaccccagaaccccaagtggagctcaggccccgtgatacgaggcgcatcagtctacgttttagaagaaggaagaaggagggcccagcacggaaaggagcggcagccatcgaagctgaggacagggaggaagaagagggggaggaagagaaagaggcccccacggggagagagaagaggccaagccgctctggaggaagagtaagggcggccgggcggcggctgcagggcttctgcctgtccctggcccagggcacatatcggccgctacggcgcttcttccacgacatcctgcacaccaagtaccgcgcagccaccgacgtctatgccctcatgttcctggctgatgttgtcgacttcatcatcatcatttttggcttctgggcctttgggaagcactcggcggccacagacatcacgtcctccctatcagacgaccaggtacccgaggctttcctggtcatgctgctgatccagttcagtaccatggtggttgaccgcgccctctacctgcgcaagaccgtgctgggcaagctggccttccaggtggcgctggtgctggccatccacctatggatgttcttcatcctgcccgccgtcactgagaggatgttcaaccagaatgtggtggcccagctctggtacttcgtgaagtgcatctacttcgccctgtccgcctaccagatccgctgcggctaccccacccgcatcctcggcaacttcctcaccaagaagtacaatcatctcaacctcttcctcttccaggggttccggctggtgccgttcctggtggagctgcgggcagtgatggactgggtgtggacggacaccacgctgtccctgtccagctggatgtgtgtggaggacatctatgccaacatcttcatcatcaaatgcagccgagagacagagaagaaatacccgcagcccaaagggcagaagaagaagaagatcgtcaagtacggcatgggtggcctcatcatcctcttcctcatcgccatcatctggttcccactgctcttcatgtcgctggtgcgctccgtggttggggttgtcaaccagcccatcgatgtcaccgtcaccctgaagctgggcggctatgagccgctgttcaccatgagcgcccagcagccgtccatcatccccttcacggcccaggcctatgaggagctgtcccggcagtttgacccccagccgctggccatgcagttcatcagccagtacagccctgaggacatcgtcacggcgcagattgagggcagctccggggcgctgtggcgcatcagtccccccagccgtgcccagatgaagcgggagctctacaacggcacggccgacatcaccctgcgcttcacctggaacttccagagggacctggcgaagggaggcactgtggagtatgccaacgagaagcacatgctggccctggcccccaacagcactgcacggcggcagctggccagcctgctcgagggcacctcggaccagtctgtggtcatccctaatctcttccccaagtacatccgtgcccccaacgggcccgaagccaaccctgtgaagcagctgcagcccaatgaggaggccgactacctcggcgtgcgtatccagctgcggagggagcagggtgcgggggccaccggcttcctcgaatggtgggtcatcgagctgcaggagtgccggaccgactgcaacctgctgcccatggtcattttcagtgacaaggtcagcccaccgagcctcggcttcctggctggctacggcatcatggggctgtacgtgtccatcgtgctggtcatcggcaagttcgtgcgcggattcttcagcgagatctcgcactccattatgttcgaggagctgccgtgcgtggaccgcatcctcaagctctgccaggacatcttcctggtgcgggagactcgggagctggagctggaggaggagttgtacgccaagctcatcttcctctaccgctcaccggagaccatgatcaagtggactcgtgagaaggagtaggagctgctgctggcgcccgagagggaaggagccggcctgctgggcagcgtggccacaaggggcggcactcctcaggccgggggagccactgccccgtccaaggccgccagctgtgatgcatcctcccggcctgcctgagccctgatgctgctgtcagagaaggacactgcgtccccacggcctgcgtggcgctgccgtcccccacgtgtactgtagagttttttttttaattaaaaaatgttttatttatacaaatgga

SEQ ID NO: 33 - DNA, 8089 bp, Homo sapiens
  source 1..8089: mol_type = genomic DNA
gggagccgccgtccggcccagctcggccccagtgagccgagcgctgcgctccgccgaggggcagggcggtcgcctgagcgagcgcgggcccgggacgtcggcaccggcggggcggccgaaggagaaggaggaagaggagaaggcggcgcgcgggtccccgcgggtcagccatggcgcgccggccccggggcccccgcaccgccccatagcgccgcggcgtccgctcggtctgggccgggccctgggccctccagccatggagccgcacgtgctcggcgcggtcctgtactggctgctgctgccctgcgcgctgctggctgcctgcctgctccgcttcagcggactctcgctggtctacctgctcttcctgctgctgctgccctggttccccggccccacccgatgcggcctccaaggtcacacaggccgcctcctgcgggcattgctgggcctcagcctgctcttcctggtggcccatctcgccctccagatctgcctgcatattgtgccccgcctggaccagctcctgggacccagctgcagccgctgggagaccctctcgcgacacataggggtcacaaggctggacctgaaggacatccccaacgccatccggctggtggcccctgacctgggcatcttggtggtctcctctgtctgcctcggcatctgcgggcgccttgcaaggaacacccggcagagcccacatccacgggagctggatgatgatgagagggatgtggatgccagcccgacggcagggctgcaggaagcagcaacgctggcccctacacggaggtcacggctggccgctcgtttccgagtcacggcccactggctgctggtggcggctgggcgggtcctggccgtaacactgcttgcactggcaggcatcgcccacccctcggccctctccagtgtctacctgctgctcttcctggccctctgcacctggtgggcctgccactttcccatcagcactcggggcttcagcagactctgcgtcgcggtggggtgcttcggcgccggccatctcatctgcctctactgctaccagatgcccttggcacaggctctgctcccgcctgccggcatctgggctagggtgctgggtctcaaggacttcgtgggtcccaccaactgctccagcccccacgcgctggtcctcaacaccggcctggactggcctgtgtatgccagccccggcgtcctcctgctgctgtgctacgccacggcctctctgcgcaagctccgcgcgtaccgcccctccggccagaggaaggaggcggcaaaggggtatgaggctcgggagctggagctagcagagctggaccagtggccccaggaacgggagtctgaccagcacgtggtgcccacagcacccgacaccgaggctgataactgcatcgtgcacgagctgaccggccagagctccgtcctgcggcggcctgtgcggcccaagcgggctgagcccagggaggcgtctccgctccacagcctgggccacctcatcatggaccagagctatgtgtgcgcgctcattgccatgatggtatggagcatcacctaccacagctggctgaccttcgtactgctgctctgggcctgcctcatctggacggtgcgcagccgccaccaactggccatgctgtgctcgccctgcatcctgctgtatgggatgacgctgtgctgcctacgctacgtgtgggccatggacctgcgccctgagctgcccaccaccctgggccccgtcagcctgcgccagctggggctggagcacacccgctacccctgtctggaccttggtgccatgttgctctacaccctgaccttctggctcctgctgcgccagtttgtgaaagagaagctgctgaagtgggcagagtctccagctgcgctgacggaggtcaccgtggcagacacagagcccacgcggacgcagacgctgttgcagagcctgggggagctggtgaagggcgtgtacgccaagtactggatctatgtgtgtgctggcatgttcatcgtggtcagcttcgccggccgcctcgtggtctacaagattgtctacatgttcctcttcctgctctgcctcaccctcttccaggtctactacagcctgtggcggaagctgctcaaggccttctggtggctcgtggtggcctacaccatgctggtcctcatcgccgtctacaccttccagttccaggacttccctgcctactggcgcaacctcactggcttcaccgacgagcagctgggggacctgggcctggagcagttcagcgtgtccgagctcttctccagcatcctggtgcccggcttcttcctcctggcctgcatcctgcagctgcactacttccacaggcccttcatgcagctcaccgacatggagcacgtgtccctgcctggcacgcgcctcccgcgctgggctcacaggcaggatgcagtgagtgggaccccactgctgcgggaggagcagcaggagcatcagcagcagcagcaggaggaggaggaggaggaggaggactccagggacgaggggctgggcgtggccactccccaccaggccacgcaggtgcctgaaggggcagccaagtggggcctggtggctgagcggctgctggagctggcagccggcttctcggacgtcctctcacgcgtgcaggtgttcctgcggcggctgctggagcttcacgttttcaagctggtggccctgtacaccgtctgggtggccctgaaggaggtgtcggtgatgaacctgctgctggtggtgctgtgggccttcgccctgccctacccacgcttccggcccatggcctcctgcctgtccaccgtgtggacctgcgtcatcatcgtgtgtaagatgctgtaccagctcaaggttgtcaacccccaggagtattccagcaactgcaccgagcccttccccaacagcaccaacttgctgcccacggagatcagccagtccctgctgtaccgggggcccgtggaccctgccaactggtttggggtgcggaaagggttccccaacctgggctacatccagaaccacctgcaagtgctgctgctgctggtattcgaggccatcgtgtaccggcgccaggagcactaccgccggcagcaccagctggccccgctgcctgcccaggccgtgtttgccagcggcacccgccagcagctggaccaggatctgctcggctgcctcaagtacttcatcaacttcttcttctacaaattcgggctggagatctgcttcctgatggccgtgaacgtgatcgggcagcgcatgaactttctggtgaccctgcacggttgctggctggtggccatcctcacccgcaggcaccgccaggccattgcccgcctctggcccaactactgcctcttcctggcgctgttcctgctgtaccagtacctgctgtgcctggggatgcccccggccctgtgcattgattatccctggcgctggagccgggccgtccccatgaactccgcactcatcaagtggctgtacctgcctgatttcttccgggcccccaactccaccaacctcatcagcgactttctcctgctgctgtgcgcctcccagcagtggcaggtgttctcagctgagcgcacagaggagtggcagcgcatggctggcgtcaacaccgaccgcctggagccgctgcggggggagcccaaccccgtgcccaactttatccactgcaggtcctaccttgacatgctgaaggtggccgtcttccgatacctgttctggctggtgctggtggtggtgtttgtcacgggggccacccgcatcagcatcttcgggctgggctacctgctggcctgcttctacctgctgctcttcggcacggccctgctgcagagggacacacgggcccgcctcgtgctgtgggactgcctcattctgtacaacgtcaccgtcatcatctccaagaacatgctgtcgctcctggcctgcgtcttcgtggagcagatgcagaccggcttctgctgggtcatccagctcttcagccttgtatgcaccgtcaagggctactatgaccccaaggagatgatggacagagaccaggactgcctgctgcctgtggaggaggctggcatcatctgggacagcgtctgcttcttcttcctgctgctgcagcgccgcgtcttccttagccattactacctgcacgtcagggccgacctccaggccaccgccctgctagcctccaggggcttcgccctctacaacgctgccaacctcaagagcattgactttcaccgcaggatagaggagaagtccctggcccagctgaaaagacagatggagcgtatccgtgccaagcaggagaagcacaggcagggccgggtggaccgcagtcgcccccaggacaccctgggccccaaggaccccggcctggagccagggcccgacagtccagggggctcctccccgccacggaggcagtggtggcggccctggctggaccacgccacagtcatccactccggggactacttcctgtttgagtccgacagtgaggaagaggaggaggctgttcctgaagacccgaggccgtcggcacagagtgccttccagctggcgtaccaggcatgggtgaccaacgcccaggcggtgctgaggcggcggcagcaggagcaggagcaggcaaggcaggaacaggcaggacagctacccacaggaggtggtcccagccaggaggtggagccagcagagggccccgaggaggcagcggcaggccggagccatgtggtgcagagggtgctgagcacggcgcagttcctgtggatgctggggcaggcgctagtggatgagctgacacgctggctgcaggagttcacccggcaccacggcaccatgagcgacgtgctgcgggcagagcgctacctcctcacacaggagctcctgcagggcggcgaagtgcacaggggcgtgctggatcagctgtacacaagccaggccgaggccacgctgccaggccccaccgaggcccccaatgccccaagcaccgtgtccagtgggctgggcgcggaggagccactcagcagcatgacagacgacatgggcagccccctgagcaccggctaccacacgcgcagtggcagtgaggaggcagtcaccgaccccggggagcgtgaggctggtgcctctctgtaccagggactgatgcggacggccagcgagctgctcctggacaggcgcctgcgcatcccagagctggaggaggcagagctgtttgcggaggggcagggccgggcgctgcggctgctgcgggccgtgtaccagtgtgtggccgcccactcggagctgctctgctacttcatcatcatcctcaaccacatggtcacggcctccgccggctcgctggtgctgcccgtgctcgtcttcctgtgggccatgctgtcgatcccgaggcccagcaagcgcttctggatgacggccatcgtcttcaccgagatcgcggtggtcgtcaagtacctgttccagtttgggttcttcccctggaacagccacgtggtgctgcggcgctacgagaacaagccctacttcccgccccgcatcctgggcctggagaagactgacggctacatcaagtacgacctggtgcagctcatggcccttttcttccaccgctcccagctgctgtgctatggcctctgggaccatgaggaggactcaccatccaaggagcatgacaagagcggcgaggaggagcagggagccgaggaggggccaggggtgcctgcggccaccaccgaagaccacattcaggtggaagccagggtcggacccacggacgggaccccagaaccccaagtggagctcaggccccgtgatacgaggcgcatcagtctacgttttagaagaaggaagaaggagggcccagcacggaaaggagcggcagccatcgaagctgaggacagggaggaagaagagggggaggaagagaaagaggcccccacggggagagagaagaggccaagccgctctggaggaagagtaagggcggccgggcggcggctgcagggcttctgcctgtccctggcccagggcacatatcggccgctacggcgcttcttccacgacatcctgcacaccaagtaccgcgcagccaccgacgtctatgccctcatgttcctggctgatgttgtcgacttcatcatcatcatttttggcttctgggcctttgggaagcactcggcggccacagacatcacgtcctccctatcagacgaccaggtacccgaggctttcctggtcatgctgctgatccagttcagtaccatggtggttgaccgcgccctctacctgcgcaagaccgtgctgggcaagctggccttccaggtggcgctggtgctggccatccacctatggatgttcttcatcctgcccgccgtcactgagaggatgttcaaccagaatgtggtggcccagctctggtacttcgtgaagtgcatctacttcgccctgtccgcctaccagatccgctgcggctaccccacccgcatcctcggcaacttcctcaccaagaagtacaatcatctcaacctcttcctcttccaggggttccggctggtgccgttcctggtggagctgcgggcagtgatggactgggtgtggacggacaccacgctgtccctgtccagctggatgtgtgtggaggacatctatgccaacatcttcatcatcaaatgcagccgagagacagagaagaaatacccgcagcccaaagggcagaagaagaagaagatcgtcaagtacggcatgggtggcctcatcatcctcttcctcatcgccatcatctggttcccactgctcttcatgtcgctggtgcgctccgtggttggggttgtcaaccagcccatcgatgtcaccgtcaccctgaagctgggcggctatgagccgctgttcaccatgagcgcccagcagccgtccatcatccccttcacggcccaggcctatgaggagctgtcccggcagtttgacccccagccgctggccatgcagttcatcagccagtacagccctgaggacatcgtcacggcgcagattgagggcagctccggggcgctgtggcgcatcagtccccccagccgtgcccagatgaagcgggagctctacaacggcacggccgacatcaccctgcgcttcacctggaacttccagagggacctggcgaagggaggcactgtggagtatgccaacgagaagcacatgctggccctggcccccaacagcactgcacggcggcagctggccagcctgctcgagggcacctcggaccagtctgtggtcatccctaatctcttccccaagtacatccgtgcccccaacgggcccgaagccaaccctgtgaagcagctgcagcccaatgaggaggccgactacctcggcgtgcgtatccagctgcggagggagcagggtgcgggggccaccggcttcctcgaatggtgggtcatcgagctgcaggagtgccggaccgactgcaacctgctgcccatggtcattttcagtgacaaggtcagcccaccgagcctcggcttcctggctggctacggcatcatggggctgtacgtgtccatcgtgctggtcatcggcaagttcgtgcgcggattcttcagcgagatctcgcactccattatgttcgaggagctgccgtgcgtggaccgcatcctcaagctctgccaggacatcttcctggtgcgggagactcgggagctggagctggaggaggagttgtacgccaagctcatcttcctctaccgctcactggagaccatgatcaagtggactcgtgagaaggagtaggagctgctgctggcgcccgagagggaaggagccggcctgctgggcagcgtggccacaaggggcggcactcctcaggccgggggagccactgccccgtccaaggccgccagctgtgatgcatcctcccggcctgcctgagccctgatgctgctgtcagagaaggacactgcgtccccacggcctgcgtggcgctgccgtcccccacgtgtactgtagagttttttttttaattaaaaaatgttttatttatacaaatggacaatcaga

SEQ ID NO: 34 - DNA, 1246 bp, Homo sapiens
  source 1..1246: mol_type = genomic DNA
atgaagatttgcccgttttcttctaaaagttctatagttttagctctgaagtttcgctctttgatccactttgagaaatacccgcagcccaaagggcagaagaagaagaagatcgtcaagtacggcatgggtggcctcatcatcctcttcctcatcgccatcatctggttcccactgctcttcatgtcgctggtgcgctccgtggttggggttgtcaaccagcccatcgatgtcaccgtcaccctgaagctgggcggctatgagccgctgttcaccatgagcgcccagcagccgtccatcatccccttcacggcccaggcctatgaggagctgtcccggcagtttgacccccagccgctggccatgcagttcatcagccagtacagccctgaggacatcgtcacggcgcagattgagggcagctccggggcgctgtggcgcatcagtccccccagccgtgcccagatgaagcgggagctctacaacggcacggccgacatcaccctgcgcttcacctggaacttccagagggacctggcgaagggaggcactgtggagtatgccaacgagaagcacatgctggccctggcccccaacagcactgcacggcggcagctggccagcctgctcgagggcacctcggaccagtctgtggtcatccctaatctcttccccaagtacatccgtgcccccaacgggcccgaagccaaccctgtgaagcagctgcagcccaatgaggaggccgactacctcggcgtgcgtatccagctgcggagggagcagggtgcgggggccaccggcttcctcgaatggtgggtcatcgagctgcaggagtgccggaccgactgcaacctgctgcccatggtcattttcagtgacaagcatcatggggctgtacgtgtccatcgtgctggtcatcggcaagttcgtgcgcggattcttcagcgagatctcgcactccattatgttcgaggagctgccgtgcgtggaccgcatcctcaagctctgccaggacatcttcctggtgcgggagactcgggagctggagctggaggaggagttgtacgccaagctcatcttcctctaccgctcactggagaccatgatcaagtggactcgtgagaaggagtaggagctgctgctggcgcccgagagggaaggagccggcctgctgggcagcgtggccacaaggggcggcactcctcaggccgggggagccactgccccgtccaaggccgccagctgt

SEQ ID NO: 35 - DNA, 6401 bp, Homo sapiens
  source 1..6401: mol_type = genomic DNA
ccatggacctgcgccctgagctgcccaccaccctgggccccgtcagcctgcgccagctggggctggagcacacccgctacccctgtctggaccttggtgccatgttgctctacaccctgaccttctggctcctgctgcgccagtttgtgaaagagaagctgctgaagtgggcagagtctccagctgcgctgacggaggtcaccgtggcagacacagagcctacgcggacgcagacgctgttgcagagcctgggggagctggtgaagggcgtgtacgccaagtactggatctatgtgtgtgctggcatgttcatcgtggtcagcttcgccggccgcctcgtggtctacaagattgtctacatgttcctcttcctgctctgcctcaccctcttccaggtctactacagcctgtggcggaagctgctcaaggccttctggtggctcgtggtggcctacaccatgctggtcctcatcgccgtctacaccttccagttccaggacttccctgcctactggcgcaacctcactggcttcaccgacgagcagctgggggacctgggcctggagcagttcagcgtatccgagctcttctccagcatcctggtgcccggcttcttcctcctggcctgcatcctgcagctgcactacttccacaggcccttcatgcagctcaccgacatggagcacgtgtccctgcctggcacgcgcctcccgcgctgggctcacaggcaggatgcagtgagtgggaccccactgctgcgggaggagcagcaggagcatcagcagcagcagcaggaggaggaggaggaggaggactccagggacgaggggctgggcgtggccactccccaccaggccacgcaggtgcctgaaggggcagccaagtggggcctggtggctgagcgcctgctggagctggcagccggcttctcggacgtcctctcacgcgtgcaggtgttcctgcggcggctgctggagcttcacgttttcaagctggtggccctgtacaccgtctgggtggccctgaaggaggtgtcggtgatgaacctgctgctggtggtgctgtgggccttcgccctgccctacccacgcttccggcccatggcctcctgcctgtccaccgtgtggacctgcgtcatcatcgtgtgtaagatgctgtaccagctcaaggttgtcaacccccaggagtattccagcaactgcaccgagcccttccccaacagcaccaacttgctgcccacggagatcagccagtccctgctgtaccgggggcccgtggaccctgccaactggtttggggtgcggaaagggttccccaacctgggctacatccagaaccacctgcaagtgctgctgctgctggtattcgaggccatcgtgtaccggcgccaggagcactaccgccggcagcaccagctggccccgctgcctgcccaggccgtgtttgccagcggcacccgccagcagctggaccaggatctgctcggctgcctcaagtacttcatcaacttcttcttctacaaattcgggctggagatctgcttcctgatggccgtgaacgtgatcgggcagcgcatgaactttctggtgaccctgcacggttgctggctggtggccatcctcacccgcaggcaccgccaggccattgcccgcctctggcccaactactgcctcttcctggcgctgttcctgctgtaccagtacctgctgtgcctggggatgcccccggccctgtgcattgattatccctggcgctggagccgggccgtccccatgaactccgcactcatcaagtggctgtacctgcctgatttcttccgggcccccaactccaccaacctcatcagcgactttctcctgctgctgtgcgcctcccagcagtggcaggtgttctcagctgagcgcacagaggagtggcagcgcatggctggcgtcaacaccgaccgcctggagccgctgcggggggagcccaaccccgtgcccaactttatccactgcaggtcctaccttgacatgctgaaggtggccgtcttccgatacctgttctggctggtgctggtggtggtgtttgtcacgggggccacccgcatcagcatcttcgggctgggctacctgctggcctgcttctacctgctgctcttcggcacggccctgctgcagagggacacacgggcccgcctcgtgctgtgggactgcctcattctgtacaacgtcaccgtcatcatctccaagaacatgctgtcgctcctggcctgcgtcttcgtggagcagatgcagaccggcttctgctgggtcatccagctcttcagccttgtatgcaccgtcaagggctactatgaccccaaggagatgatggacagagaccaggactgcctgctgcctgtggaggaggctggcatcatctgggacagcgtctgcttcttcttcctgctgctgcagcgccgcgtcttccttagccattactacctgcacgtcagggccgacctccaggccaccgccctgctagcctccaggggcttcgccctctacaacgctgccaacctcaagagcattgacttccaccgcaggatagaggagaagtccctggcccagctgaaaagacagatggagcgtatccgtgccaagcaggagaagcacaggcagggccgggtggaccgcagtcgcccccaggacaccctgggccccaaggaccccggcctggagccagggcccgacagtccagggggctcctccccgccacggaggcagtggtggcggccctggctggaccacgccacagtcatccactccggggactacttcctgtttgagtccgacagtgaggaagaggaggaggctgttcctgaagacccgaggccgtcggcacagagtgccttccagctggcgtaccaggcatgggtgaccaacgcccaggcggtgctgaggcggcggcagcaggagcaggagcaggcaaggcaggaacaggcaggacagctacccacaggaggtggtcccagccaggaggtggagccagcagagggccccgaggaggcagcggcaggccggagccatgtggtgcagagggtgctgagcacggcgcagttcctgtggatgctggggcaggcgctagtggatgagctgacacgctggctgcaggagttcacccggcaccacggcaccatgagcgacgtgctgcgggcagagcgctacctcctcacacaggagctcctgcagggcggcgaagtgcacaggggcgtgctggatcagctgtacacaagccaggccgaggccacgctgccaggccccaccgaggcccccaatgccccaagcaccgtgtccagtgggctgggcgcggaggagccactcagcagcatgacagacgacatgggcagccccctgagcaccggctaccacacgcgcagtggcagtgaggaggcagtcaccgaccccggggagcgtgaggctggtgcctctctgtaccagggactgatgcggacggccagcgagctgctcctggacaggcgcctgcgcatcccagagctggaggaggcagagctgtttgcggaggggcagggccgggcgctgcggctgctgcgggccgtgtaccagtgtgtggccgcccactcggagctgctctgctacttcatcatcatcctcaaccacatggtcacggcctccgccggctccctggtgctgcccgtgctcgtcttcctgtgggccatgctgtcgatcccgaggcccagcaagcgcttctggatgacggccatcgtcttcaccgagatcgcggtggtcgtcaagtacctgttccagtttgggttcttcccctggaacagccacgtggtgctgcggcgctacgagaacaagccctacttcccgccccgcatcctgggcctggagaagactgacggctacatcaagtacgacctggtgcagctcatggcccttttcttccaccgctcccagctgctgtgctatggcctctgggaccatgaggaggactcaccatccaaggagcatgacaagagcggcgaggaggagcagggagccgaggaggggccaggggtgcctgcggccaccaccgaagaccacattcaggtggaagcgagggtcggacccacggacgggaccccagaaccccaagtggagctcaggccccgtgatacgaggcgcatcagtctacgttttagaagaaggaagaaggagggcccagcacggaaaggagcggcagccatcgaagctgaggacagggaggaagaagagggggaggaagagaaagaggcccccacggggagagagaagaggccaagccgctctggaggaagagtaagggcggccgggcggcggctgcagggcttctgcctgtccctggcccagggcacatatcggccgctacggcgcttcttccacgacatcctgcacaccaagtaccgcgcagccaccgacgtctatgccctcatgttcctggctgatgttgtcgacttcatcatcatcatttttggcttctgggcctttgggaagcactcggcggccacagacatcacgtcctccctatcagacgaccaggtacccgaggctttcctggtcatgctgctgatccagttcagtaccatggtggttgaccgcgccctctacctgcgcaagaccgtgctgggcaagctggccttccaggtggcgctggtgctggccatccacctatggatgttcttcatcctgcccgccgtcactgagaggatgttcaaccagaatgtggtggcccagctctggtacttcgtgaagtgcatctacttcgccctgtccgcctaccagatccgctgcggctaccccacccgcatcctcggcaacttcctcaccaagaagtacaatcatctcaacctcttcctcttccaggggttccggctggtgccgttcctggtggagctgcgggcagtgatggactgggtgtggacggacaccacgctgtccctgtccagctggatgtgtgtggaggacatctatgccaacatcttcatcatcaaatgcagccgagagacagagaagaaatacccgcagcccaaagggcagaagaagaagaagatcgtcaagtacggcatgggtggcctcatcatcctcttcctcatcgccatcatctggttcccgctgctcttcatgtcgctggtgcgctccgtggttggggttgtcaaccagcccatcgatgtcaccgtcaccctcaagctgggcggctatgagccgctgttcaccatgagcgcccagcagccgtccatcatccccttcacggcccaggcctatgaggagctgtcccggcagtttgacccccagccgctggccatgcagttcatcagccagtacagccctgaggacatcgtcacggcgcagattgagggcagctccggggcgctgtggcgcatcagtccccccagccgtgcccagatgaagcgggagctctacaacggcacggccgacatcaccctgcgcttcacctggaacttccagagggacctggcgaagggaggcactgtggagtatgccaacgagaagcacatgctggccctggcccccaacagcactgcacggcggcagctggccagcctgctcgagggcacctcggaccagtctgtggtcatccccaatctcttccccaagtacatccgtgcccccaacgggcccgaagccaaccctgtgaagcagctgcagcccaatgaggaggccgactacctcggcgtgcgtatccagctgcggagggagcagggtgcgggggccaccggcttcctcgaatggtgggtcatcgagctgcaggagtgccggaccgactgcaacctgctgcccatggtcattttcagtgacaaggtcagcccaccgagcctcggcttcctggctggctacggcatcatggggctgtacgtgtccatcgtgctggtcatcggcaagttcgtgcgcggattcttcagcgagatctcgcactccattatgttcgaggagctgccgtgcgtggaccgcatcctcaagctctgccaggacatcttcctggtgcgggagactcgggagctggagctggaggaggagttgtacgccaagctcatcttcctctaccgctcactggagaccatgatcaagtggactcgtgagaaggagtaggagctgctgctggcgcccgagagggaaggagccggcctgctgggcagcgtggccacaaggggcggcactcctcaggccgggggagccactgccccgtccaaggccgccagctgtgatgcatcctcccggcctgcctgagccctgatgctgctgtcagagaaggacactgcgtccccacggcctgcgtggcgctgccgtcccccacgtgtactgtagagttttttttttaattaaaaaatgttttatttatacaaatggaaaaaaaaaaaaaaaaaaaaaaaaaaaaaaaaa

SEQ ID NO: 36 - DNA, 1655 bp, Homo sapiens
  source 1..1655: mol_type = genomic DNA
taccagatccgctgcggctaccccacccgcatcctcggcaacttcctcaccaagaagtacaatcatctcaacctcttcctcttccaggggttccggctggtgccgttcctggtggagctgcgggcagtgatggactgggtgtggacggacaccacgctgtccctgtccagctggatgtgtgtggaggacatctatgccaacatcttcatcatcaaatgcagccgagagacagagaaggttcatgaagatttgcccgttttcttctaaaagttctatagttttagctctgaagtttcgctctttgatccactttgagaaatacccgcagcccaaagggcagaagaagaagaagatcgtcaagtacggcatgggtggcctcatcatcctcttcctcatcgccatcatctggttcccgctgctcttcatgtcgctggtgcgctccgtggttggggttgtcaaccagcccatcgatgtcaccgtcaccctcaagctgggcggctatgagccgctgttcaccatgagcgcccagcagccgtccatcatccccttcacggcccaggcctatgaggagctgtcccggcagtttgacccccagccgctggccatgcagttcatcagccagtacagccctgaggacatcgtcacggcgcagattgagggcagctccggggcgctgtggcgcatcagtccccccagccgtgcccagatgaagcgggagctctacaacggcacggccgacatcaccctgcgcttcacctggaacttccagagggacctggcgaagggaggcactgtggagtatgccaacgagaagcacatgctggccctggcccccaacagcactgcacggcggcagctggccagcctgctcgagggcacctcggatcagtctgtggtcatccccaatctcttccccaagtacatccgtgcccccaacgggcccgaagccaaccctgtgaagcagctgcagcccaatgaggaggccgactacctcggcgtgcgtatccagctgcggagggagcagggtgcgggggccaccggcttcctcgaatggtgggtcatcgagctgcaggagtgccggaccgactgcaacctgctgcccatggtcattttcagtgacaagcatcatggggctgtacgtgtccatcgtgctggtcatcggcaagttcgtgcgcggattcttcagcgagatctcgcactccattatgttcgaggagctgccgtgcgtggaccgcatcctcaagctctgccaggacatcttcctggtgcgggagactcgggagctggagctggaggaggagttgtatgccaagctcatcttcctctaccgctcactggagaccatgatcaagtggactcgtgagaaggagtaggagctgctgctggcgcccgagagggaaggagccggcctgctgggcagcgtggccacaaggggcggcactcctcaggccgggggagccactgccccgtccaaggccgccagctgtgatgcatcctcccggcctgcctgagccctgatgctgctgtcagagaaggacactgcgtccccacggcctgcgtggcgctgccgtcccccacgtgtactgtagagtttttttttttaattaaaaaatgttttatttatacaaatggaaaaaaaaaaaaaaaaaaaaaaa

SEQ ID NO: 37 - DNA, 6368 bp, Homo sapiens
  source 1..6368: mol_type = genomic DNA
ccatggacctgcgccctgagctgcccaccaccctgggccccgtcagcctgcgccagctggggctggagcacacccgctacccctgtctggaccttggtgccatgttgctctacaccctgaccttctggctcctgctgcgccagtttgtgaaagagaagctgctgaagtgggcagagtctccagctgcgctgacggaggtcaccgtggcagacacagagcctacgcggacgcagacgctgttgcagagcctgggggagctggtgaagggcgtgtacgccaagtactggatctatgtgtgtgctggcatgttcatcgtggtcagcttcgccggccgcctcgtggtctacaagattgtctacatgttcctcttcctgctctgcctcaccctcttccaggtctactacagcctgtggcggaagctgctcaaggccttctggtggctcgtggtggcctacaccatgctggtcctcatcgccgtctacaccttccagttccaggacttccctgcctactggcgcaacctcactggcttcaccgacgagcagctgggggacctgggcctggagcagttcagcgtatccgagctcttctccagcatcctggtgcccggcttcttcctcctggcctgcatcctgcagctgcactacttccacaggcccttcatgcagctcaccgacatggagcacgtgtccctgcctggcacgcgcctcccgcgctgggctcacaggcaggatgcagtgagtgggaccccactgctgcgggaggagcagcaggagcatcagcagcagcagcaggaggaggaggaggaggaggactccagggacgaggggctgggcgtggccactccccaccaggccacgcaggtgcctgaaggggcagccaagtggggcctggtggctgagcgcctgctggagctggcagccggcttctcggacgtcctctcacgcgtgcaggtgttcctgcggcggctgctggagcttcacgttttcaagctggtggccctgtacaccgtctgggtggccctgaaggaggtgtcggtgatgaacctgctgctggtggtgctgtgggccttcgccctgccctacccacgcttccggcccatggcctcctgcctgtccaccgtgtggacctgcgtcatcatcgtgtgtaagatgctgtaccagctcaaggttgtcaacccccaggagtattccagcaactgcaccgagcccttccccaacagcaccaacttgctgcccacggagatcagccagtccctgctgtaccgggggcccgtggaccctgccaactggtttggggtgcggaaagggttccccaacctgggctacatccagaaccacctgcaagtgctgctgctgctggtattcgaggccatcgtgtaccggcgccaggagcactaccgccggcagcaccagctggccccgctgcctgcccaggccgtgtttgccagcggcacccgccagcagctggaccaggatctgctcggctgcctcaagtacttcatcaacttcttcttctacaaattcgggctggagatctgcttcctgatggccgtgaacgtgatcgggcagcgcatgaactttctggtgaccctgcacggttgctggctggtggccatcctcacccgcaggcaccgccaggccattgcccgcctctggcccaactactgcctcttcctggcgctgttcctgctgtaccagtacctgctgtgcctggggatgcccccggccctgtgcattgattatccctggcgctggagccgggccgtccccatgaactccgcactcatcaagtggctgtacctgcctgatttcttccgggcccccaactccaccaacctcatcagcgactttctcctgctgctgtgcgcctcccagcagtggcaggtgttctcagctgagcgcacagaggagtggcagcgcatggctggcgtcaacaccgaccgcctggagccgctgcggggggagcccaaccccgtgcccaactttatccactgcaggtcctaccttgacatgctgaaggtggccgtcttccgatacctgttctggctggtgctggtggtggtgtttgtcacgggggccacccgcatcagcatcttcgggctgggctacctgctggcctgcttctacctgctgctcttcggcacggccctgctgcagagggacacacgggcccgcctcgtgctgtgggactgcctcattctgtacaacgtcaccgtcatcatctccaagaacatgctgtcgctcctggcctgcgtcttcgtggagcagatgcagaccggcttctgctgggtcatccagctcttcagccttgtatgcaccgtcaagggctactatgaccccaaggagatgatggacagagaccaggactgcctgctgcctgtggaggaggctggcatcatctgggacagcgtctgcttcttcttcctgctgctgcagcgccgcgtcttccttagccattactacctgcacgtcagggccgacctccaggccaccgccctgctagcctccaggggcttcgccctctacaacgctgccaacctcaagagcattgacttccaccgcaggatagaggagaagtccctggcccagctgaaaagacagatggagcgtatccgtgccaagcaggagaagcacaggcagggccgggtggaccgcagtcgcccccaggacaccctgggccccaaggaccccggcctggagccagggcccgacagtccagggggctcctccccgccacggaggcagtggtggcggccctggctggaccacgccacagtcatccactccggggactacttcctgtttgagtccgacagtgaggaagaggaggaggctgttcctgaagacccgaggccgtcggcacagagtgccttccagctggcgtaccaggcatgggtgaccaacgcccaggcggtgctgaggcggcggcagcaggagcaggagcaggcaaggcaggaacaggcaggacagctacccacaggaggtggtcccagccaggaggtggagccagcagagggccccgaggaggcagcggcaggccggagccatgtggtgcagagggtgctgagcacggcgcagttcctgtggatgctggggcaggcgctagtggatgagctgacacgctggctgcaggagttcacccggcaccacggcaccatgagcgacgtgctgcgggcagagcgctacctcctcacacaggagctcctgcagggcggcgaagtgcacaggggcgtgctggatcagctgtacacaagccaggccgaggccacgctgccaggccccaccgaggcccccaatgccccaagcaccgtgtccagtgggctgggcgcggaggagccactcagcagcatgacagacgacatgggcagccccctgagcaccggctaccacacgcgcagtggcagtgaggaggcagtcaccgaccccggggagcgtgaggctggtgcctctctgtaccagggactgatgcggacggccagcgagctgctcctggacaggcgcctgcgcatcccagagctggaggaggcagagctgtttgcggaggggcagggccgggcgctgcggctgctgcgggccgtgtaccagtgtgtggccgcccactcggagctgctctgctacttcatcatcatcctcaaccacatggtcacggcctccgccggctccctggtgctgcccgtgctcgtcttcctgtgggccatgctgtcgatcccgaggcccagcaagcgcttctggatgacggccatcgtcttcaccgagatcgcggtggtcgtcaagtacctgttccagtttgggttcttcccctggaacagccacgtggtgctgcggcgctacgagaacaagccctacttcccgccccgcatcctgggcctggagaagactgacggctacatcaagtacgacctggtgcagctcatggcccttttcttccaccgctcccagctgctgtgctatggcctctgggaccatgaggaggactcaccatccaaggagcatgacaagagcggcgaggaggagcagggagccgaggaggggccaggggtgcctgcggccaccaccgaagaccacattcaggtggaagcgagggtcggacccacggacgggaccccagaaccccaagtggagctcaggccccgtgatacgaggcgcatcagtctacgttttagaagaaggaagaaggagggcccagcacggaaaggagcggcagccatcgaagctgaggacagggaggaagaagagggggaggaagagaaagaggcccccacggggagagagaagaggccaagccgctctggaggaagagtaagggcggccgggcggcggctgcagggcttctgcctgtccctggcccagggcacatatcggccgctacggcgcttcttccacgacatcctgcacaccaagtaccgcgcagccaccgacgtctatgccctcatgttcctggctgatgttgtcgacttcatcatcatcatttttggcttctgggcctttgggaagcactcggcggccacagacatcacgtcctccctatcagacgaccaggtacccgaggctttcctggtcatgctgctgatccagttcagtaccatggtggttgaccgcgccctctacctgcgcaagaccgtgctgggcaagctggccttccaggtggcgctggtgctggccatccacctatggatgttcttcatcctgcccgccgtcactgagaggatgttcaaccagaatgtggtggcccagctctggtacttcgtgaagtgcatctacttcgccctgtccgcctaccagatccgctgcggctaccccacccgcatcctcggcaacttcctcaccaagaagtacaatcatctcaacctcttcctcttccaggggttccggctggtgccgttcctggtggagctgcgggcagtgatggactgggtgtggacggacaccacgctgtccctgtccagctggatgtgtgtggaggacatctatgccaacatcttcatcatcaaatgcagccgagagacagagaagaaatacccgcagcccaaagggcagaagaagaagaagatcgtcaagtacggcatgggtggcctcatcatcctcttcctcatcgccatcatctggttcccgctgctcttcatgtcgctggtgcgctccgtggttggggttgtcaaccagcccatcgatgtcaccgtcaccctcaagctgggcggctatgagccgctgttcaccatgagcgcccagcagccgtccatcatccccttcacggcccaggcctatgaggagctgtcccggcagtttgacccccagccgctggccatgcagttcatcagccagtacagccctgaggacatcgtcacggcgcagattgagggcagctccggggcgctgtggcgcatcagtccccccagccgtgcccagatgaagcgggagctctacaacggcacggccgacatcaccctgcgcttcacctggaacttccagagggacctggcgaagggaggcactgtggagtatgccaacgagaagcacatgctggccctggcccccaacagcactgcacggcggcagctggccagcctgctcgagggcacctcggaccagtctgtggtcatccccaatctcttccccaagtacatccgtgcccccaacgggcccgaagccaaccctgtgaagcagctgcagcccaatgaggaggccgactacctcggcgtgcgtatccagctgcggagggagcagggtgcgggggccaccggcttcctcgaatggtgggtcatcgagctgcaggagtgccggaccgactgcaacctgctgcccatggtcattttcagtgacaaggtcagcccaccgagcctcggcttcctggctggctacggcatcatggggctgtacgtgtccatcgtgctggtcatcggcaagttcgtgcgcggattcttcagcgagatctcgcactccattatgttcgaggagctgccgtgcgtggaccgcatcctcaagctctgccaggacatcttcctggtgcgggagactcgggagctggagctggaggaggagttgtacgccaagctcatcttcctctaccgctcactggagaccatgatcaagtggactcgtgagaaggagtaggagctgctgctggcgcccgagagggaaggagccggcctgctgggcagcgtggccacaaggggcggcactcctcaggccgggggagccactgccccgtccaaggccgccagctgtgatgcatcctcccggcctgcctgagccctgatgctgctgtcagagaaggacactgcgtccccacggcctgcgtggcgctgccgtcccccacgtgtactgtagagttttttttttaattaaaaaatgttttatttatacaaatgg

SEQ ID NO: 38 - DNA, 6273 bp, Homo sapiens
  source 1..6273: mol_type = genomic DNA
atggtatggagcatcacctaccacagctggctgaccttcgtactgctgctctgggcctgcctcatctggacggtgcgcagccgccaccaactggccatgctgtgctcgccctgcatcctgctgtatgggatgacgctgtgctgcctacgctacgtgtgggccatggacctgcgccctgagctgcccaccaccctgggccccgtcagcctgcgccagctggggctggagcacacccgctacccctgtctggaccttggtgccatgttgctctacaccctgaccttctggctcctgctgcgccagtttgtgaaagagaagctgctgaagtgggcagagtctccagctgcgctgacggaggtcaccgtggcagacacagagcccacgcggacgcagacgctgttgcagagcctgggggagctggtgaagggcgtgtacgccaagtactggatctatgtgtgtgctggcatgttcatcgtggtcagcttcgccggccgcctcgtggtctacaagattgtctacatgttcctcttcctgctctgcctcaccctcttccaggtctactacagcctgtggcggaagctgctcaaggccttctggtggctcgtggtggcctacaccatgctggtcctcatcgccgtctacaccttccagttccaggacttccctgcctactggcgcaacctcactggcttcaccgacgagcagctgggggacctgggcctggagcagttcagcgtgtccgagctcttctccagcatcctggtgcccggcttcttcctcctggcctgcatcctgcagctgcactacttccacaggcccttcatgcagctcaccgacatggagcacgtgtccctgcctggcacgcgcctcccgcgctgggctcacaggcaggatgcagtgagtgggaccccactgctgcgggaggagcagcaggagcatcagcagcagcagcaggaggaggaggaggaggaggaggactccagggacgaggggctgggcgtggccactccccaccaggccacgcaggtgcctgaaggggcagccaagtggggcctggtggctgagcggctgctggagctggcagccggcttctcggacgtcctctcacgcgtgcaggtgttcctgcggcggctgctggagcttcacgttttcaagctggtggccctgtacaccgtctgggtggccctgaaggaggtgtcggtgatgaacctgctgctggtggtgctgtgggccttcgccctgccctacccacgcttccggcccatggcctcctgcctgtccaccgtgtggacctgcgtcatcatcgtgtgtaagatgctgtaccagctcaaggttgtcaacccccaggagtattccagcaactgcaccgagcccttccccaacagcaccaacttgctgcccacggagatcagccagtccctgctgtaccgggggcccgtggaccctgccaactggtttggggtgcggaaagggttccccaacctgggctacatccagaaccacctgcaagtgctgctgctgctggtattcgaggccatcgtgtaccggcgccaggagcactaccgccggcagcaccagctggccccgctgcctgcccaggccgtgtttgccagcggcacccgccagcagctggaccaggatctgctcggctgcctcaagtacttcatcaacttcttcttctacaaattcgggctggagatctgcttcctgatggccgtgaacgtgatcgggcagcgcatgaactttctggtgaccctgcacggttgctggctggtggccatcctcacccgcaggcaccgccaggccattgcccgcctctggcccaactactgcctcttcctggcgctgttcctgctgtaccagtacctgctgtgcctggggatgcccccggccctgtgcattgattatccctggcgctggagccgggccgtccccatgaactccgcactcatcaagtggctgtacctgcctgatttcttccgggcccccaactccaccaacctcatcagcgactttctcctgctgctgtgcgcctcccagcagtggcaggtgttctcagctgagcgcacagaggagtggcagcgcatggctggcgtcaacaccgaccgcctggagccgctgcggggggagcccaaccccgtgcccaactttatccactgcaggtcctaccttgacatgctgaaggtggccgtcttccgatacctgttctggctggtgctggtggtggtgtttgtcacgggggccacccgcatcagcatcttcgggctgggctacctgctggcctgcttctacctgctgctcttcggcacggccctgctgcagagggacacacgggcccgcctcgtgctgtgggactgcctcattctgtacaacgtcaccgtcatcatctccaagaacatgctgtcgctcctggcctgcgtcttcgtggagcagatgcagaccggcttctgctgggtcatccagctcttcagccttgtatgcaccgtcaagggctactatgaccccaaggagatgatggacagagaccaggactgcctgctgcctgtggaggaggctggcatcatctgggacagcgtctgcttcttcttcctgctgctgcagcgccgcgtcttccttagccattactacctgcacgtcagggccgacctccaggccaccgccctgctagcctccaggggcttcgccctctacaacgctgccaacctcaagagcattgacttccaccgcaggatagaggagaagtccctggcccagctgaaaagacagatggagcgtatccgtgccaagcaggagaagcacaggcagggccgggtggaccgcagtcgcccccaggacaccctgggccccaaggaccccggcctggagccagggcccgacagtccagggggctcctccccgccacggaggcagtggtggcggccctggctggaccacgccacagtcatccactccggggactacttcctgtttgagtccgacagtgaggaagaggaggaggctgttcctgaagacccgaggccgtcggcacagagtgccttccagctggcgtaccaggcatgggtgaccaacgcccaggcggtgctgaggcggcggcagcaggagcaggagcaggcaaggcaggaacaggcaggacagctacccacaggaggtggtcccagccaggaggtggagccagcagagggccccgaggaggcagcggcaggccggagccatgtggtgcagagggtgctgagcacggcgcagttcctgtggatgctggggcaggcgctagtggatgagctgacacgctggctgcaggagttcacccggcaccacggcaccatgagcgacgtgctgcgggcagagcgctacctcctcacacaggagctcctgcagggcggcgaagtgcacaggggcgtgctggatcagctgtacacaagccaggccgaggccacgctgccaggccccaccgaggcccccaatgccccaagcaccgtgtccagtgggctgggcgcggaggagccactcagcagcatgacagacgacatgggcagccccctgagcaccggctaccacacgcgcagtggcagtgaggaggcagtcaccgaccccggggagcgtgaggctggtgcctctctgtaccagggactgatgcggacggccagcgagctgctcctggacaggcgcctgcgcatcccagagctggaggaggcagagctgtttgcggaggggcagggccgggcgctgcggctgctgcgggccgtgtaccagtgtgtggccgcccactcggagctgctctgctacttcatcatcatcctcaaccacatggtcacggcctccgccggctcgctggtgctgcccgtgctcgtcttcctgtgggccatgctgtcgatcccgaggcccagcaagcgcttctggatgacggccatcgtcttcaccgagatcgcggtggtcgtcaagtacctgttccagtttgggttcttcccctggaacagccacgtggtgctgcggcgctacgagaacaagccctacttcccgccccgcatcctgggcctggagaagactgacggctacatcaagtacgacctggtgcagctcatggcccttttcttccaccgctcccagctgctgtgctatggcctctgggaccatgaggaggactcaccatccaaggagcatgacaagagcggcgaggaggagcagggagccgaggaggggccaggggtgcctgcggccaccaccgaagaccacattcaggtggaagcgagggtcggacccacggacgggaccccagaaccccaagtggagctcaggccccgtgatacgaggcgcatcagtctacgttttagaagaaggaagaaggagggcccagcacggaaaggagcggcagccatcgaagctgaggacagggaggaagaagagggggaggaagagaaagaggcccccacggggagagagaagaggccaagccgctctggaggaagagtaagggcggccgggcggcggctgcagggcttctgcctgtccctggcccagggcacatatcggccgctacggcgcttcttccacgacatcctgcacaccaagtaccgcgcagccaccgacgtctatgccctcatgttcctggctgatgttgtcgacttcatcatcatcatttttggcttctgggcctttgggaagcactcggcggccacagacatcacgtcctccctatcagacgaccaggtacccgaggctttcctggtcatgctgctgatccagttcagtaccatggtggttgaccgcgccctctacctgcgcaagaccgtgctgggcaagctggccttccaggtggcgctggtgctggccatccacctatggatgttcttcatcctgcccgccgtcactgagaggatgttcaaccagaatgtggtggcccagctctggtacttcgtgaagtgcatctacttcgccctgtccgcctaccagatccgctgcggctaccccacccgcatcctcggcaacttcctcaccaagaagtacaatcatctcaacctcttcctcttccaggggttccggctggtgccgttcctggtggagctgcgggcagtgatggactgggtgtggacggacaccacgctgtccctgtccagctggatgtgtgtggaggacatctatgccaacatcttcatcatcaaatgcagccgagagacagagaagaaatacccgcagcccaaagggcagaagaagaagaagatcgtcaagtacggcatgggtggcctcatcatcctcttcctcatcgccatcatctggttcccgctgctcttcatgtcgctggtgcgctccgtggttggggttgtcaaccagcccatcgatgtcaccgtcaccctgaagctgggcggctatgagccgctgttcaccatgagcgcccagcagccgtccatcatccccttcacggcccaggcctatgaggagctgtcccggcagtttgacccccagccgctggccatgcagttcatcagccagtacagccctgaggacatcgtcacggcgcagattgagggcagctccggggcgctgtggcgcatcagtccccccagccgtgcccagatgaagcgggagctctacaacggcacggccgacatcaccctgcgcttcacctggaacttccagagggacctggcgaagggaggcactgtggagtatgccaacgagaagcacatgctggccctggcccccaacagcactgcacggcggcagctggccagcctgctcgagggcacctcggaccagtctgtggtcatccccaatctcttccccaagtacatccgtgcccccaacgggcccgaagccaaccctgtgaagcagctgcagcccaatgaggaggccgactacctcggcgtgcgtatccagctgcggagggagcagggtgcgggggccaccggcttcctcgaatggtgggtcatcgagctgcaggagtgccggaccgactgcaacctgctgcccatggtcattttcagtgacaaggtcagcccaccgagcctcggcttcctggctggctacggcatcatggggctgtacgtgtccatcgtgctggtcatcggcaagttcgtgcgcggattcttcagcgagatctcgcactccattatgttcgaggagctgccgtgcgtggaccgcatcctcaagctctgccaggacatcttcctggtgcgggagactcgggagctggagctggaggaggagttgtacgccaagctcatcttcctctaccgctcactggagaccatgatcaagtggactcgtgagaaggagtag

SEQ ID NO: 39 - DNA, 7563 bp, Homo sapiens
  source 1..7563: mol_type = genomic DNA
atggagccgcacgtgttcggcgcggtcctgtactggctgctgctgccctgcgcgctgctggctgcctgcctgctccgcttcagcggactctcgctggtctacctgctcttcctgctgctgctgccctggttccccggccccacccgatgcggcctccaaggtcacacaggccgcctcctgcgggcattgctgggcctcagcctgctcttcctggtggcccatctcgccctccagatctgcctgcatactgtgccccgcctggaccagctcctgggacccagctgcagccgctgggagaccctctcgcgacacataggggtcacaaggctggacctgaaggacatccccaacgccatccggctggtggcccctgacctgggcatcttggtggtctcctctgtctgcctcggcatctgcgggcgccttgcaaggaacacccggcagagcccacatccacgggagctggatgatgatgagagggatgtggatgccagcccgacggcagggctgcaggaagcagcaacgctggcccctacacggaggtcacggctggccgctcgtttccgagtcacggcccactggctgctggtggcggctgggcgggtcctggccgtaacactgcttgcactggcaggcatcgcccacccctcggccctctccagtgtctacctgctgctcttcctggccctctgcacctggtgggcctgccactttcccatcagcactcggggcttcagcagactctgcgccgcggtggggtgcttcggcgccggccatctcatctgcctctactgctaccagatgcccttggcacaggctctgctcccgcctgccggcatctgggctagggtgctgggtctcaaggacttcgtgggtcccaccaactgctccagcccccacgcgctggtcctcaacaccggcctggactggcctgtgtatgccagccccggcgtcctcctgctgctgtgctacgccacggcctctctgcgcaagctccgcgcgtaccgcccctccggccagaggaaggaggcggcaaaggggtatgaggctcgggagctggagctagcagagctggaccagtggccccaggaacgggagtctgaccagcacgtggtgcccacagcacccgacaccgaggctgataactgcatcgtgcacgagctgaccggccagagctccgtcctgcggcggcctgtgcggcccaagcgggctgagcccagggaggcgtctccgctccacagcctgggccacctcatcatggaccagagctatgtgtgcgcgctcattgccatgatggtatggagcatcacctaccacagctggctgaccttcgtactgctgctctgggcctgcctcatctggacggtgcgcagccgccaccaactggccatgctgtgctcgccctgcatcctgctgtatgggatgacgctgtgctgcctacgctacgtgtgggccatggacctgcgccctgagctgcccaccaccctgggccccgtcagcctgcgccagctggggctggagcacacccgctacccctgtctggaccttggtgccatgttgctctacaccctgaccttctggctcctgctgcgccagtttgtgaaagagaagctgctgaagtgggcagagtctccagctgcgctgacggaggtcaccgtggcagacacagagcccacgcggacgcagacgctgttgcagagcctgggggagctggtgaagggcgtgtacgccaagtactggatctatgtgtgtgctggcatgttcatcgtggtcagcttcgccggccgcctcgtggtctacaagattgtctacatgttcctcttcctgctctgcctcaccctcttccaggtctactacagcctgtggcggaagctgctcaaggccttctggtggctcgtggtggcctacaccatgctggtcctcatcgccgtctacaccttccagttccaggacttccctgcctactggcgcaacctcactggcttcaccgacgagcagctgggggacctgggcctggagcagttcagcgtgtccgagctcttctccagcatcctggtgcccggcttcttcctcctggcctgcatcctgcagctgcactacttccacaggcccttcatgcagctcaccgacatggagcacgtgtccctgcctggcacgcgcctcccgcgctgggctcacaggcaggatgcagtgagtgggaccccactgctgcgggaggagcagcaggagcatcagcagcagcagcaggaggaggaggaggaggaggaggactccagggacgaggggctgggcgtggccactccccaccaggccacgcaggtgcctgaaggggcagccaagtggggcctggtggctgagcggctgctggagctggcagccggcttctcggacgtcctctcacgcgtgcaggtgttcctgcggcggctgctggagcttcacgttttcaagctggtggccctgtacaccgtctgggtggccctgaaggaggtgtcggtgatgaacctgctgctggtggtgctgtgggccttcgccctgccctacccacgcttccggcccatggcctcctgcctgtccaccgtgtggacctgcgtcatcatcgtgtgtaagatgctgtaccagctcaaggttgtcaacccccaggagtattccagcaactgcaccgagcccttccccaacagcaccaacttgctgcccacggagatcagccagtccctgctgtaccgggggcccgtggaccctgccaactggtttggggtgcggaaagggttccccaacctgggctacatccagaaccacctgcaagtgctgctgctgctggtattcgaggccatcgtgtaccggcgccaggagcactaccgccggcagcaccagctggccccgctgcctgcccaggccgtgtttgccagcggcacccgccagcagctggaccaggatctgctcggctgcctcaagtacttcatcaacttcttcttctacaaattcgggctggagatctgcttcctgatggccgtgaacgtgatcgggcagcgcatgaactttctggtgaccctgcacggttgctggctggtggccatcctcacccgcaggcaccgccaggccattgcccgcctctggcccaactactgcctcttcctggcgctgttcctgctgtaccagtacctgctgtgcctggggatgcccccggccctgtgcattgattatccctggcgctggagccgggccgtccccatgaactccgcacccatcaagtggctgtacctgcctgatttcttccgggcccccaactccaccaacctcatcagcgactttctcctgctgctgtgcgcctcccagcagtggcaggtgttctcagctgagcgcacagaggagtggcagcgcatggctggcgtcaacaccgaccgcctggagccgctgcggggggagcccaaccccgtgcccaactttatccactgcaggtcctaccttgacatgctgaagctggccgtcttccgatacctgttctggctggtgctggtggtggtgtttgtcacgggggccacccgcatcagcatcttcgggctgggctacctgctggcctgcttctacctgctgctcttcggcacggccctgctgcagagggacacacgggcccgcctcgtgctgtgggactgcctcattctgtacaacgtcaccgtcatcatctccaagaacatgctgtcgctcctggcctgcgtcttcgtggagcagatgcagaccggcttctgctgggtcatccagctcttcagccttgtatgcaccgtcaagggctactatgaccccaaggagatgatggacagagaccaggactgcctgctgcctgtggaggaggctggcatcatctgggacagcgtctgcttcttcttcctgctgctgcagcgccgcgtcttccttagccattactacctgcacgtcagggccgacctccaggccaccgccctgctagcctccaggggcttcgccctctacaacgctgccaacctcaagagcattgacttccaccgcaggatagaggagaagtccctggcccagctgaaaagacagatggagcgtatccgtgccaagcaggagaagcacaggcagggccgggtggaccgcagtcgcccccaggacaccctgggccccaaggaccccggcctggagccagggcccgacagtccagggggctcctccccgctacggaggcagtggtggcggccctggctggaccacgccacagtcatccactccggggactacttcctgtttgagtccgacagtgaggaagaggaggaggctgttcctgaagacccgaggccgtcggcacagagtgccttccagctggcgtaccaggcatgggtgaccaacgcccaggcggtgctgaggcggcggcagcaggagcaggagcaggcaaggcaggaacaggcaggacagctacccacaggaggtggtcccagccaggaggtggagccagcagagggccccgaggaggcagcggcaggccggagccatgtggtgcagagggtgctgagcacggcgcagttcctgtggatgctggggcaggcgctagtggatgagctgacacgctggctgcaggagttcacccggcaccacggcaccatgagcgacgtgctgcgggcagagcgctacctcctcacacaggagctcctgcagggcggcgaagtgcacaggggcgtgctggatcagctgtacacaagccaggccgaggccacgctgccaggccccaccgaggcccccaatgccccaagcaccgtgtccagtgggctgggcgcggaggagccactcagcagcatgacagacgacatgggcagccccctgagcaccggctaccacacgcgcagtggcagtgaggaggcagtcaccgaccccggggagcgtgaggctggtgcctctctgtaccagggactgatgcggacggccagcgagctgctcctggacaggcgcctgcgcatcccagagctggaggaggcagagctgtttgcggaggggcagggccgggcgctgcggctgctgcgggccgtgtaccagtgtgtggccgcccactcggagctgctctgctacttcatcatcatcctcaaccacatggtcacggcctccgccggctcgctggtgctgcccgtgctcgtcttcctgtgggccatgctgtcgatcccgaggcccagcaagcgcttctggatgacggccatcgtcttcaccgagatcgcggtggtcgtcaagtacctgttccagtttgggttcttcccctggaacagccacgtggtgctgcggcgctacgagaacaagccctacttcccgccccgcatcctgggcctggagaagactgacggctacatcaagtacgacctggtgcagctcatggcccttttcttccaccgctcccagctgctgtgctatggcctctgggaccatgaggaggactcaccatccaaggagcatgacaagagcggcgaggaggagcagggagccgaggaggggccaggggtgcctgcggccaccaccgaagaccacattcaggtggaagccagggtcggacccacggacgggaccccagaatcccaagtggagctcaggccccgtgatacgaggcgcatcagtctacgttttagaagaaggaaggagggcccagcacggaaaggagcggcagccatcgaagctgaggacagggaggaagaagagggggaggaagagaaagaggcccccacggggagagagaagaggccaagccgctctggaggaagagtaagggcggccgggcggcggctgcagggcttctgcctgtccctggcccagggcacatatcggccgctacggcgcttcttccacgacatcctgcacaccaagtaccgcgcagccaccgacgtctatgccctcatgttcctggctgatgttgtcgacttcatcatcatcatttttggcttctgggcctttgggaagcactcggcggccacagacatcacgtcctccctatcagacgaccaggtacccgaggctttcctggtcatgctgctgatccagttcagtaccatggtggttgaccgcgccctctacctgcgcaagaccgtgctgggcaagctggccttccaggtggcgctggtgctggccatccacctatggatgttcttcatcctgcccgccgtcactgagaggatgttcaaccagaatgtggtggcccagctctggtacttcgtgaagtgcatctacttcgccctgtccgcctaccagatccgctgcggctaccccacccgcatcctcggcaacttcctcaccaagaagtacaatcatctcaacctcttcctcttccaggggttccggctggtgccgttcctggtggagctgcgggcagtgatggactgggtgtggacggacaccacgctgtccctgtccagctggatgtgtgtggaggacatctatgccaacatcttcatcatcaaatgcagccgagagacagagaagaaatacccgcagcccaaagggcagaagaagaagaagatcgtcaagtacggcatgggtggcctcatcatcctcttcctcatcgccatcatctggttcccgctgctcttcatgtcgctggtgcgctccgtggttggggttgtcaaccagcccatcgatgtcaccgtcaccctgaagctgggcggctatgagccgctgttcaccatgagcgcccagcagccgtccatcatccccttcacggcccaggcctatgaggagctgtcccggcagtttgacccccagccgctggccatgcagttcatcagccagtacagccctgaggacgtcgtcacggcgcagattgagggcagctccggggcgctgtggcgcatcagtccccccagccgtgcccagatgaagcgggagctctacaacggcacggccgacatcaccctgcgcttcacctggaacttccagagggacctggcgaagggaggcactgtggagtatgccaacgagaagcacatgctggccctggcccccaacagcactgcacggcggcagctggccagcctgctcgagggcacctcggaccagtctgtggtcatccccaatctcttccccaagtacatccgtgcccccaacgggcccgaagccaaccctgtgaagcagctgcagcccaatgaggaggccgactacctcggcgtgcgtatccagctgcggagggagcagggtgcgggggccaccggcttcctcgaatggtgggtcatcgaactgcaggagtgccggaccgactgcaacctgctgcccatggtcattttcagtgacaaggtcagcccaccgagcctcggcttcctggctggctacggcatcatggggctgtacgtgtccatcgtgctggtcatcggcaagttcgtgcgcggattcttcagcgagatctcgcactccattatgttcgaggagctgccgtgcgtggaccgcatcctcaagctctgccaggacatcttcctggtgcgggagactcgggagctggagctggaggaggagttgtacgccaagctcatcttcctctaccgctcactggagaccatgatcaagtggactcgtgagaaggagtag

SEQ ID NO: 40 - DNA, 1635 bp, Homo sapiens
  source 1..1635: mol_type = genomic DNA
taccagatccgctgcggctaccccacccgcatcctcggcaacttcctcaccaagaagtacaatcatctcaacctcttcctcttccaggggttccggctggtgccgttcctggtggagctgcgggcagtgatggactgggtgtggacggacaccacgctgtccctgtccagctggatgtgtgtggaggacatctatgccaacatcttcatcatcaaatgcagccgagagacagagaaggttcatgaagatttgcccgttttcttctaaaagttctatagttttagctctgaagtttcgctctttgatccactttgagaaatacccgcagcccaaagggcagaagaagaagaagatcgtcaagtacggcatgggtggcctcatcatcctcttcctcatcgccatcatctggttcccactgctcttcatgtcgctggtgcgctccgtggttggggttgtcaaccagcccatcgatgtcaccgtcaccctgaagctgggcggctatgagccgctgttcaccatgagcgcccagcagccgtccatcatccccttcacggcccaggcctatgaggagctgtcccggcagtttgacccccagccgctggccatgcagttcatcagccagtacagccctgaggacatcgtcacggcgcagattgagggcagctccggggcgctgtggcgcatcagtccccccagccgtgcccagatgaagcgggagctctacaacggcacggccgacatcaccctgcgcttcacctggaacttccagagggacctggcgaagggaggcactgtggagtatgccaacgagaagcacatgctggccctggcccccaacagcactgcacggcggcagctggccagcctgctcgagggcacctcggaccagtctgtggtcatccctaatctcttccccaagtacatccgtgcccccaacgggcccgaagccaaccctgtgaagcagctgcagcccaatgaggaggccgactacctcggcgtgcgtatccagctgcggagggagcagggtgcgggggccaccggcttcctcgaatggtgggtcatcgagctgcaggagtgccggaccgactgcaacctgctgcccatggtcattttcagtgacaagcatcatggggctgtacgtgtccatcgtgctggtcatcggcaagttcgtgcgcggattcttcagcgagatctcgcactccattatgttcgaggagctgccgtgcgtggaccgcatcctcaagctctgccaggacatcttcctggtgcgggagactcgggagctggagctggaggaggagttgtacgccaagctcatcttcctctaccgctcactggagaccatgatcaagtggactcgtgagaaggagtaggagctgctgctggcgcccgagagggaaggagccggcctgctgggcagcgtggccacaaggggcggcactcctcaggccgggggagccactgccccgtccaaggccgccagctgtgatgcatcctcccggcctgcctgagccctgatgctgctgtcagagaaggacactgcgtccccacggcctgcgtggcgctgccgtcccccacgtgtactgtagagttttttttttaattaaaaaatgttttatttatacaaatggacaa

SEQ ID NO: 41 - DNA, 660 bp, Homo sapiens
  source 1..660: mol_type = genomic DNA
ggtcatccctaatctcttccccaagtacatccgtgcccccaacgggcccgaagccaaccctgtgaagcagctgcagcccaatgaggaggccgactacctcggctgacaaggtcagcccaccgagcctcggcttcctggctggctacggcatcatggggctgtacgtgtccatcgtgctggtcatcggcaagttcgtgcgcggattcttcagcgagatctcgcactccattatgttcgaggagctgccgtgcgtggaccgcatcctcaagctctgccaggacatcttcctggtgcgggagactcgggagctggagctggaggaggagttgtacgccaagctcatcttcctctaccgctcactggagaccatgatcaagtggactcgtgagaaggagtaggagctgctgctggcgcccgagagggaaggagccggcctgctgggcagcgtggccacaaggggcggcactcctcaggccgggggagccactgccccgtccaaggccgccagctgtgatgcatcctcccggcctgcctgagccctgatgctgctgtcagagaaggacactgcgtccccacggcctgcgtggcgctgccgtcccccacgtgtactgtagagttttttttttaattaaaaaatgttttatttatacaaatggacaa

SEQ ID NO: 42 - DNA, 8072 bp, Homo sapiens
  source 1..8072: mol_type = genomic DNA
cgtccggcccagctcggccccagtgagccgagcgctgcgctccgccgaggggcagggcggtcgcctgagcgagcgcgggcccgggacgtcggcaccggcggggcggccgaaggagaaggaggaagaggagaaggcggcgcgcgggtccccgcgggtcagccatggcgcgccggccccggggcccccgcaccgccccatagcgccgcggcgtccgctcggtctgggccgggccctgggccctccagccatggagccgcacgtgctcggcgcggtcctgtactggctgctgctgccctgcgcgctgctggctgcctgcctgctccgcttcagcggactctcgctggtctacctgctcttcctgctgctgctgccctggttccccggccccacccgatgcggcctccaaggtcacacaggccgcctcctgcgggcattgctgggcctcagcctgctcttcctggtggcccatctcgccctccagatctgcctgcatattgtgccccgcctggaccagctcctgggacccagctgcagccgctgggagaccctctcgcgacacataggggtcacaaggctggacctgaaggacatccccaacgccatccggctggtggcccctgacctgggcatcttggtggtctcctctgtctgcctcggcatctgcgggcgccttgcaaggaacacccggcagagcccacatccacgggagctggatgatgatgagagggatgtggatgccagcccgacggcagggctgcaggaagcagcaacgctggcccctacacggaggtcacggctggccgctcgtttccgagtcacggcccactggctgctggtggcggctgggcgggtcctggccgtaacactgcttgcactggcaggcatcgcccacccctcggccctctccagtgtctacctgctgctcttcctggccctctgcacctggtgggcctgccactttcccatcagcactcggggcttcagcagactctgcgtcgcggtggggtgcttcggcgccggccatctcatctgcctctactgctaccagatgcccttggcacaggctctgctcccgcctgccggcatctgggctagggtgctgggtctcaaggacttcgtgggtcccaccaactgctccagcccccacgcgctggtcctcaacaccggcctggactggcctgtgtatgccagccccggcgtcctcctgctgctgtgctacgccacggcctctctgcgcaagctccgcgcgtaccgcccctccggccagaggaaggaggcggcaaaggggtatgaggctcgggagctggagctagcagagctggaccagtggccccaggaacgggagtctgaccagcacgtggtgcccacagcacccgacaccgaggctgataactgcatcgtgcacgagctgaccggccagagctccgtcctgcggcggcctgtgcggcccaagcgggctgagcccagggaggcgtctccgctccacagcctgggccacctcatcatggaccagagctatgtgtgcgcgctcattgccatgatggtatggagcatcacctaccacagctggctgaccttcgtactgctgctctgggcctgcctcatctggacggtgcgcagccgccaccaactggccatgctgtgctcgccctgcatcctgctgtatgggatgacgctgtgctgcctacgctacgtgtgggccatggacctgcgccctgagctgcccaccaccctgggccccgtcagcctgcgccagctggggctggagcacacccgctacccctgtctggaccttggtgccatgttgctctacaccctgaccttctggctcctgctgcgccagtttgtgaaagagaagctgctgaagtgggcagagtctccagctgcgctgacggaggtcaccgtggcagacacagagcccacgcggacgcagacgctgttgcagagcctgggggagctggtgaagggcgtgtacgccaagtactggatctatgtgtgtgctggcatgttcatcgtggtcagcttcgccggccgcctcgtggtctacaagattgtctacatgttcctcttcctgctctgcctcaccctcttccaggtctactacagcctgtggcggaagctgctcaaggccttctggtggctcgtggtggcctacaccatgctggtcctcatcgccgtctacaccttccagttccaggacttccctgcctactggcgcaacctcactggcttcaccgacgagcagctgggggacctgggcctggagcagttcagcgtgtccgagctcttctccagcatcctggtgcccggcttcttcctcctggcctgcatcctgcagctgcactacttccacaggcccttcatgcagctcaccgacatggagcacgtgtccctgcctggcacgcgcctcccgcgctgggctcacaggcaggatgcagtgagtgggaccccactgctgcgggaggagcagcaggagcatcagcagcagcagcaggaggaggaggaggaggaggaggactccagggacgaggggctgggcgtggccactccccaccaggccacgcaggtgcctgaaggggcagccaagtggggcctggtggctgagcggctgctggagctggcagccggcttctcggacgtcctctcacgcgtgcaggtgttcctgcggcggctgctggagcttcacgttttcaagctggtggccctgtacaccgtctgggtggccctgaaggaggtgtcggtgatgaacctgctgctggtggtgctgtgggccttcgccctgccctacccacgcttccggcccatggcctcctgcctgtccaccgtgtggacctgcgtcatcatcgtgtgtaagatgctgtaccagctcaaggttgtcaacccccaggagtattccagcaactgcaccgagcccttccccaacagcaccaacttgctgcccacggagatcagccagtccctgctgtaccgggggcccgtggaccctgccaactggtttggggtgcggaaagggttccccaacctgggctacatccagaaccacctgcaagtgctgctgctgctggtattcgaggccatcgtgtaccggcgccaggagcactaccgccggcagcaccagctggccccgctgcctgcccaggccgtgtttgccagcggcacccgccagcagctggaccaggatctgctcggctgcctcaagtacttcatcaacttcttcttctacaaattcgggctggagatctgcttcctgatggccgtgaacgtgatcgggcagcgcatgaactttctggtgaccctgcacggttgctggctggtggccatcctcacccgcaggcaccgccaggccattgcccgcctctggcccaactactgcctcttcctggcgctgttcctgctgtaccagtacctgctgtgcctggggatgcccccggccctgtgcattgattatccctggcgctggagccgggccgtccccatgaactccgcactcatcaagtggctgtacctgcctgatttcttccgggcccccaactccaccaacctcatcagcgactttctcctgctgctgtgcgcctcccagcagtggcaggtgttctcagctgagcgcacagaggagtggcagcgcatggctggcgtcaacaccgaccgcctggagccgctgcggggggagcccaaccccgtgcccaactttatccactgcaggtcctaccttgacatgctgaaggtggccgtcttccgatacctgttctggctggtgctggtggtggtgtttgtcacgggggccacccgcatcagcatcttcgggctgggctacctgctggcctgcttctacctgctgctcttcggcacggccctgctgcagagggacacacgggcccgcctcgtgctgtgggactgcctcattctgtacaacgtcaccgtcatcatctccaagaacatgctgtcgctcctggcctgcgtcttcgtggagcagatgcagaccggcttctgctgggtcatccagctcttcagccttgtatgcaccgtcaagggctactatgaccccaaggagatgatggacagagaccaggactgcctgctgcctgtggaggaggctggcatcatctgggacagcgtctgcttcttcttcctgctgctgcagcgccgcgtcttccttagccattactacctgcacgtcagggccgacctccaggccaccgccctgctagcctccaggggcttcgccctctacaacgctgccaacctcaagagcattgactttcaccgcaggatagaggagaagtccctggcccagctgaaaagacagatggagcgtatccgtgccaagcaggagaagcacaggcagggccgggtggaccgcagtcgcccccaggacaccctgggccccaaggaccccggcctggagccagggcccgacagtccagggggctcctccccgccacggaggcagtggtggcggccctggctggaccacgccacagtcatccactccggggactacttcctgtttgagtccgacagtgaggaagaggaggaggctgttcctgaagacccgaggccgtcggcacagagtgccttccagctggcgtaccaggcatgggtgaccaacgcccaggcggtgctgaggcggcggcagcaggagcaggagcaggcaaggcaggaacaggcaggacagctacccacaggaggtggtcccagccaggaggtggagccagcagagggccccgaggaggcagcggcaggccggagccatgtggtgcagagggtgctgagcacggcgcagttcctgtggatgctggggcaggcgctagtggatgagctgacacgctggctgcaggagttcacccggcaccacggcaccatgagcgacgtgctgcgggcagagcgctacctcctcacacaggagctcctgcagggcggcgaagtgcacaggggcgtgctggatcagctgtacacaagccaggccgaggccacgctgccaggccccaccgaggcccccaatgccccaagcaccgtgtccagtgggctgggcgcggaggagccactcagcagcatgacagacgacatgggcagccccctgagcaccggctaccacacgcgcagtggcagtgaggaggcagtcaccgaccccggggagcgtgaggctggtgcctctctgtaccagggactgatgcggacggccagcgagctgctcctggacaggcgcctgcgcatcccagagctggaggaggcagagctgtttgcggaggggcagggccgggcgctgcggctgctgcgggccgtgtaccagtgtgtggccgcccactcggagctgctctgctacttcatcatcatcctcaaccacatggtcacggcctccgccggctcgctggtgctgcccgtgctcgtcttcctgtgggccatgctgtcgatcccgaggcccagcaagcgcttctggatgacggccatcgtcttcaccgagatcgcggtggtcgtcaagtacctgttccagtttgggttcttcccctggaacagccacgtggtgctgcggcgctacgagaacaagccctacttcccgccccgcatcctgggcctggagaagactgacggctacatcaagtacgacctggtgcagctcatggcccttttcttccaccgctcccagctgctgtgctatggcctctgggaccatgaggaggactcaccatccaaggagcatgacaagagcggcgaggaggagcagggagccgaggaggggccaggggtgcctgcggccaccaccgaagaccacattcaggtggaagccagggtcggacccacggacgggaccccagaaccccaagtggagctcaggccccgtgatacgaggcgcatcagtctacgttttagaagaaggaagaaggagggcccagcacggaaaggagcggcagccatcgaagctgaggacagggaggaagaagagggggaggaagagaaagaggcccccacggggagagagaagaggccaagccgctctggaggaagagtaagggcggccgggcggcggctgcagggcttctgcctgtccctggcccagggcacatatcggccgctacggcgcttcttccacgacatcctgcacaccaagtaccgcgcagccaccgacgtctatgccctcatgttcctggctgatgttgtcgacttcatcatcatcatttttggcttctgggcctttgggaagcactcggcggccacagacatcacgtcctccctatcagacgaccaggtacccgaggctttcctggtcatgctgctgatccagttcagtaccatggtggttgaccgcgccctctacctgcgcaagaccgtgctgggcaagctggccttccaggtggcgctggtgctggccatccacctatggatgttcttcatcctgcccgccgtcactgagaggatgttcaaccagaatgtggtggcccagctctggtacttcgtgaagtgcatctacttcgccctgtccgcctaccagatccgctgcggctaccccacccgcatcctcggcaacttcctcaccaagaagtacaatcatctcaacctcttcctcttccaggggttccggctggtgccgttcctggtggagctgcgggcagtgatggactgggtgtggacggacaccacgctgtccctgtccagctggatgtgtgtggaggacatctatgccaacatcttcatcatcaaatgcagccgagagacagagaagaaatacccgcagcccaaagggcagaagaagaagaagatcgtcaagtacggcatgggtggcctcatcatcctcttcctcatcgccatcatctggttcccactgctcttcatgtcgctggtgcgctccgtggttggggttgtcaaccagcccatcgatgtcaccgtcaccctgaagctgggcggctatgagccgctgttcaccatgagcgcccagcagccgtccatcatccccttcacggcccaggcctatgaggagctgtcccggcagtttgacccccagccgctggccatgcagttcatcagccagtacagccctgaggacatcgtcacggcgcagattgagggcagctccggggcgctgtggcgcatcagtccccccagccgtgcccagatgaagcgggagctctacaacggcacggccgacatcaccctgcgcttcacctggaacttccagagggacctggcgaagggaggcactgtggagtatgccaacgagaagcacatgctggccctggcccccaacagcactgcacggcggcagctggccagcctgctcgagggcacctcggaccagtctgtggtcatccctaatctcttccccaagtacatccgtgcccccaacgggcccgaagccaaccctgtgaagcagctgcagcccaatgaggaggccgactacctcggcgtgcgtatccagctgcggagggagcagggtgcgggggccaccggcttcctcgaatggtgggtcatcgagctgcaggagtgccggaccgactgcaacctgctgcccatggtcattttcagtgacaaggtcagcccaccgagcctcggcttcctggctggctacggcatcatggggctgtacgtgtccatcgtgctggtcatcggcaagttcgtgcgcggattcttcagcgagatctcgcactccattatgttcgaggagctgccgtgcgtggaccgcatcctcaagctctgccaggacatcttcctggtgcgggagactcgggagctggagctggaggaggagttgtacgccaagctcatcttcctctaccgctcactggagaccatgatcaagtggactcgtgagaaggagtaggagctgctgctggcgcccgagagggaaggagccggcctgctgggcagcgtggccacaaggggcggcactcctcaggccgggggagccactgccccgtccaaggccgccagctgtgatgcatcctcccggcctgcctgagccctgatgctgctgtcagagaaggacactgcgtccccacggcctgcgtggcgctgccgtcccccacgtgtactgtagagttttttttttaattaaaaaatgttttatttatacaaatgga

SEQ ID NO: 43 - protein, 2521 aa, Homo sapiens
MEPHVLGAVLYWLLLPCALLAACLLRFSGLSLVYLLFLLLLPWFPGPTRCGLQGHTGRLLRALLGLSLLFLVAHLALQICLHIVPRLDQLLGPSCSRWETLSRHIGVTRLDLKDIPNAIRLVAPDLGILVVSSVCLGICGRLARNTRQSPHPRELDDDERDVDASPTAGLQEAATLAPTRRSRLAARFRVTAHWLLVAAGRVLAVTLLALAGIAHPSALSSVYLLLFLALCTWWACHFPISTRGFSRLCVAVGCFGAGHLICLYCYQMPLAQALLPPAGIWARVLGLKDFVGPTNCSSPHALVLNTGLDWPVYASPGVLLLLCYATASLRKLRAYRPSGQRKEAAKGYEARELELAELDQWPQERESDQHVVPTAPDTEADNCIVHELTGQSSVLRRPVRPKRAEPREASPLHSLGHLIMDQSYVCALIAMMVWSITYHSWLTFVLLLWACLIWTVRSRHQLAMLCSPCILLYGMTLCCLRYVWAMDLRPELPTTLGPVSLRQLGLEHTRYPCLDLGAMLLYTLTFWLLLRQFVKEKLLKWAESPAALTEVTVADTEPTRTQTLLQSLGELVKGVYAKYWIYVCAGMFIVVSFAGRLVVYKIVYMFLFLLCLTLFQVYYSLWRKLLKAFWWLVVAYTMLVLIAVYTFQFQDFPAYWRNLTGFTDEQLGDLGLEQFSVSELFSSILVPGFFLLACILQLHYFHRPFMQLTDMEHVSLPGTRLPRWAHRQDAVSGTPLLREEQQEHQQQQQEEEEEEEDSRDEGLGVATPHQATQVPEGAAKWGLVAERLLELAAGFSDVLSRVQVFLRRLLELHVFKLVALYTVWVALKEVSVMNLLLVVLWAFALPYPRFRPMASCLSTVWTCVIIVCKMLYQLKVVNPQEYSSNCTEPFPNSTNLLPTEISQSLLYRGPVDPANWFGVRKGFPNLGYIQNHLQVLLLLVFEAIVYRRQEHYRRQHQLAPLPAQAVFASGTRQQLDQDLLGCLKYFINFFFYKFGLEICFLMAVNVIGQRMNFLVTLHGCWLVAILTRRHRQAIARLWPNYCLFLALFLLYQYLLCLGMPPALCIDYPWRWSRAVPMNSALIKWLYLPDFFRAPNSTNLISDFLLLLCASQQWQVFSAERTEEWQRMAGVNTDRLEPLRGEPNPVPNFIHCRSYLDMLKVAVFRYLFWLVLVVVFVTGATRISIFGLGYLLACFYLLLFGTALLQRDTRARLVLWDCLILYNVTVIISKNMLSLLACVFVEQMQTGFCWVIQLFSLVCTVKGYYDPKEMMDRDQDCLLPVEEAGIIWDSVCFFFLLLQRRVFLSHYYLHVRADLQATALLASRGFALYNAANLKSIDFHRRIEEKSLAQLKRQMERIRAKQEKHRQGRVDRSRPQDTLGPKDPGLEPGPDSPGGSSPPRRQWWRPWLDHATVIHSGDYFLFESDSEEEEEAVPEDPRPSAQSAFQLAYQAWVTNAQAVLRRRQQEQEQARQEQAGQLPTGGGPSQEVEPAEGPEEAAAGRSHVVQRVLSTAQFLWMLGQALVDELTRWLQEFTRHHGTMSDVLRAERYLLTQELLQGGEVHRGVLDQLYTSQAEATLPGPTEAPNAPSTVSSGLGAEEPLSSMTDDMGSPLSTGYHTRSGSEEAVTDPGEREAGASLYQGLMRTASELLLDRRLRIPELEEAELFAEGQGRALRLLRAVYQCVAAHSELLCYFIIILNHMVTASAGSLVLPVLVFLWAMLSIPRPSKRFWMTAIVFTEIAVVVKYLFQFGFFPWNSHVVLRRYENKPYFPPRILGLEKTDGYIKYDLVQLMALFFHRSQLLCYGLWDHEEDSPSKEHDKSGEEEQGAEEGPGVPAATTEDHIQVEARVGPTDGTPEPQVELRPRDTRRISLRFRRRKKEGPARKGAAAIEAEDREEEEGEEEKEAPTGREKRPSRSGGRVRAAGRRLQGFCLSLAQGTYRPLRRFFHDILHTKYRAATDVYALMFLADVVDFIIIIFGFWAFGKHSAATDITSSLSDDQVPEAFLVMLLIQFSTMVVDRALYLRKTVLGKLAFQVALVLAIHLWMFFILPAVTERMFNQNVVAQLWYFVKCIYFALSAYQIRCGYPTRILGNFLTKKYNHLNLFLFQGFRLVPFLVELRAVMDWVWTDTTLSLSSWMCVEDIYANIFIIKCSRETEKKYPQPKGQKKKKIVKYGMGGLIILFLIAIIWFPLLFMSLVRSVVGVVNQPIDVTVTLKLGGYEPLFTMSAQQPSIIPFTAQAYEELSRQFDPQPLAMQFISQYSPEDIVTAQIEGSSGALWRISPPSRAQMKRELYNGTADITLRFTWNFQRDLAKGGTVEYANEKHMLALAPNSTARRQLASLLEGTSDQSVVIPNLFPKYIRAPNGPEANPVKQLQPNEEADYLGVRIQLRREQGAGATGFLEWWVIELQECRTDCNLLPMVIFSDKVSPPSLGFLAGYGIMGLYVSIVLVIGKFVRGFFSEISHSIMFEELPCVDRILKLCQDIFLVRETRELELEEELYAKLIFLYRSPETMIKWTREKE

SEQ ID NO: 44 - protein, 2035 aa, Homo sapiens
MDLRPELPTTLGPVSLRQLGLEHTRYPCLDLGAMLLYTLTFWLLLRQFVKEKLLKWAESPAALTEVTVADTEPTRTQTLLQSLGELVKGVYAKYWIYVCAGMFIVVSFAGRLVVYKIVYMFLFLLCLTLFQVYYSLWRKLLKAFWWLVVAYTMLVLIAVYTFQFQDFPAYWRNLTGFTDEQLGDLGLEQFSVSELFSSILVPGFFLLACILQLHYFHRPFMQLTDMEHVSLPGTRLPRWAHRQDAVSGTPLLREEQQEHQQQQQEEEEEEDSRDEGLGVATPHQATQVPEGAAKWGLVAERLLELAAGFSDVLSRVQVFLRRLLELHVFKLVALYTVWVALKEVSVMNLLLVVLWAFALPYPRFRPMASCLSTVWTCVIIVCKMLYQLKVVNPQEYSSNCTEPFPNSTNLLPTEISQSLLYRGPVDPANWFGVRKGFPNLGYIQNHLQVLLLLVFEAIVYRRQEHYRRQHQLAPLPAQAVFASGTRQQLDQDLLGCLKYFINFFFYKFGLEICFLMAVNVIGQRMNFLVTLHGCWLVAILTRRHRQAIARLWPNYCLFLALFLLYQYLLCLGMPPALCIDYPWRWSRAVPMNSALIKWLYLPDFFRAPNSTNLISDFLLLLCASQQWQVFSAERTEEWQRMAGVNTDRLEPLRGEPNPVPNFIHCRSYLDMLKVAVFRYLFWLVLVVVFVTGATRISIFGLGYLLACFYLLLFGTALLQRDTRARLVLWDCLILYNVTVIISKNMLSLLACVFVEQMQTGFCWVIQLFSLVCTVKGYYDPKEMMDRDQDCLLPVEEAGIIWDSVCFFFLLLQRRVFLSHYYLHVRADLQATALLASRGFALYNAANLKSIDFHRRIEEKSLAQLKRQMERIRAKQEKHRQGRVDRSRPQDTLGPKDPGLEPGPDSPGGSSPPRRQWWRPWLDHATVIHSGDYFLFESDSEEEEEAVPEDPRPSAQSAFQLAYQAWVTNAQAVLRRRQQEQEQARQEQAGQLPTGGGPSQEVEPAEGPEEAAAGRSHVVQRVLSTAQFLWMLGQALVDELTRWLQEFTRHHGTMSDVLRAERYLLTQELLQGGEVHRGVLDQLYTSQAEATLPGPTEAPNAPSTVSSGLGAEEPLSSMTDDMGSPLSTGYHTRSGSEEAVTDPGEREAGASLYQGLMRTASELLLDRRLRIPELEEAELFAEGQGRALRLLRAVYQCVAAHSELLCYFIIILNHMVTASAGSLVLPVLVFLWAMLSIPRPSKRFWMTAIVFTEIAVVVKYLFQFGFFPWNSHVVLRRYENKPYFPPRILGLEKTDGYIKYDLVQLMALFFHRSQLLCYGLWDHEEDSPSKEHDKSGEEEQGAEEGPGVPAATTEDHIQVEARVGPTDGTPEPQVELRPRDTRRISLRFRRRKKEGPARKGAAAIEAEDREEEEGEEEKEAPTGREKRPSRSGGRVRAAGRRLQGFCLSLAQGTYRPLRRFFHDILHTKYRAATDVYALMFLADVVDFIIIIFGFWAFGKHSAATDITSSLSDDQVPEAFLVMLLIQFSTMVVDRALYLRKTVLGKLAFQVALVLAIHLWMFFILPAVTERMFNQNVVAQLWYFVKCIYFALSAYQIRCGYPTRILGNFLTKKYNHLNLFLFQGFRLVPFLVELRAVMDWVWTDTTLSLSSWMCVEDIYANIFIIKCSRETEKKYPQPKGQKKKKIVKYGMGGLIILFLIAIIWFPLLFMSLVRSVVGVVNQPIDVTVTLKLGGYEPLFTMSAQQPSIIPFTAQAYEELSRQFDPQPLAMQFISQYSPEDIVTAQIEGSSGALWRISPPSRAQMKRELYNGTADITLRFTWNFQRDLAKGGTVEYANEKHMLALAPNSTARRQLASLLEGTSDQSVVIPNLFPKYIRAPNGPEANPVKQLQPNEEADYLGVRIQLRREQGAGATGFLEWWVIELQECRTDCNLLPMVIFSDKVSPPSLGFLAGYGIMGLYVSIVLVIGKFVRGFFSEISHSIMFEELPCVDRILKLCQDIFLVRETRELELEEELYAKLIFLYRSPETMIKWTREKE

SEQ ID NO: 45 - protein, 2090 aa, Homo sapiens
MVWSITYHSWLTFVLLLWACLIWTVRSRHQLAMLCSPCILLYGMTLCCLRYVWAMDLRPELPTTLGPVSLRQLGLEHTRYPCLDLGAMLLYTLTFWLLLRQFVKEKLLKWAESPAALTEVTVADTEPTRTQTLLQSLGELVKGVYAKYWIYVCAGMFIVVSFAGRLVVYKIVYMFLFLLCLTLFQVYYSLWRKLLKAFWWLVVAYTMLVLIAVYTFQFQDFPAYWRNLTGFTDEQLGDLGLEQFSVSELFSSILVPGFFLLACILQLHYFHRPFMQLTDMEHVSLPGTRLPRWAHRQDAVSGTPLLREEQQEHQQQQQEEEEEEEDSRDEGLGVATPHQATQVPEGAAKWGLVAERLLELAAGFSDVLSRVQVFLRRLLELHVFKLVALYTVWVALKEVSVMNLLLVVLWAFALPYPRFRPMASCLSTVWTCVIIVCKMLYQLKVVNPQEYSSNCTEPFPNSTNLLPTEISQSLLYRGPVDPANWFGVRKGFPNLGYIQNHLQVLLLLVFEAIVYRRQEHYRRQHQLAPLPAQAVFASGTRQQLDQDLLGCLKYFINFFFYKFGLEICFLMAVNVIGQRMNFLVTLHGCWLVAILTRRHRQAIARLWPNYCLFLALFLLYQYLLCLGMPPALCIDYPWRWSRAVPMNSALIKWLYLPDFFRAPNSTNLISDFLLLLCASQQWQVFSAERTEEWQRMAGVNTDRLEPLRGEPNPVPNFIHCRSYLDMLKVAVFRYLFWLVLVVVFVTGATRISIFGLGYLLACFYLLLFGTALLQRDTRARLVLWDCLILYNVTVIISKNMLSLLACVFVEQMQTGFCWVIQLFSLVCTVKGYYDPKEMMDRDQDCLLPVEEAGIIWDSVCFFFLLLQRRVFLSHYYLHVRADLQATALLASRGFALYNAANLKSIDFHRRIEEKSLAQLKRQMERIRAKQEKHRQGRVDRSRPQDTLGPKDPGLEPGPDSPGGSSPPRRQWWRPWLDHATVIHSGDYFLFESDSEEEEEAVPEDPRPSAQSAFQLAYQAWVTNAQAVLRRRQQEQEQARQEQAGQLPTGGGPSQEVEPAEGPEEAAAGRSHVVQRVLSTAQFLWMLGQALVDELTRWLQEFTRHHGTMSDVLRAERYLLTQELLQGGEVHRGVLDQLYTSQAEATLPGPTEAPNAPSTVSSGLGAEEPLSSMTDDMGSPLSTGYHTRSGSEEAVTDPGEREAGASLYQGLMRTASELLLDRRLRIPELEEAELFAEGQGRALRLLRAVYQCVAAHSELLCYFIIILNHMVTASAGSLVLPVLVFLWAMLSIPRPSKRFWMTAIVFTEIAVVVKYLFQFGFFPWNSHVVLRRYENKPYFPPRILGLEKTDGYIKYDLVQLMALFFHRSQLLCYGLWDHEEDSPSKEHDKSGEEEQGAEEGPGVPAATTEDHIQVEARVGPTDGTPEPQVELRPRDTRRISLRFRRRKKEGPARKGAAAIEAEDREEEEGEEEKEAPTGREKRPSRSGGRVRAAGRRLQGFCLSLAQGTYRPLRRFFHDILHTKYRAATDVYALMFLADVVDFIIIIFGFWAFGKHSAATDITSSLSDDQVPEAFLVMLLIQFSTMVVDRALYLRKTVLGKLAFQVALVLAIHLWMFFILPAVTERMFNQNVVAQLWYFVKCIYFALSAYQIRCGYPTRILGNFLTKKYNHLNLFLFQGFRLVPFLVELRAVMDWVWTDTTLSLSSWMCVEDIYANIFIIKCSRETEKKYPQPKGQKKKKIVKYGMGGLIILFLIAIIWFPLLFMSLVRSVVGVVNQPIDVTVTLKLGGYEPLFTMSAQQPSIIPFTAQAYEELSRQFDPQPLAMQFISQYSPEDIVTAQIEGSSGALWRISPPSRAQMKRELYNGTADITLRFTWNFQRDLAKGGTVEYANEKHMLALAPNSTARRQLASLLEGTSDQSVVIPNLFPKYIRAPNGPEANPVKQLQPNEEADYLGVRIQLRREQGAGATGFLEWWVIELQECRTDCNLLPMVIFSDKVSPPSLGFLAGYGIMGLYVSIVLVIGKFVRGFFSEISHSIMFEELPCVDRILKLCQDIFLVRETRELELEEELYAKLIFLYRSPETMIKWTREKE

SEQ ID NO: 46 - protein, 105 aa, Homo sapiens [sequence omitted]

SEQ ID NO: 47 - protein, 2521 aa, Homo sapiens
MEPHVLGAVLYWLLLPCALLAACLLRFSGLSLVYLLFLLLLPWFPGPTRCGLQGHTGRLLRALLGLSLLFLVAHLALQICLHIVPRLDQLLGPSCSRWETLSRHIGVTRLDLKDIPNAIRLVAPDLGILVVSSVCLGICGRLARNTRQSPHPRELDDDERDVDASPTAGLQEAATLAPTRRSRLAARFRVTAHWLLVAAGRVLAVTLLALAGIAHPSALSSVYLLLFLALCTWWACHFPISTRGFSRLCVAVGCFGAGHLICLYCYQMPLAQALLPPAGIWARVLGLKDFVGPTNCSSPHALVLNTGLDWPVYASPGVLLLLCYATASLRKLRAYRPSGQRKEAAKGYEARELELAELDQWPQERESDQHVVPTAPDTEADNCIVHELTGQSSVLRRPVRPKRAEPREASPLHSLGHLIMDQSYVCALIAMMVWSITYHSWLTFVLLLWACLIWTVRSRHQLAMLCSPCILLYGMTLCCLRYVWAMDLRPELPTTLGPVSLRQLGLEHTRYPCLDLGAMLLYTLTFWLLLRQFVKEKLLKWAESPAALTEVTVADTEPTRTQTLLQSLGELVKGVYAKYWIYVCAGMFIVVSFAGRLVVYKIVYMFLFLLCLTLFQVYYSLWRKLLKAFWWLVVAYTMLVLIAVYTFQFQDFPAYWRNLTGFTDEQLGDLGLEQFSVSELFSSILVPGFFLLACILQLHYFHRPFMQLTDMEHVSLPGTRLPRWAHRQDAVSGTPLLREEQQEHQQQQQEEEEEEEDSRDEGLGVATPHQATQVPEGAAKWGLVAERLLELAAGFSDVLSRVQVFLRRLLELHVFKLVALYTVWVALKEVSVMNLLLVVLWAFALPYPRFRPMASCLSTVWTCVIIVCKMLYQLKVVNPQEYSSNCTEPFPNSTNLLPTEISQSLLYRGPVDPANWFGVRKGFPNLGYIQNHLQVLLLLVFEAIVYRRQEHYRRQHQLAPLPAQAVFASGTRQQLDQDLLGCLKYFINFFFYKFGLEICFLMAVNVIGQRMNFLVTLHGCWLVAILTRRHRQAIARLWPNYCLFLALFLLYQYLLCLGMPPALCIDYPWRWSRAVPMNSALIKWLYLPDFFRAPNSTNLISDFLLLLCASQQWQVFSAERTEEWQRMAGVNTDRLEPLRGEPNPVPNFIHCRSYLDMLKVAVFRYLFWLVLVVVFVTGATRISIFGLGYLLACFYLLLFGTALLQRDTRARLVLWDCLILYNVTVIISKNMLSLLACVFVEQMQTGFCWVIQLFSLVCTVKGYYDPKEMMDRDQDCLLPVEEAGIIWDSVCFFFLLLQRRVFLSHYYLHVRADLQATALLASRGFALYNAANLKSIDFHRRIEEKSLAQLKRQMERIRAKQEKHRQGRVDRSRPQDTLGPKDPGLEPGPDSPGGSSPPRRQWWRPWLDHATVIHSGDYFLFESDSEEEEEAVPEDPRPSAQSAFQLAYQAWVTNAQAVLRRRQQEQEQARQEQAGQLPTGGGPSQEVEPAEGPEEAAAGRSHVVQRVLSTAQFLWMLGQALVDELTRWLQEFTRHHGTMSDVLRAERYLLTQELLQGGEVHRGVLDQLYTSQAEATLPGPTEAPNAPSTVSSGLGAEEPLSSMTDDMGSPLSTGYHTRSGSEEAVTDPGEREAGASLYQGLMRTASELLLDRRLRIPELEEAELFAEGQGRALRLLRAVYQCVAAHSELLCYFIIILNHMVTASAGSLVLPVLVFLWAMLSIPRPSKRFWMTAIVFTEIAVVVKYLFQFGFFPWNSHVVLRRYENKPYFPPRILGLEKTDGYIKYDLVQLMALFFHRSQLLCYGLWDHEEDSPSKEHDKSGEEEQGAEEGPGVPAATTEDHIQVEARVGPTDGTPEPQVELRPRDTRRISLRFRRRKKEGPARKGAAAIEAEDREEEEGEEEKEAPTGREKRPSRSGGRVRAAGRRLQGFCLSLAQGTYRPLRRFFHDILHTKYRAATDVYALMFLADVVDFIIIIFGFWAFGKHSAATDITSSLSDDQVPEAFLVMLLIQFSTMVVDRALYLRKTVLGKLAFQVALVLAIHLWMFFILPAVTERMFNQNVVAQLWYFVKCIYFALSAYQIRCGYPTRILGNFLTKKYNHLNLFLFQGFRLVPFLVELRAVMDWVWTDTTLSLSSWMCVEDIYANIFIIKCSRETEKKYPQPKGQKKKKIVKYGMGGLIILFLIAIIWFPLLFMSLVRSVVGVVNQPIDVTVTLKLGGYEPLFTMSAQQPSIIPFTAQAYEELSRQFDPQPLAMQFISQYSPEDIVTAQIEGSSGALWRISPPSRAQMKRELYNGTADITLRFTWNFQRDLAKGGTVEYANEKHMLALAPNSTARRQLASLLEGTSDQSVVIPNLFPKYIRAPNGPEANPVKQLQPNEEADYLGVRIQLRREQGAGATGFLEWWVIELQECRTDCNLLPMVIFSDKVSPPSLGFLAGYGIMGLYVSIVLVIGKFVRGFFSEISHSIMFEELPCVDRILKLCQDIFLVRETRELELEEELYAKLIFLYRSLETMIKWTREKE

SEQ ID NO: 48 - protein, 2035 aa, Homo sapiens
MDLRPELPTTLGPVSLRQLGLEHTRYPCLDLGAMLLYTLTFWLLLRQFVKEKLLKWAESPAALTEVTVADTEPTRTQTLLQSLGELVKGVYAKYWIYVCAGMFIVVSFAGRLVVYKIVYMFLFLLCLTLFQVYYSLWRKLLKAFWWLVVAYTMLVLIAVYTFQFQDFPAYWRNLTGFTDEQLGDLGLEQFSVSELFSSILVPGFFLLACILQLHYFHRPFMQLTDMEHVSLPGTRLPRWAHRQDAVSGTPLLREEQQEHQQQQQEEEEEEDSRDEGLGVATPHQATQVPEGAAKWGLVAERLLELAAGFSDVLSRVQVFLRRLLELHVFKLVALYTVWVALKEVSVMNLLLVVLWAFALPYPRFRPMASCLSTVWTCVIIVCKMLYQLKVVNPQEYSSNCTEPFPNSTNLLPTEISQSLLYRGPVDPANWFGVRKGFPNLGYIQNHLQVLLLLVFEAIVYRRQEHYRRQHQLAPLPAQAVFASGTRQQLDQDLLGCLKYFINFFFYKFGLEICFLMAVNVIGQRMNFLVTLHGCWLVAILTRRHRQAIARLWPNYCLFLALFLLYQYLLCLGMPPALCIDYPWRWSRAVPMNSALIKWLYLPDFFRAPNSTNLISDFLLLLCASQQWQVFSAERTEEWQRMAGVNTDRLEPLRGEPNPVPNFIHCRSYLDMLKVAVFRYLFWLVLVVVFVTGATRISIFGLGYLLACFYLLLFGTALLQRDTRARLVLWDCLILYNVTVIISKNMLSLLACVFVEQMQTGFCWVIQLFSLVCTVKGYYDPKEMMDRDQDCLLPVEEAGIIWDSVCFFFLLLQRRVFLSHYYLHVRADLQATALLASRGFALYNAANLKSIDFHRRIEEKSLAQLKRQMERIRAKQEKHRQGRVDRSRPQDTLGPKDPGLEPGPDSPGGSSPPRRQWWRPWLDHATVIHSGDYFLFESDSEEEEEAVPEDPRPSAQSAFQLAYQAWVTNAQAVLRRRQQEQEQARQEQAGQLPTGGGPSQEVEPAEGPEEAAAGRSHVVQRVLSTAQFLWMLGQALVDELTRWLQEFTRHHGTMSDVLRAERYLLTQELLQGGEVHRGVLDQLYTSQAEATLPGPTEAPNAPSTVSSGLGAEEPLSSMTDDMGSPLSTGYHTRSGSEEAVTDPGEREAGASLYQGLMRTASELLLDRRLRIPELEEAELFAEGQGRALRLLRAVYQCVAAHSELLCYFIIILNHMVTASAGSLVLPVLVFLWAMLSIPRPSKRFWMTAIVFTEIAVVVKYLFQFGFFPWNSHVVLRRYENKPYFPPRILGLEKTDGYIKYDLVQLMALFFHRSQLLCYGLWDHEEDSPSKEHDKSGEEEQGAEEGPGVPAATTEDHIQVEARVGPTDGTPEPQVELRPRDTRRISLRFRRRKKEGPARKGAAAIEAEDREEEEGEEEKEAPTGREKRPSRSGGRVRAAGRRLQGFCLSLAQGTYRPLRRFFHDILHTKYRAATDVYALMFLADVVDFIIIIFGFWAFGKHSAATDITSSLSDDQVPEAFLVMLLIQFSTMVVDRALYLRKTVLGKLAFQVALVLAIHLWMFFILPAVTERMFNQNVVAQLWYFVKCIYFALSAYQIRCGYPTRILGNFLTKKYNHLNLFLFQGFRLVPFLVELRAVMDWVWTDTTLSLSSWMCVEDIYANIFIIKCSRETEKKYPQPKGQKKKKIVKYGMGGLIILFLIAIIWFPLLFMSLVRSVVGVVNQPIDVTVTLKLGGYEPLFTMSAQQPSIIPFTAQAYEELSRQFDPQPLAMQFISQYSPEDIVTAQIEGSSGALWRISPPSRAQMKRELYNGTADITLRFTWNFQRDLAKGGTVEYANEKHMLALAPNSTARRQLASLLEGTSDQSVVIPNLFPKYIRAPNGPEANPVKQLQPNEEADYLGVRIQLRREQGAGATGFLEWWVIELQECRTDCNLLPMVIFSDKVSPPSLGFLAGYGIMGLYVSIVLVIGKFVRGFFSEISHSIMFEELPCVDRILKLCQDIFLVRETRELELEEELYAKLIFLYRSLETMIKWTREKE

SEQ ID NO: 49 - protein, 2090 aa, Homo sapiens
MVWSITYHSWLTFVLLLWACLIWTVRSRHQLAMLCSPCILLYGMTLCCLRYVWAMDLRPELPTTLGPVSLRQLGLEHTRYPCLDLGAMLLYTLTFWLLLRQFVKEKLLKWAESPAALTEVTVADTEPTRTQTLLQSLGELVKGVYAKYWIYVCAGMFIVVSFAGRLVVYKIVYMFLFLLCLTLFQVYYSLWRKLLKAFWWLVVAYTMLVLIAVYTFQFQDFPAYWRNLTGFTDEQLGDLGLEQFSVSELFSSILVPGFFLLACILQLHYFHRPFMQLTDMEHVSLPGTRLPRWAHRQDAVSGTPLLREEQQEHQQQQQEEEEEEEDSRDEGLGVATPHQATQVPEGAAKWGLVAERLLELAAGFSDVLSRVQVFLRRLLELHVFKLVALYTVWVALKEVSVMNLLLVVLWAFALPYPRFRPMASCLSTVWTCVIIVCKMLYQLKVVNPQEYSSNCTEPFPNSTNLLPTEISQSLLYRGPVDPANWFGVRKGFPNLGYIQNHLQVLLLLVFEAIVYRRQEHYRRQHQLAPLPAQAVFASGTRQQLDQDLLGCLKYFINFFFYKFGLEICFLMAVNVIGQRMNFLVTLHGCWLVAILTRRHRQAIARLWPNYCLFLALFLLYQYLLCLGMPPALCIDYPWRWSRAVPMNSALIKWLYLPDFFRAPNSTNLISDFLLLLCASQQWQVFSAERTEEWQRMAGVNTDRLEPLRGEPNPVPNFIHCRSYLDMLKVAVFRYLFWLVLVVVFVTGATRISIFGLGYLLACFYLLLFGTALLQRDTRARLVLWDCLILYNVTVIISKNMLSLLACVFVEQMQTGFCWVIQLFSLVCTVKGYYDPKEMMDRDQDCLLPVEEAGIIWDSVCFFFLLLQRRVFLSHYYLHVRADLQATALLASRGFALYNAANLKSIDFHRRIEEKSLAQLKRQMERIRAKQEKHRQGRVDRSRPQDTLGPKDPGLEPGPDSPGGSSPPRRQWWRPWLDHATVIHSGDYFLFESDSEEEEEAVPEDPRPSAQSAFQLAYQAWVTNAQAVLRRRQQEQEQARQEQAGQLPTGGGPSQEVEPAEGPEEAAAGRSHVVQRVLSTAQFLWMLGQALVDELTRWLQEFTRHHGTMSDVLRAERYLLTQELLQGGEVHRGVLDQLYTSQAEATLPGPTEAPNAPSTVSSGLGAEEPLSSMTDDMGSPLSTGYHTRSGSEEAVTDPGEREAGASLYQGLMRTASELLLDRRLRIPELEEAELFAEGQGRALRLLRAVYQCVAAHSELLCYFIIILNHMVTASAGSLVLPVLVFLWAMLSIPRPSKRFWMTAIVFTEIAVVVKYLFQFGFFPWNSHVVLRRYENKPYFPPRILGLEKTDGYIKYDLVQLMALFFHRSQLLCYGLWDHEEDSPSKEHDKSGEEEQGAEEGPGVPAATTEDHIQVEARVGPTDGTPEPQVELRPRDTRRISLRFRRRKKEGPARKGAAAIEAEDREEEEGEEEKEAPTGREKRPSRSGGRVRAAGRRLQGFCLSLAQGTYRPLRRFFHDILHTKYRAATDVYALMFLADVVDFIIIIFGFWAFGKHSAATDITSSLSDDQVPEAFLVMLLIQFSTMVVDRALYLRKTVLGKLAFQVALVLAIHLWMFFILPAVTERMFNQNVVAQLWYFVKCIYFALSAYQIRCGYPTRILGNFLTKKYNHLNLFLFQGFRLVPFLVELRAVMDWVWTDTTLSLSSWMCVEDIYANIFIIKCSRETEKKYPQPKGQKKKKIVKYGMGGLIILFLIAIIWFPLLFMSLVRSVVGVVNQPIDVTVTLKLGGYEPLFTMSAQQPSIIPFTAQAYEELSRQFDPQPLAMQFISQYSPEDIVTAQIEGSSGALWRISPPSRAQMKRELYNGTADITLRFTWNFQRDLAKGGTVEYANEKHMLALAPNSTARRQLASLLEGTSDQSVVIPNLFPKYIRAPNGPEANPVKQLQPNEEADYLGVRIQLRREQGAGATGFLEWWVIELQECRTDCNLLPMVIFSDKVSPPSLGFLAGYGIMGLYVSIVLVIGKFVRGFFSEISHSIMFEELPCVDRILKLCQDIFLVRETRELELEEELYAKLIFLYRSLETMIKWTREKE

SEQ ID NO: 50 - protein, 105 aa, Homo sapiens
MRRPTTSADKVSPPSLGFLAGYGIMGLYVSIVLVIGKFVRGFFSEISHSIMFEELPCVDRILKLCQDIFLVRETRELELEEELYAKLIFLYRSLETMIKWTREKE